(19) United States
(12) Patent Application Publication
(10) Pub. No.: US 2024/0287124 A1
(43) Pub. Date: Aug. 29, 2024

(54) PRODRUGS OF 4'-SUBSTITUTED NUCLEOSIDE REVERSE TRANSCRIPTASE INHIBITORS

(71) Applicant: Merck Sharp & Dohme LLC, Rahway, NJ (US)

(72) Inventors: Mark W. Embrey, Harleysville, PA (US); Jay A. Grobler, Gwynedd, PA (US); Mark E. Layton, Harleysville, PA (US); Izzat T. Raheem, Doylestown, PA (US)

(73) Assignee: Merck Sharp & Dohme LLC, Rahway, NJ (US)

(21) Appl. No.: 18/423,652

(22) Filed: Jan. 26, 2024

Related U.S. Application Data
(60) Provisional application No. 63/607,595, filed on Dec. 8, 2023.
(60) Provisional application No. 63/482,133, filed on Jan. 30, 2023.

(51) Int. Cl. C07H 19/14; A61K 31/4439; A61K 31/7064; A61P 31/18

(52) U.S. Cl. CPC C07H 19/14; A61K 31/4439; A61K 31/7064; A61P 31/18

(57) ABSTRACT

The present invention is directed to prodrugs of compound A:

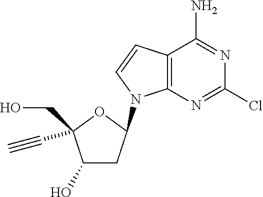 [SMILES: C#C[C@]1(CO)O[C@@H](n2ccc3c(N)nc(Cl)nc32)C[C@@H]1O]

which are nucleoside reverse transcriptase translocation inhibitors (NRTTI) and are useful in the inhibition of HIV reverse transcriptase. The present invention also relates to the use of these compounds for prophylaxis of infection by HIV, treatment of infection by HIV, and prophylaxis, treatment, and delay in the onset or progression of AIDS and/or AIDS-related complexes.

### CROSS-REFERENCE TO RELATED APPLICATIONS

This application claims the benefit of priority to U.S. Provisional Patent Application Nos. 63/482,133, filed Jan. 30, 2023, and 63/607,595, filed Dec. 8, 2023, hereby incorporated by reference in their entireties.

### BACKGROUND OF THF INVENTION

The retrovirus designated human immunodeficiency virus (HIV), particularly the strains known as HIV type-1 (HIV-1) and type-2 (HIV-2), have been etiologically linked to the immunosuppressive disease known as acquired immunodeficiency syndrome (AIDS). HIV seropositive individuals are initially asymptomatic but typically develop AIDS related complex (ARC) followed by AIDS. Affected individuals exhibit severe immunosuppression which makes them highly susceptible to debilitating and ultimately fatal opportunistic infections. Since HIV is a retrovirus, the HIV replication cycle requires transcription of the viral RNA genome into DNA via an enzyme known as reverse transcriptase (RT).

Reverse transcriptase has three known enzymatic functions: The enzyme acts as an RNA-dependent DNA polymerase, as a ribonuclease, and as a DNA-dependent DNA polymerase. In its role as an RNA-dependent DNA polymerase, RT transcribes a single-stranded DNA copy of the viral RNA. As a ribonuclease, RT destroys the original viral RNA and frees the DNA just produced from the original RNA. And as a DNA-dependent DNA polymerase, RT makes a second, complementary DNA strand using the first DNA strand as a template. The two strands form double-stranded DNA, which is integrated into the host cell's genome by the viral enzyme integrase.

It is known that compounds that inhibit enzymatic functions of HIV RT will inhibit HIV replication in infected cells. These compounds are useful in the prophylaxis or treatment of HIV infection in humans. Among the compounds approved for use in treating and/or preventing HIV infection and AIDS are nucleoside (or nucleotide) analogs, which include nucleoside reverse transcriptase inhibitors (NRTIs) and non-nucleoside reverse transcriptase inhibitors (NNRTIs). Known NRTIs include 3′-azido-3′-deoxythymidine (AZT), 2′,3′-dideoxyinosine (ddI), 2′,3′-dideoxycytidine (ddC), d4T, 3TC, abacavir, emtricitabine, and tenofovir disoproxil fumarate. A subtype of NRTI is the nucleoside reverse transcriptase translocation inhibitor (NRTTI), such as 4′-ethynyl-2-fluoro-2′-deoxyadenosine (islatravir). Known non-nucleoside RT inhibitors (nNRTI) include nevirapine, delavirdine, doravirine, and efavirenz.

While each of the foregoing RT inhibitors is effective in treating HIV infection and preventing development of AIDS, there remains a need to develop additional HIV antiviral drugs including additional RT inhibitors. A particular problem is the development of active RT inhibitors that are adapted for extended dosing, or sustained release, regimens. There remains a significant need for improved treatment and prevention of HIV infections using long-acting RT inhibitors.

### SUMMARY OF THF INVENTION

The present invention is directed to prodrugs of 4′-substituted nucleoside derivatives and their use in the inhibition of HIV reverse transcriptase. The present invention is further directed to the use of these compounds in the prophylaxis of infection by HIV, the treatment of infection by HIV, and the prophylaxis, treatment, and delay in the onset or progression of AIDS and/or ARC, in a subject in need thereof. The present invention provides compounds that are solubility-limiting, cleavable prodrugs of NRTIs that may be adapted for extended dosing regimens for a subject in need thereof.

In some aspects, the disclosure provides 4′-substituted fused heterocyclic compounds. In some aspects, the disclosure provides compounds that are cleavable prodrugs of Compound A:

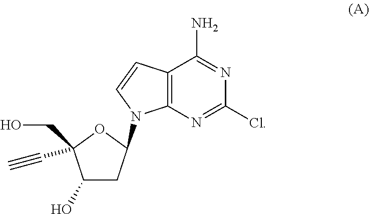 [SMILES: C#C[C@]1(CO)O[C@@H](n2ccc3c(N)nc(Cl)nc32)C[C@@H]1O]

Compound A is a 4′-substituted heterocyclic nucleoside analogue. The synthesis and ability of Compound A to inhibit HIV reverse transcriptase is described in PCT International Application WO 2015/148746, published on Oct. 1, 2015, to Merck Sharp & Dohme Corp., which is hereby incorporated by reference in its entirety.

In some aspects, provided herein are compounds having the Formula VI:

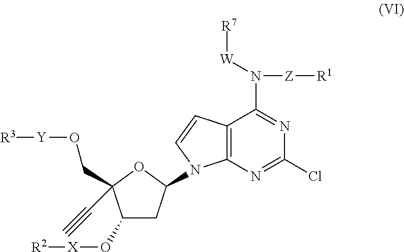

or a pharmaceutically acceptable salt thereof, wherein:
X is selected from —C(═O)—, —C(═O)—O—, —CR⁵R⁶—O—C(═O)—, —CR⁵R⁶—O—C(═O)—O—, and a bond;
Y is selected from —C(═O)—, —C(═O)—O—, —CR⁵R⁶—O—C(═O)—, —CR⁵R⁶—O—C(═O)—O—, —P(═O)(O—C₆₋₁₂ aryl)-NH—CR⁴C(═O)—O—, and a bond;
Z is selected from —C(═O)—, —C(═O)—O—, and a bond;
W is selected from —C(═O)—, —C(═O)—O—, and a bond;
R¹ is selected from the group consisting of hydrogen, C₁₋₂₁ alkyl, (CR⁵R⁶)_{z}—C₃₋₁₂cycloalkyl, (CR⁵R⁶)_{z}—C₅₋₁₂heterocyclyl, (CR⁵R⁶)_{z}—C₆₋₁₂ aryl, (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy;
R² is selected from the group consisting of hydrogen, C₁₋₂₁ alkyl, (CR⁵R⁶)_{z}—C₃₋₁₂cycloalkyl, (CR⁵R⁶)_{z}—C₅₋₁₂heterocyclyl, (CR⁵R⁶)_{z}—C₆₋₁₂ aryl, (CR⁵R⁶)_{z}—C₅₋₁₂ heteroaryl, and (CR⁵R⁶)_{z}—C₁₋₆ alkyl ester, wherein said alkyl, cycloalkyl, heterocyclyl, aryl or heteroaryl group can be optionally substituted with one to three groups independently selected from halo, oxo, C₁₋₆ alkyl, C₃₋₁₂ cycloalkyl and hydroxy;
R³ is selected from the group consisting of hydrogen, C₁₋₂₁ alkyl, (CR⁵R⁶)_{z}—C₃₋₁₂cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, or heteroaryl group can be optionally substituted with one to three groups independently selected from halo, oxo, C₁₋₆ alkyl, C₃₋₁₂ cycloalkyl, or hydroxy, and
wherein said aryl can be optionally substituted with one to three groups independently selected from halo, oxo, C₁₋₆ alkyl, C₃₋₁₂ cycloalkyl, hydroxy, and C₁₋₆ alkyl ester;
R⁴ is selected from the group consisting of hydrogen, C₁₋₃ alkyl, and (CH₂)_{z}-cycloalkyl;
R⁵ and R⁶ are each independently selected from hydrogen, a halogen, C₁₋₃ alkyl, and C₃₋₆ cycloalkyl; and
R⁷ is selected from hydrogen, C₁₋₂₁ alkyl and (CR⁵R⁶)_{z}—C₅₋₁₂cycloalkyl,
wherein at least one of R¹, R² and R³ is not hydrogen; and z is 0, 1, 2, 3, 4, 5, or 6.

In an embodiment of this invention are compounds of Formula VI having structural Formula I, or a pharmaceutically acceptable salt thereof:

Accordingly, in some aspects, provided herein are compounds having the Formula I:

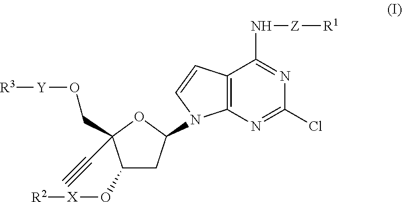

or a pharmaceutically acceptable salt thereof, wherein:
X is selected from —C(═O)—, —C(═O)—O—, —CR⁵R⁶—O—C(═O)—, —CR⁵R⁶—O—C(═O)—O— or a bond;
Y is selected from —C(═O)—, —C(═O)—O—, —CR⁵R⁶—O—C(═O)—, —CR⁵R⁶—O—C(═O)—O—, —P(═O)(O—C₆₋₁₂ aryl)-NH—CR⁴C(═O)—O— or a bond;
Z is selected from —C(═O)—, or a bond;
R¹ is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy;
R² is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy;
R³ is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy;
R⁴ is selected from the group consisting of hydrogen, C₁₋₃ alkyl or (CH₂)_{z}-cycloalkyl;
R⁵ and R⁶ are each independently selected from hydrogen, a halogen, C₁₋₃ alkyl, and C₃₋₆ cycloalkyl; and wherein at least one of R¹, R² and R³ is not hydrogen; and
z is 0, 1, 2, 3, 4, 5, or 6.

In some embodiments of Formula I and Formula VI, where X is —CR⁵R⁶—O—C(═O)— and/or Y is —CR⁵R⁶—O—C(═O)—, neither R⁵ nor R⁶ is a halogen.

In an embodiment of this invention are compounds of Formula I and Formula VI having structural Formula II or a pharmaceutically acceptable salt thereof:

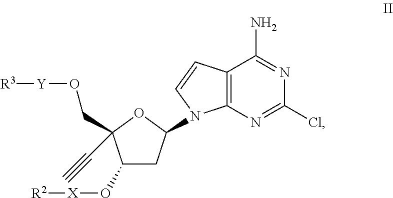

wherein all variables therein (R², R³, X, Y, etc.) are as defined in Formula I and Formula VI.

In another embodiment of this invention are compounds of Formula I and Formula VI having structural Formula III or a pharmaceutically acceptable salt thereof:

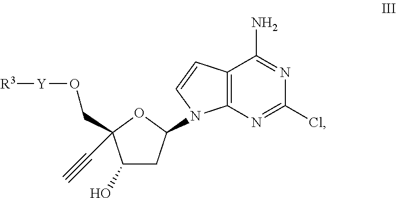

wherein all variables therein (R³, Y, etc.) are as defined in Formula I and Formula VI.

In another embodiment of this invention are compounds of Formula I and Formula VI having structural Formula IV or a pharmaceutically acceptable salt thereof:

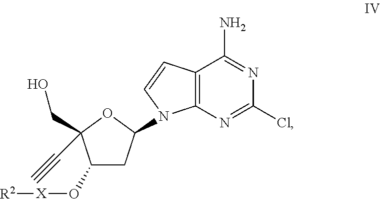

wherein all variables therein (R², X, etc.) are as defined in Formula I and Formula VI.

In another embodiment of this invention are compounds of Formula I and Formula VI having structural Formula V or a pharmaceutically acceptable salt thereof:

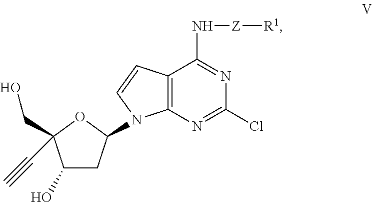

wherein all variables therein (R¹, Z, etc.) are as defined in Formula I and Formula VI.

Accordingly, in various aspects, provided herein are cleavable prodrugs of Compound A that are substituted at the 3′ (or 3-) position, the 5′ (or 5-) position, or both positions, of the tetrahydrofuran (“THF”) ring (which also may be referred to herein as the sugar moiety).

Further provided herein are cleavable prodrugs of Compound A that are substituted at the 5′ position of the THF ring, the 4-amino position of the pyrrolopyrimidine core, or both positions. Further provided herein are cleavable prodrugs of Compound A that are substituted at the 3′ position of the THF ring, the 5′ position of the THF ring, the 4-amino position of the pyrrolopyrimidine core, or all three positions.

All structural Formulas, embodiments and classes thereof described herein include the pharmaceutically acceptable salts of the compounds defined therein. Reference to the compounds of Formula I and Formula VI herein encompasses the compounds of each of Formulas I, II, III, IV, V, and VI, and all embodiments and classes thereof. Reference to the compounds of this invention as those of a specific formula or embodiment, e.g., Formula I, II, III, IV, V or VI, or embodiments thereof, or any other generic structural formula or specific compound described or claimed herein, is intended to encompass the specific compound or compounds falling within the scope of the Formula or embodiment, including salts thereof, particularly pharmaceutically acceptable salts, solvates (including hydrates) of such compounds and solvated salt forms thereof, where such forms are possible, unless specified otherwise.

The present invention includes each of the Examples described herein, and pharmaceutically acceptable salts thereof. The invention also encompasses pharmaceutical compositions comprising an effective amount of a compound of the invention or a pharmaceutically acceptable salt thereof, and a pharmaceutically acceptable carrier.

### BRIEF DESCRIPTIONS OF THF DRAWINGS

FIG. 1 shows a plasma concentration—time profile of Compound A after a single intramuscular (IM) injection of Compound A to rats.

FIG. 2 shows a plasma concentration—time profile of Compound A after a single IM injection of the compound of the compound of Example 10 to rats.

FIG. 3 shows a plasma concentration—time profile of Compound A after a single IM injection of the compound of Example 18 to rats.

FIG. 4 shows a plasma concentration—time profile of Compound A after a single IM injection of the compound of Example 20 to rats.

FIG. 5 shows a plasma concentration—time profile of Compound A after a single IM injection of the compound of Example 21 to rats.

FIG. 6 shows a plasma concentration—time profile of Compound A after a single IM injection of the compound of Example 24 to rats.

FIG. 7 shows a plasma concentration—time profile of Compound A after a single IM injection of the compound of Example 27 to rats.

FIG. 8 shows a plasma concentration—time profile of Compound A after a single IM injection of the compound of Example 34 to rats.

FIG. 9 shows a plasma concentration—time profile of Compound A after a single IM injection of the compound of Example 36 to rats.

FIG. 10 shows a plasma concentration—time profile of Compound A after a single IM injection of the compound of Example 42 to rats.

FIG. 11 shows a plasma concentration—time profile of Compound A after a single IM injection of the compound of Example 48 to rats.

FIG. 12 shows a plasma concentration—time profile of Compound A after a single IM injection of the compound of Example 60 to rats.

FIG. 13 shows a plasma concentration—time profile of Compound A after a single IM injection of the compound of Example 61 to rats.

FIG. 14 shows a plasma concentration—time profile of Compound A after a single IM injection of the compound of Example 64 to rats.

FIG. 15 shows a plasma concentration—time profile of Compound A after a single IM injection of the compound of Example 68 to rats.

### DETAILED DESCRIPTION OF THF INVENTION

The present invention is directed to prodrugs of Compound A that are useful as NRTTIs, and methods of these prodrugs for treating or preventing HIV infection in a subject. These compounds are useful as antiretrovirals (ARVs), and in particular in the prophylaxis, treatment, and delay in the onset or progression of AIDS and/or AIDS-related complexes (ARC), in a subject in need thereof. Further provided herein are pharmaceutical compositions, formulations and drugs comprising these compounds. In some embodiments, the compositions are adapted for use in long-acting injectable dosage forms.

Also provided herein are methods of treatment and prophylaxis comprising administration of any of the disclosed compounds. Further provided herein are uses of these compounds as medicaments for treatment or prophylaxis of HIV, AIDS or ARC. In certain embodiments, a compound of Formula VI or pharmaceutically acceptable salt thereof, is used in the preparation of a medicament for: (a) therapy (e.g., of the human body), (b) medicine, (c) inhibition of HIV reverse transcriptase, (d) treatment or prophylaxis of infection by HIV, or (e) treatment, prophylaxis of, or delay in the onset or progression of AIDS or ARC. In certain embodiments, a compound of Formula I or pharmaceutically acceptable salt thereof, is used in the preparation of a medicament for: (a) therapy (e.g., of the human body), (b) medicine, (c) inhibition of HIV reverse transcriptase, (d) treatment or prophylaxis of infection by HIV, or (e) treatment, prophylaxis of, or delay in the onset or progression of AIDS or ARC. In these uses, the compounds of the present invention can optionally be employed in combination with one or more anti-HIV agents, such as other ARVs.

In some embodiments, the disclosed pharmaceutical compositions and methods of treatment are adapted for long-acting injection. In some embodiments, the pharmaceutical compositions and methods of treatment are adapted for subcutaneous, intramuscular, intravenous, or other injection. In certain embodiments, these compositions may be adapted for intramuscular injection. In some embodiments, the methods of treatment are adapted for an implantable device in the body of the subject.

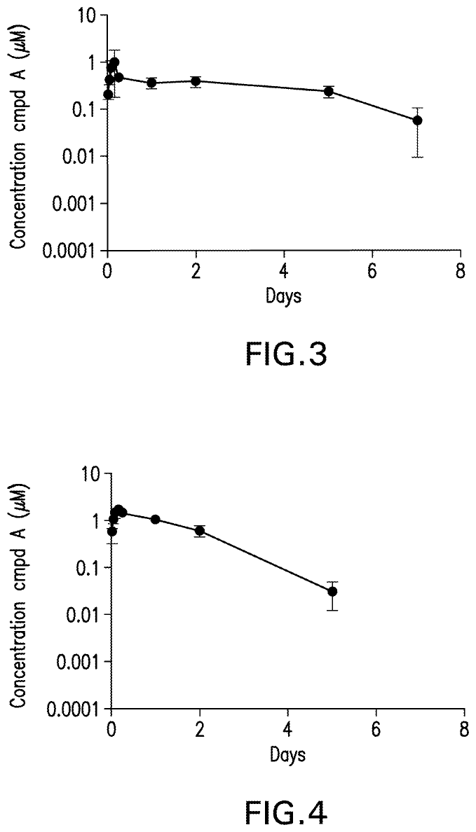

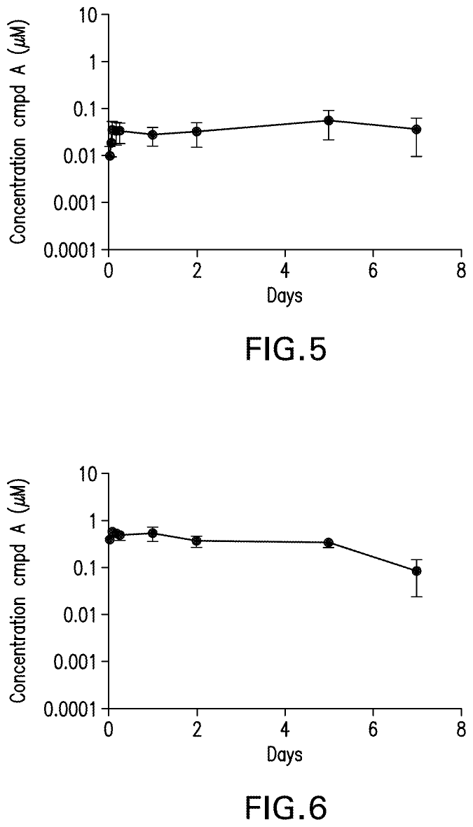

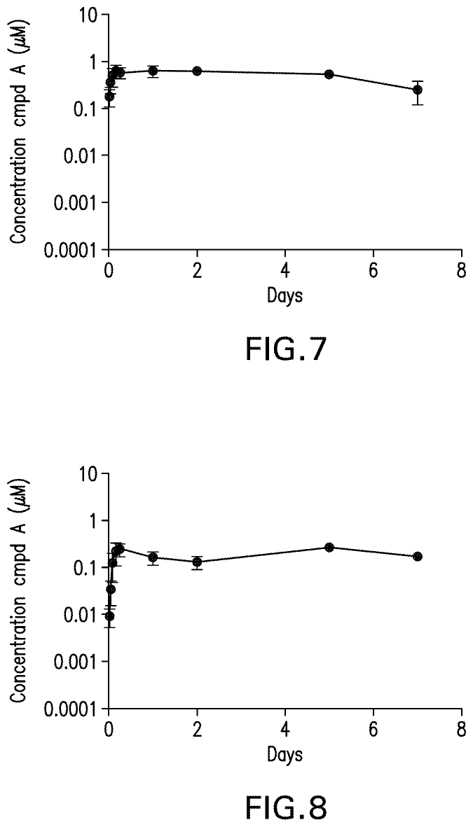

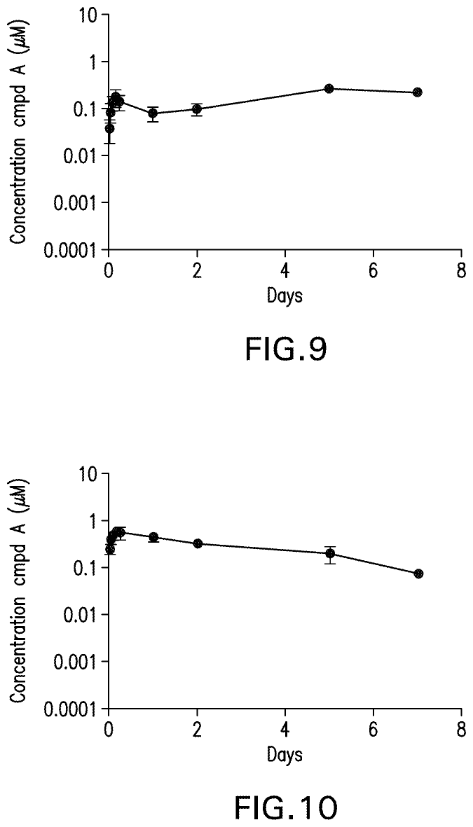

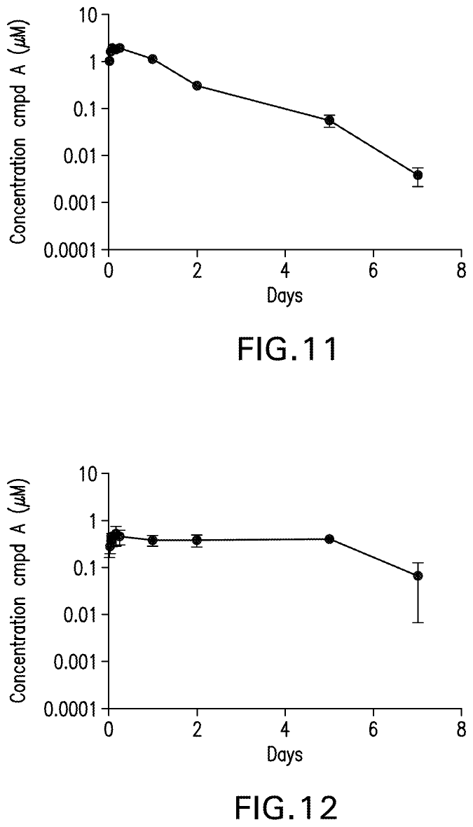

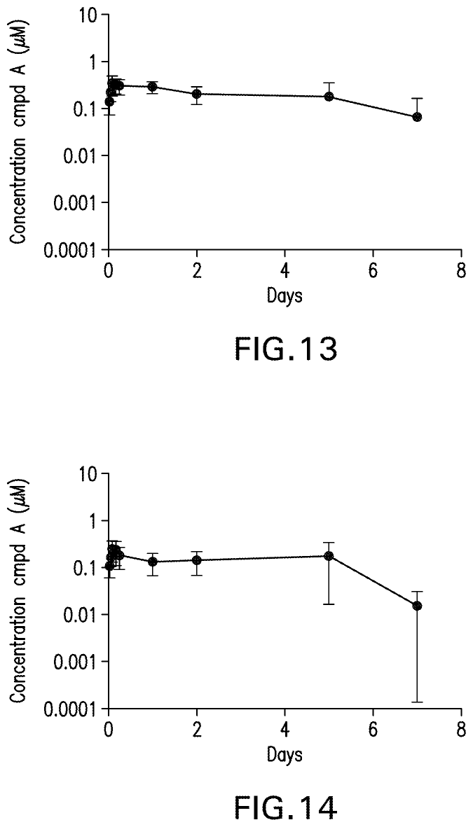

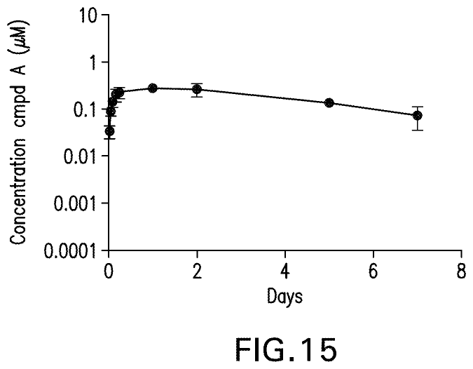

In some embodiments, any of the presently described compounds and compositions comprising these compounds can be delivered intramuscularly and provides efficient release of parent Compound A into systemic circulation in vivo. In some embodiments, intramuscular injection of any of the described compositions provides for release of Compound A into systemic circulation in mammals over 5 days, 6 days, or 7 days (see FIGS. 2-15). In some embodiments, this release is substantially elongated, slow, and/or linear over time.

In some embodiments, any of the presently described compounds, compositions and methods provide a reduction in the likelihood or severity of symptoms of HIV, AIDS, or ARC in one or more subjects. In some embodiments, any of these compounds, compositions, and methods may provide a partial or complete reduction/inhibition of one or more symptoms. Any of the disclosed compounds, compositions, and methods may provide a partial or complete inhibition of HIV infection. Any of the disclosed compounds, compositions, and methods may provide a partial or complete inhibition of HIV viral replication.

In some embodiments, any of the disclosed prodrug compounds exhibit high anti-viral potencies (e.g., inhibitory of HIV viral replication). Anti-viral potencies may be assessed by the Viral Kinetics in Green cells (VIKING) assay and may be expressed as IC₅₀ or EC₅₀. In some embodiments, any of the disclosed prodrug compounds exhibit anti-viral potencies of less than 500 nM, less than 200 nM, less than 100 nM, less than 75 nM, less than 50 nM, less than 25 nM, less than 20 nM, less than 10 nM, or less than 5 nM. In particular embodiments, the compounds exhibit potencies of less than 10 nM.

In some embodiments, the disclosed prodrug compounds have reduced solubility relative to Compound A. This reduced solubility provides for longer durations of time before the drug is metabolized and released into the body of the subject. As such, the present invention provides longer-acting and/or sustained release compounds and formulations thereof.

In some embodiments, the phase of any of the disclosed compounds is crystalline. In some embodiments, the phase of any of the disclosed compounds is amorphous.

In various aspects, provided herein are compounds of structural Formula VI:

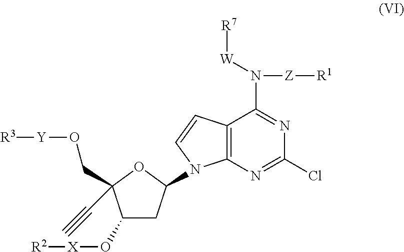

or a pharmaceutically acceptable salt thereof, wherein R¹, R², R³ and R⁷ are defined as provided above.

In some embodiments, W is a bond, and R⁷ is a hydrogen. In some embodiments, Z is a bond, and R¹ is a hydrogen. In some embodiments, X is a bond, and R² is a hydrogen. In some embodiments, Y is a bond, and R² is a hydrogen. In some embodiments, any or all of the following is true: W is a bond, and R⁷ is a hydrogen, Z is a bond, and R¹ is a hydrogen. In some embodiments, any or all of the following is true: X is a bond, R² is a hydrogen, Y is a bond, and R³ is a hydrogen.

In some embodiments, R² is (CR⁵R⁶)_{z}—C₅₋₁₂cycloalkyl, wherein R⁵ and R⁶ are defined as provided above and z is 0, 1, 2, 3, 4, 5, or. In some embodiments, R³ is (CR⁵R⁶)_{z}—C₅₋₁₂cycloalkyl, wherein z is 0, 1, 2, 3, 4, 5, or 6.

In some embodiments, R¹ is C₃₋₁₂ cycloalkyl. In some embodiments, R² is C₃₋₁₂ cycloalkyl. In some embodiments, R³ is C₃₋₁₂ cycloalkyl.

In some embodiments, R⁷ is C₁₋₁₂ alkyl.

In various aspects, provided herein are compounds of structural Formula I:

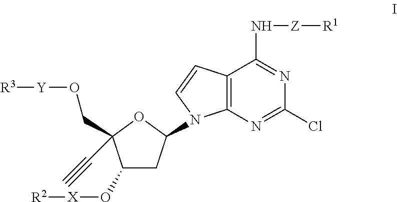

or a pharmaceutically acceptable salt thereof, wherein R¹, R², and R³ are defined as above.

In some embodiments, Z is a bond, and R¹ is a hydrogen. In some embodiments, X is a bond, and R² is a hydrogen. In some embodiments, Y is a bond, and R³ is a hydrogen. In some embodiments, any or all of the following is true: X is a bond, R² is a hydrogen, Y is a bond, and R³ is a hydrogen.

In some embodiments of Formula I and Formula VI, X is —C(═O)— and R² is an alkyl, cycloalkyl, heterocyclyl, aryl, heteroaryl, or alkyl-substituted aryl or heteroaryl group, such that the 3′ position of the tetrahydrofuran (THF) ring of Formula I is a substituted ester. In some embodiments, Y is —C(═O)— and R³ is an alkyl, cycloalkyl, heterocyclyl, aryl, heteroaryl, or alkyl-substituted aryl or heteroaryl group, such that the 5′ position of the THF ring is a substituted ester. In some embodiments, both the 3′ position and 5′ position of the THF ring are substituted (bis-substituted) esters. In any of these embodiments, Z may be a bond, and R¹ may be a hydrogen.

In some embodiments of Formula I and Formula VI, X is —C(═O)—O— and R² is an alkyl, cycloalkyl, heterocyclyl, aryl or heteroaryl group, such that the 3′ position of the THF ring of Formula I is a substituted carbonate. In some embodiments, Y is —C(═O)—O— and R³ is an alkyl, cycloalkyl, heterocyclyl, aryl or heteroaryl group, such that the 5′ position of the THF ring is a substituted carbonate. In some embodiments, both the 3′ position and 5′ position of the THF ring are substituted (bis-substituted) carbonates. In any of these embodiments, Z may be a bond, and R¹ may be a hydrogen.

In some embodiments of Formula I and Formula VI, the 5′ position of the THF ring is a substituted acetal. In any such embodiment, Z may be a bond, and R¹ may be a hydrogen. In any such embodiment, X may be a bond, and R² may be a hydrogen.

In some embodiments of Formula I and Formula VI, X is selected from —C(═O), —CR⁵R⁶—O—C(═O), and a bond; Y is selected from —C(═O), —CR⁵R⁶—O—C(═O), and a bond; Z is a bond, and R¹ is hydrogen; R² is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, (CR⁵R⁶)_{z}—C₃₋₁₂ cycloalkyl and hydroxy; and R³ is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy.

In some embodiments of Formula I and Formula VI, X is selected from —C(═O)—O—, —CR⁵R⁶—O—C(═O)—O—, and a bond; Y is selected from —C(═O)—O—, —CR⁵R⁶—O—C(═O)—O—, and a bond; Z is a bond, and R¹ is hydrogen; R² is selected from the group consisting of hydrogen, C₁₋₂₁ alkyl, (CR⁵R⁶)_{z}—C₃₋₁₂cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy; and R³ is selected from the group consisting of hydrogen, C₁₋₂₁ alkyl, (CR⁵R⁶)_{z}—C₃₋₁₂cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy.

In some embodiments of Formula I and Formula VI, Y is —P(═O)(O—C₆₋₁₂ aryl)-NH—CR⁴C(═O)—O— (phosphoramidite group). In any such embodiment, Z may be a bond, and R¹ may be a hydrogen. In any such embodiment, X may be a bond, and R² may be a hydrogen.

In some embodiments of Formula I and Formula VI, Z is —C(═O) and R¹ is an alkyl, cycloalkyl, heterocyclyl, aryl or heteroaryl group, such that the N position of Formula I (and Formula VI) is an amide. In any such embodiment, X may be a bond, and R² may be a hydrogen. In any such embodiment, Y may be a bond, and R³ may be a hydrogen.

In some embodiments of Formula I and Formula VI, R¹ is selected from (CR⁵R⁶)_{z}—C₅₋₁₂ heterocyclyl and (CR⁵R⁶)_{z}—C₆₋₁₂ aryl. In some embodiments, R² is selected from (CR⁵R⁶)_{z}—C₅₋₁₂ heterocyclyl and (CR⁵R⁶)_{z}—C₆₋₁₂ aryl. In some embodiments, R³ is selected from (CR⁵R⁶)_{z}—C₅₋₁₂ heterocyclyl and (CR⁵R⁶)_{z}—C₆₋₁₂ aryl.

In certain embodiments, X is selected from —C(═O)—, —C(═O)—O—, and a bond; Y is selected from —C(═O)—, —C(═O)—O—, and a bond; R¹ is selected from hydrogen and C₁₋₁₂ alkyl; R² is selected from C₁₋₁₀ alkyl, (CR⁵R⁶)_{z}—C₃₋₁₂ cycloalkyl, and (CR⁵R⁶)_{z}—C₆ aryl, wherein said aryl can be optionally substituted with a halo (e.g., chlorine); and R³ is selected from C₁₋₁₀ alkyl, (CR⁵R⁶)_{z}—C₃₋₁₂ cycloalkyl, and (CR⁵R⁶)_{z}—C₆ aryl, wherein said aryl can be optionally substituted with a halo, and wherein R⁵ and R⁶ are hydrogen.

In some embodiments, R² is (CR⁵R⁶)_{z}—C₅₋₁₂cycloalkyl. In some embodiments, R³ is (CR⁵R⁶)_{z}—C₅₋₁₂cycloalkyl. In some embodiments, R² is (CR⁵R⁶)_{z}—C₅₋₁₂ heteroaryl. In some embodiments, R³ is (CR⁵R⁶)_{z}—C₅₋₁₂ heteroaryl.

In some embodiments, R² is (CR⁵R⁶)_{z}—C₆₋₁₂cycloalkyl. In some embodiments, R³ is (CR⁵R⁶)_{z}—C₆₋₁₂cycloalkyl. In some embodiments, R² is (CR⁵R⁶)_{z}—C₆₋₁₂ heteroaryl. In some embodiments, R³ is (CR⁵R⁶)_{z}—C₆₋₁₂ heteroaryl. In some embodiments, R¹ is (CR⁵R⁶)_{z}—C₆₋₁₂ heteroaryl.

In some embodiments, R³ is pyridinyl. In some embodiments, R³ is pyridinyl that is substituted with one to three groups independently selected from halo, C₁₋₆ alkyl, C₃₋₁₂ cycloalkyl and hydroxy.

In some embodiments, the disclosed prodrug compounds contain a substituent (or variable group) at the 3′ position, the 5′ position, or both positions, at the tetrahydrofuran ring. These substituents may be cleavable, e.g., cleavable in vivo by one or more of the body's metabolic processes.

In various embodiments, the disclosed prodrug compounds exhibit a high degree of bioconversion (transformation) in vivo in a subject to parent Compound A. In some embodiments, the disclosed compounds exhibit a bioconversion in cellulo or in vitro to Compound A of a degree of above 50%, above 60%, above 65%, above 70%, or above 75% (of total amount of prodrug prior to any transformation). In certain embodiments, a degree of bioconversion of above 75% is exhibited. In some embodiments, the subject is a mammal. In certain embodiments, the subject is human.

In some embodiments, the disclosed prodrug compounds do not themselves have antiviral (e.g., anti-HIV) activity; however, the pharmacologically active form of these compounds that is produced in the subject in vivo following cleavage has antiviral activity.

In some embodiments, the disclosed compounds are selected from any of the 59 compounds having a structure listed in Table A. The example numbers in the “Ex.” Column refer to the particular Example in which the synthesis and properties of the compound is discussed, below. It is to be understood that any of the compounds listed in Table A may be grouped or combined with any other compound in that table.

In some embodiments, the compound contains the structure of Example 20 in Table A. In some embodiments, the compound contains the structure of Example 3. In some embodiments, the compound contains the structure of Example 10. In some embodiments, the compound contains the structure of Example 13. In some embodiments, the compound contains the structure of Example 15. In some embodiments, the compound contains the structure of Example 18. In some embodiments, the compound contains the structure of Example 20. In some embodiments, the compound contains the structure of Example 21. In some embodiments, the compound contains the structure of Example 24. In some embodiments, the compound contains the structure of Example 27. In some embodiments, the compound contains the structure of Example 34. In some embodiments, the compound contains the structure of Example 36. In some embodiments, the compound contains the structure of Example 42. In some embodiments, the compound contains the structure of Example 44. In some embodiments, the compound contains the structure of Example 48. In some embodiments, the compound contains the structure of Example 57. In some embodiments, the compound contains the structure of Example 55. In some embodiments, the compound contains the structure of Example 60, Example 61, Example 64 or Example 68.

TABLE A

| Ex. | Structure |
|---|---|
| 1 | 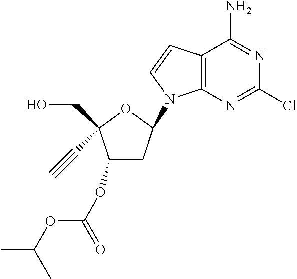 [SMILES: C#C[C@]1(CO)O[C@@H](n2ccc3c(N)nc(Cl)nc32)C[C@@H]1OC(=O)OC(C)C] |
| 2 | 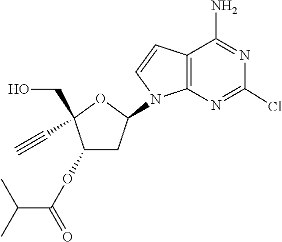 [SMILES: C#C[C@]1(CO)O[C@@H](n2ccc3c(N)nc(Cl)nc32)C[C@@H]1OC(=O)C(C)C] |
| 3 | 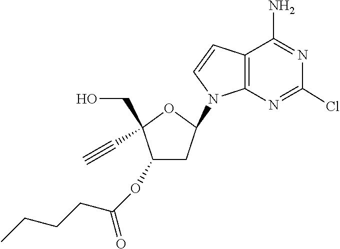 [SMILES: C#C[C@]1(CO)O[C@@H](n2ccc3c(N)nc(Cl)nc32)C[C@@H]1OC(=O)CCCC] |
| 4 | 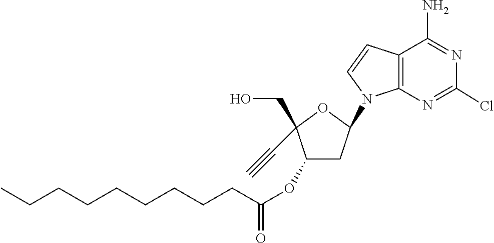 [SMILES: C#C[C@]1(CO)O[C@@H](n2ccc3c(N)nc(Cl)nc32)C[C@@H]1OC(=O)CCCCCCCCC] |
| 5 | 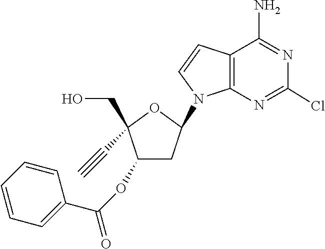 [SMILES: C#C[C@]1(CO)O[C@@H](n2ccc3c(N)nc(Cl)nc32)C[C@@H]1OC(=O)c1ccccc1] |
| 6 | 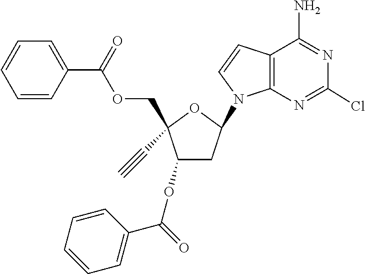 [SMILES: C#C[C@]1(COC(=O)c2ccccc2)O[C@@H](n2ccc3c(N)nc(Cl)nc32)C[C@@H]1OC(=O)c1ccccc1] |
| 7 | 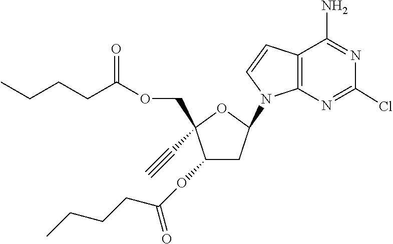 [SMILES: C#C[C@]1(COC(=O)CCCC)O[C@@H](n2ccc3c(N)nc(Cl)nc32)C[C@@H]1OC(=O)CCCC] |
| 8 | 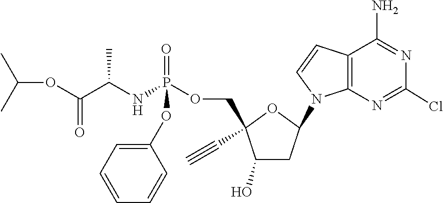 [SMILES: C#C[C@]1(CO[P@@](=O)(N[C@@H](C)C(=O)OC(C)C)Oc2ccccc2)O[C@@H](n2ccc3c(N)nc(Cl)nc32)C[C@@H]1O] |
| 9 | 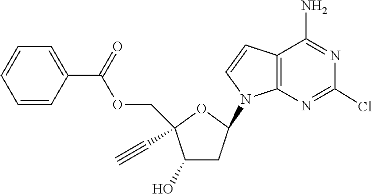 [SMILES: C#C[C@]1(COC(=O)c2ccccc2)O[C@@H](n2ccc3c(N)nc(Cl)nc32)C[C@@H]1O] |
| 10 | 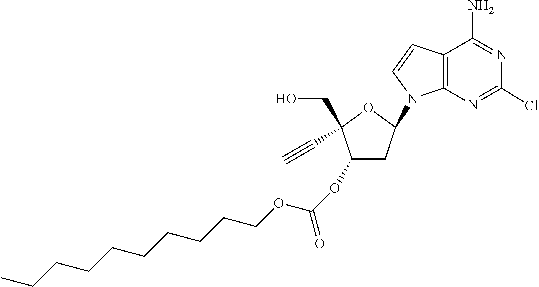 [SMILES: C#C[C@]1(CO)O[C@@H](n2ccc3c(N)nc(Cl)nc32)C[C@@H]1OC(=O)OCCCCCCCCCC] |
| 11 | 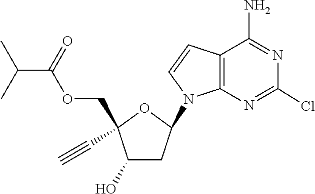 [SMILES: C#C[C@]1(COC(=O)C(C)C)O[C@@H](n2ccc3c(N)nc(Cl)nc32)C[C@@H]1O] |
| 12 | 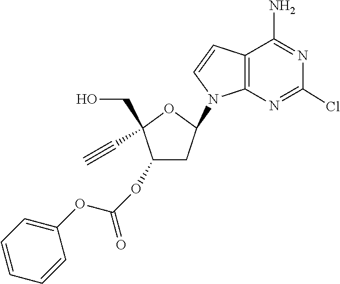 [SMILES: C#C[C@]1(CO)O[C@@H](n2ccc3c(N)nc(Cl)nc32)C[C@@H]1OC(=O)Oc1ccccc1] |
| 13 | 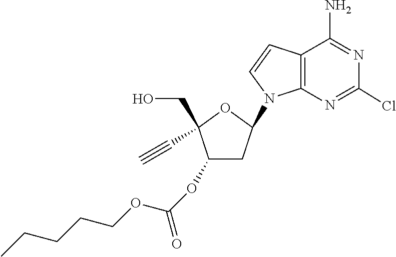 [SMILES: C#C[C@]1(CO)O[C@@H](n2ccc3c(N)nc(Cl)nc32)C[C@@H]1OC(=O)OCCCCC] |
| 14 | 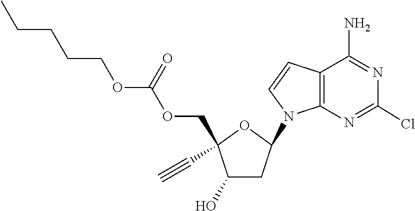 [SMILES: C#C[C@]1(COC(=O)OCCCCC)O[C@@H](n2ccc3c(N)nc(Cl)nc32)C[C@@H]1O] |
| 15 | 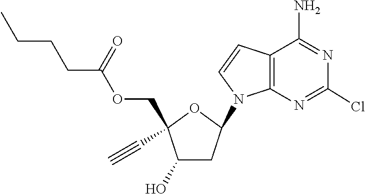 [SMILES: C#C[C@]1(COC(=O)CCCC)O[C@@H](n2ccc3c(N)nc(Cl)nc32)C[C@@H]1O] |
| 16 | 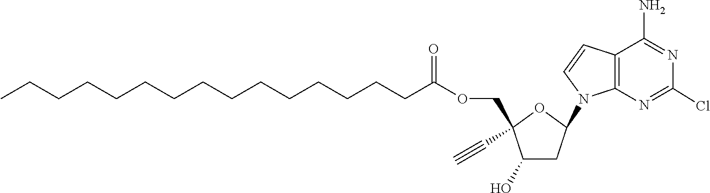 [SMILES: C#C[C@]1(COC(=O)CCCCCCCCCCCCCCC)O[C@@H](n2ccc3c(N)nc(Cl)nc32)C[C@@H]1O] |
| 17 | 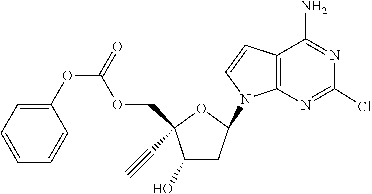 [SMILES: C#C[C@]1(COC(=O)Oc2ccccc2)O[C@@H](n2ccc3c(N)nc(Cl)nc32)C[C@@H]1O] |
| 18 | 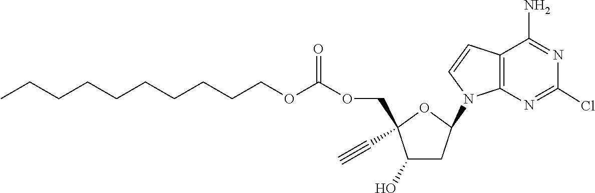 [SMILES: C#C[C@]1(COC(=O)OCCCCCCCCCC)O[C@@H](n2ccc3c(N)nc(Cl)nc32)C[C@@H]1O] |
| 19 | 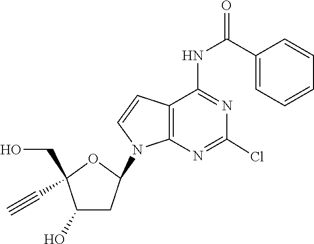 [SMILES: C#C[C@]1(CO)O[C@@H](n2ccc3c(NC(=O)c4ccccc4)nc(Cl)nc32)C[C@@H]1O] |
| 20 | 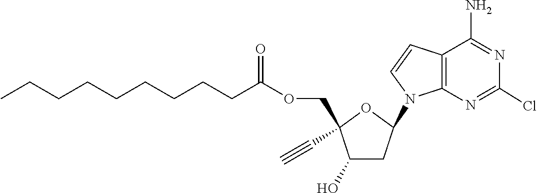 [SMILES: C#C[C@]1(COC(=O)CCCCCCCCC)O[C@@H](n2ccc3c(N)nc(Cl)nc32)C[C@@H]1O] |
| 21 | 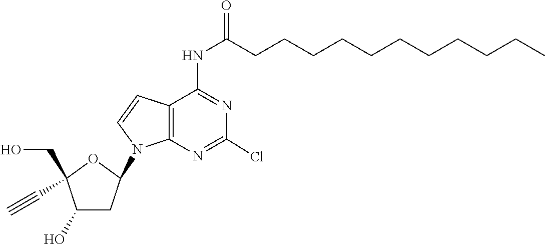 [SMILES: C#C[C@]1(CO)O[C@@H](n2ccc3c(NC(=O)CCCCCCCCCCC)nc(Cl)nc32)C[C@@H]1O] |
| 22 | 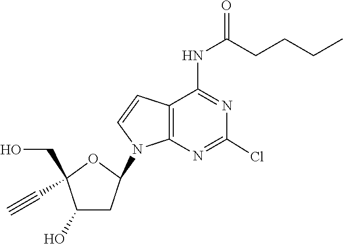 [SMILES: C#C[C@]1(CO)O[C@@H](n2ccc3c(NC(=O)CCCC)nc(Cl)nc32)C[C@@H]1O] |
| 23 | 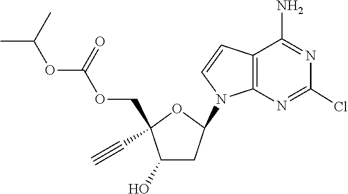 [SMILES: C#C[C@]1(COC(=O)OC(C)C)O[C@@H](n2ccc3c(N)nc(Cl)nc32)C[C@@H]1O] |
| 24 | 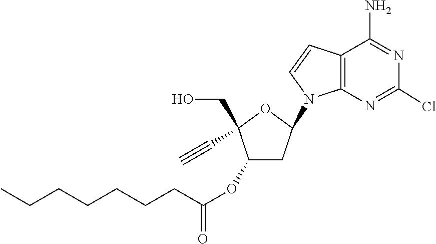 [SMILES: C#C[C@]1(CO)O[C@@H](n2ccc3c(N)nc(Cl)nc32)C[C@@H]1OC(=O)CCCCCCC] |
| 25 | 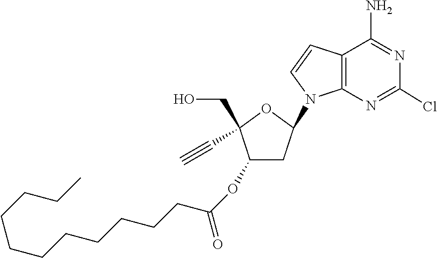 [SMILES: C#C[C@]1(CO)O[C@@H](n2ccc3c(N)nc(Cl)nc32)C[C@@H]1OC(=O)CCCCCCCCCCC] |
| 26 | 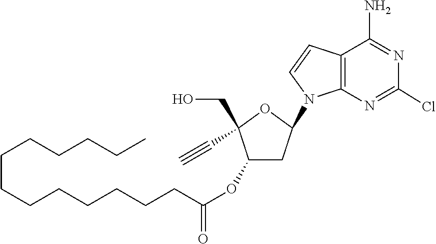 [SMILES: C#C[C@]1(CO)O[C@@H](n2ccc3c(N)nc(Cl)nc32)C[C@@H]1OC(=O)CCCCCCCCCCCCC] |
| 27 | 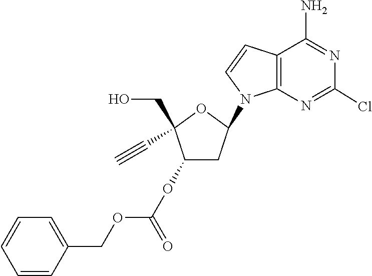 [SMILES: C#C[C@]1(CO)O[C@@H](n2ccc3c(N)nc(Cl)nc32)C[C@@H]1OC(=O)OCc1ccccc1] |
| 28 | 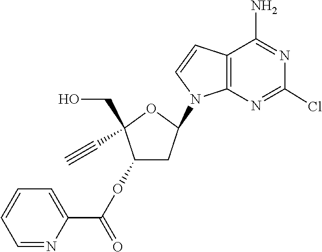 [SMILES: C#C[C@]1(CO)O[C@@H](n2ccc3c(N)nc(Cl)nc32)C[C@@H]1OC(=O)c1ccccn1] |
| 29 | 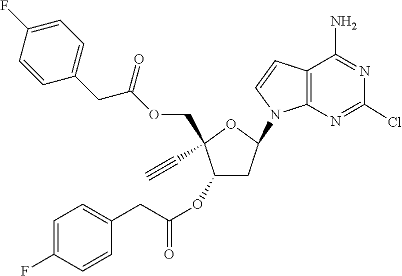 [SMILES: C#C[C@]1(COC(=O)Cc2ccc(F)cc2)O[C@@H](n2ccc3c(N)nc(Cl)nc32)C[C@@H]1OC(=O)Cc1ccc(F)cc1] |
| 30 | 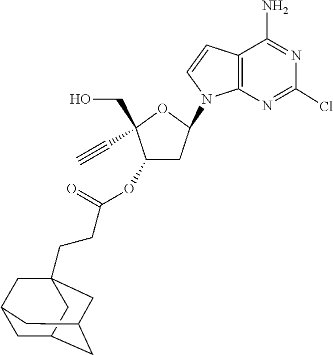 [SMILES: C#C[C@]1(CO)O[C@@H](n2ccc3c(N)nc(Cl)nc32)C[C@@H]1OC(=O)CCC12CC3CC(CC(C3)C1)C2] |
| 31 | 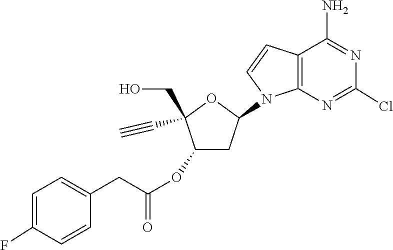 [SMILES: C#C[C@]1(CO)O[C@@H](n2ccc3c(N)nc(Cl)nc32)C[C@@H]1OC(=O)Cc1ccc(F)cc1] |
| 32 | 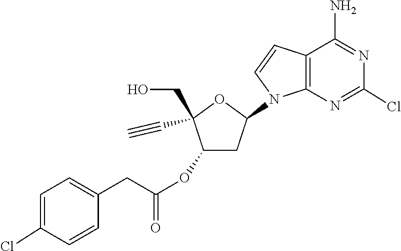 [SMILES: C#C[C@]1(CO)O[C@@H](n2ccc3c(N)nc(Cl)nc32)C[C@@H]1OC(=O)Cc1ccc(Cl)cc1] |
| 33 | 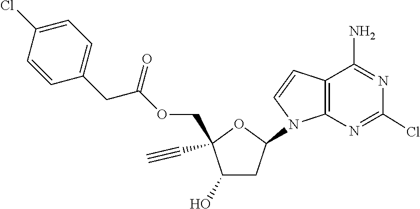 [SMILES: C#C[C@]1(COC(=O)Cc2ccc(Cl)cc2)O[C@@H](n2ccc3c(N)nc(Cl)nc32)C[C@@H]1O] |
| 34 | 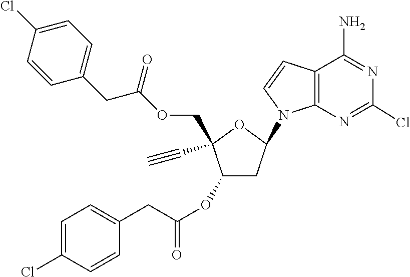 [SMILES: C#C[C@]1(COC(=O)Cc2ccc(Cl)cc2)O[C@@H](n2ccc3c(N)nc(Cl)nc32)C[C@@H]1OC(=O)Cc1ccc(Cl)cc1] |
| 35 | 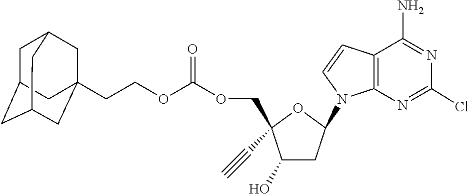 [SMILES: C#C[C@]1(COC(=O)OCCC23CC4CC(CC(C4)C2)C3)O[C@@H](n2ccc3c(N)nc(Cl)nc32)C[C@@H]1O] |
| 36 | 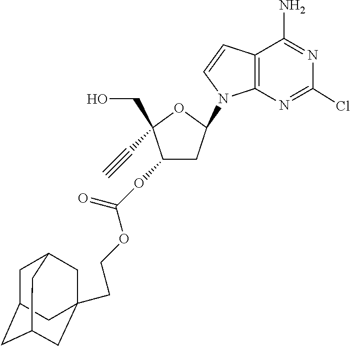 [SMILES: C#C[C@]1(CO)O[C@@H](n2ccc3c(N)nc(Cl)nc32)C[C@@H]1OC(=O)OCCC12CC3CC(CC(C3)C1)C2] |
| 37 | 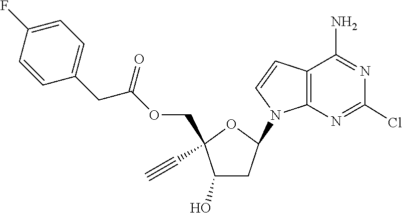 [SMILES: C#C[C@]1(COC(=O)Cc2ccc(F)cc2)O[C@@H](n2ccc3c(N)nc(Cl)nc32)C[C@@H]1O] |
| 38 | 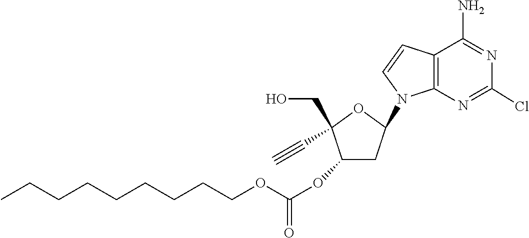 [SMILES: C#C[C@]1(CO)O[C@@H](n2ccc3c(N)nc(Cl)nc32)C[C@@H]1OC(=O)OCCCCCCCCC] |
| 39 | 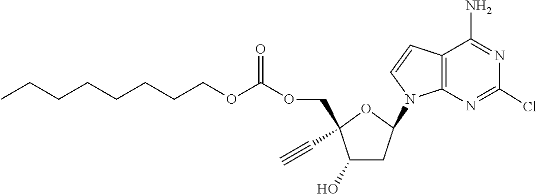 [SMILES: C#C[C@]1(COC(=O)OCCCCCCCC)O[C@@H](n2ccc3c(N)nc(Cl)nc32)C[C@@H]1O] |
| 40 | 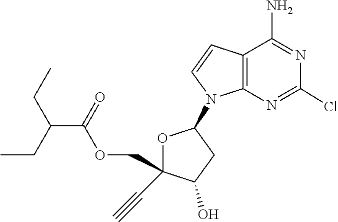 [SMILES: C#C[C@]1(COC(=O)C(CC)CC)O[C@@H](n2ccc3c(N)nc(Cl)nc32)C[C@@H]1O] |
| 41 | 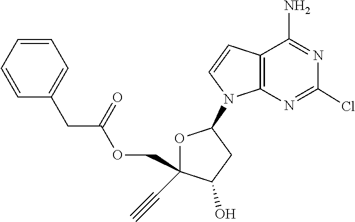 [SMILES: C#C[C@]1(COC(=O)Cc2ccccc2)O[C@@H](n2ccc3c(N)nc(Cl)nc32)C[C@@H]1O] |
| 42 | 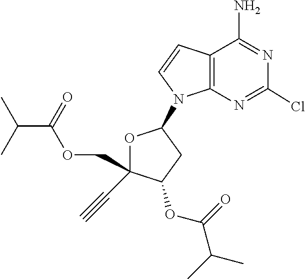 [SMILES: C#C[C@]1(COC(=O)C(C)C)O[C@@H](n2ccc3c(N)nc(Cl)nc32)C[C@@H]1OC(=O)C(C)C] |
| 43 | 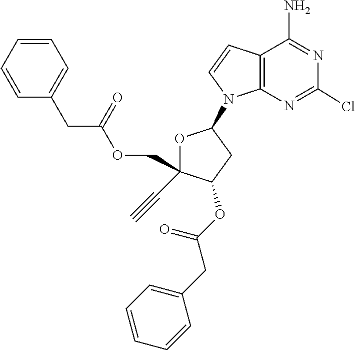 [SMILES: C#C[C@]1(COC(=O)Cc2ccccc2)O[C@@H](n2ccc3c(N)nc(Cl)nc32)C[C@@H]1OC(=O)Cc1ccccc1] |
| 44 | 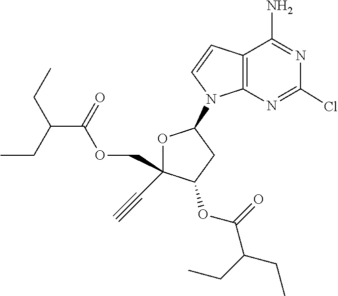 [SMILES: C#C[C@]1(COC(=O)C(CC)CC)O[C@@H](n2ccc3c(N)nc(Cl)nc32)C[C@@H]1OC(=O)C(CC)CC] |
| 45 | 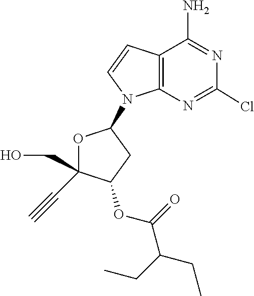 [SMILES: C#C[C@]1(CO)O[C@@H](n2ccc3c(N)nc(Cl)nc32)C[C@@H]1OC(=O)C(CC)CC] |
| 46 | 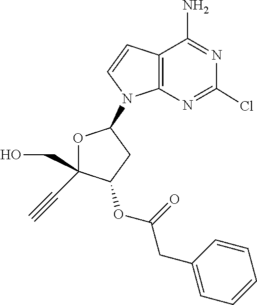 [SMILES: C#C[C@]1(CO)O[C@@H](n2ccc3c(N)nc(Cl)nc32)C[C@@H]1OC(=O)Cc1ccccc1] |
| 47 | 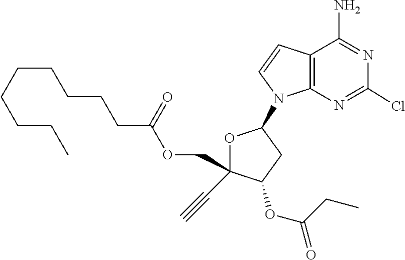 [SMILES: C#C[C@]1(COC(=O)CCCCCCCCC)O[C@@H](n2ccc3c(N)nc(Cl)nc32)C[C@@H]1OC(=O)CC] |
| 48 | 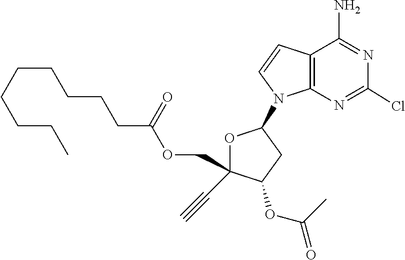 [SMILES: C#C[C@]1(COC(=O)CCCCCCCCC)O[C@@H](n2ccc3c(N)nc(Cl)nc32)C[C@@H]1OC(C)=O] |
| 49 | 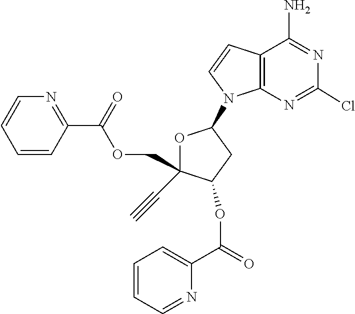 [SMILES: C#C[C@]1(COC(=O)c2ccccn2)O[C@@H](n2ccc3c(N)nc(Cl)nc32)C[C@@H]1OC(=O)c1ccccn1] |
| 50 | 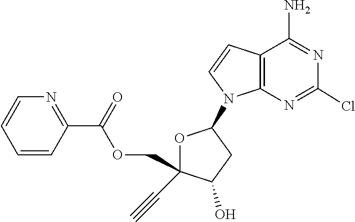 [SMILES: C#C[C@]1(COC(=O)c2ccccn2)O[C@@H](n2ccc3c(N)nc(Cl)nc32)C[C@@H]1O] |
| 51 | 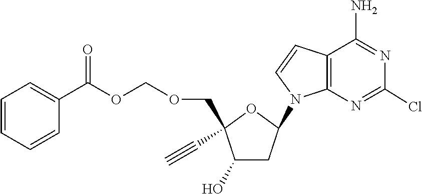 [SMILES: C#C[C@]1(COCOC(=O)c2ccccc2)O[C@@H](n2ccc3c(N)nc(Cl)nc32)C[C@@H]1O] |
| 52 | 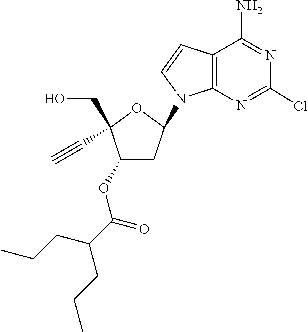 [SMILES: C#C[C@]1(CO)O[C@@H](n2ccc3c(N)nc(Cl)nc32)C[C@@H]1OC(=O)C(CCC)CCC] |
| 53 | 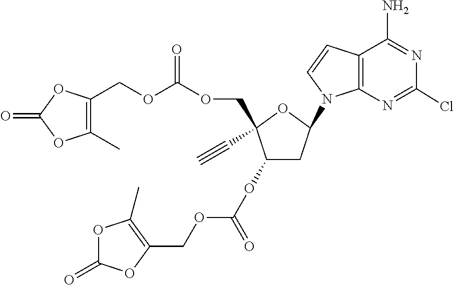 [SMILES: C#C[C@]1(COC(=O)OCc2oc(=O)oc2C)O[C@@H](n2ccc3c(N)nc(Cl)nc32)C[C@@H]1OC(=O)OCc1oc(=O)oc1C] |
| 54 | 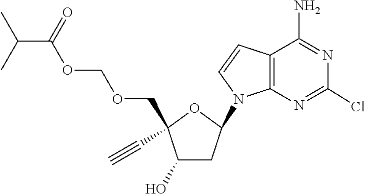 [SMILES: C#C[C@]1(COCOC(=O)C(C)C)O[C@@H](n2ccc3c(N)nc(Cl)nc32)C[C@@H]1O] |
| 55 | 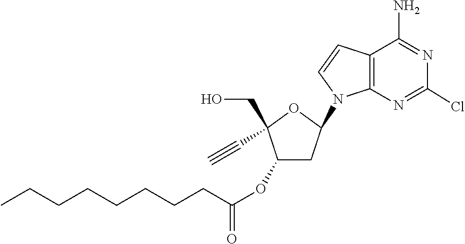 [SMILES: C#C[C@]1(CO)O[C@@H](n2ccc3c(N)nc(Cl)nc32)C[C@@H]1OC(=O)CCCCCCCC] |
| 56 | 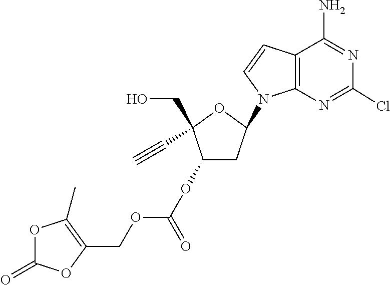 [SMILES: C#C[C@]1(CO)O[C@@H](n2ccc3c(N)nc(Cl)nc32)C[C@@H]1OC(=O)OCc1oc(=O)oc1C] |
| 57 | 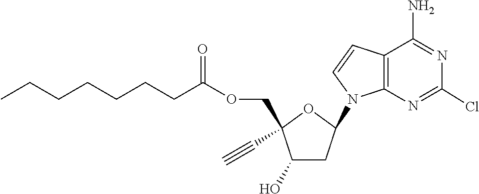 [SMILES: C#C[C@]1(COC(=O)CCCCCCC)O[C@@H](n2ccc3c(N)nc(Cl)nc32)C[C@@H]1O] |
| 58 | 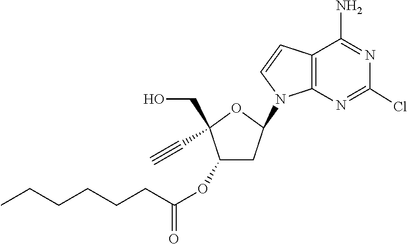 [SMILES: C#C[C@]1(CO)O[C@@H](n2ccc3c(N)nc(Cl)nc32)C[C@@H]1OC(=O)CCCCCC] |
| 59 | 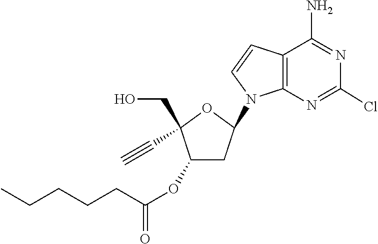 [SMILES: C#C[C@]1(CO)O[C@@H](n2ccc3c(N)nc(Cl)nc32)C[C@@H]1OC(=O)CCCCC] |

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

As used herein, and throughout this disclosure, the following terms, unless otherwise indicated, shall be understood to have the following meanings:

A “subject” is a human or non-human mammal. In one embodiment, a subject is a human. In another embodiment, a subject is a primate. In another embodiment, a subject is a monkey. In another embodiment, a subject is a chimpanzee. In still another embodiment, a subject is a rhesus monkey. In still another embodiment, a subject is a rodent, such as a rat.

The term “effective amount” as used herein, refers to an amount of compound and/or an additional therapeutic agent, or a composition thereof that is effective in inhibiting HIV replication and in producing the desired therapeutic, ameliorative, inhibitory or preventative effect when administered to a subject suffering from HIV infection or AIDS. In the combination therapies of the present invention, an effective amount can refer to each individual agent or to the combination as a whole, wherein the amounts of all agents administered are together effective, but wherein the component agent of the combination may not be present individually in an effective amount.

The terms “treating” or “treatment” as used herein with respect to an HIV viral infection, AIDS or ARC, includes inhibiting the severity of HIV infection or AIDS, e.g., arresting or reducing the development of the HIV infection or AIDS or its clinical symptoms; or relieving the HIV infection or AIDS, e.g., causing regression of the severity of HIV infection or AIDS or its clinical symptoms.

The terms “preventing,” or “prophylaxis,” as used herein with respect to an HIV viral infection or AIDS, refers to reducing the likelihood or severity of HIV infection or AIDS.

The term “alkyl,” as used herein, refers to an aliphatic hydrocarbon group having one of its hydrogen atoms replaced with a bond. An alkyl group may be straight or branched and contain from about 1 to 21 or more carbon atoms. In one embodiment, an alkyl group contains from about 1 to about 12 carbon atoms. In different embodiments, an alkyl group contains from 1 to 6 carbon atoms (C₁₋₆ alkyl) or from about 3 to about 12 carbon atoms (C₃₋₁₂ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched. Unless otherwise indicated, an alkyl group is unsubstituted.

The term “halo,” as used herein, means —F, —Cl, —Br or —I. A particular class of interest of halo substituents for compounds of Formula I and compounds of Formula VI, and embodiments thereof, is each of fluoro (—F) and chloro (—Cl). The term “haloalkyl” refers to an alkyl group as defined above in which one or more of the hydrogen atoms have been replaced with halo (i.e., —F, —Cl, —Br and/or —I). Thus, for example, “C₁₋₆ haloalkyl” (or “C₁-C₆ haloalkyl”) refers to a C₁ to C₆ linear or branched alkyl group as defined above with one or more halo substituents.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent. When any substituent or variable (e.g., R¹) occurs more than one time in any constituent or in Formula I or Formula VI, its definition on each occurrence is independent of its definition at every other occurrence, unless otherwise indicated. It should also be noted that any carbon as well as heteroatom with unsatisfied valences in the text, schemes, examples and tables herein is assumed to have the sufficient number of hydrogen atom(s) to satisfy the valences.

When a functional group in a compound is termed a “protecting group”, this means that the group is in modified form to preclude undesired side reactions at a protected site when the compound is subjected to a reaction. Suitable protecting groups will be recognized by those with ordinary skill in the art as well as by reference to standard textbooks such as, for example, T. W. Greene et al., Protective Groups in Organic Synthesis (1991), Wiley, New York.

When a moiety is noted as being “optionally substituted” in Formula I or Formula VI or any embodiment thereof, it means that Formula I or Formula VI, or the embodiment thereof, encompasses both compounds that are substituted with the noted substituent (or substituents) on the moiety and compounds that do not contain the noted substituent (or substituents) on the moiety (i.e., wherein the moiety is unsubstituted). As one example, when R¹ is a C₁₋₂₁ alkyl group that can be optionally substituted with halo, then R¹ can be C₁₋₂₁ alkyl or C₁₋₂₁ haloalkyl.

When any variable (e.g., R¹, R^{X}, R^{Y}) occurs more than one time in any constituent or in Formula I, Formula VI, or in any other formula depicting and describing compounds of the present invention, its definition on each occurrence is independent of its definition at every other occurrence. Also, combinations of substituents and/or variables are permissible only if such combinations result in stable compounds. Unless expressly stated to the contrary, substitution by a named substituent is permitted on any atom in a ring (e.g., cycloalkyl, aryl, or heteroaryl) provided such ring substitution is chemically allowed and results in a stable compound.

Unless expressly stated to the contrary, all ranges cited herein are inclusive of the recited endpoints and independently combinable. For example, the range of “between about 0.5 and about 95 percent” is inclusive of the endpoints (about) 0.5 percent and (about) 95 percent, and all intermediate values.

As used herein, a “heteroaromatic” ring is a carbon-containing aryl ring may contain 1, 2, 3 or 4 heteroatoms. For instance, a “heteroaromatic” ring may contain one or more nitrogen atoms (e.g., 1 to 3 nitrogen atoms), one or more oxygen atoms, or one or more sulfur atoms. Heteroaromatic rings may be herein expressed using subscripts that denote the total number of atoms making up the ring. In some embodiments, a heteroaromatic ring may have 5-12 ring atoms wherein each atom is selected from carbon, nitrogen, oxygen and sulfur. For instance, a 6-membered heteroaryl substituent may contain 4 carbon atoms and two oxygen atoms.

As used herein, a “heterocyclic” ring is a carbon-containing ring that may contain 1, 2, 3 or 4 heteroatoms. For instance, a “heterocyclic” ring may contain one or more nitrogen atoms (e.g., 1 to 3 nitrogen atoms), one or more oxygen atoms, or one or more sulfur atoms. As with “heteroaromatic” rings, heterocyclic rings may be herein expressed using subscripts that denote the total number of atoms making up the ring. In some embodiments, a heterocyclic ring may have 5-12 ring atoms wherein each atom is selected from carbon, nitrogen, oxygen and sulfur. For instance, a 5-membered heterocyclyl substituent may contain 3 carbon atoms and two oxygen atoms.

In some embodiments, the compounds disclosed herein contain a heteroaryl substituent containing one nitrogen atom. It is also to be understood that any range cited herein includes within its scope all of the sub-ranges within that range. Thus, for example, a “heterocyclic” ring is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, or 4 heteroatoms. For instance, the ring may contain one or more nitrogen atoms (e.g., 1 to 3 nitrogen atoms), one or more oxygen atoms, or one or more sulfur atoms. Any of the cycloalkyl, heterocyclyl, aryl and heteroaryl groups described herein may be optionally substituted with one or more groups. As used herein, “optionally substituted with one to five groups” is intended to include as aspects thereof, the cycloalkyl, heterocyclyl, aryl or heteroaryl substituted with 1 to 5 substituents, 2 to 5 substituents, 3 to 5 substituents, 4 to 5 substituents, 5 substituents, 1 to 4 substituents, 2 to 4 substituents, 3 to 4 substituents, 4 substituents, 1 to 3 substituents, 2 to 3 substituents, 3 substituents, 1 to 2 substituents, 2 substituents, and 1 substituent. Likewise, as used herein, “optionally substituted with one to three groups” is intended to include as aspects thereof, the cycloalkyl, heterocyclyl, aryl or heteroaryl substituted with 1 to 3 substituents, 2 to 3 substituents, 3 substituents, 1 to 2 substituents, 2 substituents, and 1 substituent.

As used herein, the terms “heterocyclyl,” “heteroaryl” and “cycloalkyl” are intended to encompass fused and polycyclic substituents. For example, these terms encompass fused (or polycyclic) rings having five total carbons (e.g., C₅ cycloalkyl), six total carbons (e.g., C₆ cycloalkyl), eight total carbons (e.g., C₈ cycloalkyl), or ten total carbons (e.g., C₁₀ cycloalkyl). These terms also encompass fused (or polycyclic) heterocyclyl rings having five, six, seven, eight, nine, or ten total atoms, of which at least one is carbon and at least one (e.g., 1, 2, 3 or 4) is a non-carbon atom, such as nitrogen, oxygen and/or sulfur.

As used herein, an “alkyl ester” substituent refers to an alkyl group having a terminal ester, in which the site of attachment to the relevant compound is the alcohol side of the ester.

As used herein, the term “composition” is intended to encompass a product comprising the specified ingredients in the specified amounts, as well as any product which results from combination of the specified ingredients in the specified amounts.

The term “prodrug” means a compound (e.g., a drug precursor) that is transformed in vivo to provide a pharmacologically active compound. The in vivo transformation may occur by various mechanisms (e.g., by metabolic or chemical processes), such as, for example, through hydrolysis in blood. The compounds provided herein are prodrugs of Compound A. Where Compound A contains, for example, a hydroxy group, the prodrug can be a derivative of the hydroxy group such as an ester (—OC(O)R), a carbonate ester (—OC(O)OR), a phosphate ester (—O—P(═O)(OH)₂), an ether (—OR), or a mono-phosphate prodrug such as a phosphoramidate (can be converted in vivo to the corresponding nucleoside monophosphate).

The term “salt(s)”, as used herein, denotes acidic salts formed with inorganic and/or organic acids, as well as basic salts formed with inorganic and/or organic bases. In addition, when a compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Compounds can be administered in the form of pharmaceutically acceptable salts. The term “pharmaceutically acceptable salt” refers to a salt which is not biologically or otherwise undesirable (e.g., is neither toxic nor otherwise rious to the recipient thereof).

Compounds of the present invention may exist in amorphous form and/or one or more crystalline forms, and as such all amorphous and crystalline forms and mixtures thereof of the compounds of Formula I and Formula VI are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms. Accordingly, the compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof.

### Salts, Solvates and Stereoisomers

Solvates of the disclosed Compounds of Formula I and Formula VI are contemplated herein. One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain situations, the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more Compounds of Formula I or Formula VI may optionally be converted to a solvate. Preparation of solvates is generally known. Thus, for example, M. Caira et al., J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al., AAPS PharmSciTech., 5(1), article 12 (2004); and A. L. Bingham et al., Chem. Commun., 603-604 (2001). A typical, non-limiting, process involves dissolving the compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The compounds of Formula I and Formula VI can form salts which are also within the scope of this invention. In some embodiments, the salt is a pharmaceutically acceptable salt. In another embodiment, the salt is other than a pharmaceutically acceptable salt. Salts of the compounds of Formula I and Formula VI may be formed, for example, by reacting the compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich:Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), arylalkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the compounds of Formula I and/or Formula VI may be atropisomers (e.g., substituted biaryls) and are considered as part of this invention. Enantiomers can also be directly separated using chiral chromatographic techniques.

It is also possible that the compounds of Formula I and/or Formula VI may exist in different tautomeric forms, and all such forms are embraced within the scope of the invention. For example, all keto-enol and imine-enamine forms of the compounds are included in the invention. As another example, both the hydroxypyridine and pyridinone forms of oxo-substituted pyridine substituents are encompassed within embodiments of the disclosed compounds.

Unless otherwise indicated, all stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a compound incorporates a double bond or a fused ring, both the cis- and trans-forms, as well as mixtures, are embraced within the scope of the invention.

When a substituent on a chiral carbon atom is depicted without specific stereochemistry (by using a straight-line bond to a chiral center), it is to be understood that both the alpha and beta configurations of said substituent group are to be considered part of the present invention. It is understood that a chiral center in a compound may exist in the S or R absolute configuration, or as a mixture of both. Within a molecule, each bond drawn as a straight line from a chiral center includes both the R and S stereoisomers as well as mixtures thereof. An asterisk denotes a stereocenter in a single configuration, either R or S. Absolute stereochemistry of separate stereoisomers in the examples and intermediates are not determined unless stated otherwise in an example or explicitly in the nomenclature.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” and the like, is intended to apply equally to the salt and solvate of enantiomers, stereoisomers, rotamers, tautomers or racemates of the disclosed compounds.

In the compounds of Formula I and Formula VI, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula VI and Formula I. For example, different isotopic forms of hydrogen (H) include protium (¹H) and deuterium (²H). Protium is the predominant hydrogen isotope found in nature. Enriching for deuterium may provide certain therapeutic advantages, such as increasing in vivo half-life or reducing dosage requirements, or may provide a compound useful as a standard for characterization of biological samples. Isotopically-enriched compounds of Formula I and Formula VI can be prepared without undue experimentation by conventional techniques well known to those skilled in the art or by processes analogous to those described in the Schemes and Examples herein using appropriate isotopically-enriched reagents and/or intermediates. In one embodiment, a compound of Formula I or Formula VI has one or more of its hydrogen atoms replaced with deuterium.

### Treatment or Prevention of HIV Infection

The disclosed compounds of Formula I and Formula VI may be useful in the inhibition of HIV, the inhibition of HIV reverse transcriptase, the treatment of HIV infection and/or reduction of the likelihood or severity of symptoms of HIV infection and the inhibition of HIV viral replication and/or HIV viral production in a cell-based system. For example, the Compounds of Formula I and Formula VI may be useful in treating infection by HIV after suspected past exposure to HIV by such means as blood transfusion, exchange of body fluids, bites, accidental needle stick, or exposure to subject blood during surgery or other medical procedures. In one embodiment, the compounds of Formula I and Formula VI can be inhibitors of HIV-1 viral replication. Accordingly, the compounds of Formula I and Formula VI may be useful for treating HIV infections and AIDS. In accordance with the invention, the compounds of Formula I and Formula VI can be administered to a subject in need of treatment or prevention of HIV infection.

Accordingly, in one embodiment, the invention provides methods for treating HIV infection in a subject, the methods comprising administering to the subject an effective amount of at least one disclosed compound or a pharmaceutically acceptable salt thereof. In particular embodiments, the amount administered is effective to treat or prevent infection by HIV in the subject. In some embodiments, the amount administered is effective to inhibit HIV viral replication and/or viral production in the subject. In one embodiment, the HIV infection has progressed to AIDS.

The Compounds of Formula I and Formula VI are also useful in the preparation and execution of screening assays for antiviral compounds. For example, the compounds of Formula I may be useful for identifying resistant HIV cell lines harboring mutations, which are excellent screening tools for more powerful antiviral compounds. Furthermore, the compounds of Formula I and Formula VI may be useful in establishing or determining the binding site of other antivirals to the HIV reverse transcriptase.

The compositions and combinations of the present invention may be useful for treating a subject suffering from infection related to any HIV genotype.

### Compositions and Administration

When administered to a subject, any of the disclosed Compounds of Formula I and Formula VI may be administered as a component of a composition that comprises a pharmaceutically acceptable carrier. The present invention provides pharmaceutical compositions comprising an effective amount of at least one of the disclosed compounds and a pharmaceutically acceptable carrier. In the pharmaceutical compositions and methods of the present invention, the active ingredients will typically be administered in admixture with suitable carrier materials selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any pharmaceutically acceptable inert carrier. Solid form preparations include powders, tablets, dispersible granules, capsules, sachets and suppositories. Tablets, powders, sachets and capsules may be suitable for oral administration. Powders and tablets may be comprised of between about 0.5 and about 95 percent of any of the disclosed pharmaceutical compositions.

Moreover, when desired or needed, suitable binders, glidants, lubricants, disintegrating agents and coloring agents may also be incorporated in the composition, particularly in formulations for oral administration. The compositions may be formulated for extended or controlled release. In other embodiments, the compositions are formulated for immediate or modified release.

In some embodiments, any of the disclosed pharmaceutical compositions comprise pharmaceutically acceptable carriers that are suitable or adapted for administration to the subject by injection. In some embodiments, these carriers are adapted for long-action injection. In some embodiments, these carriers are liquid form preparations that include solutions, suspensions, emulsions, or nano-emulsions for intramuscular or subcutaneous administration. In some embodiments, any of the disclosed pharmaceutical compositions are adapted for long-acting injectable formulations.

Any of the disclosed compositions may comprise pharmaceutically acceptable carriers that are suitable or adapted for administration parenterally, including subcutaneous, intravenous, intramuscular or intrasternal injection, or other infusion techniques (one or more injections or infusions may be administered at each dosing interval as needed to deliver the appropriate amount of active agent), in the form of a unit dosage of a pharmaceutical composition containing an effective amount of the compound and conventional pharmaceutically acceptable carriers, adjuvants and vehicles for the treatment of a subject infected with HIV, and/or for the prevention of HIV infection. The compositions may also be administered parenterally via an implantable drug delivery composition or device adapted to provide an effective amount of the compound over an extended period of time. In some embodiments, the composition is administered parenterally between once per month to about once per every twelve months, such as about once per every three months, once per every six months, or once per every twelve months.

Parenteral compositions can be prepared according to techniques known in the art. These compositions may employ sterile water as a carrier and optionally other ingredients. A continuous dosing regimen should be used for treatment of HIV-infected subjects. Any of the disclosed pharmaceutical preparations for parenteral injection may comprise solutions, suspensions or emulsions that may include water, a suspending agent, a viscosity modifier, a tonicity modifier, and/or a pH modifier.

Also included are solid form preparations which are intended to be converted, shortly before use, to liquid form preparations for oral or parenteral administration. Such liquid forms include solutions, suspensions, emulsions and nano-emulsions.

In particular embodiments, the compositions of the present invention may be formulated in extended dosing, or sustained release, forms to provide a rate-controlled release of any one or more of the components or active ingredients to optimize therapeutic effects, i.e., antiviral activity and the like. Suitable dosage forms for sustained release include long-acting injectable and implant dosage forms. Other suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In some embodiments, sustained release dosage forms of the disclosed compositions are facilitated by the reduced solubility of the disclosed compounds of Formula I and Formula VI relative to Compound A. In various embodiments, the compounds of any of the disclosed compositions exhibit aqueous solubilities at physiological pH (e.g., pH of 7.4, 7.2, or 7.0) less than the solubility of Compound A. In some embodiments, the compounds of any of the disclosed compositions exhibit solubilities at pH 7.4 of less than 400 μg/mL less than 350 μg/mL, less than 250 μg/mL, less than 100 μg/mL, less than 50 μg/mL less than 10 μg/mL, less than 2.5 μg/mL, or less than 1 μg/mL. In particular embodiments, the compounds of any of the disclosed compositions exhibit solubilities at pH 7.4 of less than 1 μg/mL.

In some embodiments, any of the disclosed compounds exhibit a substantially short half-life (T½) in the presence of human plasma. In some embodiments, any of the disclosed compounds exhibit a long half-life in the presence of human plasma. In some embodiments, any of these compounds exhibit a substantially long half-life (T½) in the presence of rodent plasma or non-human primate plasma. In some embodiments, any of these compounds exhibit a half-life in human plasma of less than 2.5 hr, less than 1 hr, less than 0.5 hr, or less than 0.25 hr, as measured by the percentage of drug loss over the course of 0, 0.25, 0.5, 1, and 3 hr. In some embodiments, any of these compounds a substantially longer half-life than that of Compound A.

Compositions may be prepared according to conventional mixing, granulating or coating methods, respectively, and the present compositions can contain, in one embodiment, from about 0.1% to about 99% of the compound by weight or volume. In various embodiments, the present compositions can contain, in some embodiments, from about 1% to about 70% or from about 5% to about 60% of the compound by weight or volume.

### Formulations

In some embodiments, any of the disclosed compositions are injectable, or suitable or adapted for injection. Injectable compositions can be prepared according to methods known in the art. Implantable compositions can be prepared according to methods known in the art wherein the carrier comprises the active chemical ingredient with polymers and suitable excipients, or utilizing an implantable device or subcutaneous depot for drug delivery. Further description of methods suitable for use in preparing pharmaceutical compositions for use in the present invention and of ingredients suitable for use in said compositions is provided in Remington—THE SCIENCE AND PRACTICE OF PHARMACY, 22nd Edition, published by Pharmaceutical Press and Philadelphia College of Pharmacy at University of the Sciences, 2012. Liquid preparations suitable for oral administration (e.g., suspensions, syrups, elixirs and the like) can be prepared according to techniques known in the art and can employ any of the usual media such as water, glycols, oils, alcohols and the like.

In some embodiments, any of the disclosed compounds or a pharmaceutically acceptable salt thereof, is administered via injection, using an injection device. In some embodiments, the injection device is or includes a syringe, which can be employed manually, or as part of a syringe-containing injection device. A wide variety of injection devices can be used, including, but not limited to, a handheld or wearable autoinjector, a handheld or wearable manual injector, an on-body injector, a syrette, a jet injector, or a pen injector, each of which can be reusable or disposable.

In some embodiments, the compound of Formula I, Formula II, Formula III, Formula IV, Formula V, or Formula VI, or a pharmaceutically acceptable salt thereof, can be administered using a syringe, or an autoinjector comprising a syringe, suitable for administration of the compound. In some embodiments, the syringe is disposable. In some embodiments, the syringe is reusable. In some embodiments, the syringe is pre-filled with any of these compounds.

In some embodiments, a compound of the present disclosure is administered via an implantable device, which is implanted into the subject to deliver the active agent during the interval of time from one implant to the subsequent implant. In some embodiments, the implantable device administers any of the disclosed compounds, or salts thereof, in accordance with a dosing interval range from about once per month to about once per every six to twelve months. In particular embodiments, the dosing interval range is about once per every three months, or once per every six months, or once per every twelve months. In some embodiments, the implantable device administers the compound once-monthly, once every 3 months, or less frequently. Implantable compositions can also be prepared according to methods known in the art wherein, for example, the carrier comprises the active chemical ingredient with suitable excipients (e.g., polymers), or utilizing an implantable device for drug delivery.

Solid pharmaceutical preparations suitable for oral administration (e.g., powders, pills, capsules and tablets) can be prepared according to techniques known in the art and can employ such solid excipients as starches, sugars, kaolin, lubricants, glidants, binders, disintegrating agents and the like. Formulations of compounds of Formula I and Formula VI that result in drug supersaturation and/or rapid dissolution may be utilized to facilitate oral drug absorption. Formulation approaches to cause drug supersaturation and/or rapid dissolution include, but are not limited to, nanoparticulate systems, amorphous systems, solid solutions, solid dispersions, and lipid systems. Such formulation approaches and techniques for preparing them are known in the art. For example, solid dispersions can be prepared using excipients and processes as described in reviews (e.g., Serajuddin, J Pharm Sci, 88:10, pp. 1058-1066 (1999)). Nanoparticulate systems based on both attrition and direct synthesis have also been described in reviews such as Wu et al., Advanced Drug Delivery Reviews, 59:7 pp. 631-644 (2007)).

The unit dosages of the disclosed compounds of Formula I and Formula VI may be administered at varying frequencies. In some embodiments, a unit dosage of any of these compounds is administered by intramuscular or subcutaneous injection once every month, every 2 months, every 3 months, every 4 months, every 5 months, every 6 months, or less frequently than once every 6 months. In some embodiments, a unit dosage is administered every 2 months. In some embodiments, a unit dosage is administered every 3 months. In some embodiments, a unit dosage is administered every 2-6 months. In some embodiments, a unit dosage is administered every 6 months. In some embodiments, a unit dosage is administered every 6 to 12 months. In some embodiments, a unit dosage is administered every 12 months.

In one embodiment, a unit dosage of a disclosed compound may be administered once daily. In another embodiment, a unit dosage of a disclosed compound may be administered twice weekly. In another embodiment, a unit dosage may be administered once weekly. In still another embodiment, a unit dosage may be administered once biweekly. In another embodiment, a unit dosage may be administered (e.g., by intramuscular or subcutaneous injection) once monthly. In yet another embodiment, a unit dosage may be administered once bimonthly. In still another embodiment, a unit dosage may be administered quarterly. In still another embodiment, a unit dosage may be administered once yearly.

In some aspects, an effective amount of a compound of Formula I and Formula VI for prophylactic use may be administered by injection at, for example but not limited to, once-weekly, bi-weekly, twice-monthly, once-monthly, once-quarterly, twice-yearly, once-yearly or at longer intervals, for example but not limited to, once every 18 months or bi-annually (once every two years). The longer the interval between each administration of the active agent, the greater the amount of active agent may be needed at each administration. Therefore, one or more unit dosage(s) may be administered at each dosing interval as needed to deliver the appropriate amount of active agent, for example, one or more injections or infusions of the compound of Formula I and Formula VI, or one or more implantable compositions or devices. Any dosing regimen for prophylactic use can be a continuous dosing regimen or an intermittent dosing regimen.

In one embodiment, a pharmaceutical preparation comprising at least one disclosed compound is subdivided into unit doses containing effective amounts of the active components. In some embodiments, the compounds of Formula I and Formula VI are administered in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, the compositions may be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the subject to be treated. The specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the subject undergoing therapy.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24-hour period. In still another embodiment, the total daily dosage is administered in four divided doses over a 24-hour period.

The amount and frequency of administration of the compounds of Formula I and Formula VI will be regulated according to the judgment of the attending clinician considering such factors as age, condition and size of the subject as well as severity of the symptoms being treated. The compositions of the invention can further comprise one or more additional therapeutic agents, selected from those listed above herein.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, the effect of other drugs the patient is taking while using any of the compounds or pharmaceutical compositions described herein, the severity of the particular condition, and the host undergoing therapy. In some cases, depending on the potency of the compound or the individual response, it may be necessary to deviate upwards or downwards from the given dose. The amount and frequency of administration will be regulated according to the judgment of the attending clinician considering such factors.

The compounds of this invention are also useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals.

### Combination Therapies

In some aspects, the present methods for treating or preventing HIV infection can further comprise the administration of one or more additional therapeutic agents that are not any of the disclosed compounds.

In one embodiment, the additional therapeutic agent is an antiviral agent. In another embodiment, the additional therapeutic agent is an immunomodulatory agent, such as an immunosuppressive agent.

In some embodiments, the additional therapeutic agent is an HIV capsid inhibitor. In some embodiments, the additional therapeutic agent is lenacapavir (Sunlenca®). The method of action of lenacapavir is discussed in US Publication No. 2018/0051005, published Feb. 22, 2018, which is herein incorporated by reference in its entirety. In some embodiments, the additional therapeutic agent is GS-CA1 (see Vidal, et al. Long-acting capsid inhibitor protects macaques from repeat SHIV challenges. Nature 601, 612-616 (2022), herein incorporated by reference in its entirety).

Accordingly, in one embodiment, the present invention provides methods for treating a viral infection in a subject, the method comprising administering to the subject: (i) at least one compound of Formula I or Formula VI (which may include two or more different compounds), or a pharmaceutically acceptable salt thereof, and (ii) at least one additional therapeutic agent that is other than any of the disclosed compounds of Formula I and Formula VI, wherein the amounts administered are together effective to treat or prevent a viral infection.

When administering a combination therapy of the invention to a subject, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a compound and an additional therapeutic agent may be present in fixed amounts (dosage amounts) in a single dosage unit (e.g., a capsule, a tablet and the like).

In some embodiments, any of the disclosed compounds or a pharmaceutically acceptable salt thereof, is administered via injection, and the additional therapeutic agent is further administered via injection, using one or more injection devices. In some embodiments, the one or more injection devices is or includes a syringe, which can be employed manually, or as part of a syringe-containing injection device such as an autoinjector. A wide variety of injection devices can be used, including, but not limited to, a handheld or wearable autoinjector, a handheld or wearable manual injector, an on-body injector, a syrette, a jet injector, or a pen injector, each of which can be reusable or disposable.

In one embodiment, at least one compound is administered during a time when the additional therapeutic agent(s) exert their prophylactic or therapeutic effect, or vice versa.

In another embodiment, at least one compound and the additional therapeutic agent(s) are administered in doses commonly employed when such agents are used as monotherapy for treating a viral infection.

In another embodiment, at least one compound and the additional therapeutic agent(s) are administered in doses lower than the doses commonly employed when such agents are used as monotherapy for treating a viral infection.

In still another embodiment, at least one compound and the additional therapeutic agent(s) act synergistically and are administered in doses lower than the doses commonly employed when such agents are used as monotherapy for treating a viral infection.

In one embodiment, at least one compound and the additional therapeutic agent(s) are present in the same composition. In one embodiment, this composition is suitable for subcutaneous administration. In another embodiment, this composition is suitable for intramuscular administration. In another embodiment, this composition is suitable for oral administration. In still another embodiment, this composition is suitable for intravenous administration.

Viral infections and virus-related disorders that may be treated or prevented using the combination therapy methods of the present invention include, but are not limited to, those listed above. In some embodiments, the viral infection is HIV infection. In some embodiments, the viral infection is AIDS.

The at least one compound and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy. In one embodiment, the administration of at least one compound and the additional therapeutic agent(s) may inhibit the resistance of a viral infection to these agents.

In certain embodiments, the disclosed compounds or pharmaceutically acceptable salts thereof are combined with at least one HIV long-acting therapy, such as a long-acting injectable. Examples of drugs that are being developed as long-acting regimens include cabotegravir, rilpivirine, lenacapavir, tenofovir implant, islatravir implant, doravirine, raltegravir, and long-acting dolutegravir. In some embodiments, the disclosed compounds are combined with long-acting lenacapavir.

As noted above, the present invention is also directed to use of a compound of Formula I or Formula VI with one or more anti-HIV agents. An “anti-HIV agent” is any agent which is directly or indirectly effective in the inhibition of HIV reverse transcriptase or another enzyme required for HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, anti-infectives, or vaccines useful for treating HIV infection or AIDS. Suitable HIV antivirals for use in combination with the compounds of the present invention include, for example, those listed in Table B as follows:

TABLE B

| Name | Trade Name |
|---|---|
| abacavir, ABC | Ziagen ® |
| abacavir + lamivudine | Epzicom ® |
| abacavir + lamivudine + zidovudine | Trizivir ® |
| amprenavir | Agenerase ® |
| atazanavir | Reyataz ® |
| AZT, zidovudine, azidothymidine | Retrovir ® |
| Cabotegravir | Cabenuva ®, Vocabria ® |
| darunavir | Prezista ® |
| ddC, zalcitabine, dideoxycytidine | Hivid ® |
| ddI, didanosine, dideoxyinosine | Videx ® |
| ddI (enteric coated) | Videx EC ® |
| delavirdine, DLV | Rescriptor ® |
| dolutegravir | Tivicay ® |
| Doravirine | Pifeltro ® |
| doravirine + lamivudine + tenofovir DF | Delstrigo ® |
| efavirenz, EFV | Sustiva ®, Stocrin ® |
| efavirenz + emtricitabine + tenofovir DF | Atripla ® |
| emtricitabine, FTC | Emtriva ® |
| emtricitabine + tenofovir DF | Truvada ® |
| emvirine | Coactinon ® |
| enfuvirtide | Fuzeon ® |
| enteric coated didanosine | Videx EC ® |
| etravirine, TMC-125 | Intelence ® |
| fosamprenavir calcium | Lexiva ® |
| GS-CA1 | |
| indinavir | Crixivan ® |
| islatravir (EFdA, 4′-ethynyl-2-fluoro- | |
| 2′-deoxyadenosine) | |
| lamivudine, 3TC | Epivir ® |
| lamivudine + zidovudine | Combivir ® |
| Lenacapavir | Sunlenca ® |
| Lopinavir | |
| lopinavir + ritonavir | Kaletra ® |
| maraviroc | Selzentry ® |
| nelfinavir | Viracept ® |
| nevirapine, NVP | Viramune ® |
| rilpivirine, TMC-278 | Edurant ® |
| Ritonavir | Norvir ® |
| Saquinavir | Invirase ®, Fortovase ® |
| stavudine, d4T,didehydrodeoxythymidine | Zerit ® |
| tenofovir DF (DF = disoproxil fumarate), | Viread ® |
| TDF | |
| Tipranavir | Aptivus ® |

Some of the drugs listed in the table are used in a salt form; e.g., abacavir sulfate, indinavir sulfate, atazanavir sulfate, nelfinavir mesylate.

In one embodiment, one or more anti-HIV drugs are selected from lenacapavir, GS-CA1, lamivudine, abacavir, ritonavir, darunavir, atazanavir, emtricitabine, tenofovir, rilpivirine, doravirine, islatravir and lopinavir.

In some embodiments, the compound of Formula I or Formula VI is used in combination with lenacapavir or GS-CA1.

In still another embodiment, the compound of Formula I or Formula VI is used in combination with atazanavir. In another embodiment, the compound of Formula I or Formula VI is used in combination with darunavir. In another embodiment, the compound of Formula I or Formula VI is used in combination with rilpivirine. In one embodiment, the compound of Formula I or Formula VI is used in combination with lamivudine and abacavir.

In another embodiment, the compound of Formula I or Formula VI is used in combination with islatravir. In another embodiment, the compound of Formula I or Formula VI is used in combination with emtricitabine and tenofovir. In still another embodiment, the compound of Formula I or Formula VI is used in combination with doravirine. In still another embodiment, the compound of Formula I or Formula VI is used in combination doravirine, lamivudine and tenofovir DF. In another embodiment, the compound of Formula I or Formula VI is used in combination with ritonavir and lopinavir. In one embodiment, the compound of Formula I or Formula VI is used in combination with abacavir and lamivudine. In another embodiment, the compound of Formula I or Formula VI is used in combination with lopinavir and ritonavir.

In one embodiment, the present invention provides pharmaceutical compositions comprising (i) a compound of Formula I or Formula VI or a pharmaceutically acceptable salt thereof; (ii) a pharmaceutically acceptable carrier; and (iii) one or more additional anti-HIV agents selected from lenacapavir, lamivudine, abacavir, ritonavir, islatravir, doravirine and lopinavir, or a pharmaceutically acceptable salt or prodrug thereof, wherein the amounts present of components (i) and (iii) are together effective for the treatment or prophylaxis of infection by HIV or for the treatment, prophylaxis, or delay in the onset or progression of AIDS in the subject in need thereof.

In another embodiment, the present invention provides a method for the treatment or prophylaxis of infection by HIV or for the treatment, prophylaxis, or delay in the onset or progression of AIDS in a subject in need thereof, which comprises administering to the subject (i) a compound of Formula I or Formula VI or a pharmaceutically acceptable salt thereof and (ii) one or more additional anti-HIV agents selected from lenacapavir, lamivudine, abacavir, ritonavir, islatravir, doravirine and lopinavir, or a pharmaceutically acceptable salt or prodrug thereof, wherein the amounts administered of components (i) and (ii) are together effective for the treatment or prophylaxis of infection by HIV or for the treatment, prophylaxis, or delay in the onset or progression of AIDS in the subject in need thereof.

It is understood that the scope of combinations of the compounds of this invention with anti-HIV agents is not limited to the HIV antivirals listed in Table B, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PDR, Thomson PDR, 57^{th} edition (2003), the 58^{th} edition (2004), the 59^{th} edition (2005), and the like. The dosage ranges for a compound of the invention in these combinations are the same as those set forth above.

The doses and dosage regimen of the other agents used in the combination therapies of the present invention for the treatment or prevention of HIV infection may be determined by the attending clinician, taking into consideration the approved doses and dosage regimen in the package insert; the age, sex and general health of the subject; and the type and severity of the viral infection or related disease or disorder. When administered in combination, the compound and the other agent(s) may be administered simultaneously (i.e., in the same composition or in separate compositions one right after the other) or sequentially. This is particularly useful when the components of the combination are given on different dosing schedules, e.g., one component is administered once daily and another component is administered every six hours, or when the pharmaceutical compositions are different, e.g., one is a tablet and one is a capsule. A kit comprising the separate dosage forms is therefore advantageous.

### Kits

In some aspects, the present invention provides a kit comprising a therapeutically effective amount of at least one compound, or a pharmaceutically acceptable salt thereof, and a pharmaceutically acceptable carrier, vehicle or diluent.

Further provided herein are also articles of manufacture that include any of the disclosed compounds, or a pharmaceutically acceptable salt thereof in a suitable container. The container may be a vial, jar, ampoule, preloaded syringe, implant, or intravenous bag. Any of the disclosed kits may contain any of these article of manufacture.

In another aspect the present invention provides a kit comprising an amount of at least one compound, or a pharmaceutically acceptable salt thereof, and an amount of at least one additional therapeutic agent listed above, wherein the amounts of the two or more active ingredients result in a desired therapeutic effect. In one embodiment, the one or more compounds of Formula I or Formula VI and the one or more additional therapeutic agents are provided in the same container. In one embodiment, the one or more compounds of Formula I or Formula VI and the one or more additional therapeutic agents are provided in separate containers.

Several methods for preparing the compounds of Formula I and Formula VI are described in the following Procedures and Schemes. These schemes may use readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art.

### General Procedures

Starting materials and intermediates were purchased or were prepared using known procedures described in the chemical synthetic literature or as otherwise described. The preparation of the various starting materials used herein is well within the skill of a person versed in the art. Routes applied to the synthesis of compounds of Formula I and Formula VI are described in the following schemes. In some cases, the sequence of reaction steps may be varied to facilitate reactions or to avoid unwanted reaction products. In some cases, the final product may be further modified, for example, by manipulation of substituents. These manipulations may include, but are not limited to, reduction, oxidation, alkylation, acylation, and hydrolysis reactions which are commonly known to those skilled in the art. Because the schemes are an illustration, the invention should not be construed as being limited by the chemical reactions and conditions expressed. The examples described below are provided so that the invention might be more fully understood. These examples are illustrative only and should not be construed as limiting the invention in any way

Substituent numbering as shown in the schemes does not necessarily correlate to that used in the claims and often, for clarity, a single substituent is shown attached to the compound where multiple substituents are allowed under the definitions above. Reactions used to generate the compounds of this invention are carried out by employing reactions as shown in the schemes and examples herein, in addition to other standard manipulations such as ester hydrolysis, cleavage of protecting groups, etc., as may be known in the literature or exemplified in the experimental procedures.

Reactions sensitive to moisture or air were performed under nitrogen or argon using anhydrous solvents and reagents. The progress of reactions was determined by either liquid chromatography-mass spectrometry (LCMS) or analytical thin layer chromatography (TLC) usually performed with Merck KGaA glass-backed TLC plates, silica gel 60 F₂₅₄.

Analytical LCMS was commonly performed on a Waters SQD single quadrupole mass spectrometer with electrospray ionization in positive ion detection mode (mass range set at 150-900 daltons, data collected in centroid mode and scan time set to 0.2 seconds) and a Waters Acquity UPLC system (binary solvent manager, sample manager, and TUV). The column was commonly a Waters Acquity BEH C18 1×50 mm, 1.7 μm, heated to 50° C. The mobile phases used may be modified with either acidic or basic additives. The acidic mobile phase consisted of 0.1% trifluoroacetic acid in water for Solvent A and 100% acetonitrile for Solvent B. A two-minute run was established at a flow rate of 0.3 ml/min with Initial conditions of 95% Solvent A and ramping up to 99% Solvent B at 1.60 minutes and holding at 99% Solvent B for 0.40 minutes. The injection volume was 0.5 μL using partial loop needle overfill injection mode. The TUV monitored wavelength 215 or 254 nm with a sampling rate of 20 points/second, normal filter constant and absorbance data mode. A five-minute run was established at a flow rate of 0.3 ml/min with initial conditions of 90% Solvent A and ramping up to 99% Solvent B at 4.90 minutes and holding at 99% Solvent B for 0.10 minutes.

Alternatively, a commonly used system consisted of a Shimadzu LCMS-2020™ platform with electrospray ionization in positive ion detection mode. The column was commonly a Kinetix EVO C18, 3.0×30 mm, 2.6 μm. The flow rate was 1.5 mL/min and the injection volume was 2 μL. UV detection was in the range 190-400 nm. The mobile phase consisted of solvent A (water plus 5 mM NH₄HCO₃) and solvent B (MeCN) with a gradient of 10% solvent A to 60% solvent B over 1.85 min. Alternatively, the column was commonly a XSelect HSS T3, 2.1×30 mm, 2.5 m. The flow rate was 1.2 mL/min and the injection volume was 1 μL. UV detection was in the range 190-400 nm. The mobile phase consisted of solvent A (water plus 0.05% TFA) and solvent B (MeCN plus 0.05% TFA) with a gradient of 95% solvent A changing to 100% solvent B over 1.30 min. Alternatively, the column was commonly a ShimNex HE C18-AQ, 3.0×30 mm, 3.0 m. The flow rate was 1 mL/min, and the injection volume was 0.8 μL. UV detection was in the range 190-400 nm. The mobile phase consisted of solvent A (water plus 0.1% TFA) and solvent B (MeCN plus 0.1% TFA) with a gradient of 95% solvent A changing to 100% solvent B over 1.20 min.

Preparative reverse-phase chromatography was commonly performed on a Teledyne ISCO CombiFlash Rf or Teledyne ISCO ACCQPrep HP125 or HP150 apparatus or Waters apparatus equipped with UV. The UV detector typically monitored wavelengths of 215 and 254 nm. The mobile phase consisted of solvent A (water, with or without modifiers such as FA or NH₄HCO₃) and solvent B (MeCN). Mobile phase gradients were optimized for the individual compounds.

Flash chromatography was commonly performed using an ISCO CombiFlash Rf apparatus, a Biotage® Flash Chromatography apparatus (Dyax Corp.), or an ISCO CombiFlash® Companion XL apparatus on silica gel (60 Å pore size) in pre-packed columns. Mobile phases generally consisted of mixtures of hexanes, pet. ether or dichloromethane with EtOAc, 3:1 EtOAc:EtOH, or MeOH. Mobile phase gradients were optimized for the individual compounds.

¹H NMR data were typically acquired using a Bruker NMR spectrometer at 300-500 MHz. Chemical shift values are reported in delta (δ) units, parts per million (ppm). Chemical shifts for ¹H NMR spectra are given relative to signals for residual non-deuterated solvent (CDCl₃ referenced at δ 7.26 ppm; DMSO-d₆ referenced at δ 2.50 ppm and CD₃OD referenced at δ 3.31 ppm). Multiplets are reported by the following abbreviations: s=singlet, d=doublet, t=triplet, q=quartet, dd=doublet of doublets, m=multiplet or overlap of nonequivalent resonances. Coupling constants (J) are reported in Hertz (Hz). When compounds appear as mixtures of rotamers by NMR, spectral data corresponding to the major species observed in solution are reported.

Abbreviations employed herein include the following:

TABLE C

| ACN or | | | |
|---|---|---|---|
| MeCN | acetonitrile | NP | Normal phase |
| aq. | Aqueous | PLM | polarized light microscopy |
| DCC | N,N-dicyclohexylcarbodiimide | prep | preparative |
| DIC | N′-diisopropylcarbodiimide | pet. | Petroleum |
| DIEA | N,N-diisopropylethylamine | PBS | phosphate buffer saline |
| DCM | dichloromethane | PVDF | polyvinylidene fluoride |
| DMAP | 4-dimethylaminopyridine | Py | pyridine |
| DMA | dimethylacetamide | RP | Reverse phase |
| DMF | N,N-dimethylformamide | RPMI | Roswell Park Memorial |
| | | | Institute |
| DMSO | dimethyl sulfoxide | RT or rt | room temperature (ambient, |
| | | | about 25° C.) |
| DMTr | 4,4′-dimethoxytrityl | rxn | reaction |
| EtOAc | Ethyl acetate | sat. | saturated |
| EDCI | 1-Ethyl-3-(3- | TBS | tert-butyldimethylsilyl |
| | dimethylaminopropyl)carbodiimide | | |
| FA | formic acid | TBAF | tetrabutylammonium |
| | | | fluoride |
| h or hr. | hours | TCFH | chloro-N,N,N′,N′- |
| | | | tetramethylformamidinium |
| | | | hexafluorophosphate |
| GFP | Green fluorescent protein | TLC | thin-layer chromatography |
| HPLC | high-pressure liquid | TEA | triethylamine (Et₃N) |
| | chromatography | | |
| LCMS | liquid chromatography-mass | TFA | trifluoroacetic acid |
| | spectrometry | | |
| LiTMP | 2,2,6,6- | THF | tetrahydrofuran |
| | tetramethylpiperidinylmagnesium | | |
| | chloride lithium chloride complex | | |
| mmol | millimoles | TEA•3HF | triethylamine |
| | | | trihydrofluoride |
| MMTr | 4-methoxytriphenylmethyl | T½ | half-life |
| MeOH | methanol | UPLC | ultra-performance liquid |
| | | | chromatography |
| MHz | megahertz | UV | ultraviolet |
| MS | Mass spectrometry | VIKING | Viral KINetics in Green |
| | | | cells |
| NOVO | Novozyme | XRPD | X-ray diffraction powder |
| | | | diffraction |
| NMR | nuclear magnetic resonance | anhy | anhydrous |
| | spectroscopy | | |

As illustrated in Scheme A, in general, compounds of the invention can be prepared by acylation of A-1 to provide compounds of formula A-2 and A-3. The ester coupling can preferentially occur between the 5′ alcohol in A-1 and an O-acyl propan-2-one oxime in the presence of an enzyme, such as NOVO enzyme-435, to afford compounds of the formula A-2. Alternatively, A-1 can be acylated by an acyl chloride in the presence of a base, such as pyridine, to afford compounds of the formula A-2. Bis-esterification can occur when A-1 is treated with an acyl chloride in the presence of a base, such as pyridine, or with a carboxylic acid in the presence of a coupling reagent, such as DIC, and a base, such as DMAP, to afford compounds of the formula A-3. Carboxylic acids are commercially available or may be synthesized from appropriate precursors.

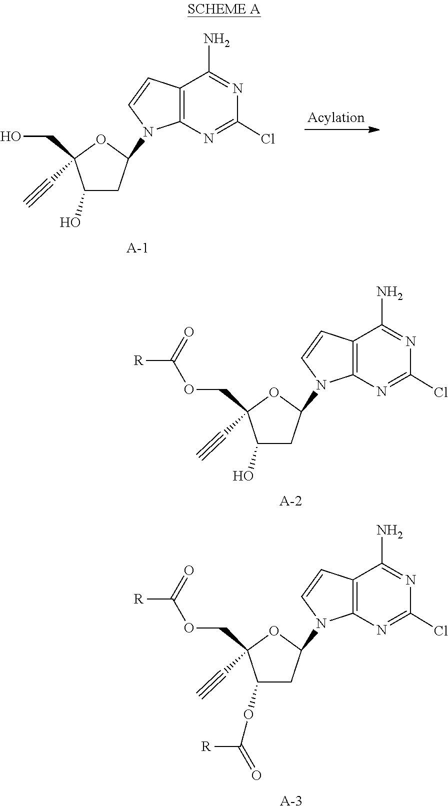

As illustrated in Scheme B, in general, compounds of the invention can be prepared by acylation of A-1 to provide compounds of formula B-1. The coupling of A-1 to a phosphoramidate such asInt-1 can occur in the presence of a base, such as LiTMP.

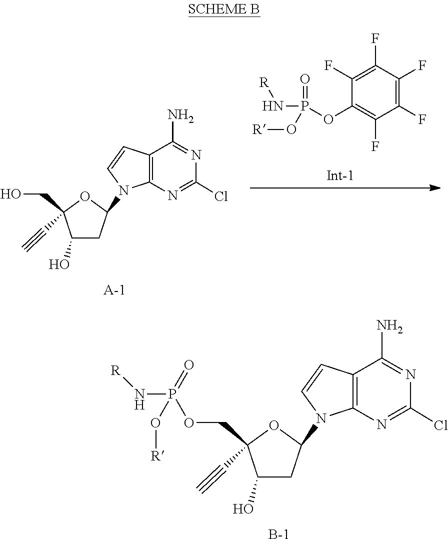

As illustrated in Scheme C, in general, compounds of the invention can be prepared by first placing a protecting group, such as a trialkylsilyl group, on the 5′ alcohol to afford C-1. The 3′ alcohol in C-1 can then be reacted with a chloro-formate or an alkyl-4-nitrophenyl carbonate in the presence of a base, such as DMAP or TEA, to afford products of formula C-2. The protecting group in C-2 can then be removed under appropriate conditions, including treatment with TBAF or triethylamine trihydrofluoride, to afford products of formula C-3.

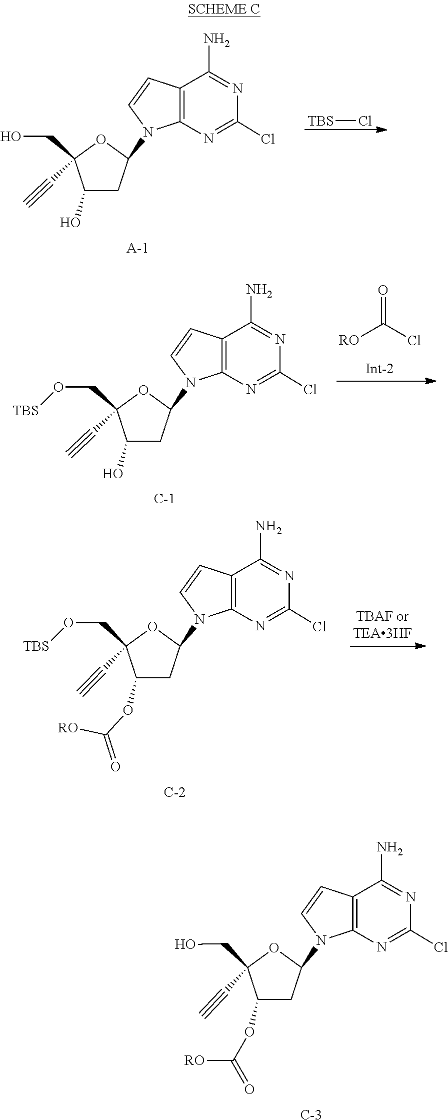

As illustrated in Scheme D, in general, compounds of the invention can be prepared by treating C-1 with an acyl chloride in the presence of a base, such as pyridine, or with a carboxylic acid in the presence of a coupling agent, such as DIC, and a base, such as DMAP, to afford products of formula D-1. The protecting group in D-1 can then be removed under appropriate conditions, including treatment with TBAF or triethylamine trihydrofluoride, to afford products of formula D-2.

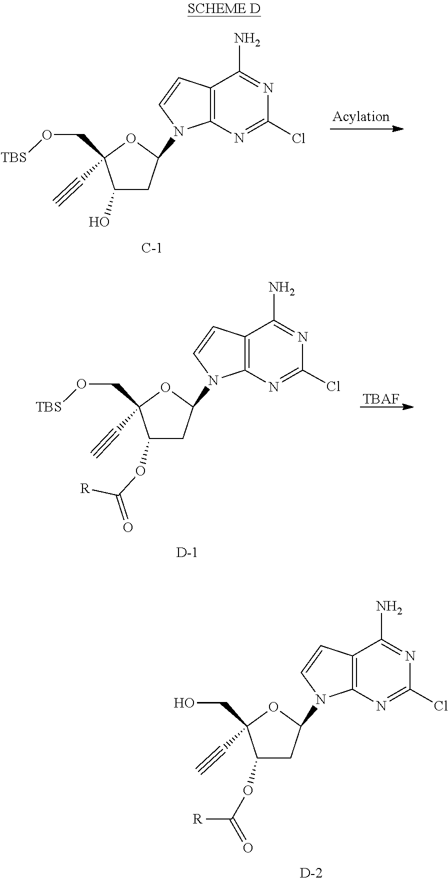

As illustrated in Scheme E, in general, compounds of the invention can be prepared by first protecting the 3′ alcohol and amino groups in C-1 with 4-methoxytrityl groups by reacting C-1 with 4-methoxytrityl chloride in the presence of an activating agent, such as silver nitrate, and a base, such as 2,4,6-trimethylpyridine to afford E-1. Selective deprotection of the 5′ alcohol of E-1 by treatment with a fluoride source, such as TBAF, affords E-2. Alcohol E-2 can be treated with a chloro-formate or an alkyl-4-nitrophenyl carbonate in the presence of a base, such as DMAP, pyridine or TEA, to afford products of formula E-3. The trityl protecting groups in E-3 can then be removed under acidic conditions, such as formic acid, aqueous TFA or acetic acid, to afford products of formula E-4.

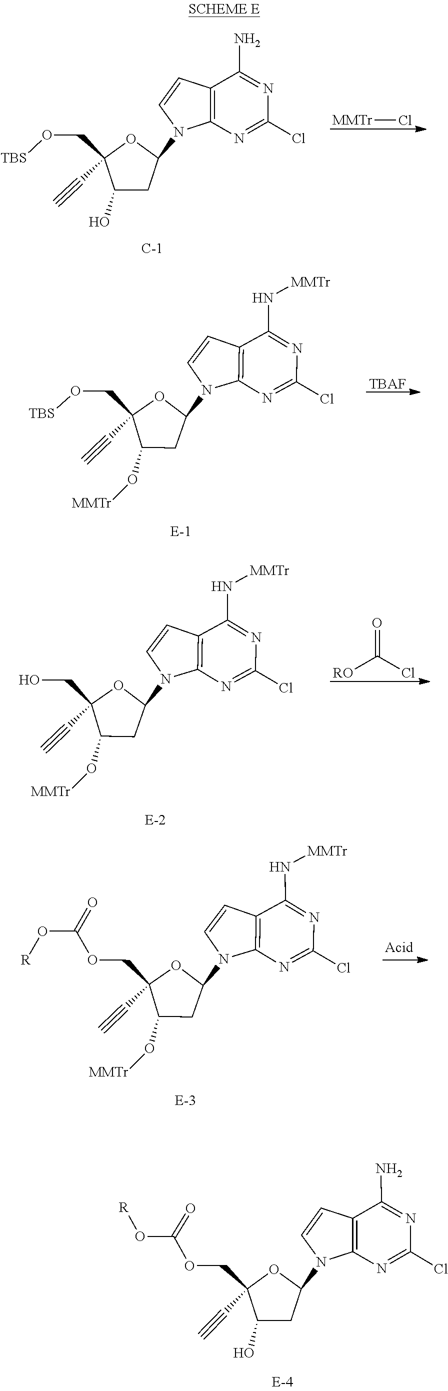

As illustrated in Scheme F, in general, compounds of the invention can be prepared by treating E-2 with an acyl chloride in the presence of a base, such as DMAP or TEA, or with a carboxylic acid in the presence of a coupling agent, such as DIC, and a base, such as DMAP, to afford products of formula F-1. The trityl protecting groups in F-1 can then be removed under acidic conditions, such as formic acid or aqueous TFA, to afford products of formula F-2.

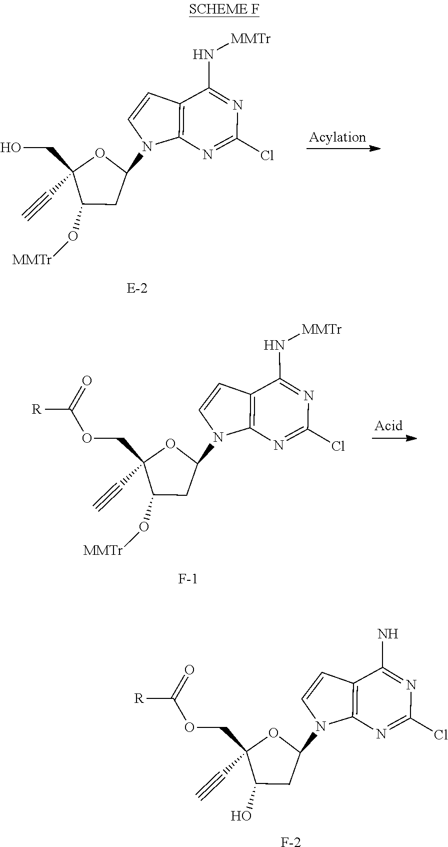

As illustrated in Scheme G, in general, compounds of the invention can be prepared by first placing protecting groups on both 3′ and 5′ alcohols, such as a trialkylsilyl groups, to afford G-1. The amine can be reacted by acylating G-1 with an acyl chloride in the presence of a base, such as DMAP or pyridine, to afford products of the formula G-2. Alternatively, it can be acylated by making a reactive intermediate of a caboxylic acid with reagents such as TCFH and imidazole or POCl₃ and a base. The protecting groups in G-2 can then be removed under appropriate conditions, including treatment with TBAF or triethylamine trihydrofluoride, to afford products of formula G-3.

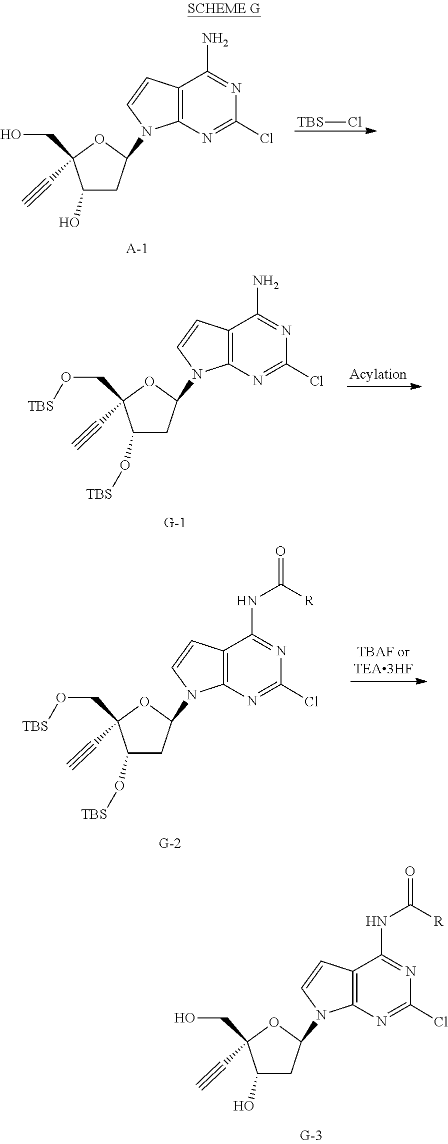

As illustrated in Scheme H, in general, compounds of the invention can be prepared by treating A-2 with an acyl chloride in the presence of a base, such as pyridine, to afford products of formula H-1.

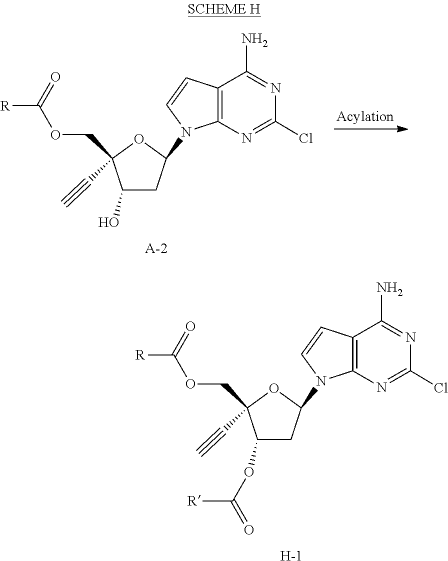

As illustrated in Scheme I, in general, compounds of the invention can be prepared by reacting the amine of G-1 with a chloro-formate, an alkyl-4-nitrophenyl carbonate, or alkyl (2,5-dioxopyrrolidin-1-yl) carbonate in the presence of a base, such as DMAP, NaH, or K₂CO₃, to afford products of the formulas I-1 and I-2. The protecting groups in formulas I-1 and I-2 can then be removed under appropriate conditions, including treatment with TBAF or triethylamine trihydrofluoride, to afford products of formulas I-3 and I-4.

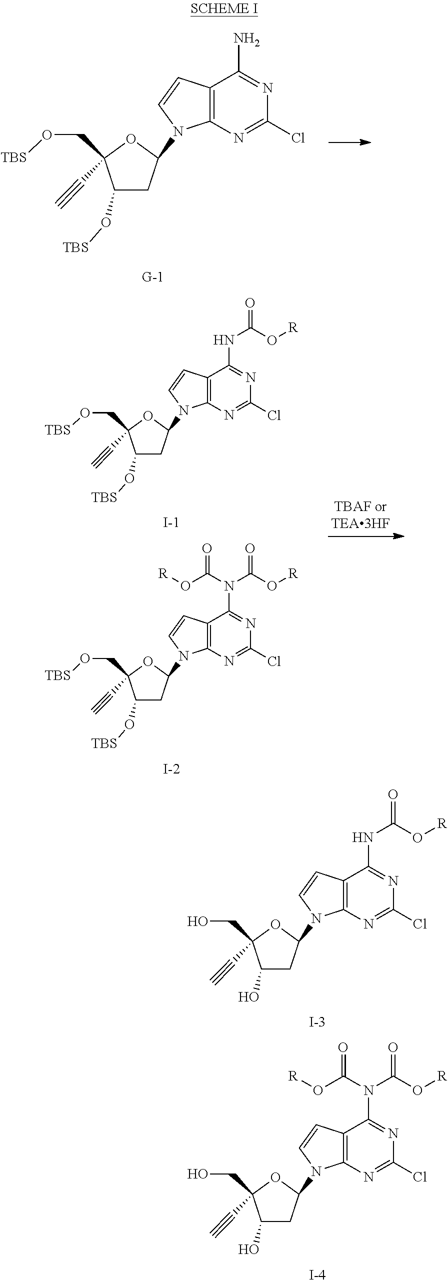

As illustrated in Scheme J, in general, compounds of the invention can be prepared by, reacting the 3′ alcohol of C-1 with 4-nitrophenyl chloroformate and a base such as pyridine or TEA to afford J-1. J-1 can then be reacted with an alcohol in the presence of a base, such as DMAP or TEA, to afford products of formula J-2. The protecting group in J-2 can then be removed under appropriate conditions, including treatment with TBAF or triethylamine trihydrofluoride, to afford products of formula J-3.

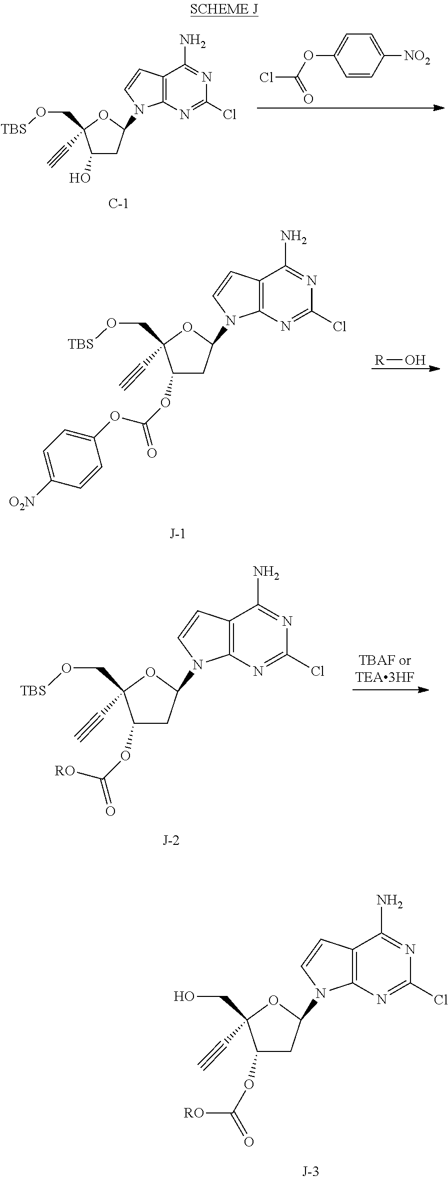

As illustrated in Scheme K, in general, compounds of the invention can be prepared by treating E-2 with a halomethyl ester or halomethyl carbonate in the presence of a base, such as NaH, to afford products of formula K-1. The trityl protecting groups in K-1 can then be removed under acidic conditions, such as formic acid or aqueous TFA, to afford products of formula K-2.

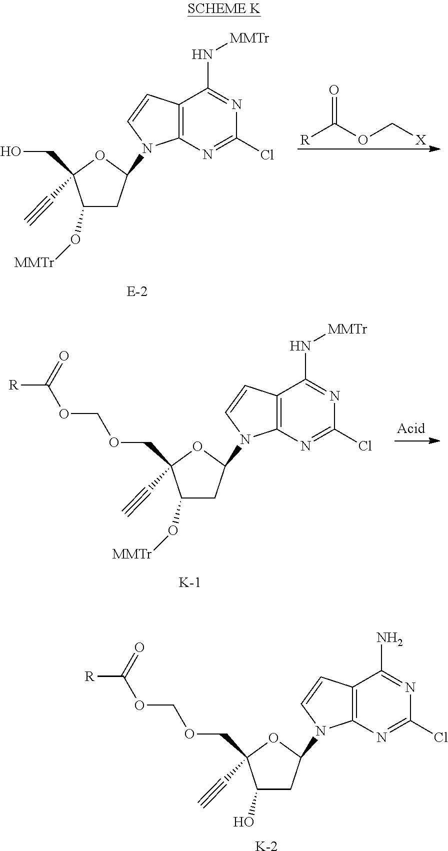

### Synthesis of Intermediates

In the section below, the preparation of certain intermediates useful in preparing the compounds of the invention are described.

### Intermediates 1 and 2

### (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) & 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (Intermediate 2)

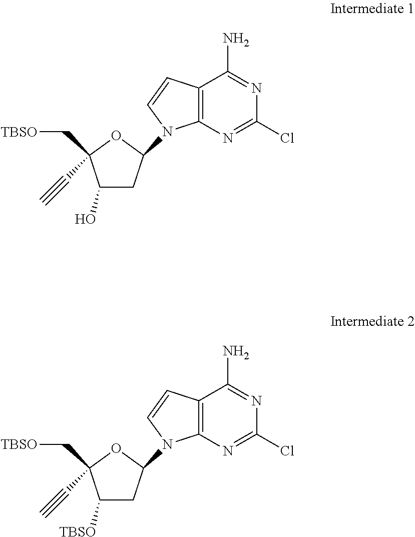 [SMILES: C#C[C@]1(CO[Si](C)(C)C(C)(C)C)O[C@@H](n2ccc3c(N)nc(Cl)nc32)C[C@@H]1O ; C#C[C@]1(CO[Si](C)(C)C(C)(C)C)O[C@@H](n2ccc3c(N)nc(Cl)nc32)C[C@@H]1O[Si](C)(C)C(C)(C)C]

To a stirred mixture of (2R,3S,5R)-5-(4-amino-2-chloro-7H-pyrrolo[2,3-d]pyrimidin-7-yl)-2-ethynyl-2-(hydroxymethyl)tetrahydrofuran-3-ol (0.5 g, 1.6 mmol) in DMF (7 mL) was added tert-butyldimethylchlorosilane (0.37 g, 2.4 mmol) and imidazole (0.44 g, 6.5 mmol) at room temperature under argon atmosphere. The resulting mixture was stirred at room temperature for 16 h. The reaction mixture was quenched by water (50 mL) and extracted with ethyl acetate (3×30 mL). The combined organic fractions was washed with brine (3×50 mL), dried over anhydrous sodium sulfate and filtered. The filtrate was concentrated under reduced pressure and the residue was purified by silica gel column chromatography, eluted with 1 to 40% EtOAc in Pet. ether to afford (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1), MS: m/z=421.25 [M−H]⁻, and 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (Intermediate 2), m/ [symbol] =537.40 [M+H]⁺.

### Intermediate 3

### ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methanol

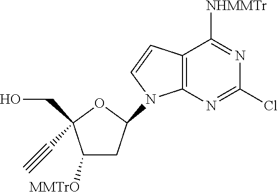

### Step 1: 7-((2R,4S,5R)-5-(((tert-butyldimethylsilyl)oxy)methyl)-5-ethynyl-4-((4-methoxyphenyl)diphenylmethoxy)tetrahydrofuran-2-yl)-2-chloro-N-((4-methoxyphenyl)diphenylmethyl)-7H-pyrrolo[2,3-d]pyrimidin-4-amine

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1, 0.24 g, 0.57 mmol) in DCM (10 mL) was added silver nitrate (0.29 g, 1.7 mmol), 4-methoxytrityl chloride (0.53 mg, 1.7 mmol) and 2,4,6-trimethylpyridine (0.41 g, 3.4 mmol) at room temperature under argon atmosphere. The resulting mixture was stirred at room temperature for 16 h. The reaction mixture was diluted with DCM (100 mL). The combined organic fractions was washed with brine (3×30 mL), dried over anhydrous sodium sulfate and filtered. The filtrate was concentrated under reduced pressure and the residue was purified by silica gel column chromatography, eluted with 1 to 40% EtOAc in Pet. ether to afford the title compound. MS: m/z=967.40 [M+H]⁺.

### Step 2: ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methanol

To a stirred mixture of 7-((2R,4S,5R)-5-(((tert-butyldimethylsilyl)oxy)methyl)-5-ethynyl-4-((4-methoxyphenyl)diphenylmethoxy)tetrahydrofuran-2-yl)-2-chloro-N-((4-methoxyphenyl)diphenylmethyl)-7H-pyrrolo[2,3-d]pyrimidin-4-amine (0.50 g, 0.52 mmol) in THF (5 mL) was added tetrabutylammonium fluoride (1 M in THF) (0.57 mL, 0.57 mmol) at room temperature under argon atmosphere. The resulting mixture was stirred at room temperature for 16 h. The reaction mixture was concentrated under reduced pressure and the residue was purified by silica gel column chromatography, eluted with 1 to 50% EtOAc in Pet. ether to afford the title compound. MS: m/ [symbol] =851.30 [M−H]⁻.

### Intermediate 4

### 2-(adamantan-1-yl)ethyl (4-nitrophenyl) carbonate

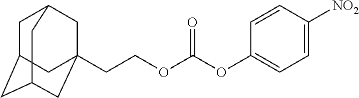 [SMILES: O=C(OCCC12CC3CC(CC(C3)C1)C2)Oc1ccc([N+](=O)[O-])cc1]

To a solution of 2-(adamantan-1-yl)ethanol (1.0 g, 5.5 mmol) and 4-nitrophenyl carbonochloridate (1.6 g, 8.2 mmol) in DCM (2 mL) were added TEA (1.2 mL, 8.2 mmol) at 0° C. under N₂ atmosphere. The resulting mixture was stirred at 25° C. for 16 h. The residue was purified by silica gel column chromatography, eluted with 0-70% of EtOAc in Pet. ether to afford the title compound. ¹H NMR (500 MHz, CDCl₃) δ 8.28 (d, J=9.1 Hz, 2H), 7.38 (d, J=9.0 Hz, 2H), 4.36 (t, J=7.4 Hz, 2H), 1.98 (s, 3H), 1.75-1.52 (m, 14H).

### Intermediate 5

### (2R,3S,5R)-5-(2-chloro-4-(((4-methoxyphenyl)diphenylmethyl)amino)-7H-pyrrolo[2,3-d]pyrimidin-7-yl)-2-ethynyl-2-(hydroxymethyl)tetrahydrofuran-3-ol

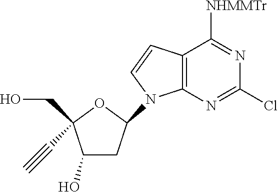

### Step 1: 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-N-((4-methoxyphenyl)diphenylmethyl)-7H-pyrrolo[2,3-d]pyrimidin-4-amine

To a solution of Intermediate 2 (1.8 g, 3.4 mmol) in DCM (20 mL) was added 4-mMethoxytriphenyl chloromethane (3.1 g, 10 mmol) and silver nitrate (1.7 g, 10 mmol), 2,4,6-collidine (2.4 g, 20 mmol) at 25° C. under N₂ atmosphere. The resulting mixture was stirred at 25° C. for 16 h. Then the mixture was quenched with saturated aqueous NH₄Cl (100 mL). The reaction mixture was extracted with ethyl acetate (3×). The combined organic layers were washed with brine (100 mL) and dried over anhydrous sodium sulfate. The filtrate was concentrated in vacuo. The residue was purified by silica gel column chromatography using 10%-60% gradient of ethyl acetate in petroleum ether as eluent. The fractions containing desired product were combined and concentrated under reduced pressure to afford the title compound. MS: m/ [symbol] =809.40 [M+H]⁺.

### Step 2: (2R,3S,5R)-5-(2-chloro-4-(((4-methoxyphenyl)diphenylmethyl)amino)-7H-pyrrolo[2,3-d]pyrimidin-7-yl)-2-ethynyl-2-(hydroxymethyl)tetrahydrofuran-3-ol

To a solution of 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-N-((4-methoxyphenyl)diphenylmethyl)-7H-pyrrolo[2,3-d]pyrimidin-4-amine (3.0 g, 3.7 mmol) in THF (30 mL) was added tetrabutylammonium fluoride (1.9 g, 7.4 mmol) at 25° C. under N₂ atmosphere. The resulting mixture was stirred at 25° C. for 2 h. Then the mixture was quenched with saturated aqueous NH₄Cl (200 mL). The reaction mixture was extracted with ethyl acetate (3×). The combined organic layers were washed with brine, dried over anhydrous sodium sulfate. The filtrate was concentrated in vacuo. The residue was purified by silica gel column chromatography using 10%-70% gradient of ethyl acetate in petroleum ether as eluent. The appropriate fractions were combined and concentrated under reduced pressure to afford the title compound. MS: m/ [symbol] =581.20 [M+H]⁺.

### Intermediate

### (2R,3S,5R)-2-((bis(4-methoxyphenyl)(phenyl)methoxy)methyl)-5-(2-chloro-4-(((4-methoxyphenyl)diphenylmethyl)amino)-7H-pyrrolo[2,3-d]pyrimidin-7-yl)-2-ethynyltetrahydrofuran-3-ol

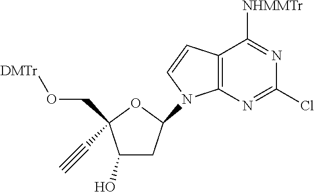

To a solution of Intermediate 5 (1.3 g, 2.2 mmol) in pyridine (20 mL) was added 4,4′-dimethoxytrityl chloride (0.91 g, 2.7 mmol) at 0° C. under N₂ atmosphere. The resulting mixture was stirred at 25° C. for 2 h. Then the mixture was concentrated in vacuo. The residue was purified by silica gel column chromatography using 10%-70% gradient of ethyl acetate in petroleum ether as eluent. The fractions containing desired product were combined and concentrated under reduced pressure to afford the title compound. MS: m/ [symbol] =883.45 [M+H]⁺.

Intermediates in the table below were made in a similar manner as Intermediate 4.

| | |
|---|---|
| Intermediate 7 | 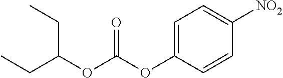 [SMILES: CCC(CC)OC(=O)Oc1ccc([N+](=O)[O-])cc1] |
| Intermediate 8 | 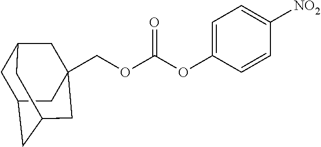 [SMILES: O=C(OCC12CC3CC(CC(C3)C1)C2)Oc1ccc([N+](=O)[O-])cc1] |
| Intermediate 9 | 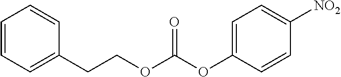 [SMILES: O=C(OCCc1ccccc1)Oc1ccc([N+](=O)[O-])cc1] |
| Intermediate 10 | 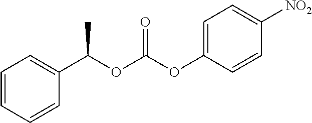 [SMILES: C[C@@H](OC(=O)Oc1ccc([N+](=O)[O-])cc1)c1ccccc1] |
| Intermediate 11 | 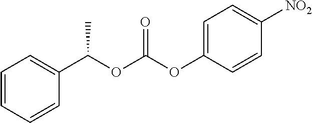 [SMILES: C[C@H](OC(=O)Oc1ccc([N+](=O)[O-])cc1)c1ccccc1] |
| Intermediate 12 | 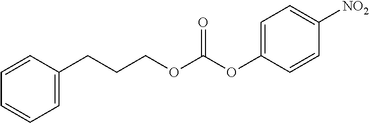 [SMILES: O=C(OCCCc1ccccc1)Oc1ccc([N+](=O)[O-])cc1] |
| Intermediate 13 | 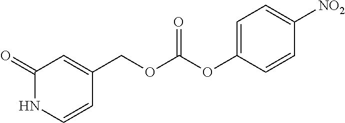 [SMILES: O=C(OCc1cc[nH]c(=O)c1)Oc1ccc([N+](=O)[O-])cc1] |
| Intermediate 14 | 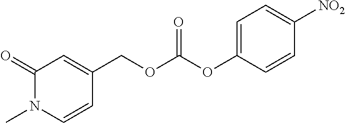 [SMILES: Cn1ccc(COC(=O)Oc2ccc([N+](=O)[O-])cc2)cc1=O] |
| Intermediate 15 | 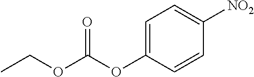 |
| Intermediate 16 | 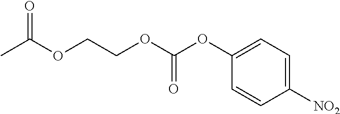 [SMILES: CC(=O)OCCOC(=O)Oc1ccc([N+](=O)[O-])cc1] |
| Intermediate 17 | 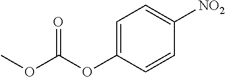 |
| Intermediate 18 | 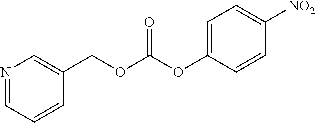 [SMILES: O=C(OCc1cccnc1)Oc1ccc([N+](=O)[O-])cc1] |
| Intermediate 19 | 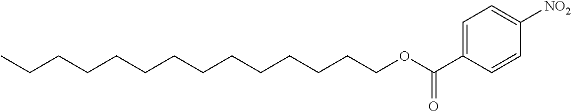 [SMILES: CCCCCCCCCCCCCCOC(=O)c1ccc([N+](=O)[O-])cc1] |
| Intermediate 20 | 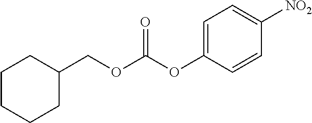 [SMILES: O=C(OCC1CCCCC1)Oc1ccc([N+](=O)[O-])cc1] |
| Intermediate 21 | 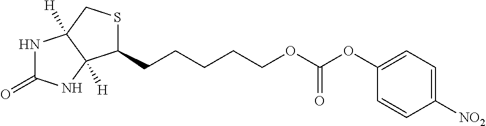 [SMILES: O=C1N[C@H]2[C@H](CS[C@H]2CCCCCOC(=O)Oc2ccc([N+](=O)[O-])cc2)N1] |
| Intermediate 22 | 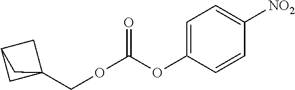 [SMILES: O=C(OCC12CC(C1)C2)Oc1ccc([N+](=O)[O-])cc1] |
| Intermediate 23 | 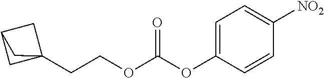 [SMILES: O=C(OCCC12CC(C1)C2)Oc1ccc([N+](=O)[O-])cc1] |

### Intermediate 24a and 24b

### (S or R) Isopropyl ((naphthalen-2-yloxy)(4-nitrophenoxy)phosphoryl)-L-alaninate and (R or S) Isopropyl ((naphthalen-2-yloxy)(4-nitrophenoxy)phosphoryl)-L-alaninate

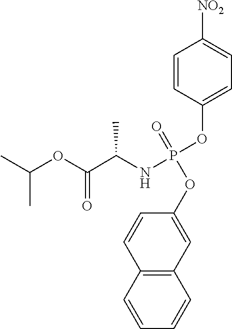 [SMILES: CC(C)OC(=O)[C@H](C)NP(=O)(Oc1ccc([N+](=O)[O-])cc1)Oc1ccc2ccccc2c1]

To a stirred mixture of 4-nitrophenyl phosphorodichloridate (0.76 g, 3.0 mmol) in DCM (5 mL) was added a solution of triethylamine (0.24 g, 2.4 mmol) and naphthalen-2-ol (0.43 g, 3.0 mmol) in DCM (5 mL) at −78° C. under argon atmosphere. The resulting mixture was stirred at −78° C. for 30 min. Then to resulting solution was added a mixture of isopropyl L-alaninate hydrochloride (0.50 g, 3.0 mmol) and triethylamine (0.76 g, 7.5 mmol) in DCM (10 mL). The resulting mixture was stirred at −78° C. for 30 min, then the mixture was allowed to warm to room temperature and stirred for 1 h. The reaction mixture was then concentrated under reduced pressure and the crude residue was subjected to silica gel chromatography eluting with 0-30% ethyl acetate in Pet. ether to afford crude product. The crude product was purified by prep-Chiral-HPLC with following condition: Column: (S, S)-Whelk-O 1 5 μm Kromasil 3*25 cm, 5 μm; Mobile Phase A: CO₂, Mobile Phase B: MEOH; Flow rate: 100 mL/min; Gradient: isocratic 30% B; Column Temperature (° C.): 35; Back Pressure (bar): 100; Wave Length: 220 nm; RT1 (min): 7.12; RT2 (min): 9.1.

The first eluting peak (RT1: 7.12 min) was combined and assumed as one compound, S or R, isopropyl ((naphthalen-2-yloxy)(4-nitrophenoxy)phosphoryl)-L-alaninate as Intermediate 24a. MS: m/ [symbol] =459.05 [M+H]⁺.

The second eluting peak (RT2: 9.1 min) was combined and assumed as one compound, R or S, isopropyl ((naphthalen-2-yloxy)(4-nitrophenoxy)phosphoryl)-L-alaninate as Intermediate 24b. MS: m/ [symbol] =459.05 [M+H]⁺.

### Intermediate 25

### Chloromethyl Dodecyl Carbonate

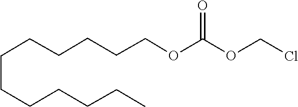 [SMILES: CCCCCCCCCCCCOC(=O)OCCl]

To a solution of chloromethyl carbonochloridate (0.69 g, 5.4 mmol) in DCM (20 mL) at 0° C. was added dropwise dodecan-1-ol (1.0 g, 5.4 mmol) and Py (0.43 mL, 5.4 mmol) in DCM (5 mL). The resulted mixture was stirred for 30 min. at 0° C. The reaction mixture was warmed to ambient temperature, diluted with DCM (20 mL) and washed with 0.5 M hydrochloric acid (3×), aqueous sodium hydrogen carbonate (3×), brine (3×), dried over Na₂SO₄ and filtered. The filtrate was concentrated under reduced pressure. The residue was purified by silica gel column chromatography, eluting with 0-4% EtOAc in Pet. ether to afford the title compound. ¹H NMR (400 MHz, CDCl₃) δ 5.73 (s, 2H), 4.24-4.10 (m, 2H), 1.75-1.64 (m, 2H), 1.44-1.18 (m, 18H), 0.90-0.83 (m, 3H).

### Intermediate 26

### Chloromethyl Phenyl Carbonate

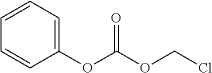 [SMILES: O=C(OCCl)Oc1ccccc1]

Intermediate 26 was made in a similar manner as Intermediate 25.

### EXAMPLES

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

### Example 1

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl isopropyl carbonate

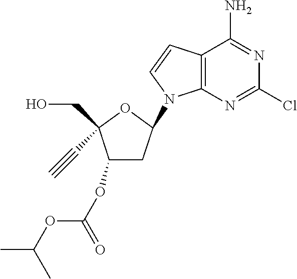 [SMILES: C#C[C@]1(CO)O[C@@H](n2ccc3c(N)nc(Cl)nc32)C[C@@H]1OC(=O)OC(C)C]

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl isopropyl carbonate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (50 mg, 0.12 mmol) in CH₂Cl₂ (2 mL) was added DMAP (14 mg, 0.12 mmol), TEA (0.016 mL, 0.12 mmol) and isopropyl chloroformate (29 mg, 0.24 mmol) at 25° C. under argon atmosphere. The resulting mixture was stirred at 25° C. for 16 h. The reaction was quenched by MeOH (0.1 mL). The reaction mixture was purified by Prep-TLC (EtOAc/Pet. ether (½) to afford the title compound. MS: m/ [symbol] =509.30 [M+H]⁺.

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl isopropyl carbonate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl isopropyl carbonate (45 mg, 0.088 mmol) in THF (2 mL) was added TBAF (0.13 mL, 0.13 mmol, 1M in THF) at 25° C. under argon atmosphere. The resulting mixture was stirred at 25° C. for 16 h. The reaction mixture was concentrated under reduced pressure and the residue was purified by RP-Flash chromatography, eluted with 0-60% acetonitrile in water to afford the title compound. MS: m/ [symbol] =395.05 [M+H]⁺. ¹H NMR (300 MHz, DMSO-d₆) δ 7.60 (s, 2H), 7.38 (d, J=3.6 Hz, 1H), 6.63 (d, J=3.6 Hz, 1H), 6.46-6.41 (m, 1H), 5.60 (t, J=6.0 Hz, 1H), 5.38-5.37 (m, 1H), 4.84-4.80 (m, 1H), 3.66-3.63 (m, 3H), 3.32 (s, 1H), 2.81-2.75 (m, 1H), 1.27 (d, J=6.3 Hz, 6H).

### Example 2

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl isobutyrate

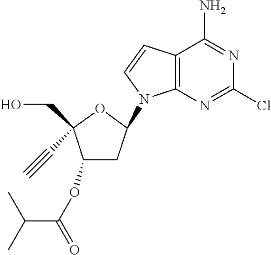 [SMILES: C#C[C@]1(CO)O[C@@H](n2ccc3c(N)nc(Cl)nc32)C[C@@H]1OC(=O)C(C)C]

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl isobutyrate

To a stirred mixture of DMAP (2.9 mg, 0.024 mmol) in DMF (3 mL) was added DIC (0.055 mL, 0.36 mmol), isobutyric acid (16 mg, 0.18 mmol) and (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (50 mg, 0.12 mmol). The resulting mixture was stirred for 16 h at room temperature. The reaction mixture was quenched by NH₄Cl (10 mL) and extracted with ethyl acetate (3×30 mL). The combined organic fractions was washed with brine (3×30 mL), dried over anhydrous sodium sulfate and filtered. The filtrate was concentrated under reduced pressure and the residue was purified by Prep-TLC (EtOAc/Pet. Ether=1:1) to afford the title compound. MS: m/ [symbol] =493.25 [M+H]⁺.

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl isobutyrate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl isobutyrate (50 mg, 0.10 mmol) in THF (3 mL) was added tetrabutylammonium fluoride (0.10 mL, 0.10 mmol, 1 M in THF). The resulting mixture was warmed to 25° C. and stirred for 4 h. The reaction mixture was purified by RP-Flash chromatography, eluted with 35-40% acetonitrile in water (aq. 0.5% FA) to afford the title compound. MS: m/ [symbol] =379.0 [M+H]⁺. ¹H-NMR (400 MHz, DMSO-d₆): δ 7.62 (s, 2H), 7.41-7.40 (m, 1H), 6.63-6.64 (m, 1H), 6.44-6.48 (m, 1H), 5.59-5.62 (m, 1H), 5.51-5.53 (m, 1H), 3.61-3.65 (m, 3H), 2.75-2.80 (m, 1H), 2.60-2.66 (m, 1H), 2.40-2.45 (m, 1H), 1.14-1.19 (m, 6H).

### Example 3

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl pentanoate

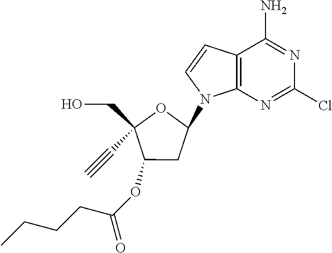 [SMILES: C#C[C@]1(CO)O[C@@H](n2ccc3c(N)nc(Cl)nc32)C[C@@H]1OC(=O)CCCC]

The title compound was made in a similar manner as Example 2 except using pentanoic acid in Step 1. MS: m/ [symbol] =393.10 [M+H]⁺. ¹H-NMR (400 MHz, DMSO-d₆): δ 7.76 (s, 2H), 7.40-7.39 (m, 1H), 6.64-6.63 (m, 1H), 6.47-6.44 (m, 1H), 5.61-5.53 (m, 2H), 3.64-3.61 (m, 2H), 3.33-3.31 (m, 1H), 2.77-2.75 (m, 1H), 2.44-2.40 (m, 3H), 1.60-1.54 (m, 2H), 1.36-1.33 (m, 2H), 0.94-0.87 (m, 3H).

### Example 4

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl decanoate

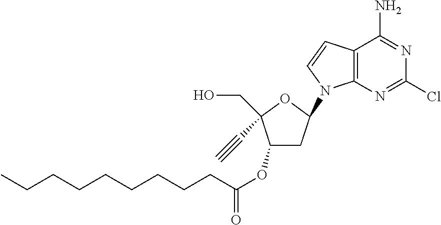 [SMILES: C#C[C@]1(CO)O[C@@H](n2ccc3c(N)nc(Cl)nc32)C[C@@H]1OC(=O)CCCCCCCCC]

The title compound was made in a similar manner as Example 2 except using decanoic acid in Step 1. MS: m/ [symbol] =463.00 [M+H]⁺. ¹H-NMR (400 MHz, CD₃OD): δ 7.33-7.30 (m, 1H), 6.59-6.47 (m, 2H), 5.67-5.64 (m, 1H), 3.89-3.78 (m, 2H), 3.13 (s, 1H), 2.90-2.83 (m, 1H), 2.51-2.42 (m, 3H), 1.70-1.60 (m, 2H), 1.40-1.25 (m, 12H), 0.91-0.81 (m, 3H).

### Example 5

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl benzoate

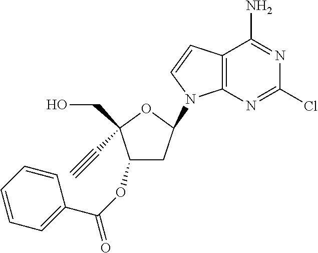 [SMILES: C#C[C@]1(CO)O[C@@H](n2ccc3c(N)nc(Cl)nc32)C[C@@H]1OC(=O)c1ccccc1]

The title compound was made in a similar manner as Example 2 except using benzoic acid in Step 1. MS: m/ [symbol] =413.00 [M+H]⁺. ¹H-NMR (400 MHz, CD₃OD): δ 8.14 (s, 2H), 7.67-7.63 (m, 1H), 7.54-7.50 (m, 2H), 7.39-7.37 (m, 1H), 6.67-6.61 (m, 2H), 5.88 (s, 1H), 3.93-3.89 (m, 2H), 3.10-2.97 (m, 2H), 2.67-2.63 (m, 1H).

### Example 6

### (2R,3S,5R)-5-(4-amino-2-chloro-7H-pyrrolo[2,3-d]pyrimidin-7-yl)-2-((benzoyloxy)methyl)-2-ethynyltetrahydrofuran-3-yl benzoate

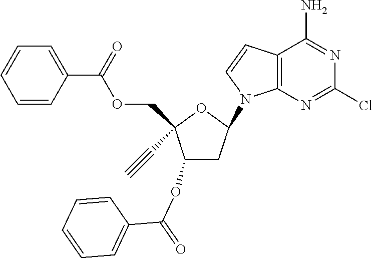 [SMILES: C#C[C@]1(COC(=O)c2ccccc2)O[C@@H](n2ccc3c(N)nc(Cl)nc32)C[C@@H]1OC(=O)c1ccccc1]

To a stirred mixture of (2R,3S,5R)-5-(4-amino-2-chloro-7H-pyrrolo[2,3-d]pyrimidin-7-yl)-2-ethynyl-2-(hydroxymethyl)tetrahydrofuran-3-ol (compound A) (30 mg, 0.097 mmol), 4-dimethylaminopyridine (4.8 mg, 0.039 mmol) and DCC (40 mg, 0.19 mmol) in DMF (2 mL) was added benzoic acid (71 mg, 0.58 mmol) at room temperature under argon atmosphere. The resulting mixture was stirred at room temperature for 5 h. The reaction mixture was quenched by NH₄Cl (10 mL) and extracted with ethyl acetate (3×30 mL). The combined organic fractions was washed with brine (3×30 mL), dried over anhydrous sodium sulfate and filtered. The filtrate was concentrated under reduced pressure and the residue was purified by prep-TLC using 40% EtOAc in Pet. ether to give the title compound. MS: m/ [symbol] =517.05 [M+H]⁺. ¹H NMR (300 MHz, CD₃Cl) δ 8.13 (d, J=1.2 Hz, 2H), 8.04 (d, J=7.2 Hz, 2H), 7.65-7.40 (m, 6H), 7.10 (d, J=3.6 Hz, 1H), 6.79 (t, J=6.6 Hz, 1H), 6.42 (d, J=3.6 Hz, 1H), 5.98-5.94 (m, 1H), 5.81 (s, 2H), 4.82 (d, J=11.7 Hz, 1H), 4.65 (d, J-=12.0 Hz, 1H), 2.99-2.81 (m, 2H), 2.69 (s, 1H).

### Example 7

### (2R,3S,5R)-5-(4-amino-2-chloro-7H-pyrrolo[2,3-d]pyrimidin-7-yl)-2-ethynyl-2-((pentanoyloxy)methyl)tetrahydrofuran-3-yl pentanoate

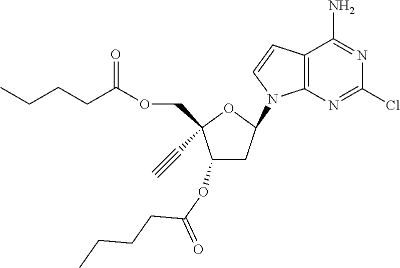 [SMILES: C#C[C@]1(COC(=O)CCCC)O[C@@H](n2ccc3c(N)nc(Cl)nc32)C[C@@H]1OC(=O)CCCC]

The title compound was made in a similar manner as Example 6 except using pentanoic acid in Step 1. MS: m/ [symbol] =477.15 [M+H]⁺. ¹H NMR (300 MHz, CD₃Cl) δ 7.12 (d, J=3.9 Hz, 1H), 6.71 (t, J=6.6 Hz, 1H), 6.43 (d, J=3.9 Hz, 1H), 5.60-5.56 (m, 1H), 5.47 (s, 2H), 4.45 (d, J=12.0 Hz, 1H), 4.35 (d, J=11.7 Hz, 1H), 2.73-2.63 (m, 3H), 2.45-2.36 (m, 4H), 1.70-1.61 (m, 4H), 1.43-1.32 (m, 4H), 0.97-0.89 (m, 6H).

### Example 8

### Isopropyl ((S)-(((2R,3S,5R)-5-(4-amino-2-chloro-7H-pyrrolo[2,3-d]pyrimidin-7-yl)-2-ethynyl-3-hydroxytetrahydrofuran-2-yl)methoxy)(phenoxy)phosphoryl)-L-alaninate

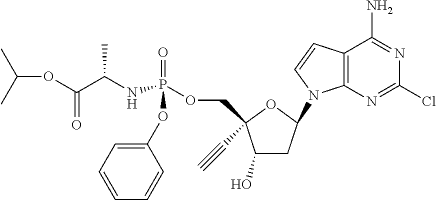

To a stirred mixture of (2R,3S,5R)-5-(4-amino-2-chloro-7H-pyrrolo[2,3-d]pyrimidin-7-yl)-2-ethynyl-2-(hydroxymethyl)tetrahydrofuran-3-ol (30 mg, 0.097 mmol) in DMF (1 mL) was added 2,2,6,6-tetramethylpiperidinylmagnesium chloride lithium chloride complex solution (24 mg, 0.097 mmol) at 0° C. under argon atmosphere. The resulting mixture was stirred at 0° C. for 0.5 h. Then a solution of isopropyl ((S)-(perfluorophenoxy)(phenoxy)phosphoryl)-L-alaninate (44 mg, 0.097 mmol) in DMF (0.2 mL) was added to the reaction. The resulting mixture was stirred at 0° C. for 1 h. The reaction was purified by RP-Flash chromatography, eluted with 0 to 30% acetonitrile in water (5 mM NH₄HCO₃) to afford the title compound. MS: m/ [symbol] =578.15 [M+H]⁺. ¹H NMR (300 MHz, CDCl₃) δ 7.32-7.29 (m, 2H), 7.23-7.15 (m, 3H), 7.07 (d, J=3.6 Hz, 1H), 6.62 (dd, J=7.5, 4.8 Hz, 1H), 6.35 (d, J=3.6 Hz, 1H), 5.25 (s, 2H), 5.05-4.96 (m, 1H), 4.73-4.71 (m, 1H), 4.35-4.30 (m, 2H), 4.00-3.94 (m, 1H), 3.72-3.65 (m, 1H), 3.29-3.27 (m, 1H), 2.71-2.60 (m, 3H), 1.38-1.36 (m, 3H), 1.26-1.20 (m, 6H). ³¹P NMR (121 MHz, CDCl₃) δ 2.86 (s, 1P).

### Example 9

### ((2R,3S,5R)-5-(4-amino-2-chloro-7H-pyrrolo[2,3-d]pyrimidin-7-yl)-2-ethynyl-3-hydroxytetrahydrofuran-2-yl)methyl benzoate

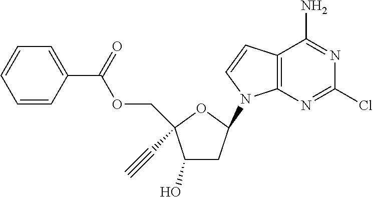 [SMILES: C#C[C@]1(COC(=O)c2ccccc2)O[C@@H](n2ccc3c(N)nc(Cl)nc32)C[C@@H]1O]

To a solution of (2R,3S,5R)-5-(4-amino-2-chloro-7H-pyrrolo[2,3-d]pyrimidin-7-yl)-2-ethynyl-2-(hydroxymethyl)tetrahydrofuran-3-ol (0.10 g, 0.32 mmol) in THF (20 mL) were added propan-2-one O-benzoyl oxime (57 mg, 0.32 mmol), immobilized NOVO enzyme-435 (400 U) and molecular sieves (1 g). The resulted mixture was stirred for 48 h at 50° C. The reaction mixture was cooled down to ambient temperature, and filtered. The filtrate was concentrated under vacuum. The residue was purified by RP-flash chromatography, eluted with 0-56% acetonitrile in water (5 mM NH₄HCO₃) to afford the title compound. MS: m/ [symbol] =411.05 [M−H]⁻. ¹H NMR (300 MHz, CD₃OD) δ 7.97-7.94 (m, 2H), 7.60-7.57 (m, 1H), 7.48-7.45 (m, 2H), 7.14 (d, J=3.9 Hz, 1H), 6.49-6.45 (m, 2H), 4.90-4.85 (m, 1H), 4.73-4.69 (m, 1H), 4.49-4.45 (m, 1H), 3.17 (s, 1H), 2.78-2.76 (m, 1H), 2.70-2.63 (m, 1H).

### Example 10

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl decyl carbonate

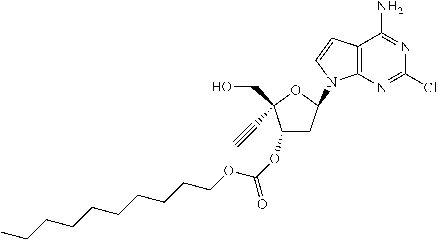 [SMILES: C#C[C@]1(CO)O[C@@H](n2ccc3c(N)nc(Cl)nc32)C[C@@H]1OC(=O)OCCCCCCCCCC]

The title compound was made in a similar manner as Example 12 except using decyl chloroformate in Step 1. MS: m/ [symbol] =493.15 [M+H]⁺. ¹H NMR (400 MHz, DMSO-d₆) δ 7.60 (s, 2H), 7.38 (d, J=3.6 Hz, 1H), 6.63 (d, J=3.6 Hz, 1H), 6.46-6.42 (m, 1H), 5.60 (s, 1H), 5.39-5.37 (m, 1H), 4.14-4.11 (m, 2H), 3.65-3.62 (m, 3H), 3.18-3.12 (m, 1H), 2.84-2.77 (m, 1H), 1.64-1.59 (m, 2H), 1.34-1.25 (m, 12H), 0.95-0.84 (m, 5H).

Alternatively, Example 10 can be prepared as follows:

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl decyl carbonate

Under an atmosphere of N₂, (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (2.2 g, 5.3 mmol) was dissolved in anhy pyridine (25 mL) and put into a cold water bath. To this solution, was added decyl carbonochloridate (1.8 mL, 8.0 mmol) dropwise and the reaction was stirred at RT for one hour. The reaction was quenched with water, concentrated under reduced pressure, and then partitioned between water and diethyl ether. The organics were washed with water (3×) and then brine (1×), dried over Na₂SO₄, filtered and concentrated under reduced pressure. The residue was redissolved in DCM and purified on NP silica gel column (120 g ISCO-gold) eluting 0-40% EtOAc/hexanes to provide the title compound. MS: m/ [symbol] =607.7 [M+H]⁺.

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl decyl carbonate

A solution of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl decyl carbonate (2.5 g, 4.1 mmol) in anhy THF (30 mL) under an atmosphere of N₂, was put into a cold water bath. To this was added TBAF (1M in THF) (4.1 mL, 4.1 mmol). The reaction was stirred at RT for 90 min, and then diluted with ether and water. The organics were washed with water (3×) and then brine (lx), dried over Na₂SO₄, filtered and concentrated under reduced pressure. The residue was redissolved in DCM and purified on NP silica gel column (120 g ISCO-gold) eluting 0-60% EtOAc/hexanes. The appropriate fractions were concentrated under reduced pressure, and then redissolved in ether and concentrated under reduced pressure (2×). The residue was recrystallized from diethyl ether to provide the title compound. MS: m/ [symbol] =493.8 [M+H]⁺. ¹H NMR (500 MHz, CDCl₃) δ 6.96 (d, J=2.5 Hz, 1H), 6.33 (d, J=2.4 Hz, 1H), 6.25 (dd, J=9.5, 5.3 Hz, 1H), 5.83 (d, J=12.0 Hz, 1H), 5.63 (d, J=6.2 Hz, 1H), 5.48 (s, 2H), 4.27-4.14 (m, 2H), 4.08-4.03 (m, 1H), 3.96 (t, J=12.3 Hz, 1H), 3.37-3.27 (m, 1H), 2.62 (s, 1H), 2.47 (dd, J=13.9, 5.4 Hz, 1H), 1.75-1.67 (m, 2H), 1.42-1.23 (m, 14H), 0.88 (t, J=6.4 Hz, 3H).

### Example 11

### ((2R,3S,5R)-5-(4-amino-2-chloro-7H-pyrrolo[2,3-d]pyrimidin-7-yl)-2-ethynyl-3-hydroxytetrahydrofuran-2-yl)methyl isobutyrate

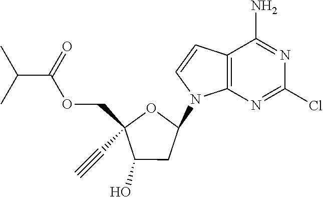 [SMILES: C#C[C@]1(COC(=O)C(C)C)O[C@@H](n2ccc3c(N)nc(Cl)nc32)C[C@@H]1O]

The title compound was made in a similar manner as Example 9 except using propan-2-one O-isobutyryl oxime in Step 1. MS: m/ [symbol] =379.15 [M+H]⁺. ¹H NMR (400 MHz, DMSO-d₆) δ 7.57 (s, 2H), 7.31 (d, J=3.6 Hz, 1H), 6.62 (d, J=3.6 Hz, 1H), 6.40-6.36 (m, 1H), 6.06 (s, 1H), 4.56-4.52 (m, 1H), 4.41-4.38 (m, 1H), 4.08-4.05 (m, 1H), 3.67 (s, 1H), 2.66-2.58 (m, 2H), 2.51-2.33 (m, 1H), 1.07-1.03 (m, 6H).

### Example 12

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl phenyl carbonate

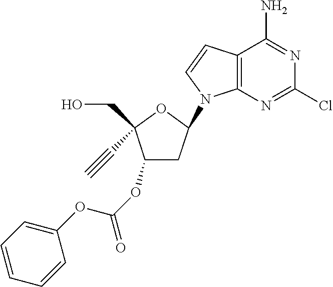 [SMILES: C#C[C@]1(CO)O[C@@H](n2ccc3c(N)nc(Cl)nc32)C[C@@H]1OC(=O)Oc1ccccc1]

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl phenyl carbonate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (50 mg, 0.118 mmol) in DCM (3 mL) were added TEA (0.025 mL, 0.177 mmol), DMAP (14.44 mg, 0.118 mmol) and phenyl chloroformate (22.21 mg, 0.142 mmol). The resulting mixture was warmed to 25° C. and stirred for 16 h. The reaction was quenched by MeOH (0.1 mL) and then concentrated under reduced pressure. The residue was purified by Prep-TLC (EtOAc/Pet. ether=1:1) to afford the title compound. MS: m/ [symbol] =543.25 [M+H]⁺.

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl phenyl carbonate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl phenyl carbonate (50 mg, 0.092 mmol) in THF (3 mL) was added triethylamine trihydrofluoride (119 mg, 0.737 mmol). The resulting mixture was warmed to 25° C. and stirred for 4 h. The reaction mixture was purified by RP-flash chromatography, eluted with 0-60% acetonitrile in water to afford the title compound. MS: m/ [symbol] =428.90 [M+H]⁺. ¹H-NMR (400 MHz, CD₃OD): δ 7.47-7.43 (m, 2H), 7.35 (d, J=3.6 Hz, 1H), 7.29-7.33 (m, 1H), 7.23-7.26 (m, 2H), 6.62 (d, J=3.6 Hz, 1H), 6.58-6.54 (m, 1H), 5.64-5.61 (m, 1H), 3.94-3.86 (m, 2H), 3.32 (s, 1H), 3.06-2.98 (m, 1H), 2.70-2.64 (m, 1H).

### Example 13

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl pentyl carbonate

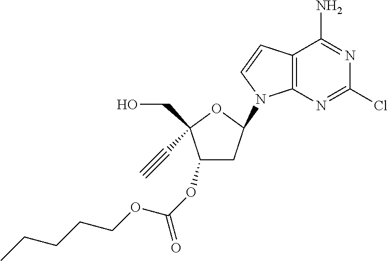 [SMILES: C#C[C@]1(CO)O[C@@H](n2ccc3c(N)nc(Cl)nc32)C[C@@H]1OC(=O)OCCCCC]

The title compound was made in a similar manner as Example 12 except using pentyl carbonochloridate in Step 1. MS: m/ [symbol] ==423.10 [M+H]⁺. ¹H NMR (400 MHz, CDCl₃) δ 7.00 (d, J=3.6 Hz, 1H), 6.37 (d, J=3.6 Hz, 1H), 6.29-6.25 (m, 1H), 5.63-5.59 (m, 4H), 4.26-4.15 (m, 2H), 4.07-4.04 (m, 1H), 3.97-3.94 (m, 1H), 3.33-3.26 (m, 1H), 2.62 (s, 1H), 2.48-2.46 (m, 1H), 1.75-1.68 (m, 2H), 1.39-1.34 (m, 4H), 0.94-0.90 (m, 3H).

### Example 14

### ((2R,3S,5R)-5-(4-amino-2-chloro-7H-pyrrolo[2,3-d]pyrimidin-7-yl)-2-ethynyl-3-hydroxytetrahydrofuran-2-yl)methyl pentyl carbonate

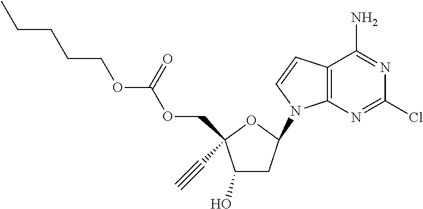 [SMILES: C#C[C@]1(COC(=O)OCCCCC)O[C@@H](n2ccc3c(N)nc(Cl)nc32)C[C@@H]1O]

### Step 1: ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methyl pentyl carbonate

To a solution of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methanol (Intermediate 3) (0.10 g, 0.12 mmol) and 4-dimethylaminopyridine (14 mg, 0.12 mmol) in DCM (5 mL) were added TEA (0.024 mL, 0.18 mmol), pentyl carbonochloridate (35 mg, 0.23 mmol) at 0° C. under N₂ atmosphere. The resulting mixture was stirred at 25° C. for 16 h. The reaction mixture was purified by prep-TLC eluted with DCM:MeOH=10:1 to afford the title compound. MS: m/ [symbol] =967.55 [M+H]⁺.

### Step 2: ((2R,3S,5R)-5-(4-amino-2-chloro-7H-pyrrolo[2,3-d]pyrimidin-7-yl)-2-ethynyl-3-hydroxytetrahydrofuran-2-yl)methyl pentyl carbonate

To a mixture of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methyl pentyl carbonate (0.10 g, 0.10 mmol) in water (1 mL) were added formic acid (4.0 mL, 0.10 mmol) at 25° C. under N₂ atmosphere. The resulting mixture was stirred at 25° C. for 16 h. The residue was purified by RP-Flash (Column: C18 40 g Column; Mobile Phase A: water (0.1% NH₄HCO₃), Mobile Phase B: MeCN; Flow rate: 50 mL/min; Gradient: 2% B to 30% B in 30 min, Detector: UV 210 nm; RT=28 min) to afford the title compound. MS: m/ [symbol] =423.10 [M+H]⁺. ¹H NMR (400 MHz, DMSO-d₆) δ 7.63-7.56 (m, 2H), 7.30-7.29 (m, 1H), 6.62-6.61 (m, 1H), 6.39-6.36 (m, 1H), 5.76-5.73 (m, 1H), 4.56-4.52 (m, 1H), 4.41-4.36 (m, 1H), 4.22-4.17 (m, 1H), 4.08-4.05 (m, 2H), 3.61-3.58 (m, 1H), 2.62-2.35 (m, 2H), 1.60-1.50 (m, 2H), 1.30-1.20 (m, 4H), 0.88-0.80 (m, 3H).

### Example 15

### ((2R,3S,5R)-5-(4-amino-2-chloro-7H-pyrrolo[2,3-d]pyrimidin-7-yl)-2-ethynyl-3-hydroxytetrahydrofuran-2-yl)methyl pentanoate

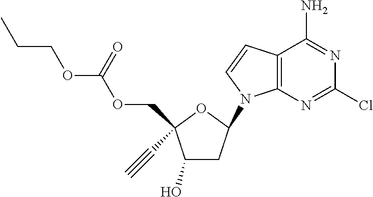 [SMILES: C#C[C@]1(COC(=O)OCCC)O[C@@H](n2ccc3c(N)nc(Cl)nc32)C[C@@H]1O]

The title compound was made in a similar manner as Example 14 except using hexanoyl chloride in Step 1. MS: m [symbol] =393.05 [M+H]⁺. ¹H-NMR (300 MHz, CD₃OD): δ 7.21-7.18 (m, 1H), 6.62-6.59 (m, 1H), 6.47-6.41 (m, 1H), 4.79-4.70 (m, 1H), 4.51-4.45 (m, 1H), 4.23-4.18 (m, 1H), 3.12 (s, 1H), 2.77-2.57 (m, 2H), 2.40-2.20 (m, 2H), 1.60-1.48 (m, 2H), 1.35-1.22 (m, 2H), 0.94-0.85 (m, 3H).

### Example 16

### ((2R,3S,5R)-5-(4-amino-2-chloro-7H-pyrrolo[2,3-d]pyrimidin-7-yl)-2-ethynyl-3-hydroxytetrahydrofuran-2-yl)methyl palmitate

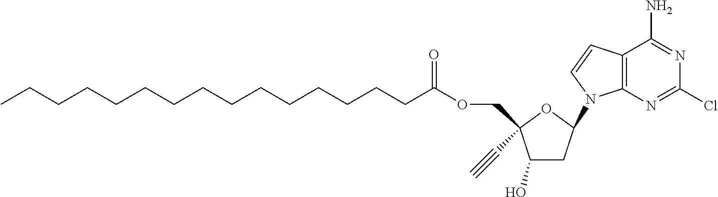 [SMILES: C#C[C@]1(COC(=O)CCCCCCCCCCCCCCC)O[C@@H](n2ccc3c(N)nc(Cl)nc32)C[C@@H]1O]

The title compound was made in a similar manner as Example 14 except using palmitoyl chloride in Step 1. MS: m/ [symbol] =547.00 [M+H]⁺. ¹H NMR (500 MHz, CDCl₃) δ 7.05 (d, J=3.6 Hz, 1H), 6.60-6.54 (m, 1H), 6.37 (d, J=3.7 Hz, 1H), 5.31 (s, 2H), 4.65 (q, J=7.2 Hz, 1H), 4.48-4.36 (m, 2H), 2.76-2.69 (m, 2H), 2.66-2.56 (m, 1H), 2.40-2.31 (m, 3H), 1.65-1.55 (m, 2H), 1.30-1.26 (m, 24H), 0.88 (t, J=6.5 Hz, 3H).

### Example 17

### ((2R,3S,5R)-5-(4-amino-2-chloro-7H-pyrrolo[2,3-d]pyrimidin-7-yl)-2-ethynyl-3-hydroxytetrahydrofuran-2-yl)methyl phenyl carbonate

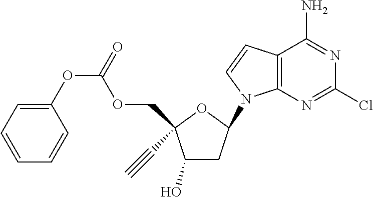 [SMILES: C#C[C@]1(COC(=O)Oc2ccccc2)O[C@@H](n2ccc3c(N)nc(Cl)nc32)C[C@@H]1O]

The title compound was made in a similar manner as Example 14 except using phenyl carbonochloridate in Step 1. MS: m/ [symbol] =428.95 [M+H]⁺. ¹H NMR (400 MHz, CD₃OD) δ 7.38-7.35 (m, 2H), 7.33-7.22 (m, 2H), 7.06-7.04 (m, 2H), 6.59-6.52 (m, 2H), 4.89-4.77 (m, 1H), 4.66-4.65 (m, 1H), 4.46-4.40 (m, 1H), 3.31-3.12 (m, 1H), 2.69-2.59 (m, 2H).

### Example 18

### ((2R,3S,5R)-5-(4-amino-2-chloro-7H-pyrrolo[2,3-d]pyrimidin-7-yl)-2-ethynyl-3-hydroxytetrahydrofuran-2-yl)methyl decyl carbonate

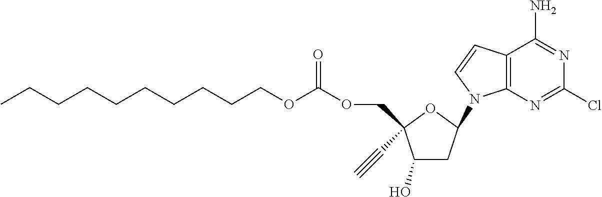 [SMILES: C#C[C@]1(COC(=O)OCCCCCCCCCC)O[C@@H](n2ccc3c(N)nc(Cl)nc32)C[C@@H]1O]

### Step 1: ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methyl decyl carbonate

To a solution of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methanol (Intermediate 3) (100 mg, 0.117 mmol) and 4-dimethylaminopyridine (14.32 mg, 0.117 mmol) in DCM (2 mL) were added TEA (0.024 mL, 0.176 mmol), decyl carbonochloridate (51.7 mg, 0.234 mmol) at 0° C. under N₂ atmosphere. The resulting mixture was stirred at 25° C. for 16 h. The reaction mixture was purified by prep-TLC eluted with DCM:MeOH=10:1 to afford the title compound. MS: m/ [symbol] =1037.55 [M+H]⁺.

### Step 2: ((2R,3S,5R)-5-(4-amino-2-chloro-7H-pyrrolo[2,3-d]pyrimidin-7-yl)-2-ethynyl-3-hydroxytetrahydrofuran-2-yl)methyl decyl carbonate

To a solution of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methyl decyl carbonate (100 mg, 0.096 mmol) in water (1 mL) were added TFA (4 mL, 51.9 mmol) at 25° C. under N₂ atmosphere. The resulting mixture was stirred at 25° C. for 16 h. The residue was purified by RP-Flash (Column: C18 40 g Column; Mobile Phase A: water (0.1% NH₄HCO₃), Mobile Phase B: MeCN; Flow rate: 40 mL/min; Gradient: 2% B to 30% B in 30 min, Detector: UV 210 nm; RT=28 min). The fractions containing desired product were combined and concentrated under reduced pressure to afford the title compound. MS: m/ [symbol] =493.05 [M+H]⁺. ¹H NMR (400 MHz, CD₃OD) δ 7.21-7.20 (m, 1H), 6.59-6.58 (m, 1H), 6.53-6.50 (m, 1H), 4.68-4.65 (m, 1H), 4.58-4.48 (m, 1H), 4.36-4.28 (m, 1H), 4.17-4.03 (m, 2H), 3.21-3.14 (m, 1H), 2.63-2.55 (m, 2H), 1.65-1.62 (m, 2H), 1.41-1.19 (m, 14H), 0.96-0.86 (m, 3H).

Alternatively, Example 18 can be prepared as follows:

In a flame dried flask under an atmosphere of N₂, (2R,3S,5R)-5-(4-amino-2-chloro-7H-pyrrolo[2,3-d]pyrimidin-7-yl)-2-ethynyl-2-(hydroxymethyl)tetrahydrofuran-3-ol (compound A) (2.5 g, 8.1 mmol) was dissolved in anhy pyridine (50 mL) and put into an ice bath. To this was added decyl carbonochloridate (2.1 mL, 8.9 mmol) dropwise over 5 mins and then stirred at 0° C. for 80 mins. The reaction was quenched with anhy methanol (5 mL) and stirred for 5 mins. The reaction was concentrated under reduced pressure and then partitioned between water and ether. The organics were washed with water (3×) and brine (lx), dried over Na₂SO₄, filtered, and concentrated under reduced pressure. The residue was redissolved in DCM/toluene and purified on NP silica gel column (120 g ISCO-gold) eluting 0-50% EtOAc/hexanes. The appropriate fractions were concentrated under reduced pressure, and then redissolved in ether and concentrated under reduced pressure (2×). The residue was recrystallized from diethyl ether to provide the title compound. MS: m/ [symbol] =493.5[M+H]⁺. ¹H NMR (500 MHz, CDCl₃) δ 7.13 (d, J=3.6 Hz, 1H), 6.66 (t, J=6.3 Hz, 1H), 6.37 (d, J=3.6 Hz, 1H), 5.39 (s, 2H), 4.67 (q, J=6.5 Hz, 1H), 4.51 (d, J=11.6 Hz, 1H), 4.41 (d, J=11.6 Hz, 1H), 4.16 (t, J=6.7 Hz, 2H), 2.79 (s, 1H), 2.75-2.68 (m, 1H), 2.63-2.56 (m, 1H), 2.40 (d, J=6.4 Hz, 1H), 1.71-1.64 (m, 2H), 1.40-1.22 (m, 14H), 0.88 (t, J=6.7 Hz, 3H).

### Example 19

### N-(2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)benzamide

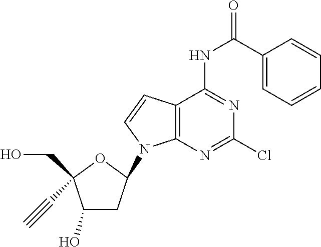 [SMILES: C#C[C@]1(CO)O[C@@H](n2ccc3c(NC(=O)c4ccccc4)nc(Cl)nc32)C[C@@H]1O]

### Step 1: N-(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)benzamide

To a stirred mixture of 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (Intermediate 2) (50 mg, 0.093 mmol) in pyrdine (1 mL) was added DMAP (23 mg, 0.19 mmol) and benzoyl chloride (20 mg, 0.14 mmol) at room temperature. The resulting mixture was stirred at room temperature for 16 h. The reaction was diluted with EtOAc (50 mL) and washed with water (2×50 mL) and brine (sat., 2×50 mL), dried over Na₂SO₄ and concentrated. The residue was purified by silica gel column chromatography, eluting with 0-50% EtOAc in Pet. Ether to afford the title compound. MS: m/ [symbol] =641.35 [M+H]⁺.

### Step 2: N-(2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)benzamide

To a mixture of N-(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)benzamide (33 mg, 0.051 mmol) in THF (1 mL) was added tetrabutylammonium fluoride (0.062 mL, 0.062 mmol). The resulting mixture was stirred for 16 h at room temperature. The reaction mixture was concentrated under reduced pressure and the residue was purified by RP-Combiflash with the following conditions: Column: C18 40 g Column, 60 Å, 20-35 μm; Mobile Phase A: water, Mobile Phase B: MeCN; Flow rate: 50 mL/min; 0% B to 100% B in 15 min; Detector: UV 254 & 210 nm to afford the title compound. MS: m/ [symbol] =411.00[M−H]⁻. ¹H-NMR (400 MHz, CD₃OD) δ 7.92-7.91 (m, 2H), 7.57-7.43 (m, 4H), 6.80-6.78 (m, 1H), 6.58-6.56 (m, 1H), 4.63-4.59 (m, 1H), 3.76-3.66 (m, 2H), 2.98 (s, 1H), 2.60-2.47 (m, 2H).

### Example 20

### ((2R,3S,5R)-5-(4-amino-2-chloro-7H-pyrrolo[2,3-d]pyrimidin-7-yl)-2-ethynyl-3-hydroxytetrahydrofuran-2-yl)methyl decanoate

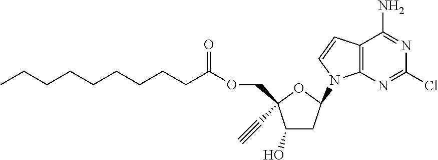 [SMILES: C#C[C@]1(COC(=O)CCCCCCCCC)O[C@@H](n2ccc3c(N)nc(Cl)nc32)C[C@@H]1O]

### Step 1: ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methyl decanoate

To a stirred mixture of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methanol (Intermediate 3) (100 mg, 0.117 mmol) in DMF (1.5 mL) were added DIC (0.082 mL, 0.527 mmol), DMAP (4.29 mg, 0.035 mmol) and decanoic acid (60.6 mg, 0.352 mmol). The resulting mixture was warmed to 25° C. and stirred for 18 h. The reaction mixture was quenched by water (50 mL) and extracted with ethyl acetate (3×40 mL). The combined organic fractions was washed with brine (3×30 mL), dried over anhydrous sodium sulfate and filtered. The filtrate was concentrated under reduced pressure and the residue was purified by Prep-TLC (EtOAc/Pet. ether=1:2) to afford the title compound. MS: m/ [symbol] 1007.60 [M+H]⁺.

### Step 2: ((2R,3S,5R)-5-(4-amino-2-chloro-7H-pyrrolo[2,3-d]pyrimidin-7-yl)-2-ethynyl-3-hydroxytetrahydrofuran-2-yl)methyl decanoate

To a stirred mixture of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methyl decanoate (150 mg, 0.149 mmol) in water (1 mL) was added formic acid (4 mL). The resulting mixture was warmed to 25° C. and stirred for overnight. The reaction mixture was purified by RP-flash chromatography, eluted with 0-80% acetonitrile in water to afford the title compound. MS: m/ [symbol] =463.15 [M+H]⁺. ¹H-NMR (400 MHz, CD₃OD): δ 7.20 (d, J=3.6 Hz, 1H), 6.59 (d, J=3.6 Hz, 1H), 6.46-6.43 (m, 1H), 4.74 (t, J=7.6 Hz, 1H), 4.48-4.45 (m, 1H), 4.24-4.21 (m, 1H), 3.13 (s, 1H), 2.70-2.59 (m, 2H), 2.32-2.27 (m, 2H), 1.53 (s, 2H), 1.25 (s, 12H), 0.88 (t, J=7.2 Hz, 3H).

Alternatively, Example 20 can be prepared as follows: In a flame dried flask under an atmosphere of N₂, (2R,3S,5R)-5-(4-amino-2-chloro-7H-pyrrolo[2,3-d]pyrimidin-7-yl)-2-ethynyl-2-(hydroxymethyl)tetrahydrofuran-3-ol (compound A) (2.0 g, 6.5 mmol) was dissolved in anhy pyridine (40 mL) and put into an ice bath. To this was added decanoyl chloride (1.5 mL, 7.1 mmol) dropwise over 5 mins and then stirred at 0° C. for 150 mins. The reaction was quenched with anhy methanol (5 mL) and stirred for 5 mins. The reaction was concentrated under reduced pressure and then partitioned between water and ether. The organics were washed with water (2×) and brine (1×), dried over Na₂SO₄, filtered, and concentrated under reduced pressure. The residue was redissolved in DCM/toluene and purified on NP silica gel column (120 g ISCO-gold) eluting 0-65% EtOAc/hexanes. The appropriate fractions were concentrated under reduced pressure, and then redissolved in ether and concentrated under reduced pressure (2×). The residue was recrystallized from diethyl ether to provide the title compound. MS: m/ [symbol] =463.5[M+H]⁺. ¹H NMR (500 MHz, CDCl₃) δ 7.05 (d, J=3.5 Hz, 1H), 6.60-6.54 (m, 1H), 6.38 (d, J=3.5 Hz, 1H), 5.36 (s, 2H), 4.65 (q, J=7.0 Hz, 1H), 4.45 (d, J=11.8 Hz, 1H), 4.39 (d, J=11.9 Hz, 1H), 2.78-2.69 (m, 2H), 2.65-2.58 (m, 1H), 2.42-2.30 (m, 3H), 1.66-1.59 (m, 2H), 1.35-1.19 (m, 12H), 0.88 (t, J=6.7 Hz, 3H).

### Example 21

### N-(2-chloro-7-((2R,4S,5R)-5-ethynyl-4-hydroxy-5-(hydroxymethyl)tetrahydrofuran-2-yl)-7H-pyrrolo[2,3-d]pyrimidin-4yl)dodecanamide

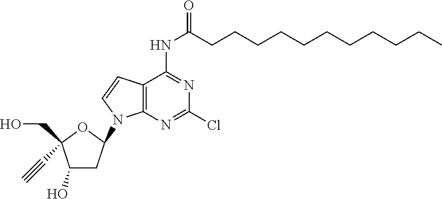 [SMILES: C#C[C@]1(CO)O[C@@H](n2ccc3c(NC(=O)CCCCCCCCCCC)nc(Cl)nc32)C[C@@H]1O]

The title compound was made in a similar manner as Example 19 except using dodecanoyl chloride in Step 1 and triethylamine trihydrofluoride in Step 2 (example 12-step 2). MS: m/ [symbol] =491.15 [M+H]⁺. ¹H-NMR (400 MHz, CD₃OD) δ 7.59-7.45 (m, 1H), 6.95-6.89 (m, 1H), 6.70-6.60 (m, 1H), 4.71-4.65 (m, 1H), 3.85-3.70 (m, 2H), 3.07-3.05 (m, 1H), 2.70-2.50 (m, 4H), 1.80-1.69 (m, 2H), 1.44-1.25 (m, 16H), 0.93-0.82 (m, 3H).

Alternatively, Example 21 can be prepared as follows:

### Step 1: N-(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)-N-dodecanoyldodecanamide—

To a solution of 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (1.0 g, 1.9 mmol) (Intermediate 2) in anhydrous dichloroethane (14 mL) was added DIEA (1.6 mL, 9.3 mmol) and dodecanoyl chloride (1.7 mL, 7.4 mmol). The reaction was heated in the microwave at 130° C. for 4 hours. The reaction was cooled and ammonia (9.3 mL, 18 mmol, 2 M in MeOH) was added and the reaction was stirred for two hours at room temperature. The reaction was partitioned between water and diethyl ether. The organics were washed with water (3×) and then brine (lx), dried over Na₂SO₄, filtered and concentrated under reduced pressure. The residue was redissolved in DCM and purified on NP silica gel column (120 g ISCO-gold) eluting 0-100% EtOAc/hexanes to provide the title compound. MS: m/ [symbol] =719.5 [M+H]⁺.

### Step 2: N-(2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)dodecanamide

A mixture of N-(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)dodecanamide (3.7 g, 5.1 mmol) in THF (100 mL) was cooled in an ice bath and tetrabutylammonium fluoride (10 mL, 10 mmol, 1 M in THF) was added. The resulting mixture was warmed to room temperature and stirred for three hours. The reaction was partitioned between water and diethyl ether. The organics were washed with water (3×) and then brine (1×), dried over Mg₂SO₄, filtered and concentrated under reduced pressure. The residue was redissolved in DCM and purified on NP silica gel column (330 g ISCO-gold) eluting 0-50% EtOAc/hexanes. The appropriate fractions were concentrated under reduced pressure, and then redissolved in ether and concentrated under reduced pressure (2×). The residue was recrystallized from diethyl ether to provide the title compound. MS: m/ [symbol] =491.5 [M+H]⁺. ¹H NMR (500 MHz, CDCl₃) δ 7.99 (s, 1H), 7.12 (d, J=3.8 Hz, 1H), 7.05 (d, J=3.8 Hz, 1H), 6.38 (dd, J=8.5, 6.0 Hz, 1H), 4.85 (dd, J=11.4, 3.0 Hz, 1H), 4.72 (dt, J=5.9, 3.0 Hz, 1H), 4.06 (dd, J=12.4, 3.0 Hz, 1H), 3.92-3.83 (m, 1H), 3.16-3.06 (m, 1H), 2.80 (s, 1H), 2.53-2.40 (m, 3H), 2.37 (dd, J=3.4, 1.2 Hz, 1H), 1.80-1.70 (m, 2H), 1.43-1.35 (m, 2H), 1.28 (d, J=18.2 Hz, 14H), 0.88 (t, J=7.0 Hz, 3H).

### Example 22

### N-(2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)pentanamide

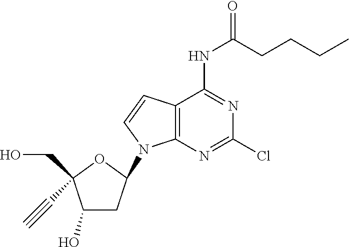 [SMILES: C#C[C@]1(CO)O[C@@H](n2ccc3c(NC(=O)CCCC)nc(Cl)nc32)C[C@@H]1O]

The title compound was made in a similar manner as Example 20 except using pentanoyl chloride in Step 1 and triethylamine trihydrofluoride in Step 2 (example 12-step 2). MS: m/ [symbol] =393.10 [M+H]⁺. ¹H-NMR (400 MHz, CD₃OD): δ 7.54-7.52 (m, 1H), 6.91-6.89 (m, 1H), 6.65-6.62 (m, 1H), 4.71-4.67 (m, 1H), 3.84-3.81 (m, 1H), 3.77-3.74 (m, 1H), 3.07 (s, 1H), 2.63-2.52 (m, 4H), 1.73-1.67 (m, 2H), 1.46-1.13 (m, 2H), 0.97 (t, J=7.2 Hz, 3H).

### Example 23

### ((2R,3S,5R)-5-(4-amino-2-chloro-7H-pyrrolo[2,3-d]pyrimidin-7-yl)-2-ethynyl-3-hydroxytetrahydrofuran-2-yl)methyl isopropyl carbonate

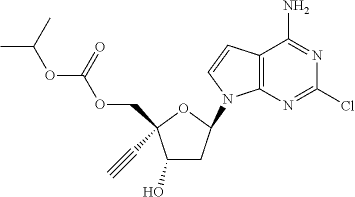 [SMILES: C#C[C@]1(COC(=O)OC(C)C)O[C@@H](n2ccc3c(N)nc(Cl)nc32)C[C@@H]1O]

### Step 1: ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methanol

To a solution of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methanol (Intermediate 3)(0.10 g, 0.12 mmol) and 4-dimethylaminopyridine (14 mg, 0.12 mmol) in DCM (2 mL) were added TEA (0.024 mL, 0.18 mmol) and isopropyl carbonochloridate (29 mg, 0.23 mmol) at 0° C. under N₂ atmosphere. The resulting mixture was stirred at 25° C. for 16 h. The reaction mixture was purified by prep-TLC, developed using DCM: MeOH (10:1) to afford the title compound. MS: m/ [symbol] =939.45 [M+H]⁺.

### Step 2: ((2R,3S,5R)-5-(4-amino-2-chloro-7H-pyrrolo[2,3-d]pyrimidin-7-yl)-2-ethynyl-3-hydroxytetrahydrofuran-2-yl)methyl isopropyl carbonate

To a solution of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methyl isopropyl carbonate (0.10 g, 0.11 mmol) in water (1 mL) was added formic acid (4.0 mL, 0.11 mmol) at 25° C. under N₂ atmosphere. The resulting mixture was stirred at 25° C. for 16 h. The residue was purified by RP-Flash with the following conditions: C18 40 g Column; Mobile Phase A: water (0.1% NH₄HCO₃, Mobile Phase B: MeCN; Flow rate: 50 mL/min; Gradient: 2% B to 30% B in 30 min, Detector: UV 210 nm) to afford the title compound. MS: m/ [symbol] =393.05 [M−H]⁻. ¹H NMR (400 MHz, DMSO-d₆) δ 7.61-7.56 (m, 2H), 7.28-7.26 (m, 1H), 6.63-6.62 (m, 1H), 6.42-6.38 (m, 1H), 5.81 (s, 1H), 4.76-4.69 (m, 1H), 4.51-4.38 (m, 2H), 4.18-4.15 (m, 1H), 3.63-3.61 (m, 1H), 2.62-2.51 (m, 1H), 2.45-2.38 (m, 1H), 1.21-1.18 (m, 6H).

### Example 24

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl octanoate

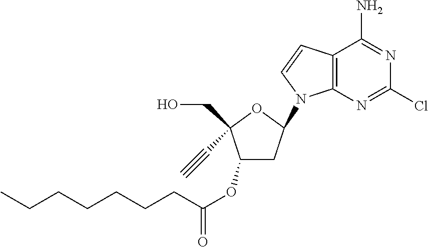 [SMILES: C#C[C@]1(CO)O[C@@H](n2ccc3c(N)nc(Cl)nc32)C[C@@H]1OC(=O)CCCCCCC]

The title compound was made in a similar manner as Example 1 except using octanoyl chloride in Step 1. MS: m/ [symbol] =435.10 [M+H]⁺. 1H-NMR (300 MHz, CDCl₃): δ 7.03 (s, 1H), 6.37 (s, 1H), 6.30-6.27 (m, 1H), 5.79 (s, 1H), 5.55 (s, 3H), 4.07-3.97 (m, 2H), 3.35-3.19 (m, 1H), 2.59 (s, 1H), 2.44-2.39 (m, 3H), 1.90-1.78 (m, 2H), 1.30 (s, 8H), 1.15 (s, 3H).

Alternatively, Example 24 can be prepared as follows:

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl octanoate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (2.5 g, 5.9 mmol), TEA (0.90 g, 8.9 mmol) and DMAP (0.72 g, 5.9 mmol) in DCM (25 mL) was added octanoyl chloride (1.4 g, 8.9 mmol) at room temperature under an atmosphere of argon. The resulting mixture was stirred for 16 hr at room temperature. The solution was concentrated under reduce pressure and the residue was purified on NP silica gel column chromatography, eluting with 0-73% EtOAc/Pet. Ether to provide the title compound. MS: m/ [symbol] =549.2 [M+H]⁺.

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl octanoate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl octanoate (2.0 g, 3.6 mmol) in THF (5 mL) under an atmosphere of argon was added triethylamine trihydrofluoride (5.9 g, 36 mmol) at room temperature. The resulting mixture was stirred for 16 h at room temperature. The reaction was purified by RP-flash chromatography, eluting with 0-48% acetonitrile in water and then recrystallized from ether-heptane to provide the title compound. MS: m/ [symbol] =435.4 [M+H]⁺. ¹H NMR (500 MHz, CDCl₃) δ 7.04-6.99 (m, 1H), 6.37-6.32 (m, 1H), 6.31-6.25 (m, 1H), 5.79 (d, J=6.4 Hz, 1H), 5.57 (d, J=11.6 Hz, 1H), 5.42 (s, 2H), 4.04 (d, J=12.2 Hz, 1H), 3.94 (t, J=12.1 Hz, 1H), 3.30-3.21 (m, 1H), 2.59 (s, 1H), 2.45-2.35 (m, 3H), 1.72-1.64 (m, 2H), 1.35-1.28 (m, 8H), 0.92-0.86 (m, 3H).

### Example 25

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl dodecanoate

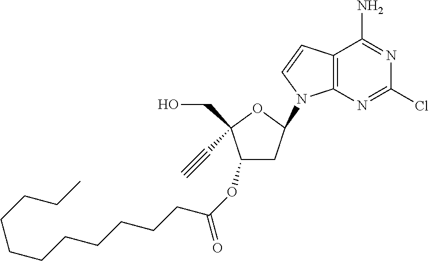 [SMILES: C#C[C@]1(CO)O[C@@H](n2ccc3c(N)nc(Cl)nc32)C[C@@H]1OC(=O)CCCCCCCCCCC]

The title compound was made in a similar manner as Example 12 except using dodecanoyl chloride in Step 1. MS: m/ [symbol] =490.95 [M+H]⁺. ¹H NMR (300 MHz, DMSO-d₆) δ 7.60 (s, 2H), 7.39 (s, 1H), 6.64-6.479 (m, 1H), 6.46-6.43 (m, 1H), 5.59-5.52 (m, 2H), 3.64-3.57 (m, 3H), 2.76-2.72 (m, 1H), 2.51-2.34 (m, 3H), 1.61-1.54 (m, 2H), 1.27-1.24 (m, 16H), 0.87-0.82 (m, 3H).

### Example 26

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl tetradecanoate

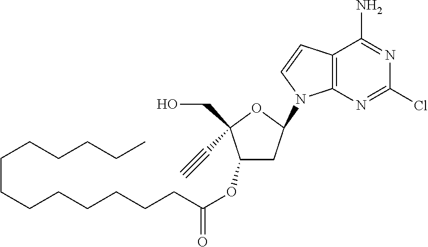 [SMILES: C#C[C@]1(CO)O[C@@H](n2ccc3c(N)nc(Cl)nc32)C[C@@H]1OC(=O)CCCCCCCCCCCCC]

The title compound was made in a similar manner as Example 12 except using tetradecanoyl chloride in Step 1. MS: m/ [symbol] =519.00 [M+H]⁺. ¹H NMR (300 MHz, DMSO-d₆) δ 7.59 (s, 2H), 7.40-7.38 (s, 1H), 6.64-6.46 (m, 1H), 6.45-5.59 (m, 1H), 5.57-5.52 (m, 2H), 3.66-3.59 (m, 3H), 2.81-2.72 (m, 1H), 2.25-2.37 (m, 3H), 1.61-1.54 (m, 2H), 1.27-1.23 (m, 20H), 0.87-0.83 (m, 3H).

### Example 27

### (2R,3S,5R)-5-(4-amino-2-chloro-7H-pyrrolo[2,3-d]pyrimidin-7-yl)-2-ethynyl-2-(hydroxymethyl)tetrahydrofuran-3-ylbenzyl carbonate

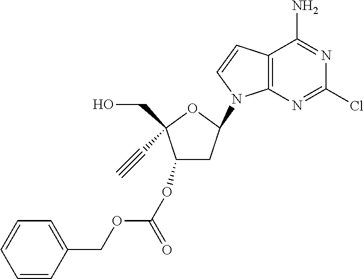 [SMILES: C#C[C@]1(CO)O[C@@H](n2ccc3c(N)nc(Cl)nc32)C[C@@H]1OC(=O)OCc1ccccc1]

The title compound was made in a similar manner as Example 12 except using benzyl carbonochloridate in Step 1. MS: m/ [symbol] =443.05 [M+H]⁺. ¹H-NMR (400 MHz, CD₃OD): δ 7.41-7.32 (m, 6H), 6.60 (s, 1H), 6.50-6.46 (m, 1H), 5.53-5.51 (m, 1H), 5.25-5.17 (m, 2H), 3.87-3.78 (m, 2H), 2.95-2.88 (m, 2H), 2.58-2.52 (m, 1H).

Alternatively, Example 27 can be prepared as follows.

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl benzyl carbonate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (2.5 g, 5.9 mmol), TEA (0.90 g, 8.9 mmol) and DMAP (0.72 g, 5.9 mmol) in DCM (25 mL) was added benzyl carbonochloridate (2.0 g, 12 mmol) at room temperature under an atmosphere of argon. The resulting mixture was stirred for 16 h at 40° C. Then the solution was concentrated under reduced pressure. The residue was purified by silica gel column chromatography, eluting with 0-56% EtOAc/Pet. ether to provide the title compound. MS: m/ [symbol] =557.2 [M+H]⁺.

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl benzyl carbonate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl benzyl carbonate (2.0 g, 3.6 mmol) in THF (2 mL) was added triethylamine trihydrofluoride (5.8 g, 36 mmol) at room temperature under an argon atmosphere. The resulting mixture was stirred for 16 h at room temperature. The reaction was purified by RP-flash chromatography, eluting with 0-48% acetonitrile in water and then recrystallized from EtOAc-heptanes to provide the title compound. MS: m/ [symbol] =443.4 [M+H]⁺. ¹H NMR (500 MHz, CDCl₃) δ 7.46-7.34 (m, 5H), 6.95 (d, J=3.7 Hz, 1H), 6.32 (d, J=3.7 Hz, 1H), 6.22 (dd, J=9.9, 5.3 Hz, 1H), 5.85 (dd, J=12.1, 2.4 Hz, 1H), 5.64 (d, J=6.0 Hz, 1H), 5.32 (s, 2H), 5.28 (d, J=12.0 Hz, 1H), 5.18 (d, J=12.0 Hz, 1H), 4.04 (dd, J=12.3, 2.4 Hz, 1H), 3.94 (t, J=12.3 Hz, 1H), 3.38-3.28 (m, 1H), 2.45 (dd, J=14.0, 5.4 Hz, 1H), 2.33 (s, 1H).

### Example 28

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl picolinate

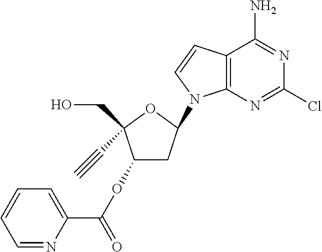 [SMILES: C#C[C@]1(CO)O[C@@H](n2ccc3c(N)nc(Cl)nc32)C[C@@H]1OC(=O)c1ccccn1]

The title compound was made in a similar manner as Example 12 except using picolinoyl chloride in Step 1. MS: m/ [symbol] =414.05 [M+H]⁺. 1H-NMR (400 MHz, CD₃OD): δ 8.73 (s, 1H), 8.34-8.32 (m, 1H), 8.09-8.07 (m, 1H), 7.70-7.67 (m, 1H), 7.37 (s, 1H), 6.73-6.61 (m, 2H), 5.97-5.95 (m, 1H), 3.93 (s, 2H), 3.07-3.00 (m, 2H), 2.76-2.67 (m, 1H).

### Example 29

### (2R,3S,5R)-5-(4-amino-2-chloro-7H-pyrrolo[2,3-d]pyrimidin-7-yl)-2-ethynyl-2-((2-(4-fluorophenyl)acetoxy)methyl)tetrahydrofuran-3-yl 2-(4-fluorophenyl)acetate

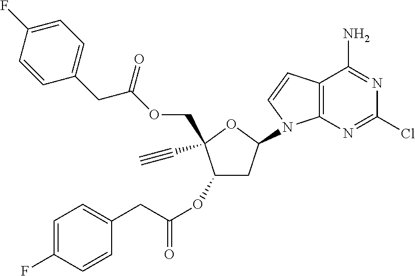 [SMILES: C#C[C@]1(COC(=O)Cc2ccc(F)cc2)O[C@@H](n2ccc3c(N)nc(Cl)nc32)C[C@@H]1OC(=O)Cc1ccc(F)cc1]

The title compound was made in a similar manner as Example 6 except using 2-(4-fluorophenyl)acetic acid in Step 1. MS: m/ [symbol] =581.15 [M+H]⁺. ¹H NMR (400 MHz, DMSO-d₆) δ 7.62 (s, 2H), 7.39-7.35 (m, 4H), 7.29-7.07 (m, 5H), 6.64-6.63 (m, 1H), 6.48-6.45 (m, 1H), 5.65-5.45 (m, 1H), 4.37-4.22 (m, 2H), 3.83-3.72 (m, 5H), 2.85-2.76 (m, 1H), 2.53-2.49 (m, 1H). ¹⁹H NMR (376 MHz, DMSO-d₆) δ−115.93.

### Example 30

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl 3-((adamantan-1-yl)propanoate

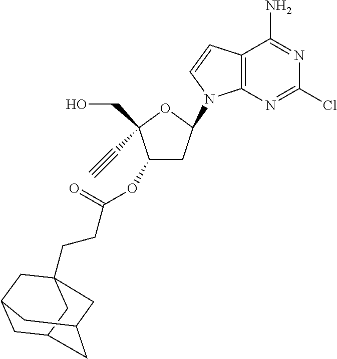 [SMILES: C#C[C@]1(CO)O[C@@H](n2ccc3c(N)nc(Cl)nc32)C[C@@H]1OC(=O)CCC12CC3CC(CC(C3)C1)C2]

The title compound was made in a similar manner as Example 2 except using 3-(adamantan-1-yl)propanoic acid in Step 1 and triethylamine trihydrofluoride in Step 2 (example 12, step 2). MS: m/z=499.15 [M+H]⁺. ¹H-NMR (400 MHz, CD₃OD-d₄): δ 7.32 (s, 1H), 6.60-6.59 (m, 1H), 6.53-6.48 (m, 1H), 5.65-5.62 (m, 1H), 3.86-3.78 (m, 2H), 3.12 (s, 1H), 2.90-2.83 (m, 1H), 2.52-2.37 (m, 3H), 1.96 (s, 3H), 1.78-1.67 (m, 6H), 1.54-1.46 (m, 8H).

### Example 31

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl 2-(4-fluorophenyl)acetate

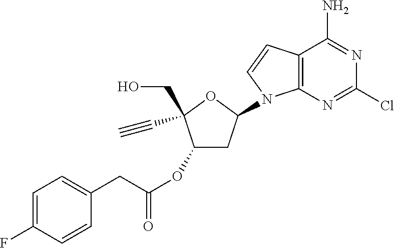 [SMILES: C#C[C@]1(CO)O[C@@H](n2ccc3c(N)nc(Cl)nc32)C[C@@H]1OC(=O)Cc1ccc(F)cc1]

The title compound was made in a similar manner as Example 2 except using 2-(4-fluorophenyl)acetic acid in Step 1 and triethylamine trihydrofluoride in Step 2 (example 12, step 2). MS: m/z=445.05 [M+H]⁺. ¹H-NMR (400 MHz, CD₃OD): δ 7.39-7.36 (m, 3H), 7.10-7.06 (m, 2H), 6.63-6.62 (m, 1H), 6.56-6.52 (m, 1H), 5.59-5.66 (m, 1H), 3.84-3.79 (m, 4H), 3.06 (s, 1H), 2.92-2.85 (m, 1H), 2.54-2.48 (m, 1H). ¹⁹F NMR (376 MHz, CD₃OD) δ−118.03.

### Example 32

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl 2-(4-chlorophenyl)acetate

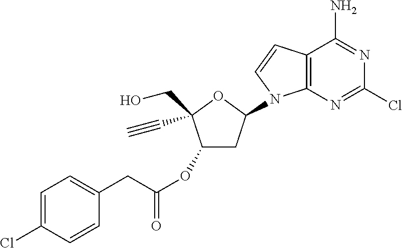 [SMILES: C#C[C@]1(CO)O[C@@H](n2ccc3c(N)nc(Cl)nc32)C[C@@H]1OC(=O)Cc1ccc(Cl)cc1]

The title compound was made in a similar manner as Example 2 except using 2-(4-chlorophenyl)acetic acid in Step 1 and triethylamine trihydrofluoride in Step 2 (example 12, step 2). MS: m/z=461.05 [M+H]⁺. ¹H-NMR (400 MHz, CD₃OD): δ 7.34-7.32 (m, 5H), 6.60-6.49 (m, 2H), 5.67-5.64 (m, 1H), 3.84-3.73 (m, 4H), 3.04 (s, 1H), 2.88-2.83 (m, 1H), 2.51-2.46 (m, 1H).

### Example 33

### ((2R,3S,5R)-5-(4-amino-2-chloro-7H-pyrrolo[2,3-d]pyrimidin-7-yl)-2-ethynyl-3-hydroxytetrahydrofuran-2-yl)methyl 2-(4-chlorophenyl)acetate

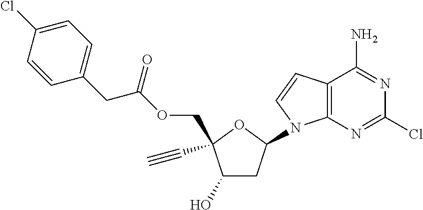 [SMILES: C#C[C@]1(COC(=O)Cc2ccc(Cl)cc2)O[C@@H](n2ccc3c(N)nc(Cl)nc32)C[C@@H]1O]

The title compound was made in a similar manner as Example 20 except using 2-(4-chlorophenyl)acetic acid in Step 1. MS: m/ [symbol] =461.05 [M+H]⁺. ¹H-NMR (400 MHz, CD₃OD): δ 7.26-7.24 (m, 2H), 7.17-7.15 (m, 2H), 7.10-7.09 (m, 1H), 6.62-6.61 (m, 1H), 6.47-6.44 (m, 1H), 4.79-4.71 (m, 1H), 4.51-4.48 (m, 1H), 4.28-4.25 (m, 1H), 3.68-3.46 (m, 2H), 3.14 (s, 1H), 2.68-2.54 (m, 2H).

### Example 34

### (2R,3S,5R)-5-(4-amino-2-chloro-7H-pyrrolo[2,3-d]pyrimidin-7-yl)-2-((2-(4-chlorophenyl)acetoxy)methyl)-2-ethynyltetrahydrofuran-3-yl 2-(4-chlorophenyl)acetate

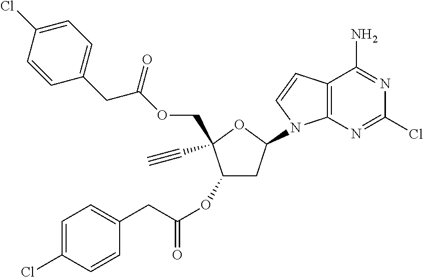 [SMILES: C#C[C@]1(COC(=O)Cc2ccc(Cl)cc2)O[C@@H](n2ccc3c(N)nc(Cl)nc32)C[C@@H]1OC(=O)Cc1ccc(Cl)cc1]

The title compound was made in a similar manner as Example 6 except using 2-(4-chlorophenyl)acetic acid in Step 1. MS: m/ [symbol] =613.05 [M+H]⁺. ¹H-NMR (400 MHz, CD₃OD): δ 7.36-7.19 (m, 8H), 7.14-7.00 (m, 1H), 6.59-6.49 (m, 2H), 5.66-5.63 (m, 1H), 4.43-4.31 (m, 2H), 3.82-3.71 (m, 4H), 3.16 (s, 1H), 2.79-2.72 (m, 1H), 2.59-2.56 (m, 1H).

Alternatively, Example 34 can be prepared as follows. In a flame-dried flask under an atmosphere of N₂, (2R,3S,5R)-5-(4-amino-2-chloro-7H-pyrrolo[2,3-d]pyrimidin-7-yl)-2-ethynyl-2-(hydroxymethyl)tetrahydrofuran-3-ol (compound A) (2.0 g, 6.5 mmol), and 2-(4-chlorophenyl)acetic acid (2.8 g, 16 mmol) was dissolved in anhydrous DMF (7.0 mL). To this was added anhydrous DCM (70 mL), DIEA (5.6 mL, 32 mmol) and 7-Azabenzotriazol-1-yloxytris(dimethylamino)phosphoniumhexafluorophosphate (7.2 g, 16 mmol). The resulting solution was stirred at room temperature for 16 hours. The reaction was quenched with water and extracted with DCM (3×). The combined organic fractions were washed with brine, dried over anhydrous sodium sulfate, and filtered. The filtrate was concentrated under reduced pressure and the residue was purified by silica gel column chromatography, eluting with 0-50% EtOAc/hexanes. The appropriate fractions were concentrated under reduced pressure and then recrystallized from iPrOH to afford the title compound. MS: m/ [symbol] =613.5 [M+H]⁺. ¹H NMR (500 MHz, CDCl₃) δ 7.35-7.30 (m, 2H), 7.29-7.24 (m, 4H), 7.21-7.16 (m, 2H), 6.77-6.73 (m, 1H), 6.62 (t, J=6.7 Hz, 1H), 6.36-6.32 (m, 1H), 5.55-5.49 (m, 1H), 5.44 (s, 2H), 4.45 (d, J=11.9 Hz, 1H), 4.35 (d, J=11.8 Hz, 1H), 3.70 (s, 2H), 3.65 (s, 2H), 2.60-2.53 (m, 2H), 2.49-2.45 (m, 1H).

### Example 35

### 2-(adamantan-1-yl)ethyl (((2R,3S,5R)-5-(4-amino-2-chloro-7H-pyrrolo[2,3-d]pyrimidin-7-yl)-2-ethynyl-3-hydroxytetrahydrofuran-2-yl)methyl) carbonate

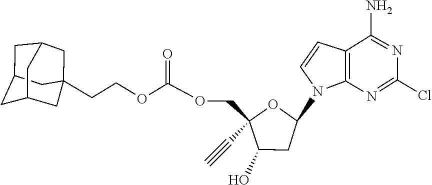 [SMILES: C#C[C@]1(COC(=O)OCCC23CC4CC(CC(C4)C2)C3)O[C@@H](n2ccc3c(N)nc(Cl)nc32)C[C@@H]1O]

The title compound was made in a similar manner as Example 39 except using 2-(adamantan-1-yl)ethyl (4-nitrophenyl)carbonate (Intermediate 4) in Step 1. MS: m/ [symbol] =515.10 [M+H]⁺. ¹H-NMR (400 MHz, CD₃OD): δ 7.20-7.19 (m, 1H), 6.59-6.50 (m, 2H), 4.84-4.70 (m, 1H), 4.52-4.47 (m, 1H), 4.33-4.28 (m, 1H), 4.16-4.12 (m, 2H), 3.16 (s, 1H), 2.62-2.58 (m, 2H), 2.01-1.92 (m, 3H), 1.75-1.72 (m, 6H), 1.68-1.65 (m, 6H), 1.54-1.53 (m, 2H).

### Example 36

### 2-(adamantan-1-yl)ethyl ((2R,3S,5R)-5-(4-amino-2-chloro-7H-pyrrolo[2,3-d]pyrimidin-7-yl)-2-ethynyl-2-(hydroxymethyl)tetrahydrofuran-3-yl) carbonate

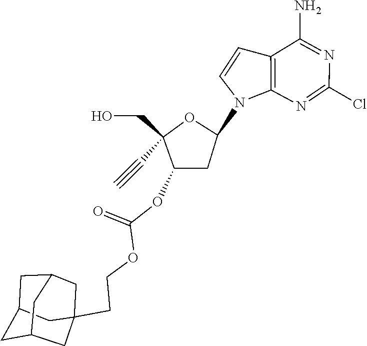 [SMILES: C#C[C@]1(CO)O[C@@H](n2ccc3c(N)nc(Cl)nc32)C[C@@H]1OC(=O)OCCC12CC3CC(CC(C3)C1)C2]

The title compound was made in a similar manner as Example 12 except using 2-(adamantan-1-yl)ethyl (4-nitrophenyl)carbonate (Intermediate 4) in Step 1. MS: m/z=515.10 [M+H]⁺. ¹H-NMR (400 MHz, CD₃OD-d₄): δ 7.30 (s, 1H), 6.59-6.51 (m, 1H), 6.48-6.47 (m, 1H), 5.52-5.50 (m, 1H), 4.28-4.23 (m, 2H), 3.89-3.81 (m, 2H), 3.11 (s, 1H), 2.96-2.88 (m, 1H), 2.58-2.54 (m, 1H), 2.03-1.95 (m, 3H), 1.78-1.68 (m, 6H), 1.60-1.58 (m, 6H), 1.51-1.47 (m, 2H).

Alternatively, Example 36 can be prepared as follows.

### Step 1: 2-(adamantan-1-yl)ethyl ((2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl) carbonate

To a solution of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (5.0 g, 12 mmol) and Intermediate 4 (6.1 g, 18 mmol) dissolved in anhydrous pyridine (90 mL) under N₂, was added DMAP (1.4 g, 12 mmol) and triethylamine (3.9 mL, 28 mmol). The reaction was put into a preheated oil bath at 75° C. and stirred for 13 hours. The reaction was concentrated under reduced pressure. The residue was partitioned between water and ether and the organic layer was washed with water (2×), dilute HCl (pH 4), and brine. The organic fraction was dried over anhydrous sodium sulfate, and filtered. The filtrate was concentrated under reduced pressure and the residue was purified by silica gel column chromatography, eluting with 0-45% EtOAc/hexane to provide the title compound. MS: m/z =629.7 [M+H]⁺.

### Step 2: 2-(adamantan-1-yl)ethyl ((2R,3S,5R)-5-(4-amino-2-chloro-7H-pyrrolo[2,3-d]pyrimidin-7-yl)-2-ethynyl-2-(hydroxymethyl)tetrahydrofuran-3-yl) carbonate

To a cold solution of 2-(adamantan-1-yl)ethyl ((2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl) carbonate (6.8 g, 11 mmol) in anhydrous THF (100 mL) under N₂, was added tetrabutylammonium fluoride (1M in THF) (11 mL, 11 mmol). The reaction was stirred at RT for 45 min. The reaction was diluted with ether and water and the organic layer was washed with water (4×) and brine. The organic fraction was dried over anhydrous sodium sulfate, and filtered. The filtrate was concentrated under reduced pressure and the residue was purified by silica gel column chromatography, eluting with 0-55% EtOAc/hexanes. The appropriate fractions were concentrated under reduced pressure and then recrystallized from ether to provide the title compound. MS: m/z =515.6 [M+H]⁺. ¹H NMR (500 MHz, CDCl₃) δ 6.99-6.94 (m, 1H), 6.35-6.31 (m, 1H), 6.29-6.22 (m, 1H), 5.83 (d, J=12.1 Hz, 1H), 5.63 (d, J=6.1 Hz, 1H), 5.49 (s, 2H), 4.34-4.21 (m, 2H), 4.06 (d, J=12.4 Hz, 1H), 3.96 (t, J=12.3 Hz, 1H), 3.37-3.27 (m, 1H), 2.62 (s, 1H), 2.47 (dd, J=13.9, 5.4 Hz, 1H), 1.96 (s, 3H), 1.74-1.47 (m, 14H).

### Example 37

### ((2R,3S,5R)-5-(4-amino-2-chloro-7H-pyrrolo[2,3-d]pyrimidin-7-yl)-2-ethynyl-3-hydroxytetrahydrofuran-2-yl)methyl 2-(4-fluorophenyl)acetate

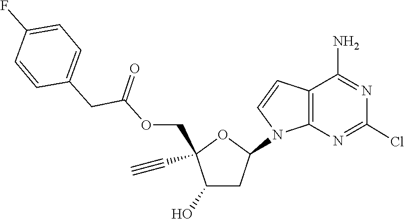 [SMILES: C#C[C@]1(COC(=O)Cc2ccc(F)cc2)O[C@@H](n2ccc3c(N)nc(Cl)nc32)C[C@@H]1O]

The title compound was made in a similar manner as Example 20 except using 2-(4-fluorophenyl)acetic acid in Step 1. MS: m/ [symbol] =445.00 [M+H]⁺. ¹H-NMR (400 MHz, CD₃OD): δ 7.20-7.17 (m, 2H), 7.10-7.08 (m, 1H), 7.03-6.97 (m, 2H), 6.59-6.58 (m, 1H), 6.46-6.43 (m, 1H), 4.73-4.71 (m, 1H), 4.69-4.50 (m, 1H), 4.47-4.25 (m, 1H), 3.69-3.59 (m, 2H), 3.13 (s, 1H), 2.68-2.54 (m, 2H).

### Example 38

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl nonyl carbonate

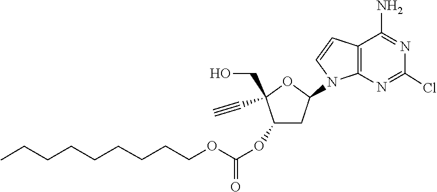 [SMILES: C#C[C@]1(CO)O[C@@H](n2ccc3c(N)nc(Cl)nc32)C[C@@H]1OC(=O)OCCCCCCCCC]

The title compound was made in a similar manner as Example 12 except using nonyl carbonochloridate in Step 1. MS: m/ [symbol] =479.15 [M+H]⁺¹H NMR (300 MHz, CDCl₃) δ 6.98 (d, J=3.0 Hz, 1H), 6.35 (d, J=3.0 Hz, 1H), 6.29-6.24 (m, 1H), 5.64-5.59 (m, 3H), 4.25-4.16 (m, 2H), 4.08-4.04 (m, 1H), 3.98-3.93 (m, 1H), 3.33-3.28 (m, 1H), 2.62 (s, 1H), 2.51-2.45 (m, 1H), 1.75-1.70 (m, 3H), 1.39-1.20 (m, 12H), 1.02-0.68 (m, 3H).

### Example 39

### ((2R,3S,5R)-5-(4-amino-2-chloro-7H-pyrrolo[2,3-d]pyrimidin-7-yl)-2-ethynyl-3-hydroxytetrahydrofuran-2-yl)methyl octyl carbonate

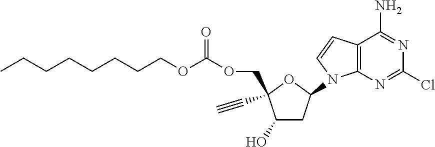 [SMILES: C#C[C@]1(COC(=O)OCCCCCCCC)O[C@@H](n2ccc3c(N)nc(Cl)nc32)C[C@@H]1O]

### Step 1: ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methyl octyl carbonate

To a stirred mixture of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methanol (Intermediate 3) (120 mg, 0.141 mmol) and 4-dimethylaminopyridine (25.8 mg, 0.211 mmol) in Py (2 mL) was added octyl carbonochloridate (54.2 mg, 0.281 mmol) at room temperature under argon atmosphere. The resulting mixture was stirred for 16 h at 80° C. The reaction mixture was concentrated, the reaction was diluted with water (10 mL), extracted with EtOAc (50 mL), the organic layer was washed with brine (20 mL×2), dried over anhydrous Na₂SO₄, filtered and the filtrate was concentrated under reduced pressure. The residue was purified by silica gel column chromatography, eluted with 0-30% of EtOAc in Pet. ether to afford the title compound. MS: m/ [symbol] =1010.35 [M+H]⁺.

### Step 2: ((2R,3S,5R)-5-(4-amino-2-chloro-7H-pyrrolo[2,3-d]pyrimidin-7-yl)-2-ethynyl-3-hydroxytetrahydrofuran-2-yl)methyl octyl carbonate

The mixture of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methyl octyl carbonate (110 mg, 0.109 mmol) in 80% aq. formic acid (2.5 mL, 0.109 mmol) was stirred for 16 h at room temperature. The mixture was concentrated under reduced pressure. The residue was purified by RP-Flash, eluted with 0-60% acetonitrile in water to afford the title compound. MS: m/ [symbol] =465.20 [M+H]⁺. ¹H-NMR (400 MHz, CD₃OD): δ 7.20 (s, 1H), 6.59-6.49 (m, 2H), 4.89-4.84 (m, 1H), 4.71-4.49 (m, 2H), 4.32-4.04 (m, 2H), 3.16 (s, 1H), 2.65-2.55 (m, 2H), 1.64-1.57 (m, 2H), 1.23 (s, 10H), 0.97-0.86 (m, 3H).

### Example 40

### ((2R,3S,5R)-5-(4-amino-2-chloro-7H-pyrrolo[2,3-d]pyrimidin-7-yl)-2-ethynyl-3-hydroxytetrahydrofuran-2-yl)methyl 2-ethylbutanoate

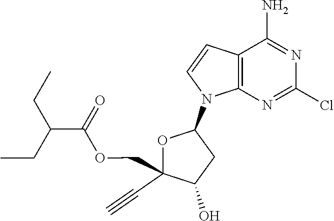 [SMILES: C#C[C@]1(COC(=O)C(CC)CC)O[C@@H](n2ccc3c(N)nc(Cl)nc32)C[C@@H]1O]

In a flame-dried flask under an atmosphere of nitrogen, (2R,3S,5R)-5-(4-amino-2-chloro-7H-pyrrolo[2,3-d]pyrimidin-7-yl)-2-ethynyl-2-(hydroxymethyl)tetrahydrofuran-3-ol (50 mg, 0.162 mmol) was dissolved in anhydrous pyridine (2 ml). The solution was cooled in an ice bath, and 2-ethylbutanoyl chloride (23 μL, 0.168 mmol) was added dropwise. The result was stirred at 0° C. for 30 mins, quenched with methanol (100 μL, 2.472 mmol) and then stirred for an additional 5 mins. The reaction was then concentrated under reduced pressure and partitioned between water and EtOAc. The organics were washed with water (3×) and then brine (lx), dried over Na₂SO₄, filtered and concentrated under reduced pressure. The residue was redissolved in DCM and purified on NP silica gel column (24 g ISCO-gold) eluting 0-80% EtOAc/hexanes to afford the title compound. MS: m/ [symbol] =407.4 [M+H]⁺. ¹H NMR (500 MHz, CDCl₃) δ 7.06 (d, J=3.7 Hz, 1H), 6.63-6.57 (m, 1H), 6.38 (d, J=3.7 Hz, 1H), 5.26 (s, 2H), 4.62 (q, J=6.9 Hz, 1H), 4.47-4.37 (m, 2H), 2.78-2.70 (m, 2H), 2.66-2.57 (m, 1H), 2.37-2.33 (m, 1H), 2.32-2.24 (m, 1H), 1.69-1.60 (m, 2H), 1.60-1.51 (m, 2H), 0.89 (q, J=7.3 Hz, 6H).

### Example 41

### ((2R,3S,5R)-5-(4-amino-2-chloro-7H-pyrrolo[2,3-d]pyrimidin-7-yl)-2-ethynyl-3-hydroxytetrahydrofuran-2-yl)methyl 2-phenylacetate

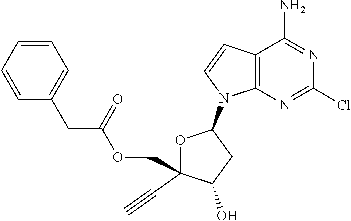 [SMILES: C#C[C@]1(COC(=O)Cc2ccccc2)O[C@@H](n2ccc3c(N)nc(Cl)nc32)C[C@@H]1O]

The title compound was made in a similar manner as Example 40 except using 2-phenylacetyl chloride and was further purified by RP chromatography (10-50% ACN-water (5 mM NH₄CO₃) over 14 min and held at 50% ACN on Waters Xbridge 30×150 mm). The appropriate fractions were combined, diluted with brine, and extracted with DCM (3×). The combined organics were dried over Na₂SO₄, filtered and concentrated under reduced pressure to afford the title compound. MS: m/ [symbol] =427.4 [M+H]⁺. ¹H NMR (500 MHz, CDCl₃) δ 7.36-7.27 (m, 5H), 6.84 (d, J=3.7 Hz, 1H), 6.59-6.52 (m, 1H), 6.34 (d, J=3.7 Hz, 1H), 5.31 (s, 2H), 4.56-4.51 (m, 1H), 4.43 (s, 2H), 3.70 (s, 2H), 2.73 (s, 1H), 2.65-2.49 (m, 2H), 2.26 (d, J=5.7 Hz, 1H).

### Example 42

### (2R,3S,5R)-5-(4-amino-2-chloro-7H-pyrrolo[2,3-d]pyrimidin-7-yl)-2-ethynyl-2-((isobutyryloxy)methyl)tetrahydrofuran-3-yl isobutyrate

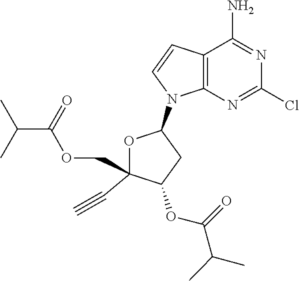 [SMILES: C#C[C@]1(COC(=O)C(C)C)O[C@@H](n2ccc3c(N)nc(Cl)nc32)C[C@@H]1OC(=O)C(C)C]

In a flame-dried flask under an atmosphere of nitrogen, (2R,3S,5R)-5-(4-amino-2-chloro-7H-pyrrolo[2,3-d]pyrimidin-7-yl)-2-ethynyl-2-(hydroxymethyl)tetrahydrofuran-3-ol (305 mg, 0.988 mmol) was dissolved in anhydrous pyridine (12 ml). The solution was cooled in an ice bath, and 2-ethylbutanoyl chloride (300 μL, 2.86 mmol) was added dropwise over 5 mins. The result was stirred at 0° C. for 10 mins and then quenched with methanol (400 μL, 9.88 mmol) and stirred for an additional 5 mins. The reaction was then concentrated under reduced pressure and partitioned between water and EtOAc. The organics were washed with water (3×) and then brine (lx), dried over Na₂SO₄, filtered and concentrated under reduced pressure. The residue was redissolved in DCM and purified on NP silica gel column (24 g ISCO-gold) eluting 0-55% EtOAc/hexanes to afford the title compound. MS: m/ [symbol] =449.4 [M+H]⁺. ¹H NMR (500 MHz, CDCl₃) δ 7.12 (d, J=3.7 Hz, 1H), 6.71 (t, J=6.7 Hz, 1H), 6.41 (d, J=3.7 Hz, 1H), 5.59-5.53 (m, 1H), 5.40 (s, 2H), 4.44 (d, J=11.8 Hz, 1H), 4.36 (d, J=11.8 Hz, 1H), 2.78-2.70 (m, 1H), 2.70-2.59 (m, 4H), 1.27-1.17 (m, 12H).

Alternatively, the compound of Example 42 can be further recrystallized from ether-heptanes MS: m/ [symbol] =449.4 [M+H]⁺.

### Example 43

### (2R,3S,5R)-5-(4-amino-2-chloro-7H-pyrrolo[2,3-d]pyrimidin-7-yl)-2-ethynyl-2-((2-phenylacetoxy)methyl)tetrahydrofuran-3-yl 2-phenylacetate

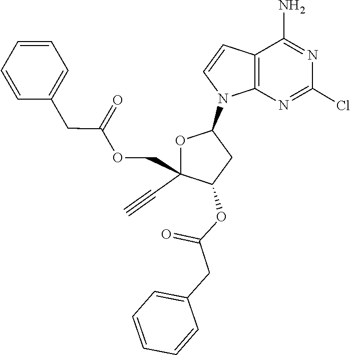 [SMILES: C#C[C@]1(COC(=O)Cc2ccccc2)O[C@@H](n2ccc3c(N)nc(Cl)nc32)C[C@@H]1OC(=O)Cc1ccccc1]

The title compound was made in a similar manner as Example 42 except using 2-phenylacetyl chloride, and was further purified by RP chromatography (25-80% ACN-water (5 mM NH₄CO₃) over 14 min and held at 68% ACN on Waters Xbridge 30×150 mm). The appropriate fractions were lyophilized overnight. The resulting solid was redissolved between DCM and diluted brine, and extracted with DCM (3×). The combined organics were dried over Na₂SO₄, filtered and concentrated under reduced pressure to afford the title compound. MS: m/ [symbol] =545.5 [M+H]⁺. ¹H NMR (500 MHz, CDCl₃) δ 7.38-7.26 (m, 10H), 6.73 (d, J=3.7 Hz, 1H) [symbol] 6.65 (t, J=6.9 Hz, 1H), 6.31 (d, J=3.7 Hz, 1H), 5.49-5.43 (m, 1H), 5.29 (s, 2H), 4.43 (d, J=12.0 Hz, 1H), 4.36 (d, J=12.0 Hz, 1H), 3.74-3.65 (m, 4H), 2.54-2.36 (m, 3H).

### Example 44

### (2R,3S,5R)-5-(4-amino-2-chloro-7H-pyrrolo[2,3-d]pyrimidin-7-yl)-2-(((2-ethylbutanoyl)oxy)methyl)-2-ethynyltetrahydrofuran-3-yl 2-ethylbutanoate

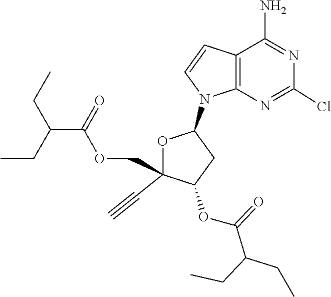 [SMILES: C#C[C@]1(COC(=O)C(CC)CC)O[C@@H](n2ccc3c(N)nc(Cl)nc32)C[C@@H]1OC(=O)C(CC)CC]

The title compound was made in a similar manner as Example 42 except using 2-ethylbutanoyl chloride. MS: m/ [symbol] =505.5 [M+H]⁺. ¹H NMR (500 MHz, CDCl₃) δ 7.14 (d, J=3.6 Hz, 1H), 6.71 (t, J=6.4 Hz, 1H), 6.41 (d, J=3.6 Hz, 1H), 5.57 (t, J=6.6 Hz, 1H), 5.43 (s, 2H), 4.46-4.36 (m, 2H), 2.82-2.73 (m, 1H), 2.69-2.60 (m, 2H), 2.38-2.24 (m, 2H), 1.76-1.49 (m, 8H), 0.97-0.85 (m, 12H).

### Example 45

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl 2-ethylbutanoate

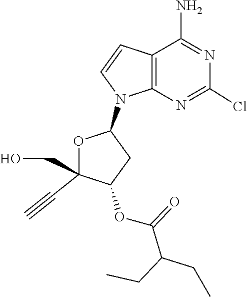 [SMILES: C#C[C@]1(CO)O[C@@H](n2ccc3c(N)nc(Cl)nc32)C[C@@H]1OC(=O)C(CC)CC]

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl 2-ethylbutanoate

In a flame-dried flask under an atmosphere of nitrogen, (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (1.1 g, 0.24 mmol) was dissolved in anhydrous pyridine (3 mL). The solution was cooled solution in an ice bath, and 2-ethylbutanoyl chloride (36 μL, 0.263 mmol) was added. The result was stirred at 0° C. for 20 mins and then at room temperature for one hour. 2-ethylbutanoyl chloride (36 μL, 0.263 mmol) was then added, and the result was stirred at room temperature for 30 mins. This step was repeated twice more. The reaction was then quenched with methanol (2 mL) and stirred for an additional 5 mins. The reaction was then concentrated under reduced pressure and partitioned between water and EtOAc. The organics were washed with water (3×) and then brine (1×), dried over Na₂SO₄, filtered and concentrated under reduced pressure. The residue was redissolved in DCM and purified on NP silica gel column (24 g ISCO-gold) eluting 0-40% EtOAc/hexanes to afford the title compound. MS: m/ [symbol] =521.5 [M+H]⁺. ¹H NMR (500 MHz, CDCl₃) δ 7.33 (d, J=3.7 Hz, 1H), 6.78 (t, J=6.7 Hz, 1H), 6.37 (d, J=3.7 Hz, 1H), 5.66-5.60 (m, 1H), 5.23 (s, 2H), 3.96 (d, J=10.9 Hz, 1H), 3.89 (d, J=11.0 Hz, 1H), 2.70-2.60 (m, 1H), 2.58-2.49 (m, 2H), 2.36-2.30 (m, 1H), 1.77-1.65 (m, 2H), 1.64-1.54 (m, 2H), 0.97-0.91 (m, 15H), 0.12 (d, J=4.6 Hz, 6H).

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl 2-ethylbutanoate

To a solution of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl 2-ethylbutanoate (95 mg, 0.18 mmol) dissolved in anhydrous THF (4 ml) was added TBAF (1M THF) (0.255 ml, 0.255 mmol). Stirred reaction at room temperature for one hour and then diluted with water and brine and extd with DCM (3×). The organics were dried over Na₂SO₄, filtered, and concentrated under reduced pressure. The residue was redissolved in DCM and purified on NP silica gel column (24 g ISCO-gold) eluting 0-80% EtOAc/hexanes to afford the title compound. MS: m/z=407.4 [M+H]⁺. ¹H NMR (500 MHz, CDCl₃) δ 7.03 (d, J=3.6 Hz, 1H), 6.34 (d, J=3.6 Hz, 1H), 6.32-6.26 (m, 1H), 5.81-5.75 (m, 1H), 5.56-5.42 (m, 3H), 4.08-4.01 (m, 1H), 3.97-3.89 (m, 1H), 3.29-3.19 (m, 1H), 2.59 (s, 1H), 2.43-2.28 (m, 2H), 1.80-1.66 (m, 2H), 1.66-1.53 (m, 2H), 0.99-0.91 (m, 6H).

### Example 46

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl 2-phenylacetate

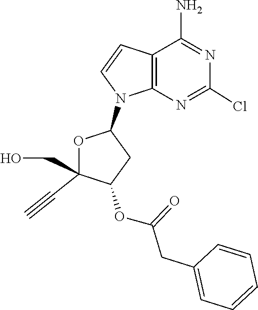 [SMILES: C#C[C@]1(CO)O[C@@H](n2ccc3c(N)nc(Cl)nc32)C[C@@H]1OC(=O)Cc1ccccc1]

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl 2-phenylacetate

In a flame-dried flask under an atmosphere of nitrogen, (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (100 mg, 0.236 mmol) was dissolved in anhydrous pyridine (3 ml). The solution was cooled solution in an ice bath, and 2-phenylacetyl chloride (63 μL, 0.476 mmol) was added. The result was stirred at room temperature for one hour. Additional 2-ethylbutanoyl chloride (63 μL, 0.476 mmol) was added. The result was stirred at room temperature for 60 minutes, then quenched with methanol (2 mL) and then stirred for an additional 5 minutes. The reaction was then concentrated under reduced pressure and partitioned between water and EtOAc. The organics were washed with water (3×) and then brine (lx), dried over Na₂SO₄, filtered and concentrated under reduced pressure. The residue was redissolved in DCM and purified on NP silica gel column (40 g ISCO-gold) eluting 0-60% EtOAc/hexanes to afford the title compound. MS: m/ [symbol] =541.5 [M+H]⁺.

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl 2-phenylacetate

To a solution of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl 2-phenylacetate (79 mg, 0.146 mmol) dissolved in anhydrous THF (4 ml) was added TBAF (1M THF) (0.204 ml, 0.204 mmol). Stirred reaction at room temperature for one hour and then diluted with water and brine and extd with DCM (3×). The organics were dried over Na₂SO₄, filtered, and concentrated under reduced pressure. The residue was purified by RP chromatography (15-60% ACN-water (5 mM NH₄CO₃) over 14 min and held at 50% ACN on Waters Xbridge 30×150 mm). The appropriate fractions were combined, diluted with brine, and extracted with DCM (3×). The combined organics were washed with brine, dried over Na₂SO₄, filtered and concentrated under reduced pressure. The residue was further purified on NP silica gel column (4 g ISCO-gold) eluting 0-100% EtOAc/hexanes to afford the title compound. MS: m/z=427.4 [M+H]⁺. ¹H NMR (500 MHz, CDCl₃) δ 7.38-7.24 (m, 5H), 6.99 (d, J=3.6 Hz, 1H), 6.34 (d, J=3.6 Hz, 1H), 6.26-6.20 (m, 1H), 5.78 (d, J=6.1 Hz, 1H), 5.68-5.59 (m, 3H), 4.04-3.98 (m, 1H), 3.95-3.87 (m, 1H), 3.73 (s, 2H), 3.30-3.21 (m, 1H), 2.41-2.33 (m, 2H).

### Example 47

### ((2R,3S,5R)-5-(4-amino-2-chloro-7H-pyrrolo[2,3-d]pyrimidin-7-yl)-2-ethynyl-3-(propionyloxy)tetrahydrofuran-2-yl)methyl decanoate

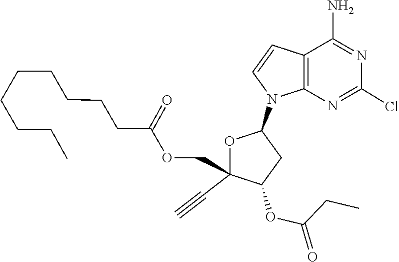 [SMILES: C#C[C@]1(COC(=O)CCCCCCCCC)O[C@@H](n2ccc3c(N)nc(Cl)nc32)C[C@@H]1OC(=O)CC]

Under an atmosphere of nitrogen, ((2R,3S,5R)-5-(4-amino-2-chloro-7H-pyrrolo[2,3-d]pyrimidin-7-yl)-2-ethynyl-3-hydroxytetrahydrofuran-2-yl)methyl decanoate (Example 20) (60 mg, 0.130 mmol) was dissolved in anhydrous pyridine (3 ml). To this solution was added propionyl chloride (14 μL, 0.160 mmol) and stirred at room temperature overnight. Afterward, more propionyl chloride (28 μL, 0.32 mmol) was added, and the solution was stirred for another hour at room temperature. This reaction was quenched with methanol (2 mL), and the solution was stirred for an additional 20 mins. The reaction was then concentrated under reduced pressure and partitioned between water and diethyl ether. The organics were washed with water (3×) and then brine (lx), dried over Na₂SO₄, filtered and concentrated under reduced pressure. The residue was redissolved in DCM and purified on NP silica gel column (40 g ISCO-gold) eluting 0-60% EtOAc/hexanes. The appropriate fractions were concentrated under reduced pressure, redissolved in ether and concentrated under reduced pressure (2×). The residue was recrystallized in ether/hexanes, with gentle warming to remove most of the ether and with slow addition of hexanes. The solid was filtered with 10:1 hexane-ether rinse to provide the title compound. MS: m/z=519.5 [M+H]⁺. ¹H NMR (500 MHz, CDCl₃) δ 7.14-7.10 (m, 1H), 6.75-6.69 (m, 1H), 6.42-6.39 (m, 1H), 5.61-5.56 (m, 1H), 5.27 (s, 2H), 4.45 (d, J=11.8 Hz, 1H), 4.36 (d, J=11.5 Hz, 1H), 2.75-2.68 (m, 1H), 2.67-2.60 (m, 2H), 2.49-2.42 (m, 2H), 2.41-2.34 (m, 2H), 1.68-1.61 (m, 2H), 1.35-1.17 (m, 15H), 0.91-0.84 (m, 3H).

### Example 48

### ((2R,3S,5R)-3-acetoxy-5-(4-amino-2-chloro-7H-pyrrolo[2,3-d]pyrimidin-7-yl)-2-ethynyltetrahydrofuran-2-yl)methyl decanoate

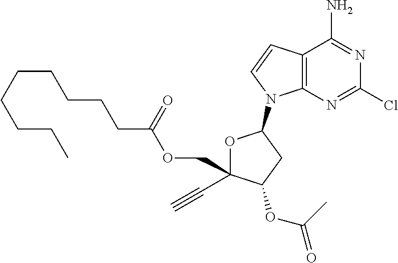 [SMILES: C#C[C@]1(COC(=O)CCCCCCCCC)O[C@@H](n2ccc3c(N)nc(Cl)nc32)C[C@@H]1OC(C)=O]

The title compound was made in a similar manner as Example 47 except using acetyl chloride. MS: m/ [symbol] =505.5 [M+H]⁺. ¹H NMR (500 MHz, CDCl₃) δ 7.11 (d, J=3.7 Hz, 1H), [symbol] 6.72 (t, J=6.7 Hz, 1H), 6.40 (d, J=3.8 Hz, 1H), 5.60-5.54 (m, 1H), 5.25 (s, 2H), 4.45 (d, J=11.8 Hz, 1H), 4.36 (d, J=11.8 Hz, 1H), 2.76-2.59 (m, 3H), 2.42-2.34 (m, 2H), 2.18 (s, 3H), 1.67-1.61 (m, 2H), 1.35-1.22 (m, 12H), 0.88 (t, J=6.9 Hz, 3H).

### Examples 49 and 50

### Example 49:(2R,3S,5R)-5-(4-amino-2-chloro-7H-pyrrolo[2,3-d]pyrimidin-7-yl)-2-ethynyl-2-((picolinoyloxy)methyl)tetrahydrofuran-3-yl picolinate—

### Example 50: ((2R,3S,5R)-5-(4-amino-2-chloro-7H-pyrrolo[2,3-d]pyrimidin-7-yl)-3-hydroxy-2-methyltetrahydrofuran-2-yl)methyl picolinate

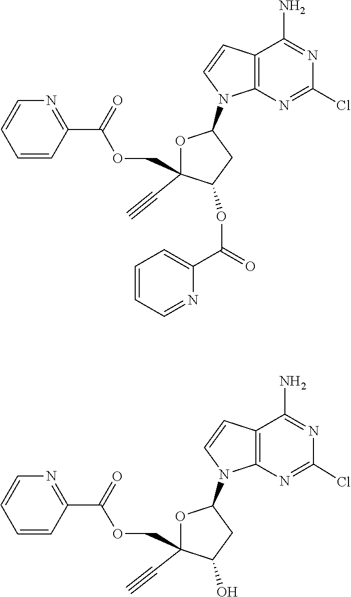 [SMILES: C#C[C@]1(COC(=O)c2ccccn2)O[C@@H](n2ccc3c(N)nc(Cl)nc32)C[C@@H]1OC(=O)c1ccccn1 ; C#C[C@]1(COC(=O)c2ccccn2)O[C@@H](n2ccc3c(N)nc(Cl)nc32)C[C@@H]1O]

The title compounds were made in a similar manner as Example 6 except using picolinic acid in Step 1 and isolating both the bis-ester Example 49 and mono-ester Example 50.

Example 49: MS: m/ [symbol] =519.05 [M+H]⁺. ¹H NMR (300 MHz, DMSO-d₆) δ 8.83-8.77 (m, 2H), 8.22-8.20 (m, 1H), 8.16-8.13 (m, 1H), 8.09-8.02 (m, 2H), 7.74-7.69 (m, 2H), 7.66-7.61 (m, 3H), 6.71-6.68 (m, 1H), 6.60 (d, J=3.9 Hz, 1H), 6.01-5.98 (m, 1H), 4.67 (s, 2H), 3.77 (s, 1H), 3.25-3.16 (m, 1H), 2.83-2.73 (m, 1H).

Example 50: MS: m/ [symbol] =414.00 [M+H]⁺. ¹H NMR (400 MHz, DMSO-d₆) δ 8.73-8.72 (m, 1H), 8.05-7.95 (m, 2H), 7.69-7.68 (m, 1H), 7.40-7.38 (m, 1H), 6.52-6.50 (m, 1H), 6.44-6.41 (m, 1H), 4.69-4.64 (m, 2H), 4.43-4.40 (m, 1H), 3.57-3.50 (m, 1H), 2.74-2.62 (m, 1H), 2.48-2.45 (m, 1H).

### Example 51

### ((2R,3S,5R)-5-(4-amino-2-chloro-7H-pyrrolo[2,3-d]pyrimidin-7-yl)-3-hydroxy-2-methyltetrahydrofuran-2-yl)methyl picolinate

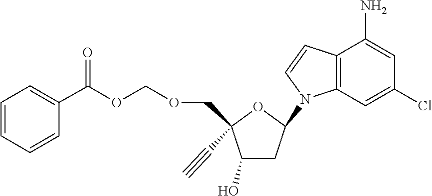 [SMILES: C#C[C@]1(COCOC(=O)c2ccccc2)O[C@@H](n2ccc3c(N)cc(Cl)cc32)C[C@@H]1O]

### Step 1: (((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methoxy)methyl benzoate

A mixture of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)-tetrahydrofuran-2-yl)methanol (0.20 g, 0.23 mmol) and NaH (17 mg, 0.70 mmol) in DMF (2 mL) was formed under argon atmosphere at 0° C. The reaction was stirred for 10 min at 0° C. Then was added iodomethyl benzoate (92 mg, 0.35 mmol) at 0° C. and stirred for 15 min at 25° C. The reaction was diluted with NH₄Cl (3 mL), extracted with EtOAc (100 mL), washed with brine (3×50 mL), dried over Na₂SO₄ and concentrated. The residue as purified by prep-TLC (DCM) to afford the title compound. MS: m/ [symbol] =987.45 [M+H]⁺.

### Step 2: ((2R,3S,5R)-5-(4-amino-2-chloro-7H-pyrrolo[2,3-d]pyrimidin-7-yl)-3-hydroxy-2-methyltetrahydrofuran-2-yl)methyl picolinate

A mixture of (((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)-tetrahydrofuran-2-yl)methoxy)methyl benzoate (0.15 g, 0.15 mmol) in water (0.50 mL), THF (1.0 mL) and formic acid (2.0 mL) was stirred for 30 min at 25° C. The reaction was diluted with toluene (5 mL) and concentrated. The reaction was purified by Prep-HPLC with the following condition: Column: SunFire C18 OBD Prep Column 19*150 mm, 5 m; Mobile Phase A: Water (0.1% FA), Mobile Phase B: ACN; Flow rate: 20 mL/min mL/min; Gradient: 30% B to 40% B in 15 min; Wave Length: UV 254 nm/210 nm nm; RT1: 5.5 min to afford the title compound. MS: m/ [symbol] =443.05 [M+H]⁺. ¹H-NMR (300 MHz, CD₃OD): δ 7.98-7.95 (m, 2H), 7.59-7.54 (m, 1H), 7.44-7.39 (m, 2H), 7.25 (d, J=3.6 Hz, 1H), 6.56 (d, J=3.9 Hz, 1H), 6.50 (t, J=‘6.6 Hz, 1H), 5.55-5.50 (m, 2H), 4.70 (t, J=7.5 Hz, 1H), 4.06-4.05 (m, 2H), 3.10 (s, 1H), 2.59-2.54 (m, 2H).

### Example 52

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl 2-propylpentanoate

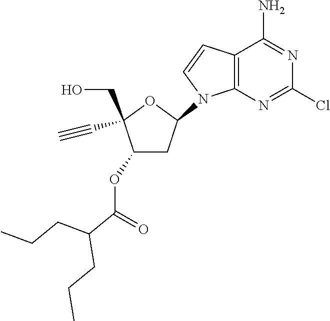 [SMILES: C#C[C@]1(CO)O[C@@H](n2ccc3c(N)nc(Cl)nc32)C[C@@H]1OC(=O)C(CCC)CCC]

The title compound was made in a similar manner as Example 12 except using 2-propylpentanoyl chloride in Step 1. MS: m/ [symbol] =435.10 [M+H]⁺. ¹H NMR (300 MHz, DMSO-d₆) δ 7.59-7.39 (m, 3H), 6.63-6.45 (m, 2H), 5.57-5.55 (m, 2H), 3.65-3.61 (m, 3H), 2.86-2.75 (m, 1H), 2.50-2.42 (m, 2H), 1.58-1.28 (m, 8H), 0.89 (s, 6H).

### Example 53

### ((2R,3S,5R)-5-(4-amino-2-chloro-7H-pyrrolo[2,3-d]pyrimidin-7-yl)-2-ethynyl-3-((((5-methyl-2-oxo-1,3-dioxol-4-yl)methoxy)carbonyl)oxy)tetrahydrofuran-2-yl)methyl ((5-methyl-2-oxo-1,3-dioxol-4-yl)methyl) carbonate

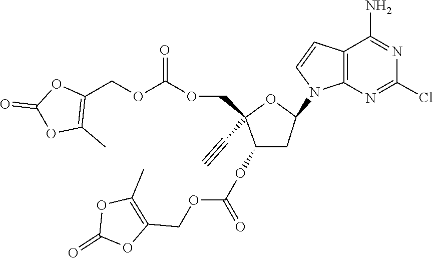

To a stirred mixture of (2R,3S,5R)-5-(4-amino-2-chloro-7H-pyrrolo[2,3-d]pyrimidin-7-yl)-2-ethynyl-2-(hydroxymethyl)tetrahydrofuran-3-ol (50 mg, 0.16 mmol) in DCM/DMF (2.5 mL, 2:1) was added (5-methyl-2-oxo-1,3-dioxol-4-yl)methyl (4-nitrophenyl) carbonate (96 mg, 0.32 mmol) and TEA (0.045 mL, 0.32 mmol) at room temperature. The resulting mixture was warmed to 40° C. and stirred for 16 h. The reaction mixture was concentrated under reduced pressure and the residue was purified by RP-Combiflash with the following conditions: Column: C18 40 g Column, 60 Å, 20-35 μm; Mobile Phase A: water, Mobile Phase B: MeCN; Flow rate: 50 mL/min; 0% B to 100% B in 20 min; Detector: UV 254 & 210 nm to afford the title compound. MS: m/ [symbol] =621.05 [M+H]⁺. ¹H-NMR (400 MHz, CD₃OD) δ 7.20 (d, J=3.6 Hz, 1H), 6.70-6.46 (m, 2H), 5.65-5.50 (m, 1H), 5.09-4.91 (m, 4H), 4.60-4.51 (m, 1H), 4.50-4.30 (m, 1H), 3.26 (s, 1H), 3.00-2.81 (m, 1H), 2.79-2.51 (m, 1H), 2.27-2.10 (m, 6H).

### Example 54

### (((2R,3S,5R)-5-(4-amino-2-chloro-7H-pyrrolo[2,3-d] pyrimidin-7-yl)-2-ethynyl-3-hydroxytetrahydrofuran-2-yl)methoxy)methyl isobutyrate

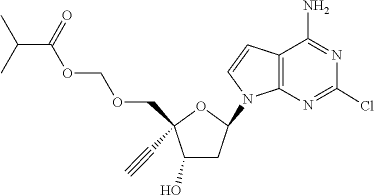 [SMILES: C#C[C@]1(COCOC(=O)C(C)C)O[C@@H](n2ccc3c(N)nc(Cl)nc32)C[C@@H]1O]

The title compound was made in a similar manner as Example 51 except using chloromethyl isobutyrate in Step 1. MS: m/ [symbol] =409.05 [M+H]⁺. ¹H-NMR (400 MHz, DMSO-d₆): δ 7.56-7.30 (m, 2H), 7.30-7.28 (m, 1H), 6.62 (d, J=3.6 Hz, 1H), 6.42-6.39 (m, 1H), 5.70-5.68 (m, 1H), 5.25-5.21 (m, 2H), 4.77-4.45 (m, 2H), 3.85-3.56 (m, 3H), 2.56-2.50 (m, 1H), 2.50-2.36 (m, 1H), 1.07-1.06 (m, 6H).

### Example 55

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl nonanoate

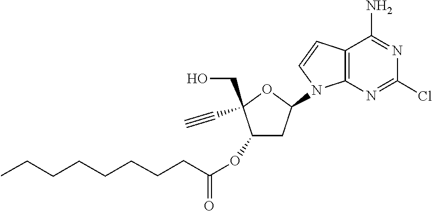 [SMILES: C#C[C@]1(CO)O[C@@H](n2ccc3c(N)nc(Cl)nc32)C[C@@H]1OC(=O)CCCCCCCC]

The title compound was made in a similar manner as Example 12 except using nonanoyl chloride in Step 1. MS: m/ [symbol] =449.10 [M+H]⁺. 1H NMR (300 MHz, Methanol-d4) δ 7.32 (d, J=3.9 Hz, 1H), 6.59 (d, J=3.9 Hz, 1H), 6.51 (dd, J=8.1, 6.0 Hz, 1H), 5.65 (dd, J=6.9, 3.3 Hz, 1H), 3.90-3.75 (m, 2H), 3.10 (s, 1H), 2.92-2.82 (m, 1H), 2.55-2.38 (m, 3H), 1.70-1.63 (m, 2H), 1.45-1.25 (m, 10H), 0.92-0.88 (m, 3H).

### Example 56

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl((5-methyl-2-oxo-1,3-dioxol-4-yl)methyl) carbonate

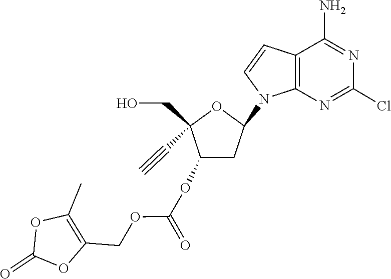 [SMILES: C#C[C@]1(CO)O[C@@H](n2ccc3c(N)nc(Cl)nc32)C[C@@H]1OC(=O)OCc1oc(=O)oc1C]

The title compound was made in a similar manner as Example 12 except using (5-methyl-2-oxo-1,3-dioxol-4-yl)methyl (4-nitrophenyl) carbonate in Step 1. MS: m/ [symbol] =465.05[M+H]⁺. ¹H-NMR (400 MHz, CD₃OD) δ 7.30 (d, J=4.0 Hz, 1H), 6.59 (d, J=4.0 Hz, 1H), 6.50-6.46 (m, 1H), 5.60-5.40 (m, 1H), 5.10-5.00 (m, 2H), 3.91-3.72 (m, 2H), 3.11 (s, 1H), 3.01-2.85 (m, 1H), 2.64-2.50 (m, 1H), 2.25 (s, 3H).

### Example 57

### ((2R,3S,5R)-5-(4-amino-2-chloro-7H-pyrrolo[2,3-d]pyrimidin-7-yl)-2-ethynyl-3-hydroxytetrahydrofuran-2-yl)methyl octanoate

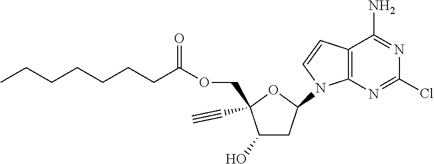 [SMILES: C#C[C@]1(COC(=O)CCCCCCC)O[C@@H](n2ccc3c(N)nc(Cl)nc32)C[C@@H]1O]

The title compound was made in a similar manner as Example 14 except using octanoyl chloride in Step 2. MS: m/ [symbol] =435.10 [M+H]⁺. ¹H NMR (300 MHz, DMSO-d₆) δ 7.55 (s, 2H), 7.29 (d, J=3.6 Hz, 1H), 6.61 (d, J=3.6 Hz, 1H), 6.39-6.35 (m, 1H), 5.77 (s, 1H), 4.54 (t, J=6.9 Hz, 1H), 4.40-4.36 (m, 1H), 4.10-4.06 (m, 1H), 3.60 (s, 1H), 2.45-2.38 (m, 2H), 2.29-2.23 (m, 2H), 1.48-1.44 (m, 2H), 1.24-1.20 (m, 8H), 0.86-0.81 (m, 3H).

### Example 58

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl heptanoate

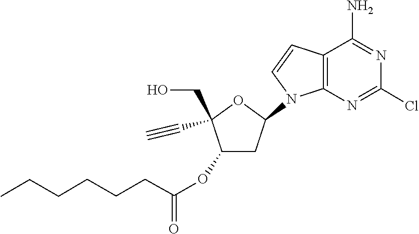 [SMILES: C#C[C@]1(CO)O[C@@H](n2ccc3c(N)nc(Cl)nc32)C[C@@H]1OC(=O)CCCCCC]

The title compound was made in a similar manner as Example 12 except using heptanoyl chloride in Step 1. MS: m/ [symbol] =421.10 [M+H]⁺. ¹H NMR (300 MHz, DMSO-d₆) δ 7.60 (s, 2H), 7.40 (d, J=3.6 Hz, 1H), 6.63 (d, J=3.6 Hz, 1H), 6.48-6.43 (m, 1H), 5.59-5.53 (m, 2H), 3.66-3.57 (m, 3H), 2.81-2.72 (m, 1H), 2.46-2.37 (m, 3H), 1.64-1.54 (m, 2H), 1.36-1.23 (m, 6H), 0.92-0.82 (m, 3H).

### Example 59

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl hexanoate

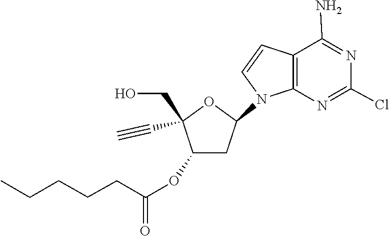 [SMILES: C#C[C@]1(CO)O[C@@H](n2ccc3c(N)nc(Cl)nc32)C[C@@H]1OC(=O)CCCCC]

The title compound was made in a similar manner as Example 12 except using hexanoyl chloride in Step 1. MS: m/ [symbol] =407.10 [M+H]⁺. ¹H-NMR (400 MHz, CD₃OD) δ 7.32 (d, J=4.0 Hz, 1H), 6.65-6.57 (m, 1H), 6.57-6.49 (m, 1H), 5.70-5.60 (m, 1H), 3.90-3.72 (m, 2H), 3.13 (s, 1H), 2.92-2.80 (m, 1H), 2.52-2.40 (m, 3H), 1.80-1.60 (m, 2H), 1.42-1.29 (m, 4H), 1.01-0.82 (m, 3H).

### Example 60

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl 4-phenylbutanoate

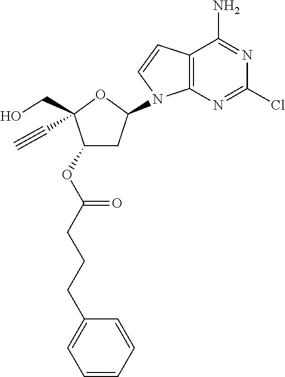 [SMILES: C#C[C@]1(CO)O[C@@H](n2ccc3c(N)nc(Cl)nc32)C[C@@H]1OC(=O)CCCc1ccccc1]

The title compound was made in a similar manner as Example 12 except using 4-phenylbutanoyl chloride in Step 1. MS: m/ [symbol] =455.05 [M+H]⁺. ¹H-NMR (400 MHz, CD₃OD): δ 7.32-7.31 (m, 1H), 7.29-7.20 (m, 2H), 7.18-7.16 (m, 3H), 6.59 (d, J=4.0 Hz, 1H). 6.52-6.48 (m, 1H), 5.66-5.64 (m, 1H), 3.84-3.82 (m, 2H), 3.08 (s, 1H), 2.99-2.82 (m, 1H), 2.69 (d, J=7.6 Hz, 2H), 2.47-2.43 (m, 3H), 2.03-1.98 (m, 2H).

Alternatively, Example 60 can be prepared as follows.

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl 4-phenylbutanoate—

Under an atmosphere of nitrogen, (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (2.0 g. 4.5 mmol) was dissolved in anhydrous pyridine (22 mL). The solution was cooled in an ice bath, and 4-phenylbutanoyl chloride (1.5 mL, 9.2 mmol) was added dropwise. The resulting mixture was stirred at 0° C. for 20 mins and then at room temperature for one hour. The reaction was cooled back to 0° C. and additional 4-phenylbutanoyl chloride (0.75 mL, 4.6 mmol) was added. The resulting mixture was warmed to room temperature and stirred for an additional 30 mins. The reaction was quenched with methanol (5 mL) and stirred for an additional 5 mins. The reaction was then concentrated under reduced pressure. The residue was redissolved in DCM and purified on NP silica gel column (120 g ISCO-gold) eluting 0-50% 3:1 EtOAc:EtOH/hexanes to afford the title compound. MS: m/ [symbol] =569.5 [M+H]⁺.

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl 4-phenylbutanoate

A mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl 4-phenylbutanoate (2.4 g, 4.3 mmol) in THF (85 mL) was cooled in an ice bath and tetrabutylammonium fluoride (4.5 mL, 4.5 mmol, 1 M in THF) was added. The resulting mixture was warmed to room temperature and stirred for 30 mins. The reaction was partitioned between water and diethyl ether. The organics were washed with water (3×) and then brine (1×), dried over Mg₂SO₄, filtered and concentrated under reduced pressure. The residue was redissolved in DCM and purified on NP silica gel column (330 g ISCO-gold) eluting 0-50% 3:1 EtOAc:EtOH/hexanes. The appropriate fractions were concentrated under reduced pressure, and then redissolved in ether and concentrated under reduced pressure (2×). The residue was recrystallized by stirring in heptane/EtOAc (85:15). After overnight stirring, crystals crashed out and the solid was filtered with heptane:EtOAc rinse to provide the title compound. MS: m/ [symbol] =455.4 [M+H]⁺. ¹H NMR (500 MHz, Chloroform-d) δ 7.33-7.27 (m, 3H), 7.21 (t, J=6.8 Hz, 3H), 7.00 (d, J=3.7 Hz, 1H), 6.33 (d, J=3.7 Hz, 1H), 6.25 (dd, J=9.5, 5.5 Hz, 1H), 5.78 (d, J=5.1 Hz, 1H), 5.59 (dd, J=11.7, 2.8 Hz, 1H), 5.33 (s, 2H), 4.04 (dd, J=12.4, 2.8 Hz, 1H), 3.94 (t, J=12.1 Hz, 1H), 2.71 (t, J=7.5 Hz, 2H), 2.55 (s, 1H), 2.45 (t, J=7.5 Hz, 2H), 2.40-2.30 (m, 1H), 2.10-2.00 (m, 2H).

### Example 61

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl hexyl carbonate

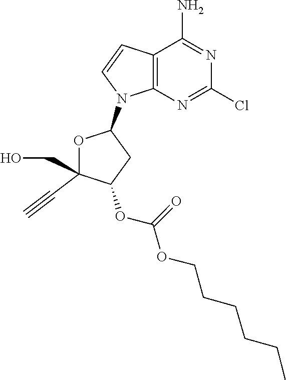 [SMILES: C#C[C@]1(CO)O[C@@H](n2ccc3c(N)nc(Cl)nc32)C[C@@H]1OC(=O)OCCCCCC]

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl hexyl carbonate

Under an atmosphere of nitrogen, (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (2.3 g, 5.4 mmol) was dissolved in anhydrous pyridine (25 mL). The solution was put into a cold water bath, and hexyl carbonochloridate (1.2 mL, 7.4 mmol) was added dropwise. The reaction was stirred at RT overnight. Additional hexyl carbonochloridate (0.4 mL) was added and the reaction was stirred at room temperature for two hours. The reaction was quenched with methanol (5 mL) and stirred for an additional 5 mins. The reaction was then concentrated under reduced pressure. The residue was partitioned between water and diethyl ether. The organics were washed with water (3×) and then brine (lx), dried over Na₂SO₄, filtered, and concentrated under reduced pressure. The residue was redissolved in DCM and purified on NP silica gel column (120 g ISCO-gold) eluting 0-50% EtOAc/hexanes to afford the title compound. MS: m/ [symbol] =551.5 [M+H]⁺.

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl hexyl carbonate

A mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl hexyl carbonate (2.5 g, 4.6 mmol) in anhydrous THF (20 mL) was put into a cold water bath and tetrabutylammonium fluoride (4.8 mL, 4.8 mmol, 1 M in THF) was added. The reaction was warmed to room temperature and stirred for 90 mins. The reaction was partitioned between water and diethyl ether. The organics were washed with water (3×) and then brine (lx), dried over Na₂SO₄, filtered and concentrated under reduced pressure. The residue was redissolved in DCM and purified on NP silica gel column (80 g ISCO-gold) eluting 0-60% EtOAc/hexanes and then recrystallized from iPrOH to provide the title compound. MS: m/ [symbol] =437.5 [M+H]⁺. ¹H-NMR (500 MHz, CDCl₃): δ 6.97 (d, J=3.6 Hz, 1H), 6.33 (d, J=3.6 Hz, 1H), 6.26 (dd, J=9.7, 5.4 Hz, 1H), 5.83 (dd, J=12.0, 2.3 Hz, 1H), 5.63 (d, J=6.2 Hz, 1H), 5.43 (s, 2H), 4.28-4.14 (m, 2H), 4.06 (dd, J=12.3, 2.3 Hz, 1H), 3.96 (t, J=12.2 Hz, 1H), 3.36-3.29 (m, 1H), 2.62 (s, 1H), 2.47 (dd, J=13.9, 5.4 Hz, 1H), 1.75-1.67 (m, 2H), 1.43-1.35 (m, 2H), 1.35-1.29 (m, 4H), 0.95-0.88 (m, 3H).

### Example 62

### ((2R,3S,5R)-5-(4-amino-2-chloro-7H-pyrrolo[2,3-d]pyrimidin-7-yl)-2-ethynyl-3-hydroxytetrahydrofuran-2-yl)methyl 3-(adamantan-1-yl)propanoate

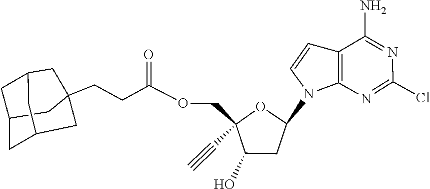 [SMILES: C#C[C@]1(COC(=O)CCC23CC4CC(CC(C4)C2)C3)O[C@@H](n2ccc3c(N)nc(Cl)nc32)C[C@@H]1O]

### Step 1: ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methyl 3-(adamantan-1-yl)propanoate

To a solution of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methanol (0.10 g, 0.12 mmol) in DCM (3 mL) under an atmosphere of N₂, was added 3-(adamantan-1-yl)propanoic acid (49 mg, 0.23 mmol), DMAP (14 mg, 0.12 mmol), and DCC (48 mg, 0.23 mmol). The resulting mixture was stirred at 25° C. for 16 h. The residue was purified by prep-TLC, developed using ethyl acetate/petroleum ether (1/1) to afford the title compound. MS: m/ [symbol] =1043.5 [M+H]⁺.

### Step 2: ((2R,3S,5R)-5-(4-amino-2-chloro-7H-pyrrolo[2,3-d]pyrimidin-7-yl)-2-ethynyl-3-hydroxytetrahydrofuran-2-yl)methyl 3-(adamantan-1-yl)propanoate—

To a solution of ((2R,3S,5R)-5-(2-chloro-4-(((4-methoxyphenyl)diphenylmethyl)amino)-7H-pyrrolo[2,3-d]pyrimidin-7-yl)-2-ethynyl-3-((4-methoxyphenyl)diphenylmethoxy)tetrahydrofuran-2-yl)methyl 3-(adamantan-1-yl)propanoate (0.15 g, 0.14 mmol) in water (1 mL) under N₂ atmosphere, was added formic acid (4.0 mL, 0.14 mmol). The resulting mixture was stirred at 25° C. for 16 h. The residue was purified by RP-Flash (Column: C18 40 g Column; Mobile Phase A: water, Mobile Phase B: MeCN; Flow rate: 40 mL/min; Gradient: 2% B to 30% B in 30 min, Detector: UV 210 nm; RT=28 min). The fractions containing desired product were combined and concentrated under reduced pressure to afford the title compound MS: m/z=499.25 [M+H]⁺. ¹H NMR (400 MHz, CD₃OD) δ 7.24-7.19 (m, 1H), 6.60-6.59 (m, 1H), 6.45-6.42 (m, 1H), 4.85-4.76 (m, 1H), 4.46-4.43 (m, 1H), 4.23-4.20 (m, 1H), 3.16 (s, 1H), 2.78-2.59 (m, 2H), 2.35-2.17 (m, 2H), 1.94 (s, 3H), 1.74-1.70 (m, 3H), 1.65-1.62 (m, 3H), 1.50-1.39 (m, 6H), 1.37-1.29 (m, 2H).

### Example 63

### Tetradecyl (2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)carbamate

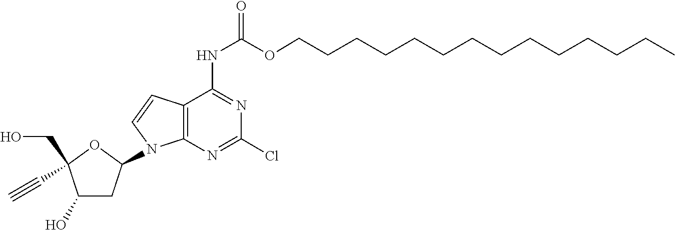 [SMILES: C#C[C@]1(CO)O[C@@H](n2ccc3c(NC(=O)OCCCCCCCCCCCCCC)nc(Cl)nc32)C[C@@H]1O]

### Step 1: Tetradecyl (7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)carbamate

To a mixture of 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (Intermediate 2) (0.50 g, 0.93 mmol) in anhydrous DMF (6 mL) was added sodium hydride (23 mg, 0.94 mmol, washed with hexane). The resulted mixture was stirred for 30 min at room temperature. To this was added Intermediate 19 (0.42 g, 1.1 mmol). The resultant mixture was stirred for 2 hr at room temperature. The reaction was diluted with ethyl acetate and washed with brine (3×), dried over Na₂SO₄, filtered, and the solvent was concentrated under reduced pressure. The residue was purified by column chromatography on silica gel, eluting with EA/PE to provide the title compound MS: m/ [symbol] =777.60 [M+H]⁺.

### Step 2: Tetradecyl (2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)carbamate

To a mixture of tetradecyl (7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)carbamate (0.30 g, 0.39 mmol) in THF (3 mL) was added TEA.3HF (1.0 mL, 6.3 mmol). The resulted mixture was stirred overnight at 50° C. The reaction was purified by RP-CombiFlash, eluting with acetonitrile/water to provide the title compound. MS: m/ [symbol] =549.35 [M+H]⁺. ¹H NMR (400 MHz, DMSO-d₆) δ 10.54 (br, 1H), 7.63 (d, J=3.6 Hz, 1H), 6.90 (d, J=3.6 Hz, 1H), 6.55-6.52 (m, 1H), 5.56 (d, J=5.6 Hz, 1H), 5.27-5.25 (m, 1H), 4.52-4.50 (m, 1H), 4.14 (t, J=6.4 Hz, 2H), 3.61-3.55 (m, 2H), 3.50 (s, 1H), 2.53-2.42 (m, 2H), 1.65-1.61 (m, 2H), 1.36-1.31 (m, 22H), 0.90-0.79 (m, 3H).

### Example 64

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl phenethyl carbonate

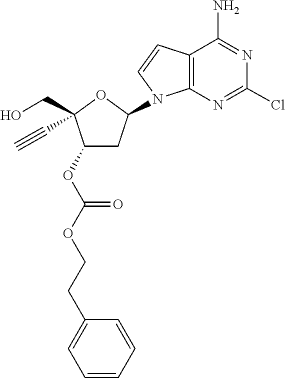 [SMILES: C#C[C@]1(CO)O[C@@H](n2ccc3c(N)nc(Cl)nc32)C[C@@H]1OC(=O)OCCc1ccccc1]

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl phenethyl carbonate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (0.10 g, 0.24 mmol) in pyridine (0.5 mL), was added TEA (0.066 mL, 0.47 mmol), DMAP (29 mg, 0.24 mmol) and 4-nitrophenyl phenethyl carbonate (Intermediate 9) (0.14 g, 0.47 mmol) at room temperature. The resulting mixture was stirred at 80° C. for 16 h and then concentrated under reduced pressure. The residue was diluted with DCM and the solution was purified by TLC (developed by Pet. ether/ethyl acetate (2:1)) to afford the title compound. MS: m/z=571.15 [M+H]⁺.

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl phenethyl carbonate

To a mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl phenethyl carbonate (0.11 g, 0.19 mmol) in THF (2 mL) was added triethylamine trihydrofluoride (1.0 mL, 6.3 mmol) at room temperature under a nitrogen atmosphere. The reaction was stirred at room temperature for 16 h. The mixture was filtered and the filtrate was concentrated under reduced pressure. The residue was purified by RP-combiflash with the following conditions: C18, 40 g, Mobile Phase A: water (1‰ FA), Mobile Phase B: acetonitrile, gradient: 0˜100% B in 30 min, Flow rate: 40 mL/min, UV Detector: 254 nm to afford the title compound. MS: m/z=457.05 [M+H]⁺. ¹H NMR (400 MHz, CDCl₃) δ 7.36-7.20 (m, 5H), 6.97 (d, J=3.6 Hz, 1H), 6.34 (d, J=3.6 Hz, 1H), 6.26-6.22 (m, 1H), 5.78 (s, 1H), 5.61 (d, J=6.0 Hz, 1H), 5.53 (s, 2H), 4.51-4.35 (m, 2H), 4.05-4.02 (m, 1H), 3.94-3.87 (m, 1H), 3.34-3.26 (m, 1H), 3.03 (t, J=6.8 Hz, 2H), 2.48-2.46 (m, 2H).

Step 2 can alternatively be performed as follows:

A solution of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl phenethyl (2.9 g, 5.1 mmol) in anhy THF (40 mL) under an atmosphere of N₂ was put into a cold water bath. To this was added TBAF (5.1 mL, 5.1 mmol, 1 M in THF) and the reaction was stirred at RT for 45 min. The reaction was diluted with ether and washed with water (3×) and brine (1×). The organic layer was dried over Na₂SO₄, filtered, and concentrated under reduced pressure. The residue was dissolved in DCM and purified on NP silica gel column (120 g ISCO-gold) eluting 0-60% EtOAc-hexanes. The appropriate fractions were concentrated under reduced pressure and then recrystallized from 1:1 EtOAc/heptane to afford the title compound. MS: m/ [symbol] =457.4 [M+H]⁺. ¹H NMR (500 MHz, CDCl₃) δ 7.35-7.29 (m, 2H), 7.28-7.24 (m, 3H), 6.98-6.94 (m, 1H), 6.35-6.31 (m, 1H), 6.27-6.20 (m, 1H), 5.82 (d, J=12.0 Hz, 1H), 5.62 (d, J=6.0 Hz, 1H), 5.49 (s, 2H), 4.52-4.43 (m, 1H), 4.43-4.35 (m, 1H), 4.04 (d, J=12.5 Hz, 1H), 3.93 (t, J=12.3 Hz, 1H), 3.36-3.26 (m, 1H), 3.03 (t, J=6.9 Hz, 2H), 2.48-2.42 (m, 2H).

### Example 65

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl propionate

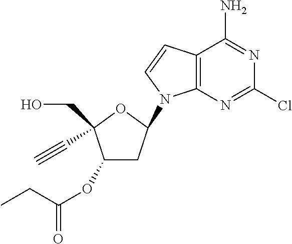 [SMILES: C#C[C@]1(CO)O[C@@H](n2ccc3c(N)nc(Cl)nc32)C[C@@H]1OC(=O)CC]

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl propionate

To a mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (0.30 g, 0.71 mmol), DCC (0.29 g, 1.4 mmol), DMAP (87 mg, 0.71 mmol) in DMF (2 mL) was added propionic acid (0.11 g, 1.4 mmol) at room temperature. The reaction was warmed to 50° C. and stirred for 16 h. The reaction was diluted with EtOAc (20 mL) and washed with water (2×) and brine (2×), dried over Na₂SO₄ and concentrated under reduced pressure. The residue was purified by silica gel column chromatography, eluting with 0-50% EtOAc in Pet. ether to afford the title compound. MS: m/ [symbol] =479.20[M+H]⁺.

### Step 2: (2R,3S,5R)-5-(4-amino-2-chloro-7H-pyrrolo[2,3-d]pyrimidin-7-yl)-2-ethynyl-2-(hydroxymethyl)tetrahydrofuran-3-yl propionate—

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl propionate (0.10 g, 0.21 mmol) in THF (2 mL) was added triethylamine trihydrofluoride (1.0 mL, 6.3 mmol) at 25° C. The reaction was stirred at 25° C. for 16 h and then concentrated under reduced pressure. The residue was purified by RP-Combiflash with the following conditions: Column: C18 Column, 40 g, 60 Å, 20-35 μm; Mobile Phase A: water, Mobile Phase B: MeCN; Flow rate: 50 mL/min; 0% B to 100% B in 15 min; Detector: UV 254 & 210 nm to afford the title compound. MS: m/ [symbol] =364.95 [M+H]⁺. ¹H-NMR (400 MHz, CD₃OD) δ 7.33 (d, J=3.6 Hz, 1H), 6.59-6.49 (m, 2H), 5.67-5.65 (m, 1H), 3.87-3.79 (m, 2H), 3.11 (s, 1H), 2.91-2.86 (m, 1H), 2.51-2.43 (m, 3H), 1.28-1.18 (m, 3H).

### Example 66

### N-(2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)octanamide

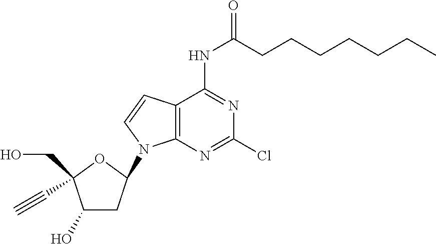 [SMILES: C#C[C@]1(CO)O[C@@H](n2ccc3c(NC(=O)CCCCCCC)nc(Cl)nc32)C[C@@H]1O]

### Step 1: N-(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)octanamide

To a stirred mixture of 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (Intermediate 2) (0.15 g, 0.28 mmol) and DMAP (34 mg, 0.28 mmol) in pyridine (1 mL) was added octanoyl chloride (68 mg, 0.42 mmol) at room temperature. The reaction was stirred at 80° C. for 16 h. The reaction was diluted with EtOAc (50 mL) and washed with water (2×) and brine (2×), dried over Na₂SO₄, and concentrated under reduced pressure. The residue was purified by silica gel column chromatography, eluting with 0-30% EtOAc in Pet. ether to afford the title compound. MS: m/ [symbol] =663.50 [M+H]⁺.

### Step 2: N-(2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)octanamide—

To a stirred mixture of N-(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)octanamide (0.11 g, 0.17 mmol) in THF (2 mL) was added triethylamine trihydrofluoride (1.0 mL, 6.3 mmol) at 25° C. The resulting mixture was stirred at 25° C. for 16 h. The reaction mixture was concentrated under reduced pressure and the residue was purified by RP-Combiflash with the following conditions: Column: C18 Column, 40 g, 60 Å, 20-35 μm; Mobile Phase A: water, Mobile Phase B: MeCN; Flow rate: 50 mL/min; 0% B to 100% B in 15 min; Detector: UV 254 & 210 nm to afford the title compound. MS: m/ [symbol] =435.05 [M+H]⁺. ¹H-NMR (400 MHz, CD₃OD) δ 7.52 (d, J=4.0 Hz, 1H), 6.89 (d, J=4.0 Hz, 1H), 6.63 (t, J=6.8 Hz, 1H), 4.88-4.71 (m, 1H), 4.85-3.73 (m, 2H), 3.05 (s, 1H), 2.76-2.51 (m, 4H), 1.75-1.68 (m, 2H), 1.55-1.32 (m, 8H), 0.88 (t, J=6.4 Hz, 3H).

### Example 67

### (2R,3S,5R)-5-(4-amino-2-chloro-7H-pyrrolo[2,3-d]pyrimidin-7-yl)-2-ethynyl-2-(hydroxymethyl)tetrahydrofuran-3-yl ((1-methyl-2-oxo-1,2-dihydropyridin-3-yl)methyl) carbonate

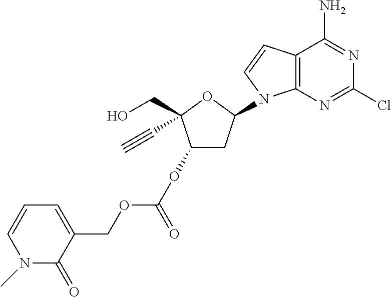 [SMILES: C#C[C@]1(CO)O[C@@H](n2ccc3c(N)nc(Cl)nc32)C[C@@H]1OC(=O)OCc1cccn(C)c1=O]

### Step 1: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl (4-nitrophenyl) carbonate—

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-ol (Intermediate 1) (0.20 g, 0.47 mmol) in DCM (2 mL) and pyridine (0.2 mL) was added 4-nitrophenyl chloroformate (0.19 g, 0.95 mmol). The reaction was stirred for 3 hr at room temperature and then concentrated under reduced pressure. The residue was purified by silica gel column chromatography, eluting with 0-50% EtOAc in Pet. ether to afford the title compound. MS: m/ [symbol] =588.20 [M+H]⁺.

### Step 2: (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl ((1-methyl-2-oxo-1,2-dihydropyridin-3-yl)methyl) carbonate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl (4-nitrophenyl) carbonate (0.16 g, 0.27 mmol) in pyridine (2 mL) was added 3-(hydroxymethyl)-1-methylpyridin-2(1H)-one (76 mg, 0.55 mmol), TEA (0.057 mL, 0.41 mmol) and DMAP (67 mg, 0.55 mmol). The reaction was stirred at room temperature for 2 h. The residue was purified by silica gel column chromatography, eluting with 0-100% EtOAc in Pet. ether to afford the title compound. MS: m/ [symbol] 588.30 [M+H]⁺.

### Step 3: (2R,3S,5R)-5-(4-amino-2-chloro-7H-pyrrolo[2,3-d]pyrimidin-7-yl)-2-ethynyl-2-(hydroxymethyl)tetrahydrofuran-3-yl ((1-methyl-2-oxo-1,2-dihydropyridin-3-yl)methyl) carbonate

To a stirred mixture of (2R,3S,5R)-5-(4-amino-2-chloro-7H-pyrrolo[2,3-d]pyrimidin-7-yl)-2-(((tert-butyldimethylsilyl)oxy)methyl)-2-ethynyltetrahydrofuran-3-yl ((1-methyl-2-oxo-1,2-dihydropyridin-3-yl)methyl) carbonate (80 mg, 0.14 mmol) in THF (1 mL) was added triethylamine trihydrofluoride (0.50 mL, 3.1 mmol) at 25° C. under an argon atmosphere. The reaction was stirred at 25° C. for 5 h. The reaction mixture was concentrated under reduced pressure and the residue was purified by RP-Combiflash with the following conditions: Column: C18 Column, 40 g, 60 Å, 20-35 μm; Mobile Phase A: water, Mobile Phase B: MeCN; Flow rate: 50 mL/min; 0% B to 100% B in 30 min; Detector: UV 254 & 210 nm to provide the title compound. MS: m/ [symbol] =474.15 [M+H]⁺. ¹H-NMR (400 MHz, CD₃OD) δ 7.67-7.64 (m, 2H), 7.31 (d, J=3.6 Hz, 1H), 6.58 (d, J=3.6 Hz, 1H), 6.51-6.47 (m, 1H), 6.40 (t, J=7.2 Hz, 1H), 5.54-5.52 (m, 1H), 5.12 (s, 2H), 3.88-3.80 (m, 2H), 3.59 (s, 3H), 3.08 (s, 1H), 2.96-2.91 (m, 1H), 2.60-2.55 (m, 1H).

### Example 68

### ((2R,3S,5R)-5-(4-amino-2-chloro-7H-pyrrolo[2,3-d]pyrimidin-7-yl)-2-ethynyl-3-(((hexyloxy)carbonyl)oxy)tetrahydrofuran-2-yl)methylbenzyl carbonate

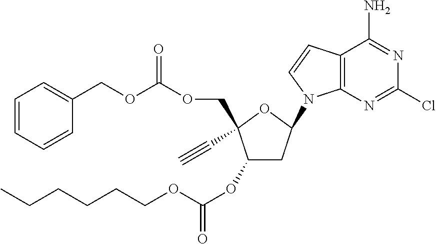 [SMILES: C#C[C@]1(COC(=O)OCc2ccccc2)O[C@@H](n2ccc3c(N)nc(Cl)nc32)C[C@@H]1OC(=O)OCCCCCC]

To a stirred mixture of ((2R,3S,5R)-5-(4-amino-2-chloro-7H-pyrrolo[2,3-d]pyrimidin-7-yl)-2-ethynyl-3-hydroxytetrahydrofuran-2-yl)methyl benzyl carbonate (Example 114) (1.1 g, 2.5 mmol), TEA (0.33 g, 3.2 mmol) and DMAP (0.30 g, 2.5 mmol) in DCM (15 mL) was added hexyl carbonochloridate (0.41 g, 2.5 mmol) at room temperature under an atmosphere of argon. The reaction was stirred for 16 hr at room temperature. The reaction mixture was concentrated under reduced pressure and the residue was purified by RP-flash chromatography, eluting with 0-77% acetonitrile in water to afford the title compound. MS: m/ [symbol] =571.00 [M+H]⁺. ¹H NMR (300 MHz, Methanol-d₄) δ 7.37-7.34 (m, 5H), 7.19 (d, J=3.9 Hz, 1H), 6.60-6.55 (m, 2H), 5.55 (dd, J=4.8, 7.2 Hz, 1H), 5.17 (s, 2H), 4.56 (d, J=11.4 Hz, 1H), 4.44 (d, J=11.4 Hz, 1H), 4.20-4.15 (m, 2H), 3.22 (s, 1H), 2.85-2.78 (m, 1H), 2.68-2.61 (m, 1H), 1.71-1.64 (m, 2H), 1.48-1.31 (m, 6H), 0.99-0.92 (m, 3H).

### Example 69

### 3-(adamantan-1-yl)-N-(2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)propanamide

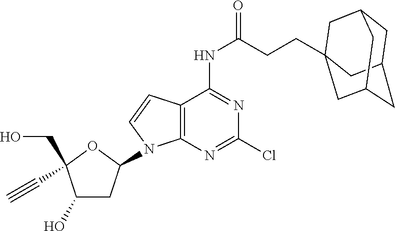 [SMILES: C#C[C@]1(CO)O[C@@H](n2ccc3c(NC(=O)CCC45CC6CC(CC(C6)C4)C5)nc(Cl)nc32)C[C@@H]1O]

### Step 1: 3-(adamantan-1-yl)-N-(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)propanamide

To a mixture of 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (Intermediate 2) (0.50 g, 0.93 mmol) and 3-(1-adamantyl)propanoic acid (0.29 g, 1.4 mmol) in DMA (5 mL) was added TCFH (0.52 g, 1.9 mmol) and 1-methylimidazole (0.23 g, 2.8 mmol) at room temperature. The resultant mixture was warmed to 120° C. and stirred for 4 hr. The reaction mixture was cooled to room temperature, quenched with water (10 mL) and extracted with ethyl acetate (3×). The combined organics were washed with brine, dried over anhydrous sodium sulfate, and filtered. The residue was purified by silica gel column chromatography, eluting with 0-50% EtOAc in Pet. ether to afford the title compound. MS: m/ [symbol] =727.45 [M+H]⁺.

### Step 2: 3-(adamantan-1-yl)-N-(2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)propanamide

To a stirred mixture of 3-(adamantan-1-yl)-N-(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)propanamide (0.20 g, 0.28 mmol) in THF (2 mL) was added triethylamine trihydrofluoride (1.0 mL, 6.3 mmol) at 25° C. under an argon atmosphere. The reaction was stirred at room temperature and then concentrated under reduced pressure. The residue was purified by RP-Combiflash with the following conditions: Column: C18 Column, 40 g, 60 Å, 20-35 μm; Mobile Phase A: water, Mobile Phase B: MeCN; Flow rate: 50 mL/min; 0% B to 100% B in 30 min; Detector: UV 254 & 210 nm to afford the title compound. MS: m/ [symbol] =499.20 [M+H]⁺. ¹H-NMR (400 MHz, CD₃OD) δ 7.52 (d, J=3.6 Hz, 1H), 6.88 (d, J=3.6 Hz, 1H), 6.64-6.62 (m, 1H), 4.69 (t, J=7.2 Hz, 1H), 3.86-3.70 (m, 2H), 3.07 (s, 1H), 2.66-2.50 (m, 4H), 1.96 (d, J=3.6 Hz, 3H), 1.78-1.67 (m, 6H), 1.56-1.53 (m, 8H).

### Example 70

### N-(2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)tetradecanamide

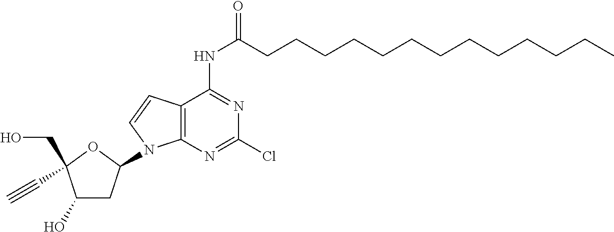 [SMILES: C#C[C@]1(CO)O[C@@H](n2ccc3c(NC(=O)CCCCCCCCCCCCC)nc(Cl)nc32)C[C@@H]1O]

### Step 1: N-(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)tetradecanamide

To a stirred mixture of 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (Intermediate 2) (0.30 g, 0.56 mmol) in pyridine (4 mL) was added tetradecanoic acid (0.19 g, 0.84 mmol) and phosphorus(V) oxychloride (0.26 g, 1.7 mmol) at 0° C. under argon atmosphere. The resulting mixture was stirred for 3 hr. The reaction mixture was quenched with water (70 mL) and extracted with ethyl acetate (3×). The combined organic fractions were washed with brine (3×), dried over anhydrous sodium sulfate, and filtered. The filtrate was concentrated under reduced pressure and the residue was purified by silica gel column chromatography, eluting with 1-10% ethyl acetate in Petroleum ether to afford the title compound. MS: m/ [symbol] =747.50 [M−H]⁻.

### Step 2: N-(h oro-7-((2R,4S,5R)-5-ethynyl-4-hydroxy-5-(hydroxymethyl)tetrahydrofuran-2-yl)-7H-pyrrolo[2,3-d]pyrimidin-4-yl)tetradecanamide—

To a stirred mixture of N-(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)tetradecanamide (0.24 g, 0.32 mmol) in THF (3 mL) was added triethylamine trihydrofluoride (0.52 mL, 3.3 mmol). The reaction was warmed to 25° C. and stirred for 16 hours. The reaction was concentrated under reduced pressure and the residue purified by RP-flash chromatography, eluting with 0-100% acetonitrile in water to afford the title compound. MS: m/ [symbol] =519.30 [M+H]⁺. ¹H NMR (300 MHz, DMSO-d₆) δ 11.01 (s, 1H), 7.63 (d, J=3.6 Hz, 1H), 6.86 (d, J=3.9 Hz, 1H), 6.55 (t, J=6.6 Hz, 1H), 5.56 (d, J=5.4 Hz, 1H), 5.27 (t, J=6.0 Hz, 1H), 4.54-4.48 (m, 1H), 3.65-3.52 (m, 2H), 3.50 (s, 1H), 2.66-2.53 (m, 2H), 2.47-2.34 (m, 2H), 1.62-1.58 (m, 2H), 1.27-1.23 (m, 20H), 0.87-0.84 (m, 3H).

### Example 71

### Decyl(2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)carbamate

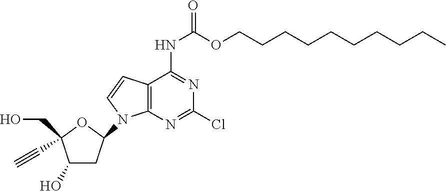 [SMILES: C#C[C@]1(CO)O[C@@H](n2ccc3c(NC(=O)OCCCCCCCCCC)nc(Cl)nc32)C[C@@H]1O]

### Step 1: Decyl(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)carbamate—

To a mixture of 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (intermediate 2) (0.30 g, 0.56 mmol) and triethylamine (0.56 g, 5.6 mmol) in DCM (5 mL) was added DMAP (68 mg, 0.56 mmol) and decyl chloroformate (0.62 g, 2.8 mmol) at room temperature. The resultant mixture was warmed to 40° C. and stirred for 16 h. The reaction was purified by silica gel column chromatography, eluting with 0-50% EtOAc in Pet. ether to afford the title compound. MS: m/ [symbol] =721.50[M+H]⁺.

### Step 2: Decyl(2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)carbamate—

To a stirred mixture of decyl (7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)carbamate (0.14 g, 0.19 mmol) in THF (2 mL) was added triethylamine trihydrofluoride (1.0 mL, 6.3 mmol) at 25° C. under argon atmosphere. The resultant mixture was stirred at 50° C. for 2 hr. The reaction mixture was concentrated under reduced pressure and the residue was purified by RP-Combiflash with the following conditions: Column: C18 Column, 40 g, 60 Å, 20-35 μm; Mobile Phase A: water, Mobile Phase B: MeCN; Flow rate: 50 mL/min; 0% B to 100% B in 30 min; Detector: UV 254 & 210 nm to afford the title compound. MS: m/ [symbol] =493.20 [M+H]⁺. ¹H-NMR (400 MHz, CD₃OD) δ 7.52 (d, J=4.0 Hz, 1H), 6.99 (d, J=3.6 Hz, 1H), 6.64-6.62 (m, 1H), 4.69 (t, J=7.2 Hz, 1H), 4.22 (t, J=6.8 Hz, 2H), 3.87-3.71 (m, 2H), 3.07 (s, 1H), 2.72-2.52 (m, 2H), 1.74-1.68 (m, 2H), 1.45-1.29 (m, 14H), 0.91-0.87 (m, 3H).

### Example 72

### benzyl (2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)carbamate

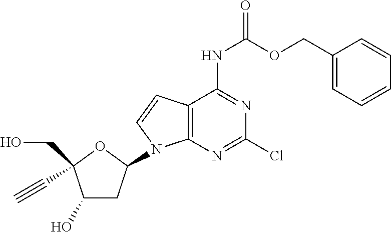 [SMILES: C#C[C@]1(CO)O[C@@H](n2ccc3c(NC(=O)OCc4ccccc4)nc(Cl)nc32)C[C@@H]1O]

### Step 1: Benzyl(7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)carbamate

To a mixture of 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (intermediate 2) (0.40 g, 0.74 mmol) and benzyl (2,5-dioxopyrrolidin-1-yl) carbonate (1.9 g, 7.5 mmol) in DMF (30 mL) was added K₂CO₃ (2.1 g, 15 mmol) at room temperature. The resultant mixture was warmed to 80° C. and stirred for 16 h. The reaction mixture was cooled to room temperature, quenched with water and extracted with ethyl acetate (3×). The combined organic fractions were washed with brine, dried over anhydrous sodium sulfate, filtered, and concentrated under reduced pressure. The residue was purified by silica gel column chromatography, eluting with 0-50% EtOAc in Pet. ether to afford the title compound. MS: m/ [symbol] =671.35 [M+H]⁺.

### Step 2: Benzyl (2-chloro-7-((2R,4S,5R)-5-ethynyl-4-hydroxy-5-(hydroxymethyl)tetrahydrofuran-2-yl)-7H-pyrrolo[2,3-d]pyrimidin-4-yl)carbamate—

To a stirred mixture of benzyl (7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-yl)carbamate (20 mg, 0.030 mmol) in THF (1 mL) was added TEA.3HF (0.50 mL, 3.1 mmol) at 25° C. under argon atmosphere. The reaction was stirred at 50° C. for 2 h. The reaction mixture was concentrated under reduced pressure and the residue was purified by RP-Combiflash with the following conditions: Column: C18 Column, 40 g, 60 Å, 20-35 μm; Mobile Phase A: water, Mobile Phase B: MeCN; Flow rate: 50 mL/min; 0% B to 100% B in 30 min; Detector: UV 254 & 210 nm to afford the title compound. MS: m/ [symbol] =440.95[M−H]⁻. ¹H-NMR (400 MHz, CD₃OD) δ 7.50-7.45 (m, 3H), 7.41-7.27 (m, 3H), 6.98 (d, J=3.6 Hz, 1H), 6.63-6.61 (m, 1H), 5.26 (s, 2H), 4.69 (t, J=7.2 Hz, 1H), 3.84-3.72 (m, 2H), 3.07 (s, 1H), 2.66-2.55 (m, 2H).

### Example 73

### ((2R,3S,5R)-5-(4-amino-2-chloro-7H-pyrrolo[2,3-d]pyrimidin-7-yl)-2-ethynyl-3-hydroxytetrahydrofuran-2-yl)methyl 2-(4-bromophenyl)acetate—

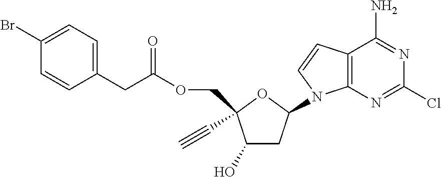 [SMILES: C#C[C@]1(COC(=O)Cc2ccc(Br)cc2)O[C@@H](n2ccc3c(N)nc(Cl)nc32)C[C@@H]1O]

To a stirred mixture of (2R,3S,5R)-5-(4-amino-2-chloro-7H-pyrrolo[2,3-d]pyrimidin-7-yl)-2-ethynyl-2-(hydroxymethyl)tetrahydrofuran-3-ol (compound A) (0.10 g, 0.32 mmol) in pyridine (5 mL) was added DMAP (40 mg, 0.32 mmol), 2-(4-bromophenyl)acetyl chloride (0.23 g, 0.97 mmol) and TEA (0.090 mL, 0.65 mmol) at room temperature under argon atmosphere. The resulting mixture was warmed to 80° C. and stirred for 16 h. The reaction mixture was purified by RP-flash chromatography, eluted with 0-100% acetonitrile in water to afford the title compound. MS: m/ [symbol] =505.05 [M+H]⁺. ¹H NMR (300 MHz, DMSO-d₆) δ 7.58 (s, 2H), 7.48 (d, J=3.2 Hz, 2H), 7.26 (d, J=3.9 Hz, 1H), 7.18-7.15 (m, 2H), 6.63 (d, J=3.6 Hz, 1H), 6.40-6.36 (m, 1H), 5.78 (d, J=5.4 Hz, 1H), 4.58-4.51 (m, 1H), 4.40 (d, J=11.7 Hz, 1H), 4.12 (d, J=11.7 Hz, 1H), 3.74-3.61 (m, 3H), 2.82-2.75 (m, 1H), 2.50-2.40 (m, 1H).

The Examples in the table below were prepared using procedures similar to those described in Example 6, from the appropriate starting material.

| | | Observed |
|---|---|---|
| | | m/z |
| Example | Structure | [M + H]⁺ |
| 74 | 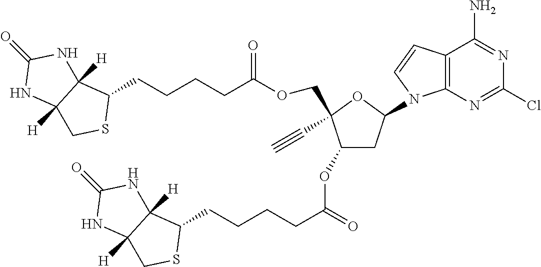 [SMILES: C#C[C@]1(COC(=O)CCCC[C@@H]2SC[C@@H]3NC(=O)N[C@@H]32)O[C@@H](n2ccc3c(N)nc(Cl)nc32)C[C@@H]1OC(=O)CCCC[C@@H]1SC[C@@H]2NC(=O)N[C@@H]21] | 761.05 |

The Examples in the table below were prepared using procedures similar to those described in Example 8, using Intermediate 24b in Example 75 and Intermediate 24a in Example 76 and replacing 2,2,6,6-tetramethylpiperidinylmagnesium chloride lithium chloride complex solution with tert-butylmagnesium chloride (1 M in THF).

| | | Observed |
|---|---|---|
| | | m/z |
| Example | Structure | [M + H]⁺ |
| 75 | 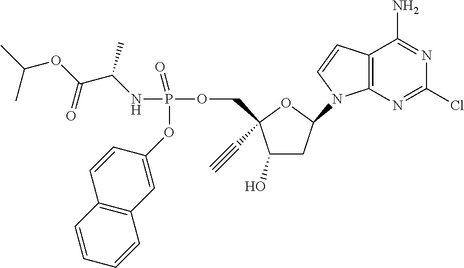 [SMILES: C#C[C@]1(COP(=O)(N[C@@H](C)C(=O)OC(C)C)Oc2ccc3ccccc3c2)O[C@@H](n2ccc3c(N)nc(Cl)nc32)C[C@@H]1O] | 628.25 |
| 76 | 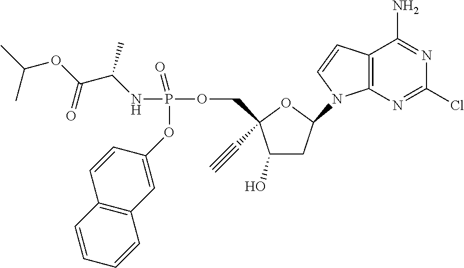 [SMILES: C#C[C@]1(COP(=O)(N[C@@H](C)C(=O)OC(C)C)Oc2ccc3ccccc3c2)O[C@@H](n2ccc3c(N)nc(Cl)nc32)C[C@@H]1O] | 628.25 |

The Examples in the table below were prepared using procedures similar to those described in Example 12, from the appropriate starting material.

| | | Observed |
|---|---|---|
| | | m/z |
| Example | Structure | [M + H]⁺ |
| 77 | 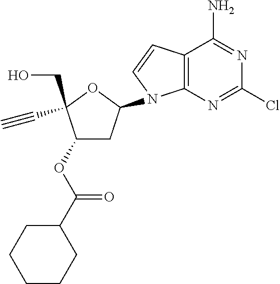 [SMILES: C#C[C@]1(CO)O[C@@H](n2ccc3c(N)nc(Cl)nc32)C[C@@H]1OC(=O)C1CCCCC1] | 417.1 [M − H]− |
| 78 | 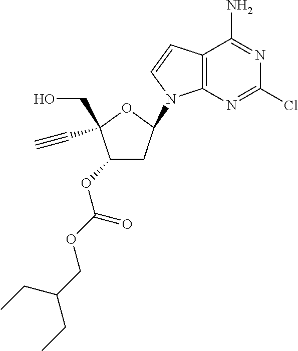 [SMILES: C#C[C@]1(CO)O[C@@H](n2ccc3c(N)nc(Cl)nc32)C[C@@H]1OC(=O)OCC(CC)CC] | 437.15 |
| 79 | 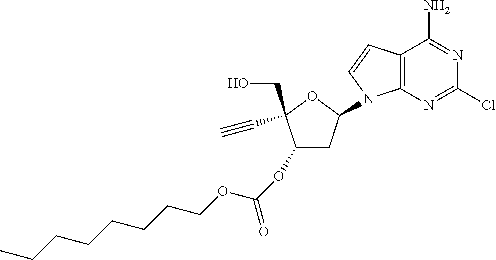 [SMILES: C#C[C@]1(CO)O[C@@H](n2ccc3c(N)nc(Cl)nc32)C[C@@H]1OC(=O)OCCCCCCCC] | 465.20 |
| 80 | 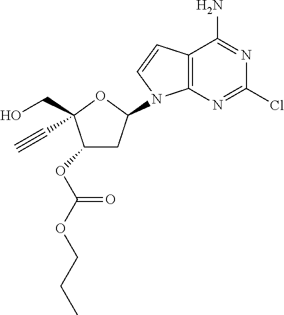 [SMILES: C#C[C@]1(CO)O[C@@H](n2ccc3c(N)nc(Cl)nc32)C[C@@H]1OC(=O)OCCC] | 395.05 |
| 81 | 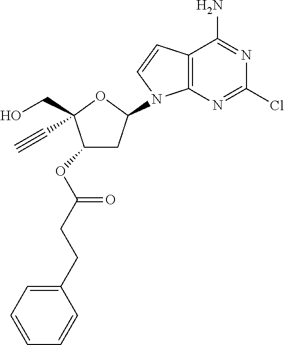 [SMILES: C#C[C@]1(CO)O[C@@H](n2ccc3c(N)nc(Cl)nc32)C[C@@H]1OC(=O)CCc1ccccc1] | 441.00 |
| 82 | 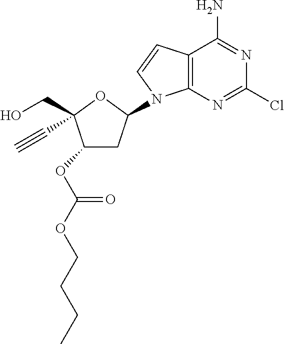 [SMILES: C#C[C@]1(CO)O[C@@H](n2ccc3c(N)nc(Cl)nc32)C[C@@H]1OC(=O)OCCCC] | 409.05 |
| 83 | 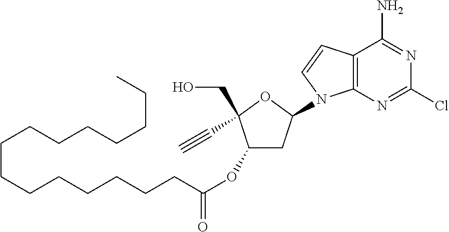 [SMILES: C#C[C@]1(CO)O[C@@H](n2ccc3c(N)nc(Cl)nc32)C[C@@H]1OC(=O)CCCCCCCCCCCCCCC] | 547.40 |
| 84 | 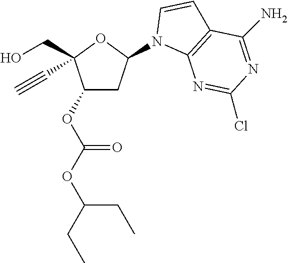 [SMILES: C#C[C@]1(CO)O[C@@H](n2ccc3c(N)nc(Cl)nc32)C[C@@H]1OC(=O)OC(CC)CC] | 423.05 |

The Examples in the table below were prepared using procedures similar to those described in Example 12, step 1, from the appropriate starting material.

| | | Observed |
|---|---|---|
| | | m/z |
| Example | Structure | [M + H]⁺ |
| 85 | 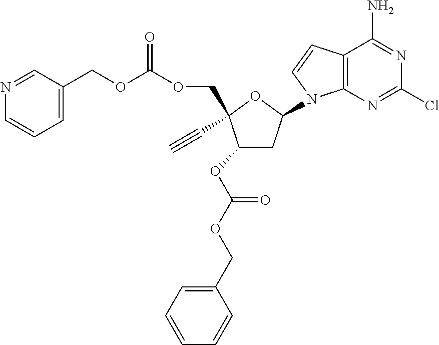 [SMILES: C#C[C@]1(COC(=O)OCc2cccnc2)O[C@@H](n2ccc3c(N)nc(Cl)nc32)C[C@@H]1OC(=O)OCc1ccccc1] | 577.95 |

The Examples in the table below were prepared using procedures similar to those described in Example 14, from the appropriate starting material.

| | | Observed |
|---|---|---|
| | | m/z |
| Example | Structure | [M + H]⁺ |
| 86 | 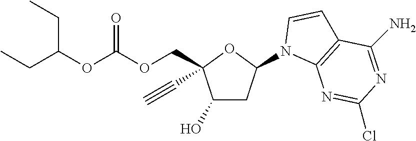 [SMILES: C#C[C@]1(COC(=O)OC(CC)CC)O[C@@H](n2ccc3c(N)nc(Cl)nc32)C[C@@H]1O] | 423.00 |
| 87 | 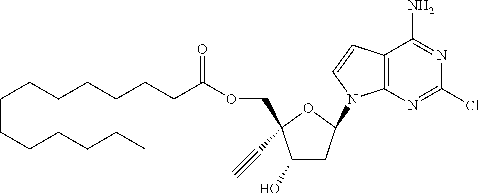 [SMILES: C#C[C@]1(COC(=O)CCCCCCCCCCCCC)O[C@@H](n2ccc3c(N)nc(Cl)nc32)C[C@@H]1O] | 519.05 |
| 88 | 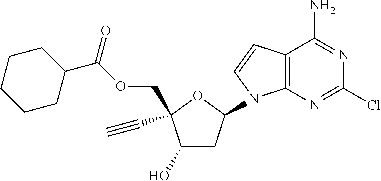 [SMILES: C#C[C@]1(COC(=O)C2CCCCC2)O[C@@H](n2ccc3c(N)nc(Cl)nc32)C[C@@H]1O] | 419.10 |
| 89 | 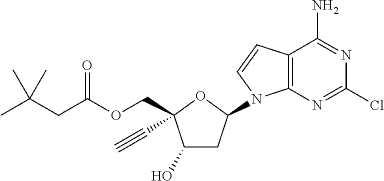 [SMILES: C#C[C@]1(COC(=O)CC(C)(C)C)O[C@@H](n2ccc3c(N)nc(Cl)nc32)C[C@@H]1O] | 407.05 |
| 90 | 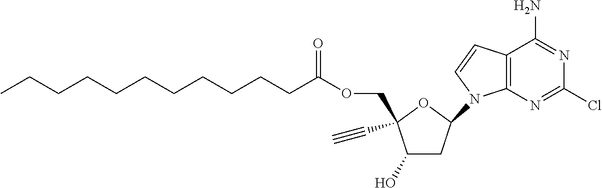 [SMILES: C#C[C@]1(COC(=O)CCCCCCCCCCC)O[C@@H](n2ccc3c(N)nc(Cl)nc32)C[C@@H]1O] | 491.00 |
| 91 | 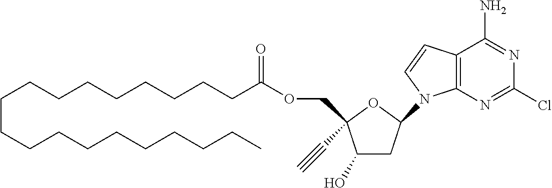 [SMILES: C#C[C@]1(COC(=O)CCCCCCCCCCCCCCCCCCC)O[C@@H](n2ccc3c(N)nc(Cl)nc32)C[C@@H]1O] | 603.10 |

The Examples in the table below were prepared using procedures similar to those described in the alternative procedure for Example 18, from the appropriate starting material.

| | | Observed |
|---|---|---|
| | | m/z |
| Example | Structure | [M + H]⁺ |
| 92 | 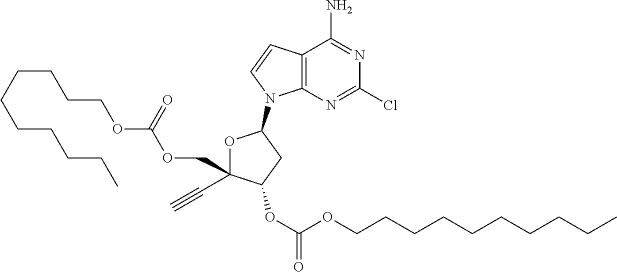 [SMILES: C#C[C@]1(COC(=O)OCCCCCCCCCC)O[C@@H](n2ccc3c(N)nc(Cl)nc32)C[C@@H]1OC(=O)OCCCCCCCCCC] | 677.70 |

The Examples in the table below were prepared using procedures similar to those described in the alternative procedure for Example 27, Step 1, from the appropriate starting material.

| | | Observed m/z |
|---|---|---|
| Example | Structure | [M + H]⁺ |
| 93 | 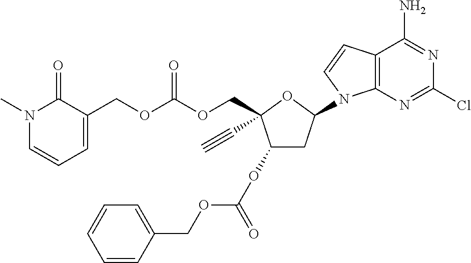 [SMILES: C#C[C@]1(COC(=O)OCc2cccn(C)c2=O)O[C@@H](n2ccc3c(N)nc(Cl)nc32)C[C@@H]1OC(=O)OCc1ccccc1] | 608.05 |
| 94 | 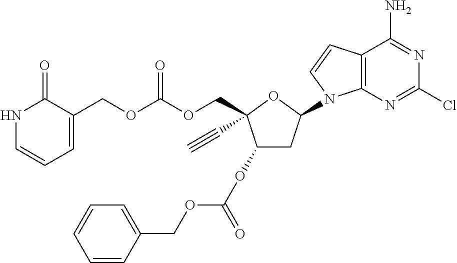 [SMILES: C#C[C@]1(COC(=O)OCc2ccc[nH]c2=O)O[C@@H](n2ccc3c(N)nc(Cl)nc32)C[C@@H]1OC(=O)OCc1ccccc1] | 594.05 |

The Examples in the table below were prepared using procedures similar to those described in in the alternative procedure for Example 34, from the appropriate starting material.

| | | Observed m/z |
|---|---|---|
| Example | Structure | [M + H]⁺ |
| 95 | 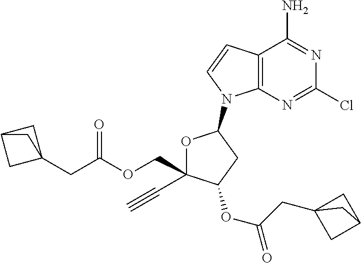 [SMILES: C#C[C@]1(COC(=O)CC23CC(C2)C3)O[C@@H](n2ccc3c(N)nc(Cl)nc32)C[C@@H]1OC(=O)CC12CC(C1)C2] | 525.40 |
| 96 | 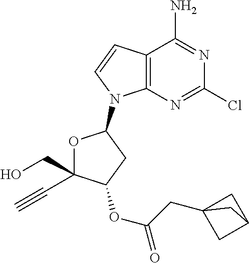 [SMILES: C#C[C@]1(CO)O[C@@H](n2ccc3c(N)nc(Cl)nc32)C[C@@H]1OC(=O)CC12CC(C1)C2] | 417.30 |
| 97 | 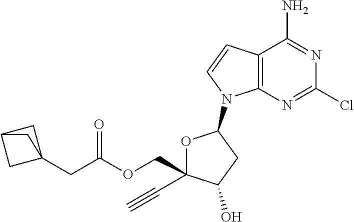 [SMILES: C#C[C@]1(COC(=O)CC23CC(C2)C3)O[C@@H](n2ccc3c(N)nc(Cl)nc32)C[C@@H]1O] | 417.30 |
| 98 | 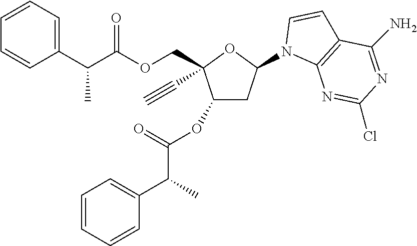 [SMILES: C#C[C@]1(COC(=O)[C@H](C)c2ccccc2)O[C@@H](n2ccc3c(N)nc(Cl)nc32)C[C@@H]1OC(=O)[C@H](C)c1ccccc1] | 573.50 |
| 99 | 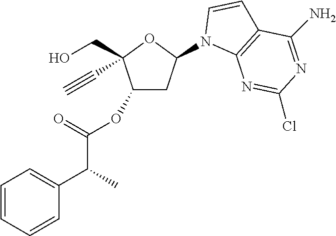 [SMILES: C#C[C@]1(CO)O[C@@H](n2ccc3c(N)nc(Cl)nc32)C[C@@H]1OC(=O)[C@H](C)c1ccccc1] | 441.50 |
| 100 | 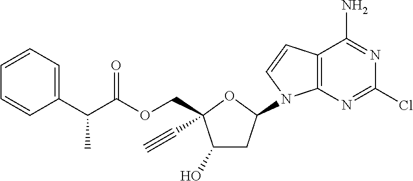 [SMILES: C#C[C@]1(COC(=O)[C@H](C)c2ccccc2)O[C@@H](n2ccc3c(N)nc(Cl)nc32)C[C@@H]1O] | 441.05 |
| 101 | 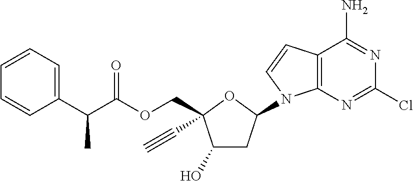 [SMILES: C#C[C@]1(COC(=O)[C@@H](C)c2ccccc2)O[C@@H](n2ccc3c(N)nc(Cl)nc32)C[C@@H]1O] | 441.05 |
| 102 | 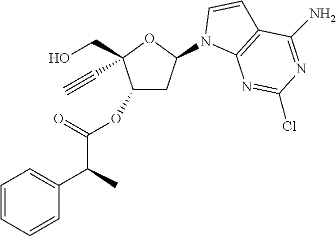 [SMILES: C#C[C@]1(CO)O[C@@H](n2ccc3c(N)nc(Cl)nc32)C[C@@H]1OC(=O)[C@@H](C)c1ccccc1] | 441.50 |
| 103 | 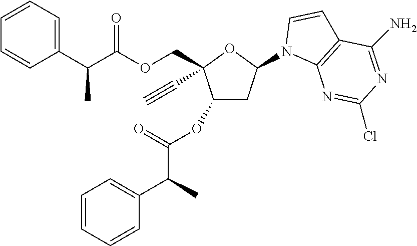 [SMILES: C#C[C@]1(COC(=O)[C@@H](C)c2ccccc2)O[C@@H](n2ccc3c(N)nc(Cl)nc32)C[C@@H]1OC(=O)[C@@H](C)c1ccccc1] | 573.60 |
| 104 | 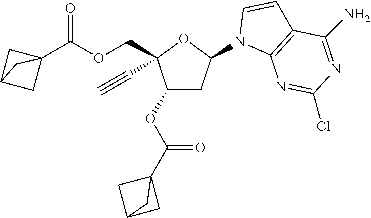 [SMILES: C#C[C@]1(COC(=O)C23CC(C2)C3)O[C@@H](n2ccc3c(N)nc(Cl)nc32)C[C@@H]1OC(=O)C12CC(C1)C2] | 497.50 |
| 105 | 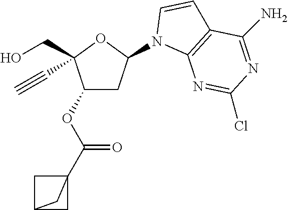 [SMILES: C#C[C@]1(CO)O[C@@H](n2ccc3c(N)nc(Cl)nc32)C[C@@H]1OC(=O)C12CC(C1)C2] | 403.50 |
| 106 | 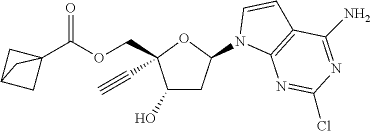 [SMILES: C#C[C@]1(COC(=O)C23CC(C2)C3)O[C@@H](n2ccc3c(N)nc(Cl)nc32)C[C@@H]1O] | 403.50 |
| 107 | 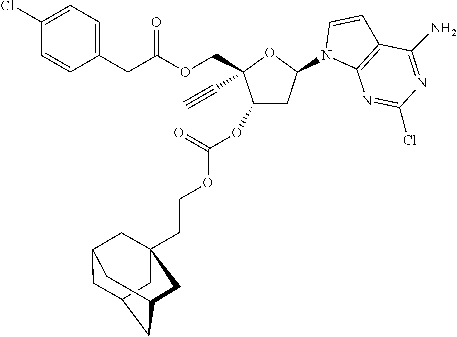 | 667.10 |
| 108 | 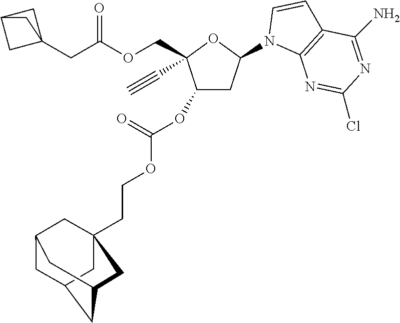 | 623.20 |
| 109 | 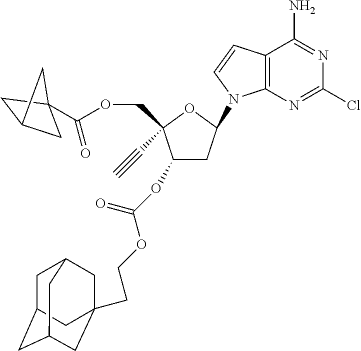 [SMILES: C#C[C@]1(COC(=O)C23CC(C2)C3)O[C@@H](n2ccc3c(N)nc(Cl)nc32)C[C@@H]1OC(=O)OCCC12CC3CC(CC(C3)C1)C2] | 609.20 |

The Examples in the table below were prepared using procedures similar to those described in the alternative procedure for Example 36, from the appropriate starting material.

| | | Observed |
|---|---|---|
| | | m/z |
| Example | Structure | [M + H]⁺ |
| 110 | 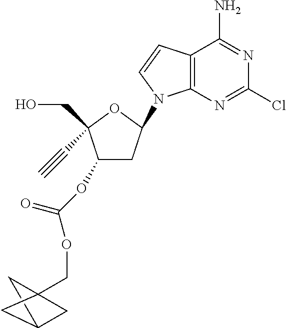 [SMILES: C#C[C@]1(CO)O[C@@H](n2ccc3c(N)nc(Cl)nc32)C[C@@H]1OC(=O)OCC12CC(C1)C2] | 433.20 |
| 111 | 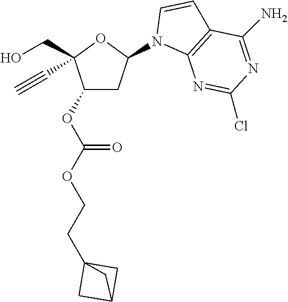 [SMILES: C#C[C@]1(CO)O[C@@H](n2ccc3c(N)nc(Cl)nc32)C[C@@H]1OC(=O)OCCC12CC(C1)C2] | 447.10 |

The Examples in the table below were prepared using procedures similar to those described in the alternative procedure for Example 36, step 1, from the appropriate starting material.

| | | Observed m/z |
|---|---|---|
| Example | Structure | [M + H]⁺ |
| 112 | 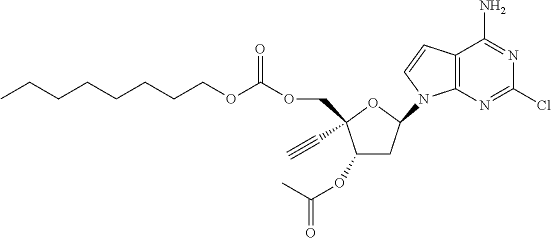 [SMILES: C#C[C@]1(COC(=O)OCCCCCCCC)O[C@@H](n2ccc3c(N)nc(Cl)nc32)C[C@@H]1OC(C)=O] | 507.05 |
| 113 | 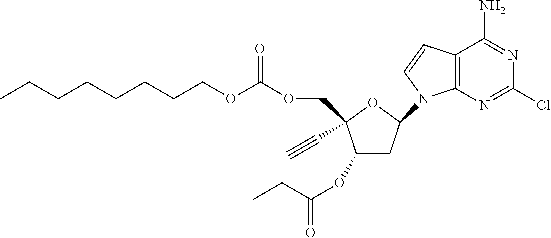 [SMILES: C#C[C@]1(COC(=O)OCCCCCCCC)O[C@@H](n2ccc3c(N)nc(Cl)nc32)C[C@@H]1OC(=O)CC] | 523.15 |

The Examples in the table below were prepared using procedures similar to those described in Example 39, from the appropriate starting material.

| | | Observed |
|---|---|---|
| | | m/z |
| Example | Structure | [M + H]⁺ |
| 114 | 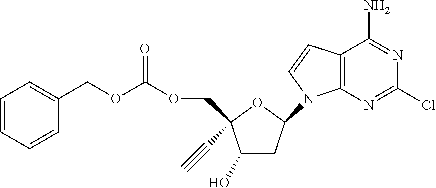 [SMILES: C#C[C@]1(COC(=O)OCc2ccccc2)O[C@@H](n2ccc3c(N)nc(Cl)nc32)C[C@@H]1O] | 442.95 |
| 115 | 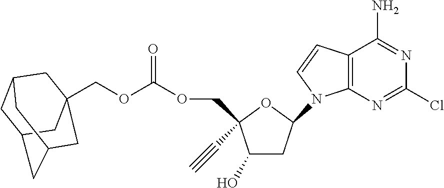 [SMILES: C#C[C@]1(COC(=O)OCC23CC4CC(CC(C4)C2)C3)O[C@@H](n2ccc3c(N)nc(Cl)nc32)C[C@@H]1O] | 501.05 |
| 116 | 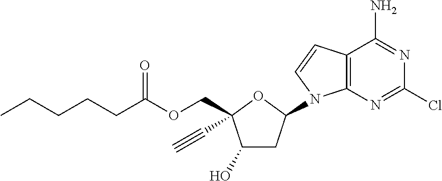 [SMILES: C#C[C@]1(COC(=O)CCCCC)O[C@@H](n2ccc3c(N)nc(Cl)nc32)C[C@@H]1O] | 407.00 |
| 117 | 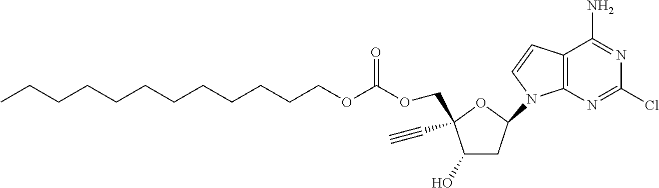 [SMILES: C#C[C@]1(COC(=O)OCCCCCCCCCCCC)O[C@@H](n2ccc3c(N)nc(Cl)nc32)C[C@@H]1O] | 521.20 |
| 118 | 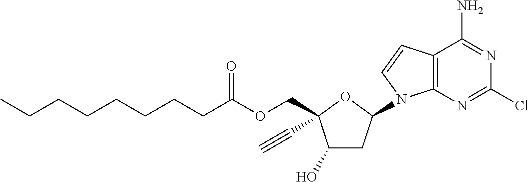 [SMILES: C#C[C@]1(COC(=O)CCCCCCCC)O[C@@H](n2ccc3c(N)nc(Cl)nc32)C[C@@H]1O] | 449.00 |

The Examples in the table below were prepared using procedures similar to those described in Example 39, from the appropriate starting material and with 2 eq. of TEA.

| | | Observed |
|---|---|---|
| | | m/z |
| Example | Structure | [M + H]⁺ |
| 119 | 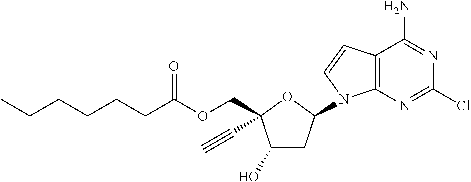 [SMILES: C#C[C@]1(COC(=O)CCCCCC)O[C@@H](n2ccc3c(N)nc(Cl)nc32)C[C@@H]1O] | 420.95 |
| 120 | 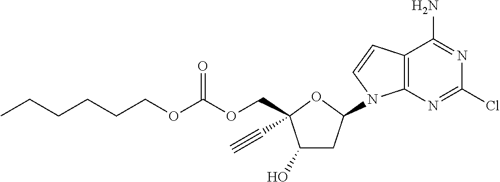 [SMILES: C#C[C@]1(COC(=O)OCCCCCC)O[C@@H](n2ccc3c(N)nc(Cl)nc32)C[C@@H]1O] | 436.95 |
| 121 | 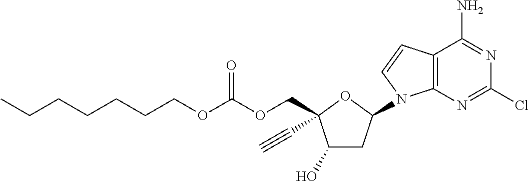 [SMILES: C#C[C@]1(COC(=O)OCCCCCCC)O[C@@H](n2ccc3c(N)nc(Cl)nc32)C[C@@H]1O] | 451.15 |
| 122 | 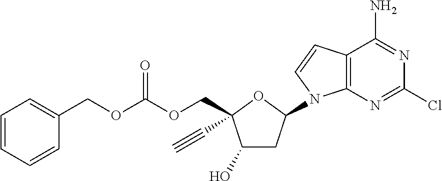 [SMILES: C#C[C@]1(COC(=O)OCc2ccccc2)O[C@@H](n2ccc3c(N)nc(Cl)nc32)C[C@@H]1O] | 440.90 |
| 123 | 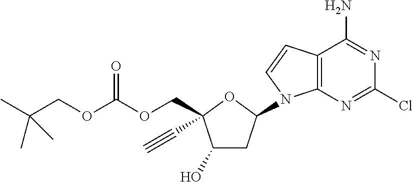 [SMILES: C#C[C@]1(COC(=O)OCC(C)(C)C)O[C@@H](n2ccc3c(N)nc(Cl)nc32)C[C@@H]1O] | 423.10 |
| 124 | 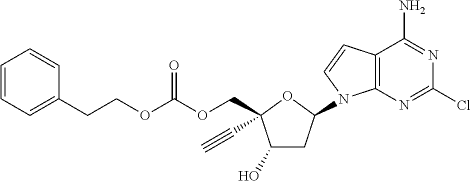 [SMILES: C#C[C@]1(COC(=O)OCCc2ccccc2)O[C@@H](n2ccc3c(N)nc(Cl)nc32)C[C@@H]1O] | 457.00 |
| 125 | 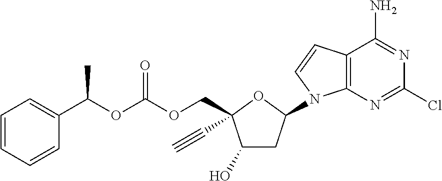 [SMILES: C#C[C@]1(COC(=O)O[C@H](C)c2ccccc2)O[C@@H](n2ccc3c(N)nc(Cl)nc32)C[C@@H]1O] | 457.00 |
| 126 | 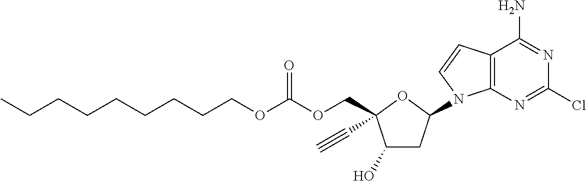 [SMILES: C#C[C@]1(COC(=O)OCCCCCCCCC)O[C@@H](n2ccc3c(N)nc(Cl)nc32)C[C@@H]1O] | 479.10 |
| 127 | 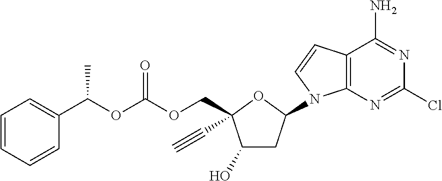 [SMILES: C#C[C@]1(COC(=O)O[C@@H](C)c2ccccc2)O[C@@H](n2ccc3c(N)nc(Cl)nc32)C[C@@H]1O] | 457.15 |
| 128 | 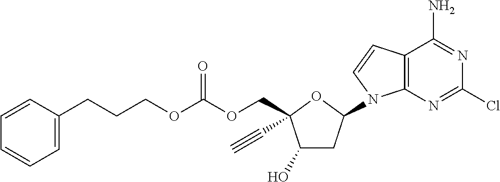 [SMILES: C#C[C@]1(COC(=O)OCCCc2ccccc2)O[C@@H](n2ccc3c(N)nc(Cl)nc32)C[C@@H]1O] | 471.05 |
| 129 | 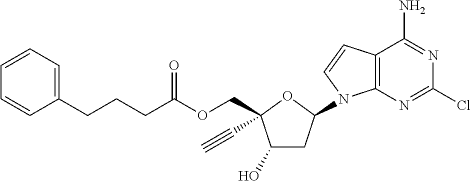 [SMILES: C#C[C@]1(COC(=O)CCCc2ccccc2)O[C@@H](n2ccc3c(N)nc(Cl)nc32)C[C@@H]1O] | 455.05 |
| 130 | 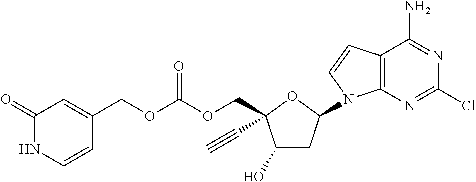 [SMILES: C#C[C@]1(COC(=O)OCc2cc[nH]c(=O)c2)O[C@@H](n2ccc3c(N)nc(Cl)nc32)C[C@@H]1O] | 460.15 |
| 131 | 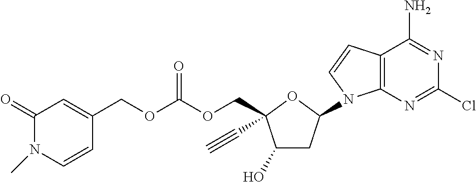 [SMILES: C#C[C@]1(COC(=O)OCc2ccn(C)c(=O)c2)O[C@@H](n2ccc3c(N)nc(Cl)nc32)C[C@@H]1O] | 474.15 |
| 132 | 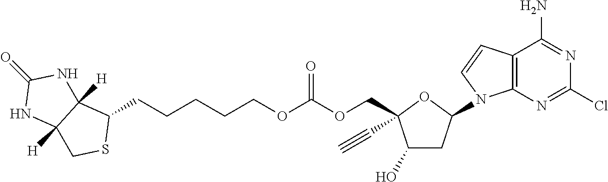 [SMILES: C#C[C@]1(COC(=O)OCCCCC[C@@H]2SC[C@@H]3NC(=O)N[C@@H]32)O[C@@H](n2ccc3c(N)nc(Cl)nc32)C[C@@H]1O] | 565.05 |

The Examples in the table below were prepared using procedures similar to those described in Example 51, from the appropriate starting material.

| | | Observed |
|---|---|---|
| | | m/z |
| Example | Structure | [M + H]⁺ |
| 133 | 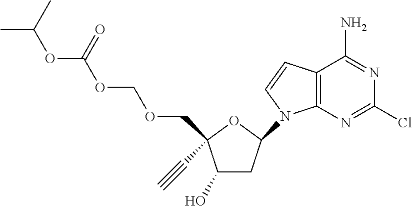 [SMILES: C#C[C@]1(COCOC(=O)OC(C)C)O[C@@H](n2ccc3c(N)nc(Cl)nc32)C[C@@H]1O] | 424.90 |
| 134 | 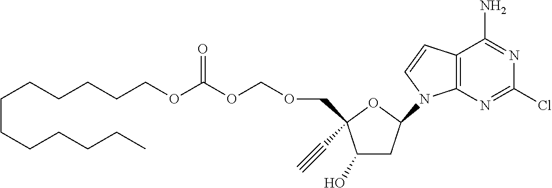 [SMILES: C#C[C@]1(COCOC(=O)OCCCCCCCCCCCC)O[C@@H](n2ccc3c(N)nc(Cl)nc32)C[C@@H]1O] | 551.15 |
| 135 | 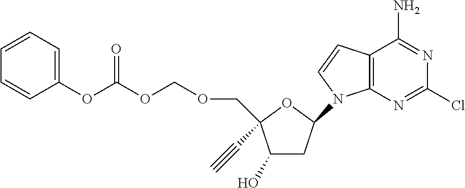 [SMILES: C#C[C@]1(COCOC(=O)Oc2ccccc2)O[C@@H](n2ccc3c(N)nc(Cl)nc32)C[C@@H]1O] | 459.05 |
| 136 | 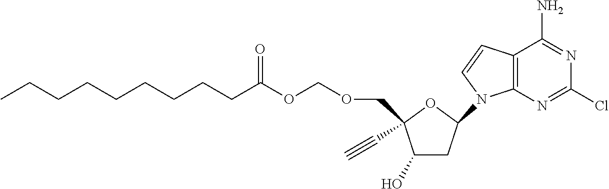 [SMILES: C#C[C@]1(COCOC(=O)CCCCCCCCC)O[C@@H](n2ccc3c(N)nc(Cl)nc32)C[C@@H]1O] | 493.25 |
| 137 | 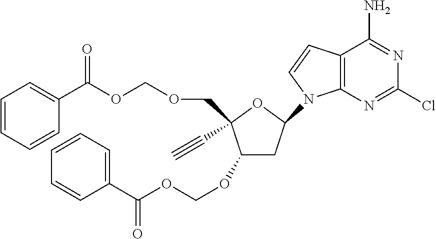 [SMILES: C#C[C@]1(COCOC(=O)c2ccccc2)O[C@@H](n2ccc3c(N)nc(Cl)nc32)C[C@@H]1OCOC(=O)c1ccccc1] | 577.05 |
| 138 | 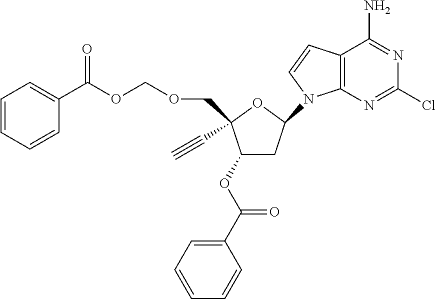 [SMILES: C#C[C@]1(COCOC(=O)c2ccccc2)O[C@@H](n2ccc3c(N)nc(Cl)nc32)C[C@@H]1OC(=O)c1ccccc1] | 547.15 |
| 139 | 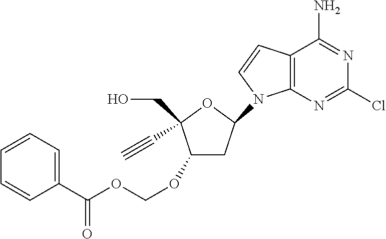 [SMILES: C#C[C@]1(CO)O[C@@H](n2ccc3c(N)nc(Cl)nc32)C[C@@H]1OCOC(=O)c1ccccc1] | 443.05 |
| 140 | 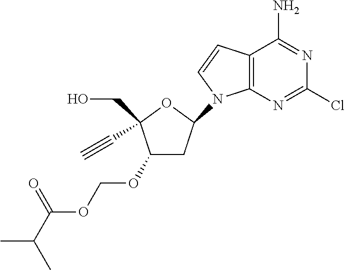 [SMILES: C#C[C@]1(CO)O[C@@H](n2ccc3c(N)nc(Cl)nc32)C[C@@H]1OCOC(=O)C(C)C] | 409.00 |
| 141 | 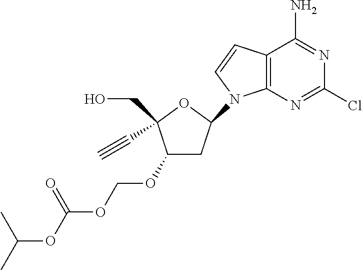 [SMILES: C#C[C@]1(CO)O[C@@H](n2ccc3c(N)nc(Cl)nc32)C[C@@H]1OCOC(=O)OC(C)C] | 425.00 |

The Examples in the table below were prepared using procedures similar to those described in Example 53, from the appropriate starting material.

| | | Observed m/z |
|---|---|---|
| Example | Structure | [M + H]⁺ |
| 142 | 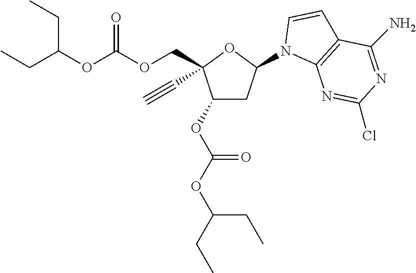 [SMILES: C#C[C@]1(COC(=O)OC(CC)CC)O[C@@H](n2ccc3c(N)nc(Cl)nc32)C[C@@H]1OC(=O)OC(CC)CC] | 537.00 |

The Examples in the table below were prepared using procedures similar to those described in Example 60, from the appropriate starting material.

| | | Observed m/z |
|---|---|---|
| Example | Structure | [M + H]⁺ |
| 143 | 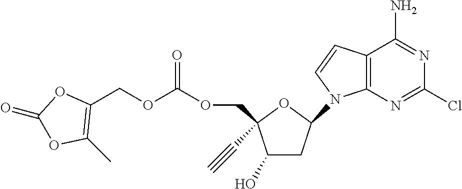 [SMILES: C#C[C@]1(COC(=O)OCc2oc(=O)oc2C)O[C@@H](n2ccc3c(N)nc(Cl)nc32)C[C@@H]1O] | 465.00 |

The Examples in the table below were prepared using procedures similar to those described in Example 61 step 1, from the appropriate starting material.

| | | Observed m/z |
|---|---|---|
| Example | Structure | [M + H]⁺ |
| 144 | 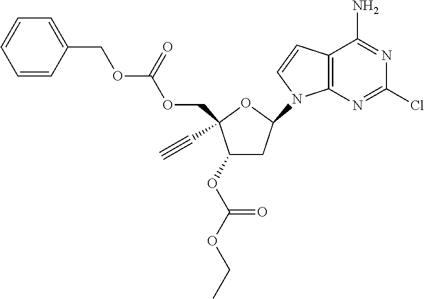 [SMILES: C#C[C@]1(COC(=O)OCc2ccccc2)O[C@@H](n2ccc3c(N)nc(Cl)nc32)C[C@@H]1OC(=O)OCC] | 516.80 |

The Examples in the table below were prepared using procedures similar to those described in Example 62, from the appropriate starting material and replacing DMF with NMP.

| | | Observed m/z |
|---|---|---|
| Example | Structure | [M + H]⁺ |
| 145 | 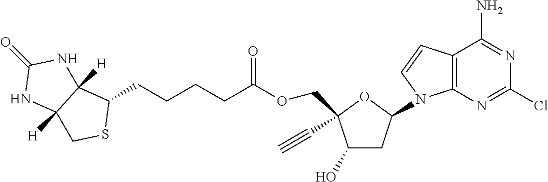 [SMILES: C#C[C@]1(COC(=O)CCCC[C@@H]2SC[C@@H]3NC(=O)N[C@@H]32)O[C@@H](n2ccc3c(N)nc(Cl)nc32)C[C@@H]1O] | 535.45 |

The Examples in the table below were prepared using procedures similar to those described in Example 62, step 1, from the appropriate starting material.

| | | Observed m/z |
|---|---|---|
| Example | Structure | [M + H]⁺ |
| 146 | 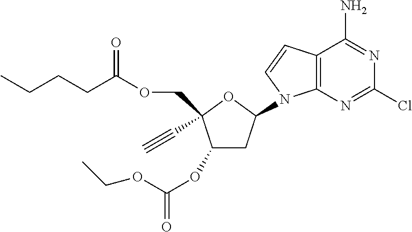 [SMILES: C#C[C@]1(COC(=O)CCCC)O[C@@H](n2ccc3c(N)nc(Cl)nc32)C[C@@H]1OC(=O)OCC] | 465.10 |
| 147 | 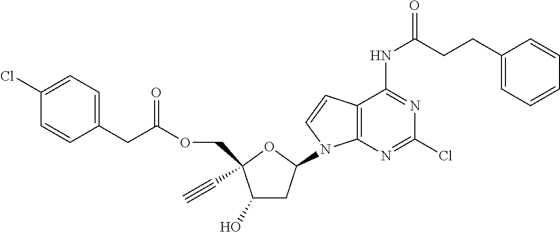 [SMILES: C#C[C@]1(COC(=O)Cc2ccc(Cl)cc2)O[C@@H](n2ccc3c(NC(=O)CCc4ccccc4)nc(Cl)nc32)C[C@@H]1O] | 593.15 |

The Examples in the table below were prepared using procedures similar to those described in Example 63, from the appropriate starting material.

| | | Observed m/z |
|---|---|---|
| Example | Structure | [M + H]⁺ |
| 148 | 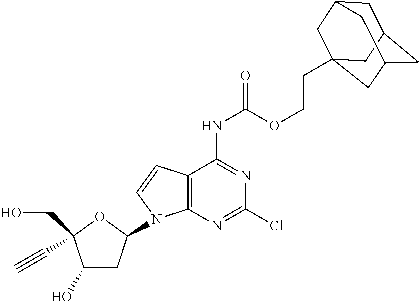 [SMILES: C#C[C@]1(CO)O[C@@H](n2ccc3c(NC(=O)OCCC45CC6CC(CC(C6)C4)C5)nc(Cl)nc32)C[C@@H]1O] | 515.30 |
| 149 | 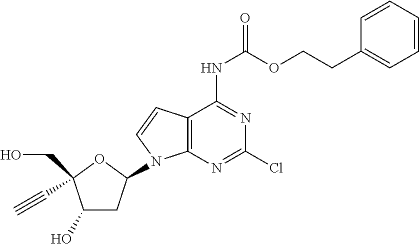 [SMILES: C#C[C@]1(CO)O[C@@H](n2ccc3c(NC(=O)OCCc4ccccc4)nc(Cl)nc32)C[C@@H]1O] | 457.05 |
| 150 | 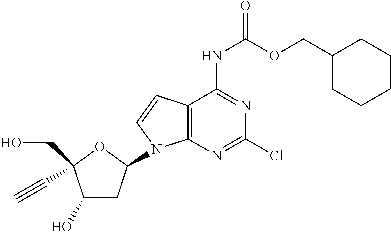 [SMILES: C#C[C@]1(CO)O[C@@H](n2ccc3c(NC(=O)OCC4CCCCC4)nc(Cl)nc32)C[C@@H]1O] | 449.10 |
| 151 | 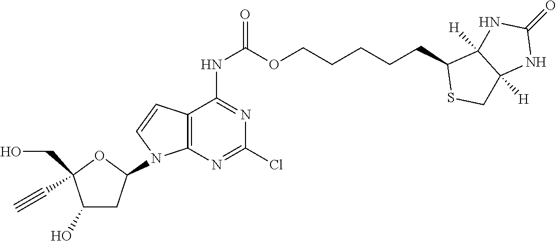 [SMILES: C#C[C@]1(CO)O[C@@H](n2ccc3c(NC(=O)OCCCCC[C@@H]4SC[C@@H]5NC(=O)N[C@@H]54)nc(Cl)nc32)C[C@@H]1O] | 565.20 |

The Examples in the table below were prepared using procedures similar to those described in Example 64, from the appropriate starting material.

| | | Observed m/z |
|---|---|---|
| Example | Structure | [M + H]⁺ |
| 152 | 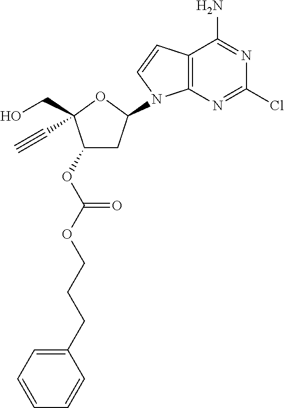 [SMILES: C#C[C@]1(CO)O[C@@H](n2ccc3c(N)nc(Cl)nc32)C[C@@H]1OC(=O)OCCCc1ccccc1] | 471.05 |
| 153 | 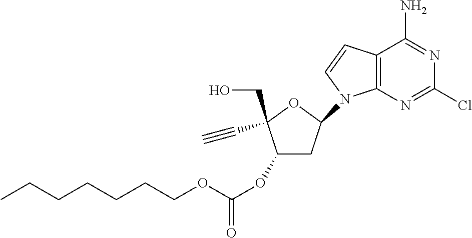 [SMILES: C#C[C@]1(CO)O[C@@H](n2ccc3c(N)nc(Cl)nc32)C[C@@H]1OC(=O)OCCCCCCC] | 451.05 |
| 154 | 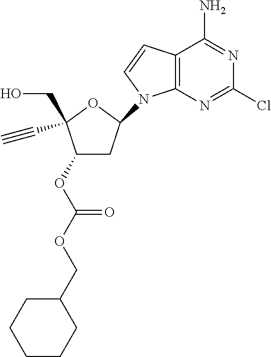 [SMILES: C#C[C@]1(CO)O[C@@H](n2ccc3c(N)nc(Cl)nc32)C[C@@H]1OC(=O)OCC1CCCCC1] | 449.00 |
| 155 | 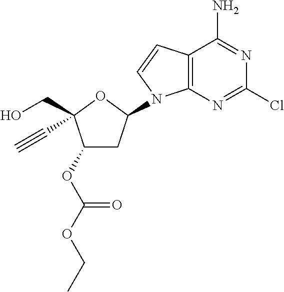 [SMILES: C#C[C@]1(CO)O[C@@H](n2ccc3c(N)nc(Cl)nc32)C[C@@H]1OC(=O)OCC] | 380.90 |
| 156 | 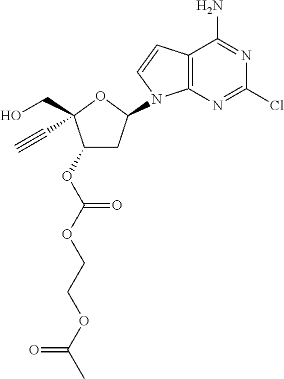 [SMILES: C#C[C@]1(CO)O[C@@H](n2ccc3c(N)nc(Cl)nc32)C[C@@H]1OC(=O)OCCOC(C)=O] | 438.95 |
| 157 | 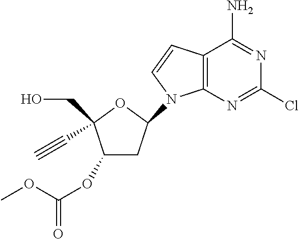 [SMILES: C#C[C@]1(CO)O[C@@H](n2ccc3c(N)nc(Cl)nc32)C[C@@H]1OC(=O)OC] | 366.95 |
| 158 | 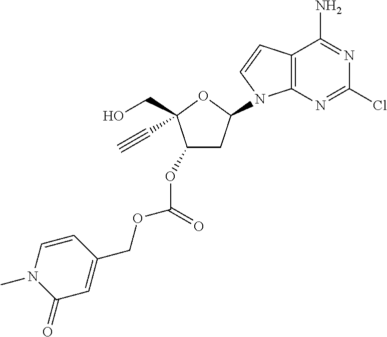 [SMILES: C#C[C@]1(CO)O[C@@H](n2ccc3c(N)nc(Cl)nc32)C[C@@H]1OC(=O)OCc1ccn(C)c(=O)c1] | 474.00 |
| 159 | 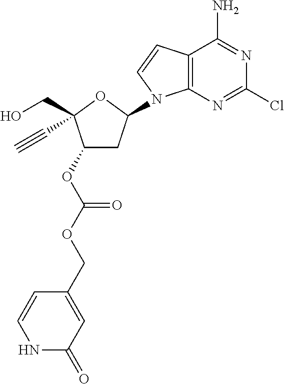 [SMILES: C#C[C@]1(CO)O[C@@H](n2ccc3c(N)nc(Cl)nc32)C[C@@H]1OC(=O)OCc1cc[nH]c(=O)c1] | 460.05 |
| 160 | 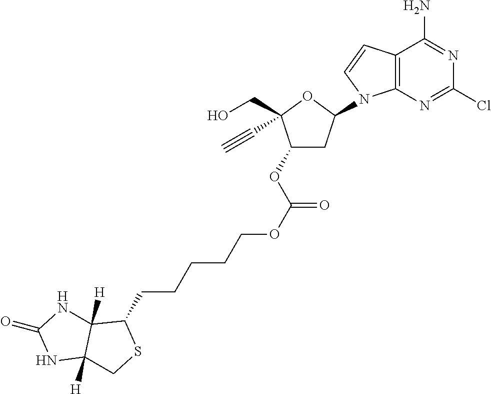 [SMILES: C#C[C@]1(CO)O[C@@H](n2ccc3c(N)nc(Cl)nc32)C[C@@H]1OC(=O)OCCCCC[C@@H]1SC[C@@H]2NC(=O)N[C@@H]21] | 565.05 |

The Examples in the table below were prepared using procedures similar to those described in Example 64 step 1, from the appropriate starting material.

| | | Observed |
|---|---|---|
| | | m/z |
| Example | Structure | [M + H]⁺ |
| 161 | 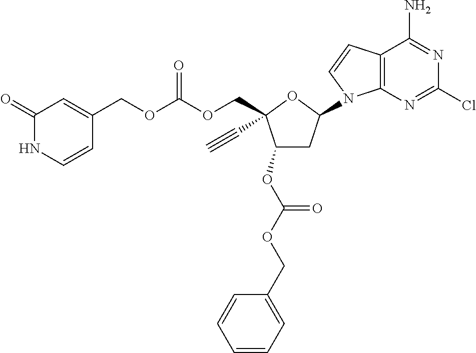 [SMILES: C#C[C@]1(COC(=O)OCc2cc[nH]c(=O)c2)O[C@@H](n2ccc3c(N)nc(Cl)nc32)C[C@@H]1OC(=O)OCc1ccccc1] | 594.10 |
| 162 | 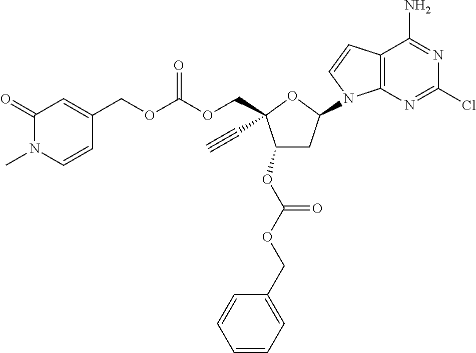 [SMILES: C#C[C@]1(COC(=O)OCc2ccn(C)c(=O)c2)O[C@@H](n2ccc3c(N)nc(Cl)nc32)C[C@@H]1OC(=O)OCc1ccccc1] | 608.25 |
| 163 | 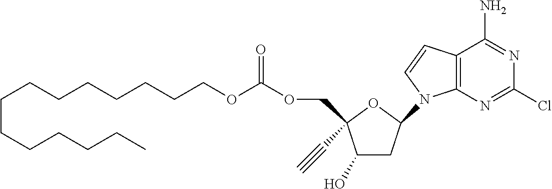 [SMILES: C#C[C@]1(COC(=O)OCCCCCCCCCCCCCC)O[C@@H](n2ccc3c(N)nc(Cl)nc32)C[C@@H]1O] | 549.70 |
| 164 | 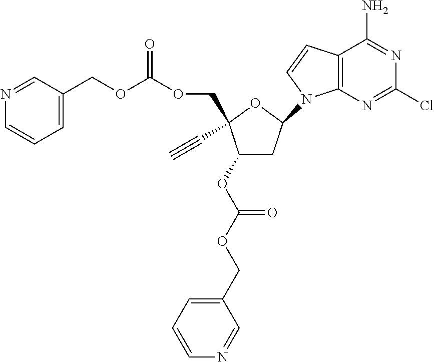 [SMILES: C#C[C@]1(COC(=O)OCc2cccnc2)O[C@@H](n2ccc3c(N)nc(Cl)nc32)C[C@@H]1OC(=O)OCc1cccnc1] | 577.15 |
| 165 | 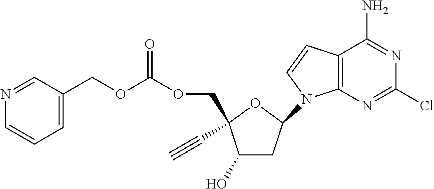 [SMILES: C#C[C@]1(COC(=O)OCc2cccnc2)O[C@@H](n2ccc3c(N)nc(Cl)nc32)C[C@@H]1O] | 444.15 |
| 166 | 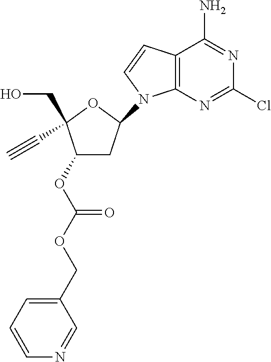 [SMILES: C#C[C@]1(CO)O[C@@H](n2ccc3c(N)nc(Cl)nc32)C[C@@H]1OC(=O)OCc1cccnc1] | 443.95 |
| 167 | 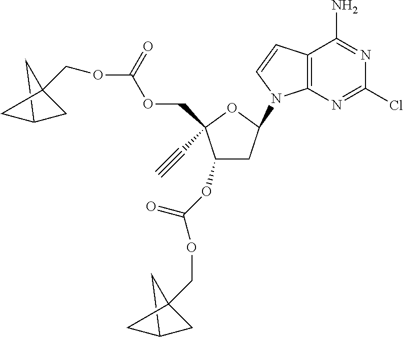 [SMILES: C#C[C@]1(COC(=O)OCC23CC(C2)C3)O[C@@H](n2ccc3c(N)nc(Cl)nc32)C[C@@H]1OC(=O)OCC12CC(C1)C2] | 557.40 |
| 168 | 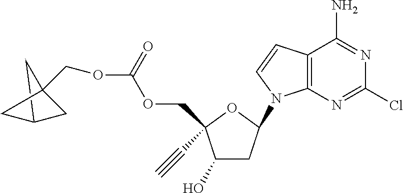 [SMILES: C#C[C@]1(COC(=O)OCC23CC(C2)C3)O[C@@H](n2ccc3c(N)nc(Cl)nc32)C[C@@H]1O] | 433.20 |
| 169 | 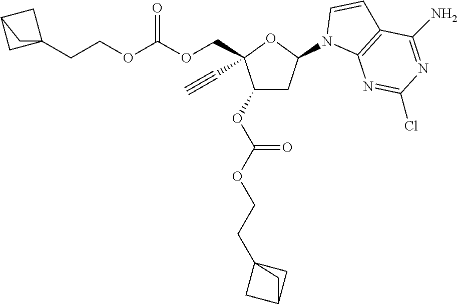 [SMILES: C#C[C@]1(COC(=O)OCCC23CC(C2)C3)O[C@@H](n2ccc3c(N)nc(Cl)nc32)C[C@@H]1OC(=O)OCCC12CC(C1)C2] | 585.20 |
| 170 | 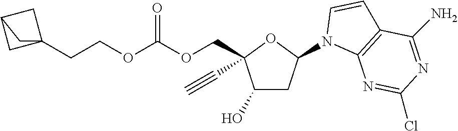 [SMILES: C#C[C@]1(COC(=O)OCCC23CC(C2)C3)O[C@@H](n2ccc3c(N)nc(Cl)nc32)C[C@@H]1O] | 447.10 |
| 171 | 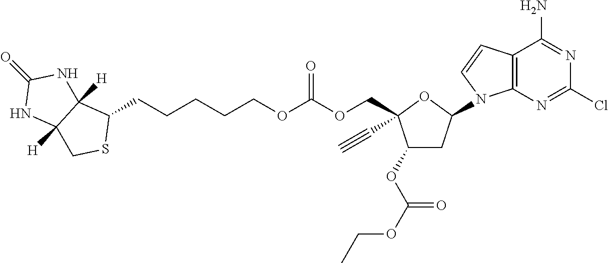 [SMILES: C#C[C@]1(COC(=O)OCCCCC[C@@H]2SC[C@@H]3NC(=O)N[C@@H]32)O[C@@H](n2ccc3c(N)nc(Cl)nc32)C[C@@H]1OC(=O)OCC] | 637.10 |
| 172 | 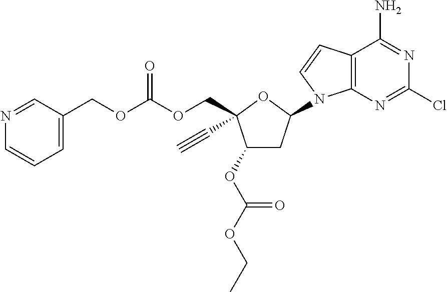 [SMILES: C#C[C@]1(COC(=O)OCc2cccnc2)O[C@@H](n2ccc3c(N)nc(Cl)nc32)C[C@@H]1OC(=O)OCC] | 514.10 |
| 173 | 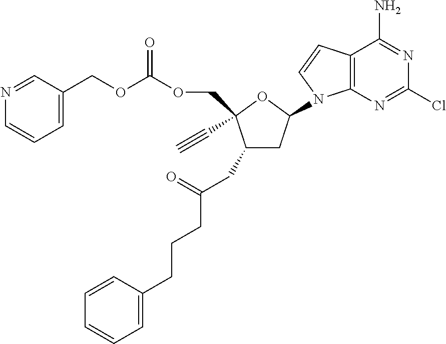 [SMILES: C#C[C@]1(COC(=O)OCc2cccnc2)O[C@@H](n2ccc3c(N)nc(Cl)nc32)C[C@@H]1CC(=O)CCCc1ccccc1] | 589.90 |
| 174 | 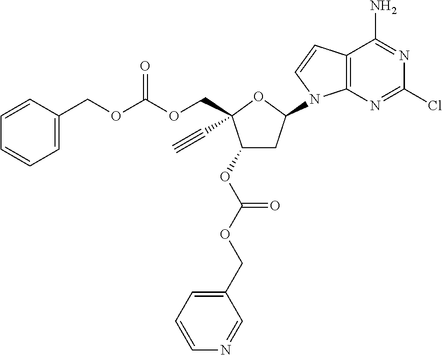 [SMILES: C#C[C@]1(COC(=O)OCc2ccccc2)O[C@@H](n2ccc3c(N)nc(Cl)nc32)C[C@@H]1OC(=O)OCc1cccnc1] | 578.02 |
| 175 | 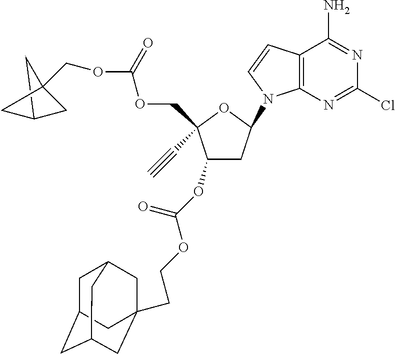 [SMILES: C#C[C@]1(COC(=O)OCC23CC(C2)C3)O[C@@H](n2ccc3c(N)nc(Cl)nc32)C[C@@H]1OC(=O)OCCC12CC3CC(CC(C3)C1)C2] | 639.20 |
| 176 | 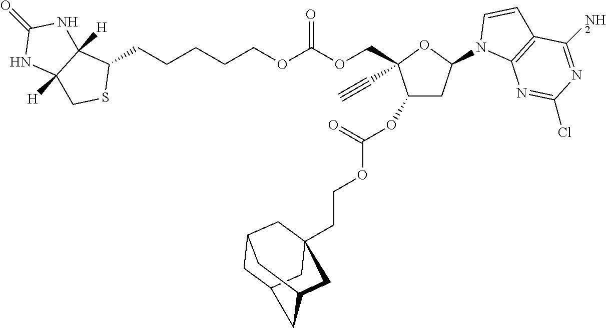 | 771.30 |

The Examples in the table below were prepared using procedures similar to those described in Example 65, from the appropriate starting material.

| | | Observed |
|---|---|---|
| | | m/z |
| Example | Structure | [M + H]⁺ |
| 177 | 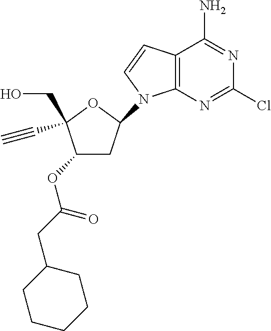 [SMILES: C#C[C@]1(CO)O[C@@H](n2ccc3c(N)nc(Cl)nc32)C[C@@H]1OC(=O)CC1CCCCC1] | 433.05 |
| 178 | 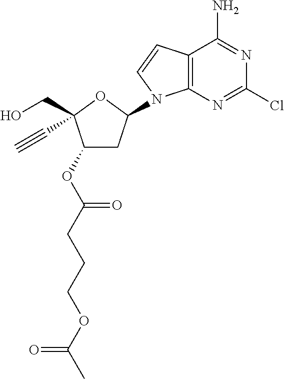 [SMILES: C#C[C@]1(CO)O[C@@H](n2ccc3c(N)nc(Cl)nc32)C[C@@H]1OC(=O)CCCOC(C)=O] | 436.95 |
| 179 | 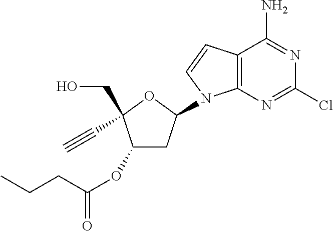 [SMILES: C#C[C@]1(CO)O[C@@H](n2ccc3c(N)nc(Cl)nc32)C[C@@H]1OC(=O)CCC] | 379.00 |
| 180 | 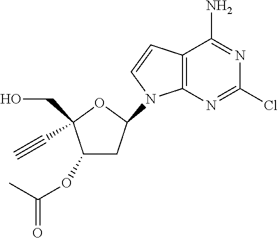 [SMILES: C#C[C@]1(CO)O[C@@H](n2ccc3c(N)nc(Cl)nc32)C[C@@H]1OC(C)=O] | 350.95 |
| 181 | 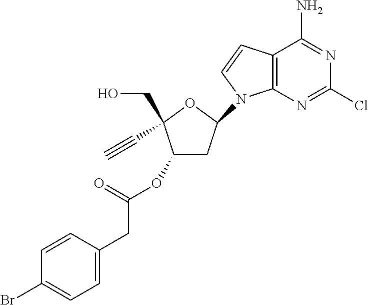 [SMILES: C#C[C@]1(CO)O[C@@H](n2ccc3c(N)nc(Cl)nc32)C[C@@H]1OC(=O)Cc1ccc(Br)cc1] | 504.90 |
| 182 | 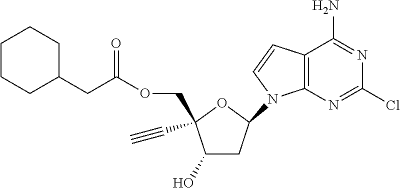 [SMILES: C#C[C@]1(COC(=O)CC2CCCCC2)O[C@@H](n2ccc3c(N)nc(Cl)nc32)C[C@@H]1O] | 431.1 [M − H]⁻ |
| 183 | 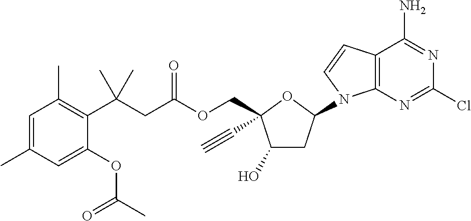 [SMILES: C#C[C@]1(COC(=O)CC(C)(C)c2c(C)cc(C)cc2OC(C)=O)O[C@@H](n2ccc3c(N)nc(Cl)nc32)C[C@@H]1O] | 555.15 |
| 184 | 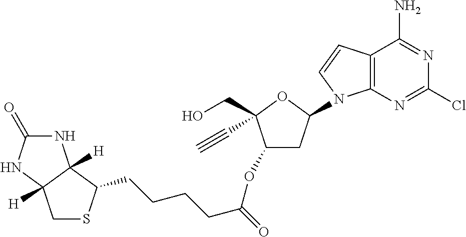 [SMILES: C#C[C@]1(CO)O[C@@H](n2ccc3c(N)nc(Cl)nc32)C[C@@H]1OC(=O)CCCC[C@@H]1SC[C@@H]2NC(=O)N[C@@H]21] | 535.40 |

The Examples in the table below were prepared using procedures similar to those described in Example 65 step 1, from the appropriate starting material.

| | | Observed |
|---|---|---|
| | | m/z |
| Example | Structure | [M + H]⁺ |
| 185 | 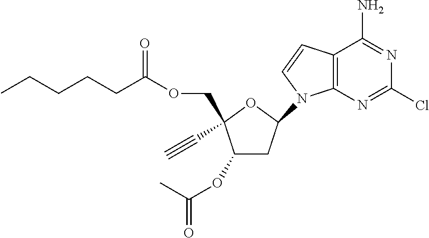 [SMILES: C#C[C@]1(COC(=O)CCCCC)O[C@@H](n2ccc3c(N)nc(Cl)nc32)C[C@@H]1OC(C)=O] | 449.00 |
| 186 | 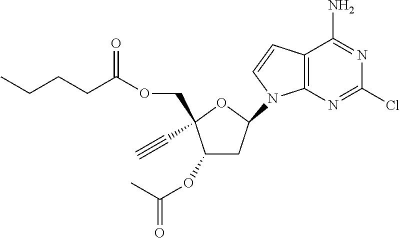 [SMILES: C#C[C@]1(COC(=O)CCCC)O[C@@H](n2ccc3c(N)nc(Cl)nc32)C[C@@H]1OC(C)=O] | 435.00 |
| 187 | 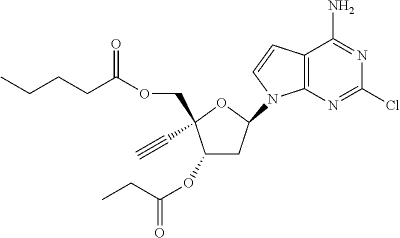 [SMILES: C#C[C@]1(COC(=O)CCCC)O[C@@H](n2ccc3c(N)nc(Cl)nc32)C[C@@H]1OC(=O)CC] | 449.05 |
| 188 | 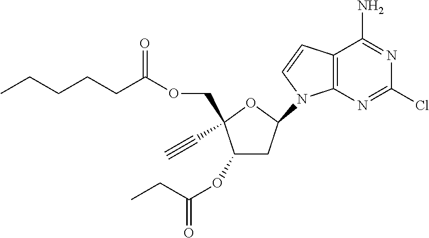 [SMILES: C#C[C@]1(COC(=O)CCCCC)O[C@@H](n2ccc3c(N)nc(Cl)nc32)C[C@@H]1OC(=O)CC] | 465.10 |
| 189 | 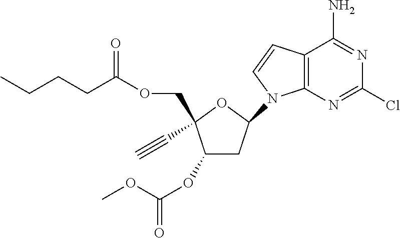 [SMILES: C#C[C@]1(COC(=O)CCCC)O[C@@H](n2ccc3c(N)nc(Cl)nc32)C[C@@H]1OC(=O)OC] | 451.20 |
| 190 | 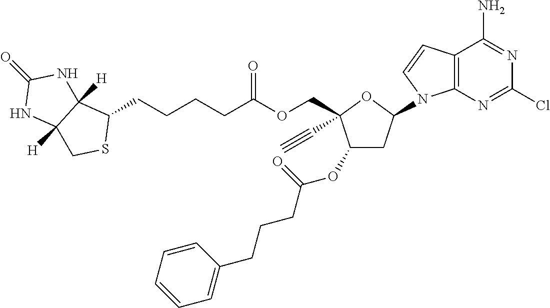 [SMILES: C#C[C@]1(COC(=O)CCCC[C@@H]2SC[C@@H]3NC(=O)N[C@@H]32)O[C@@H](n2ccc3c(N)nc(Cl)nc32)C[C@@H]1OC(=O)CCCc1ccccc1] | 681.05 |
| 191 | 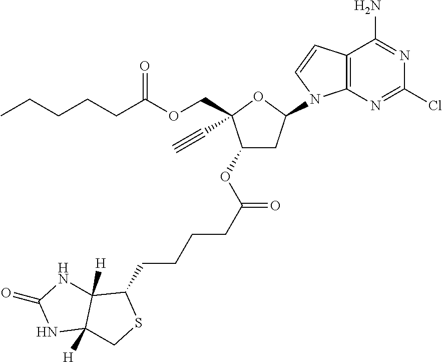 [SMILES: C#C[C@]1(COC(=O)CCCCC)O[C@@H](n2ccc3c(N)nc(Cl)nc32)C[C@@H]1OC(=O)CCCC[C@@H]1SC[C@@H]2NC(=O)N[C@@H]21] | 633.30 |
| 192 | 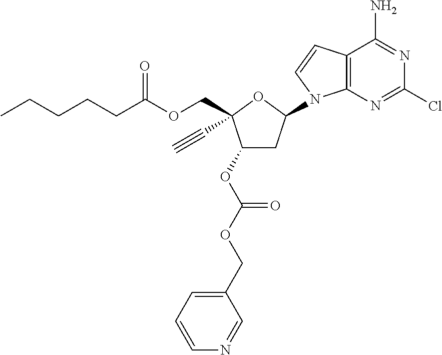 [SMILES: C#C[C@]1(COC(=O)CCCCC)O[C@@H](n2ccc3c(N)nc(Cl)nc32)C[C@@H]1OC(=O)OCc1cccnc1] | 542.25 |
| 193 | 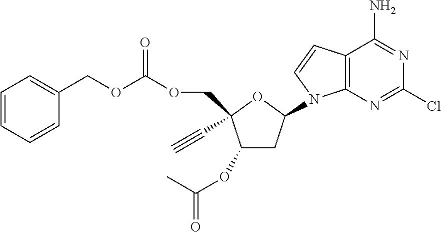 [SMILES: C#C[C@]1(COC(=O)OCc2ccccc2)O[C@@H](n2ccc3c(N)nc(Cl)nc32)C[C@@H]1OC(C)=O] | 485.10 |
| 194 | 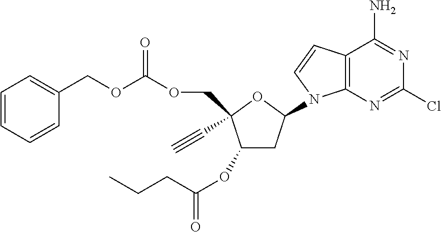 [SMILES: C#C[C@]1(COC(=O)OCc2ccccc2)O[C@@H](n2ccc3c(N)nc(Cl)nc32)C[C@@H]1OC(=O)CCC] | 513.25 |
| 195 | 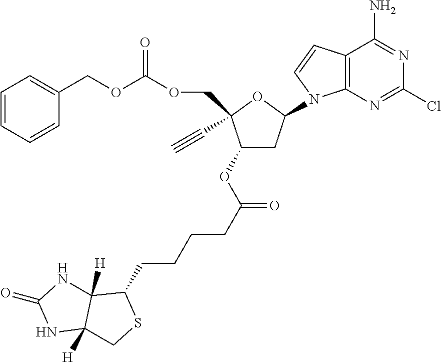 [SMILES: C#C[C@]1(COC(=O)OCc2ccccc2)O[C@@H](n2ccc3c(N)nc(Cl)nc32)C[C@@H]1OC(=O)CCCC[C@@H]1SC[C@@H]2NC(=O)N[C@@H]21] | 669.30 |
| 196 | 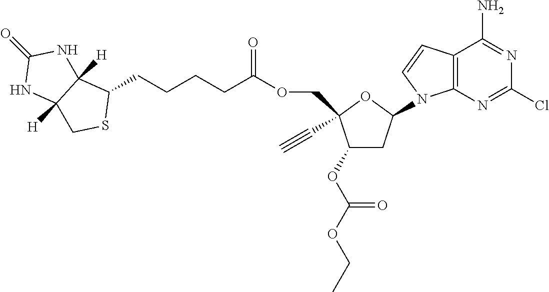 [SMILES: C#C[C@]1(COC(=O)CCCC[C@@H]2SC[C@@H]3NC(=O)N[C@@H]32)O[C@@H](n2ccc3c(N)nc(Cl)nc32)C[C@@H]1OC(=O)OCC] | 607.20 |

The Examples in the table below were prepared using procedures similar to those described in Example 66, from the appropriate starting material.

| | | Observed |
|---|---|---|
| | | m/z |
| Example | Structure | [M + H]⁺ |
| 197 | 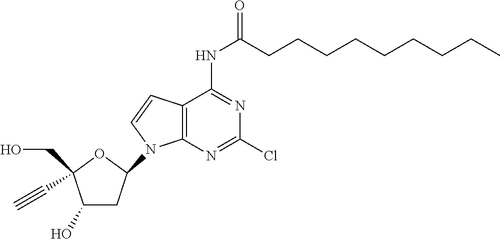 [SMILES: C#C[C@]1(CO)O[C@@H](n2ccc3c(NC(=O)CCCCCCCCC)nc(Cl)nc32)C[C@@H]1O] | 463.05 |
| 198 | 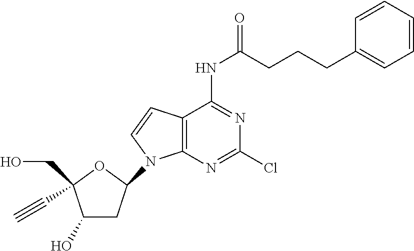 [SMILES: C#C[C@]1(CO)O[C@@H](n2ccc3c(NC(=O)CCCc4ccccc4)nc(Cl)nc32)C[C@@H]1O] | 455.20 |
| 199 | 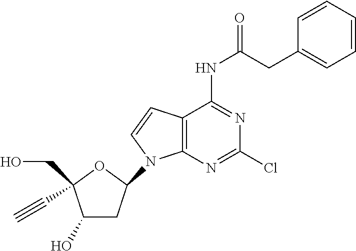 [SMILES: C#C[C@]1(CO)O[C@@H](n2ccc3c(NC(=O)Cc4ccccc4)nc(Cl)nc32)C[C@@H]1O] | 427.10 |
| 200 | 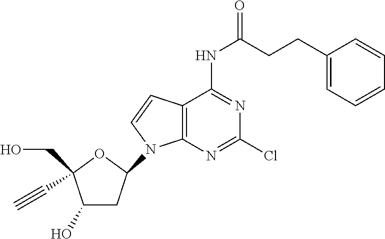 [SMILES: C#C[C@]1(CO)O[C@@H](n2ccc3c(NC(=O)CCc4ccccc4)nc(Cl)nc32)C[C@@H]1O] | 441.30 |

The Examples in the table below were prepared using procedures similar to those described in Example 67, from the appropriate starting material.

| | | Observed m/z |
|---|---|---|
| Example | Structure | [M + H]⁺ |
| 201 | 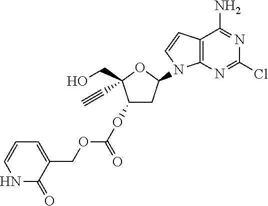 [SMILES: C#C[C@]1(CO)O[C@@H](n2ccc3c(N)nc(Cl)nc32)C[C@@H]1OC(=O)OCc1ccc[nH]c1=O] | 460.40 |

The Examples in the table below were prepared using procedures similar to those described in Example 67, Steps 1 and 2, from the appropriate starting material.

| | | Observed |
|---|---|---|
| | | m/z |
| Example | Structure | [M + H]⁺ |
| 202 | 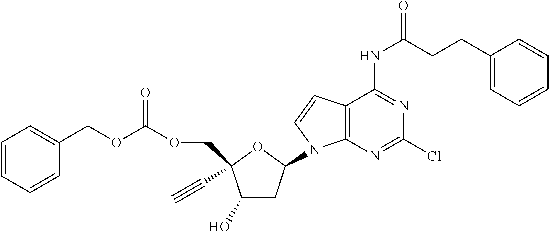 [SMILES: C#C[C@]1(COC(=O)OCc2ccccc2)O[C@@H](n2ccc3c(NC(=O)CCc4ccccc4)nc(Cl)nc32)C[C@@H]1O] | 575.20 |
| 203 | 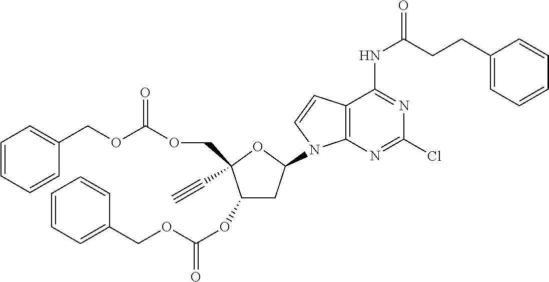 [SMILES: C#C[C@]1(COC(=O)OCc2ccccc2)O[C@@H](n2ccc3c(NC(=O)CCc4ccccc4)nc(Cl)nc32)C[C@@H]1OC(=O)OCc1ccccc1] | 709.20 |
| 204 | 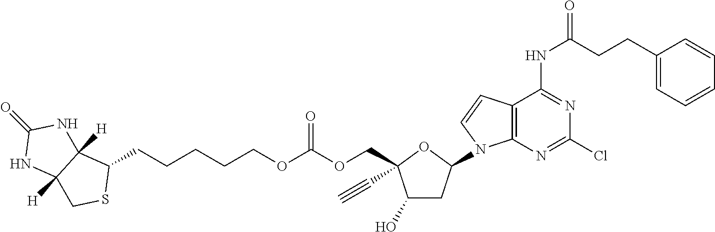 [SMILES: C#C[C@]1(COC(=O)OCCCCC[C@@H]2SC[C@@H]3NC(=O)N[C@@H]32)O[C@@H](n2ccc3c(NC(=O)CCc4ccccc4)nc(Cl)nc32)C[C@@H]1O] | 697.25 |
| 205 | 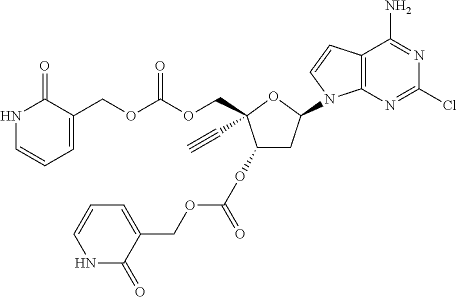 [SMILES: C#C[C@]1(COC(=O)OCc2ccc[nH]c2=O)O[C@@H](n2ccc3c(N)nc(Cl)nc32)C[C@@H]1OC(=O)OCc1ccc[nH]c1=O] | 610.95 |
| 206 | 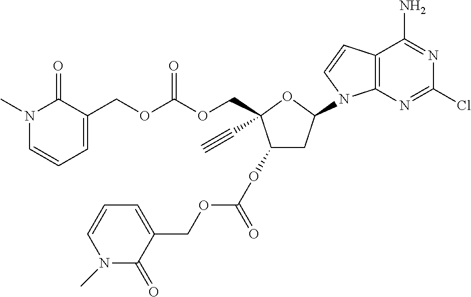 [SMILES: C#C[C@]1(COC(=O)OCc2cccn(C)c2=O)O[C@@H](n2ccc3c(N)nc(Cl)nc32)C[C@@H]1OC(=O)OCc1cccn(C)c1=O] | 638.90 |
| 207 | 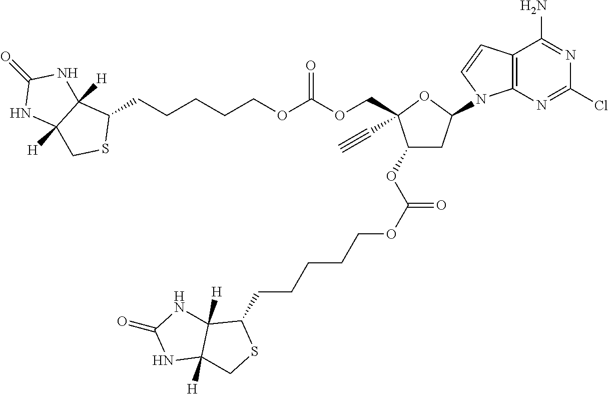 [SMILES: C#C[C@]1(COC(=O)OCCCCC[C@@H]2SC[C@@H]3NC(=O)N[C@@H]32)O[C@@H](n2ccc3c(N)nc(Cl)nc32)C[C@@H]1OC(=O)OCCCCC[C@@H]1SC[C@@H]2NC(=O)N[C@@H]21] | 821.10 |

The Examples in the table below were prepared using procedures similar to those described in Example 67 steps 1 and 2 and replacing step 3 with Example 23 step 2, from the appropriate starting material.

| | | Observed |
|---|---|---|
| | | m/z |
| Example | Structure | [M + H]⁺ |
| 208 | 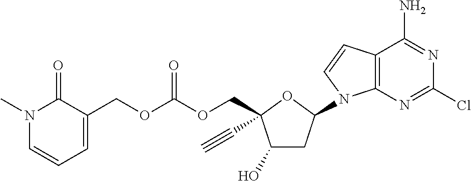 [SMILES: C#C[C@]1(COC(=O)OCc2cccn(C)c2=O)O[C@@H](n2ccc3c(N)nc(Cl)nc32)C[C@@H]1O] | 474.05 |
| 209 | 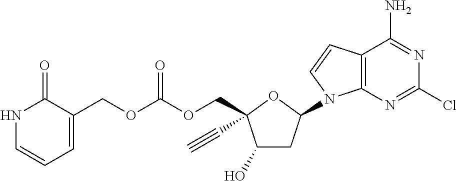 [SMILES: C#C[C@]1(COC(=O)OCc2ccc[nH]c2=O)O[C@@H](n2ccc3c(N)nc(Cl)nc32)C[C@@H]1O] | 459.95 |

The Examples in the table below were prepared using procedures similar to those described in Example 69, from the appropriate starting material.

| | | Observed |
|---|---|---|
| | | m/z |
| Example | Structure | [M + H]⁺ |
| 210 | 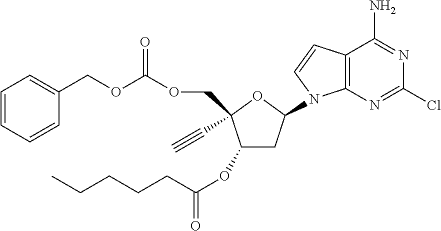 [SMILES: C#C[C@]1(COC(=O)OCc2ccccc2)O[C@@H](n2ccc3c(N)nc(Cl)nc32)C[C@@H]1OC(=O)CCCCC] | 541.00 |
| 211 | 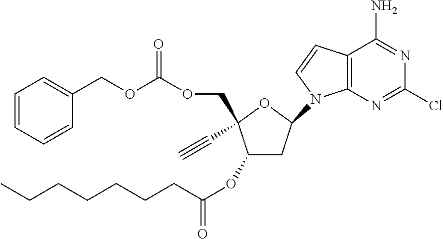 [SMILES: C#C[C@]1(COC(=O)OCc2ccccc2)O[C@@H](n2ccc3c(N)nc(Cl)nc32)C[C@@H]1OC(=O)CCCCCCC] | 569.15 |
| 212 | 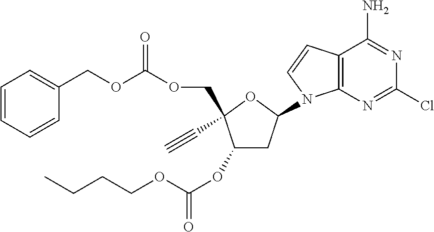 [SMILES: C#C[C@]1(COC(=O)OCc2ccccc2)O[C@@H](n2ccc3c(N)nc(Cl)nc32)C[C@@H]1OC(=O)OCCCC] | 543.20 |
| 213 | 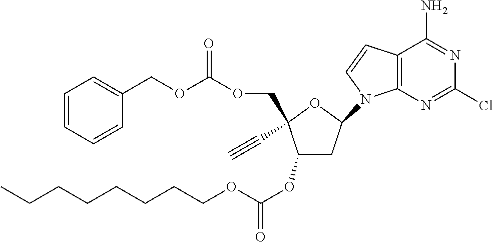 [SMILES: C#C[C@]1(COC(=O)OCc2ccccc2)O[C@@H](n2ccc3c(N)nc(Cl)nc32)C[C@@H]1OC(=O)OCCCCCCCC] | 599.30 |
| 214 | 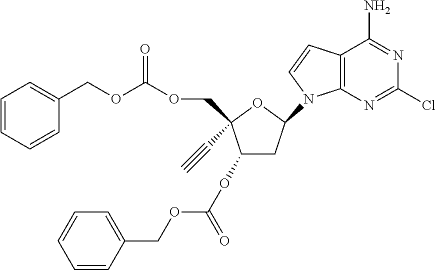 [SMILES: C#C[C@]1(COC(=O)OCc2ccccc2)O[C@@H](n2ccc3c(N)nc(Cl)nc32)C[C@@H]1OC(=O)OCc1ccccc1] | 577.40 |
| 215 | 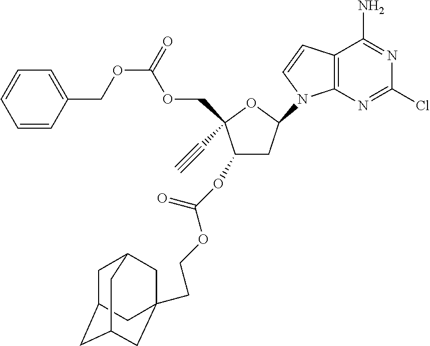 [SMILES: C#C[C@]1(COC(=O)OCc2ccccc2)O[C@@H](n2ccc3c(N)nc(Cl)nc32)C[C@@H]1OC(=O)OCCC12CC3CC(CC(C3)C1)C2] | 649.30 |
| 216 | 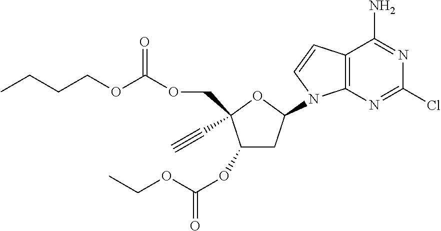 [SMILES: C#C[C@]1(COC(=O)OCCCC)O[C@@H](n2ccc3c(N)nc(Cl)nc32)C[C@@H]1OC(=O)OCC] | 479.10 |

The Examples in the table below were prepared using procedures similar to those described in Example 69, from the appropriate starting material.

| | | Observed |
|---|---|---|
| | | m/z |
| Example | Structure | [M + H]⁺ |
| 217 | 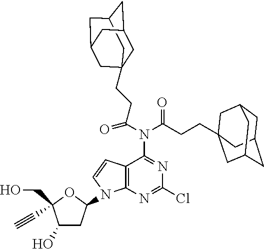 [SMILES: C#C[C@]1(CO)O[C@@H](n2ccc3c(N(C(=O)CCC45CC6CC(CC(C6)C4)C5)C(=O)CCC45CC6CC(CC(C6)C4)C5)nc(Cl)nc32)C[C@@H]1O] | 689.40 |

The Examples in the table below were prepared using procedures similar to those described in Example 71, from the appropriate starting material.

| | | Observed |
|---|---|---|
| | | m/z |
| Example | Structure | [M + H]⁺ |
| 218 | 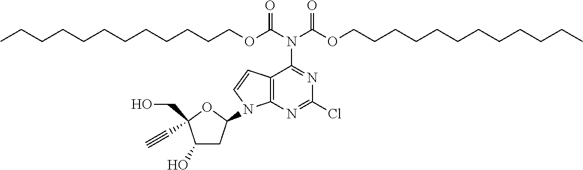 [SMILES: C#C[C@]1(CO)O[C@@H](n2ccc3c(N(C(=O)OCCCCCCCCCCCC)C(=O)OCCCCCCCCCCCC)nc(Cl)nc32)C[C@@H]1O] | 733.40 |
| 219 | 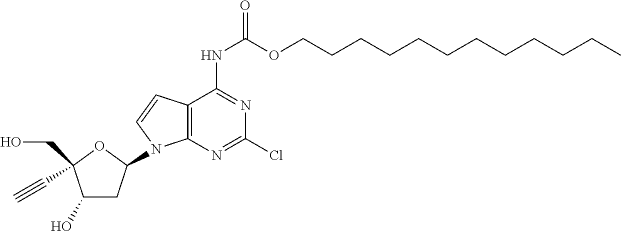 [SMILES: C#C[C@]1(CO)O[C@@H](n2ccc3c(NC(=O)OCCCCCCCCCCCC)nc(Cl)nc32)C[C@@H]1O] | 521.25 |
| 220 | 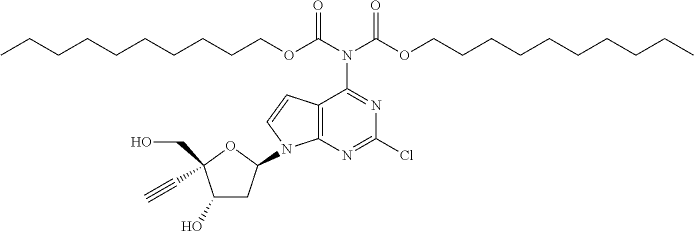 [SMILES: C#C[C@]1(CO)O[C@@H](n2ccc3c(N(C(=O)OCCCCCCCCCC)C(=O)OCCCCCCCCCC)nc(Cl)nc32)C[C@@H]1O] | 677.30 |
| 221 | 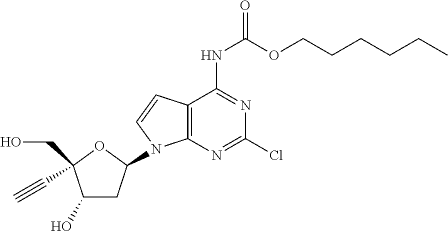 [SMILES: C#C[C@]1(CO)O[C@@H](n2ccc3c(NC(=O)OCCCCCC)nc(Cl)nc32)C[C@@H]1O] | 437.15 |
| 222 | 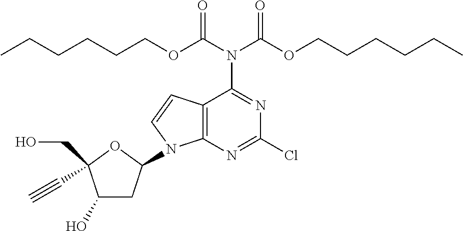 [SMILES: C#C[C@]1(CO)O[C@@H](n2ccc3c(N(C(=O)OCCCCCC)C(=O)OCCCCCC)nc(Cl)nc32)C[C@@H]1O] | 565.15 |
| 223 | 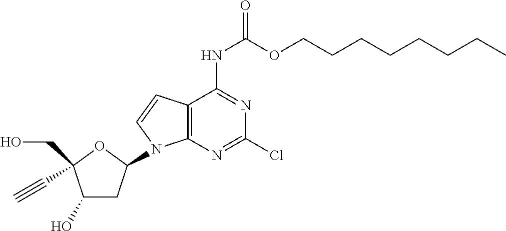 [SMILES: C#C[C@]1(CO)O[C@@H](n2ccc3c(NC(=O)OCCCCCCCC)nc(Cl)nc32)C[C@@H]1O] | 465.20 |
| 224 | 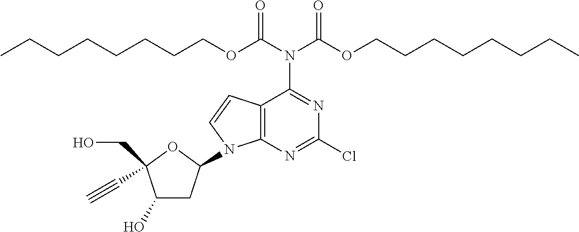 [SMILES: C#C[C@]1(CO)O[C@@H](n2ccc3c(N(C(=O)OCCCCCCCC)C(=O)OCCCCCCCC)nc(Cl)nc32)C[C@@H]1O] | 621.45 |

The Examples in the table below were prepared using procedures similar to those described in Example 73, from the appropriate starting material.

| | | Observed |
|---|---|---|
| | | m/z |
| Example | Structure | [M + H]⁺ |
| 225 | 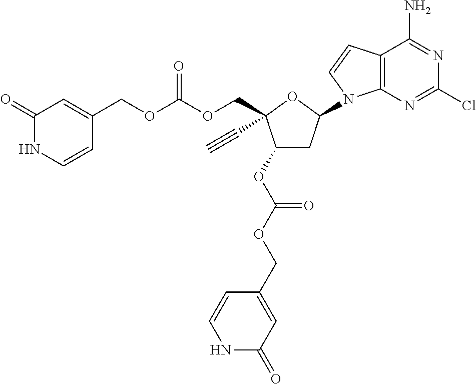 [SMILES: C#C[C@]1(COC(=O)OCc2cc[nH]c(=O)c2)O[C@@H](n2ccc3c(N)nc(Cl)nc32)C[C@@H]1OC(=O)OCc1cc[nH]c(=O)c1] | 611.00 |
| 226 | 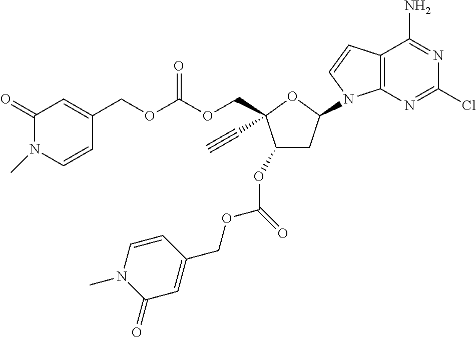 [SMILES: C#C[C@]1(COC(=O)OCc2ccn(C)c(=O)c2)O[C@@H](n2ccc3c(N)nc(Cl)nc32)C[C@@H]1OC(=O)OCc1ccn(C)c(=O)c1] | 639.05 |
| 227 | 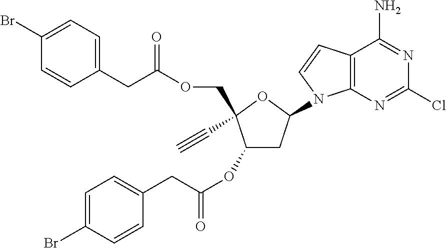 [SMILES: C#C[C@]1(COC(=O)Cc2ccc(Br)cc2)O[C@@H](n2ccc3c(N)nc(Cl)nc32)C[C@@H]1OC(=O)Cc1ccc(Br)cc1] | 703.05 |

### Aqueous Solubility of Compounds

Aqueous solubility of the compounds was determined by suspending excess solid (typically 0.3-0.5 mg) in PBS, pH 7.4, at a concentration of 0.5 mg/mL. The suspension was vortexed for 10-30 seconds to wet the material and stirred at 600 rpm for 24 hours. For analysis by UPLC, 150 μL aliquot sample was transferred into a 0.45 m PVDF spin filter and centrifuged at 14,000 rpm for 5 minutes. 100 μL of the filtrate was diluted with 100 μL 1:1 Acetonitrile (ACN):H₂O. The resultant samples were analyzed using UPLC and quantified against a standard curve of the compound of interest dissolved in 1:1 ACN:H₂O. The retains of the solids were checked by XRPD and PLM to determine if the phase of the material was crystalline or amorphous. Compounds in Table 1 exhibited lower aqueous solubility than the parent Compound A (0.5 mg/mL in water).

TABLE 1
Aqueous solubility (μg/mL)
for Examples in pH 7.4
phosphate buffered saline as
amorphous or crystalline material.

| | Aqu. Solubility |
|---|---|
| Example | (μg/mL) |
| 1 | 42 |
| 2 | 136 |
| 3 | 27 |
| 4 | <1 |
| 5 | <1 |
| 6 | <1 |
| 7 | <1 |
| 8 | 304 |
| 9 | 182 |
| 10 | <1 |
| 11 | 285 |
| 12 | 2.9 |
| 13 | 1.9 |
| 14 | 113 |
| 15 | 55 |
| 16 | <1 |
| 17 | 164 |
| 18 | 0.45 |
| 19 | 34 |
| 20 | <1 |
| 21 | <1 |
| 22 | 362 |
| 23 | 351 |
| 24 | <1 |
| 25 | 2.0 |
| 26 | <1 |
| 27 | <1 |
| 28 | 68 |
| 29 | <1 |
| 30 | <1 |
| 31 | 11 |
| 32 | 3.7 |
| 33 | 41 |
| 34 | <1 |
| 35 | <1 |
| 36 | <1 |
| 37 | 118 |
| 38 | <1 |
| 39 | <1 |
| 40 | 282 |
| 42 | <1 |
| 44 | <1 |
| 45 | 45 |
| 47 | <1 |
| 48 | <1 |
| 49 | 49 |
| 50 | 239 |
| 51 | 309 |
| 52 | 2.4 |
| 53 | <1 |
| 54 | 247 |
| 55 | <1 |
| 56 | 122 |
| 57 | 5 |
| 58 | <1 |
| 59 | 16 |
| 60 | <1 |
| 61 | <1 |
| 62 | <1 |
| 64 | <1 |
| 65 | 224 |
| 68 | <1 |
| 73 | 24 |
| 74 | <1 |
| 75 | 30 |
| 76 | 23 |
| 77 | 14 |
| 78 | 2 |
| 79 | <1 |
| 80 | 305 |
| 81 | 2 |
| 82 | 11 |
| 84 | 69 |
| 86 | 145 |
| 87 | <1 |
| 88 | 157 |
| 89 | 365 |
| 90 | <1 |
| 100 | 72 |
| 101 | 19 |
| 113 | <1 |
| 114 | 66 |
| 115 | <1 |
| 116 | 39 |
| 117 | <1 |
| 119 | 57 |
| 120 | 108 |
| 121 | 7 |
| 122 | 107 |
| 124 | 71 |
| 126 | 21 |
| 129 | 37 |
| 133 | 341 |
| 134 | <1 |
| 136 | <1 |
| 142 | <1 |
| 143 | 229 |
| 145 | 38 |
| 146 | 7 |
| 152 | <1 |
| 153 | <1 |
| 154 | <1 |
| 156 | 66 |
| 160 | <1 |
| 161 | <1 |
| 163 | <1 |
| 171 | 1.9 |
| 173 | <1 |
| 174 | <1 |
| 177 | <1 |
| 178 | 143 |
| 179 | 27 |
| 180 | 257 |
| 181 | <1 |
| 182 | 124 |
| 184 | 161 |
| 185 | 4.2 |
| 188 | 2.4 |
| 189 | 23 |
| 190 | <1 |
| 193 | 1.2 |
| 194 | 2.3 |
| 197 | <1 |
| 199 | 51 |
| 200 | 307 |
| 207 | 1.2 |
| 210 | <1 |
| 212 | <1 |
| 213 | <1 |
| 214 | <1 |
| 216 | <1 |
| 225 | 70 |
| 226 | 19 |
| 227 | <1 |

### Potency of the Compounds

Antiviral potency of the compounds was determined based on their ability to block HIV-1 replication in a GFP reporter cell line; this assay is referred to as Viral KINetics in Green cells or VIKING assay. HIV-1 replication was monitored using MT4-gag-GFP clone D3 (hereafter designate MT4-GFP) cells, which are MT4 cells modified to harbor a GFP reporter gene, the expression of which is dependent on the HIV-1 proteins tat and rev. (MT4 is a human T-cell line that is particularly sensitive to HIV-1 replication.) HIV-1 infection of MT4-GFP cells results in GFP expression approximately 24 h post-infection. MT4-GFP cells were maintained at 37° C./5% CO₂/90% relative humidity in RPMI 1640 supplemented with 10% fetal bovine serum, 100 U/mL penicillin/streptomycin, and 400 mg/mL G418 to maintain the reporter gene. For infections, MT4-GFP cells were placed in the same medium lacking G418 and infected overnight with HIV-1 wild-type (R8) virus at an approximate multiplicity of infection of 0.01 in the same incubation conditions. Cells were then washed and resuspended in RPMI 1640 containing 10% normal human serum at 2×10⁵ cells/mL. Compound plates were prepared by dispensing compounds dissolved in DMSO into wells of 384-well poly D lysine-coated plates (0.2 μl/well) using an ECHO acoustic dispenser. Each compound was tested in a 10-point serial 3-fold dilution (typical final concentrations: 4 μM-0.2 nM). Controls included no inhibitor (DMSO only) and a combination of three antiviral agents as a positive control (efavirenz, indinavir, and an integrase strand transfer inhibitor at final concentrations of 4 μM each). Infected cells were added (50 μL/well) to compound plates and were maintained at 37° C./5% CO₂/90% relative humidity. Infected cells were quantified at two time points, approximately 48 h and 72 h post-infection, by counting the number of green cells in each well using an Acumen eX3 scanner. The increase in the number of green cells over ˜24 h period gives the reproductive ratio, R, which is calculated by dividing the number of green cells at 72 h over those at 48 h post-infection. The percent inhibition caused by a test compound is calculated using the following formula:

$$\% inhibition = [ \text{} 1 \text{} - \text{} ( {R}_{testcompound} - {R}_{positivecontrol} ) \text{} / ( {R}_{DMSOonly} - {R}_{positivecontrol} ) ] \times 100 \%$$

The dose-response curves of each test compound were plotted as % inhibition versus the compound concentration. IC₅₀ values were determined by non-linear 4-parameter fitting of the dose-response curves. The IC₅₀ of parent Compound A was 3.3 nM in the Viking assay.

TABLE 2
EC₅₀values (nM) for
Examples in the HIV Viking
assay with 10% normal
human serum (NHS).

| | Viking |
|---|---|
| | (10% NHS), |
| Example | IC₅₀ (nM) |
| 1 | 74 |
| 2 | 41 |
| 3 | 3.7 |
| 4 | 2.9 |
| 5 | 35 |
| 6 | 44 |
| 7 | 2.9 |
| 8 | 42 |
| 9 | 2.8 |
| 10 | 9.9 |
| 11 | 1.9 |
| 12 | 2.9 |
| 13 | 2.2 |
| 14 | 2.5 |
| 15 | 2.3 |
| 16 | 5.6 |
| 17 | 1.4 |
| 18 | 2.1 |
| 19 | 382 |
| 20 | 2.8 |
| 21 | 18 |
| 22 | 199 |
| 23 | 2.2 |
| 24 | 1.7 |
| 25 | 3.5 |
| 26 | 7.0 |
| 27 | 3.1 |
| 28 | 4.3 |
| 29 | 5.8 |
| 30 | 3.6 |
| 31 | 4.6 |
| 32 | 5.1 |
| 33 | 2.4 |
| 34 | 6.1 |
| 35 | 6.2 |
| 36 | 5.5 |
| 37 | 2.3 |
| 38 | 8.0 |
| 39 | 3.0 |
| 40 | 5.5 |
| 42 | 10.9 |
| 44 | 351 |
| 45 | 143 |
| 47 | 34 |
| 48 | 35 |
| 49 | 7.5 |
| 50 | 1.6 |
| 51 | 2.2 |
| 52 | 337 |
| 53 | 1.7 |
| 54 | 1.5 |
| 55 | 2.2 |
| 56 | 2.3 |
| 57 | 2.6 |
| 58 | 1.9 |
| 59 | 2.8 |
| 60 | 3.5 |
| 61 | 4.5 |
| 62 | 3.9 |
| 63 | 1490 |
| 64 | 4.6 |
| 65 | 26 |
| 66 | 197 |
| 67 | 6.4 |
| 68 | 6.5 |
| 69 | 10 |
| 70 | 58 |
| 71 | 785 |
| 72 | 544 |
| 73 | 3.2 |
| 74 | 69 |
| 75 | 47 |
| 76 | 31 |
| 77 | 5.0 |
| 78 | 6.7 |
| 79 | 4.3 |
| 80 | 10 |
| 81 | 3.3 |
| 82 | 4.0 |
| 83 | 25 |
| 84 | 129 |
| 85 | 3.2 |
| 86 | 13 |
| 87 | 3.8 |
| 88 | 4.4 |
| 89 | 29 |
| 90 | 3.9 |
| 91 | 135 |
| 92 | 3510 |
| 93 | 7.8 |
| 94 | 6.1 |
| 95 | 8.6 |
| 96 | 7.1 |
| 97 | 1.6 |
| 98 | 91 |
| 99 | 29 |
| 100 | 2.5 |
| 101 | 3.4 |
| 102 | 43 |
| 103 | 405 |
| 104 | 4.1 |
| 105 | 3.5 |
| 106 | 2.5 |
| 107 | 74 |
| 108 | 20 |
| 109 | 21 |
| 110 | 2.7 |
| 111 | 5.2 |
| 112 | 28 |
| 113 | 26 |
| 114 | 3.9 |
| 115 | 5.7 |
| 116 | 4.2 |
| 117 | 4.0 |
| 118 | 4.3 |
| 119 | 3.5 |
| 120 | 3.8 |
| 121 | 3.1 |
| 122 | 4.0 |
| 123 | 4.1 |
| 124 | 3.6 |
| 125 | 3.7 |
| 126 | 2.6 |
| 127 | 3.8 |
| 128 | 3.0 |
| 129 | 3.1 |
| 130 | 2.9 |
| 131 | 3.5 |
| 132 | 3.0 |
| 133 | 2.7 |
| 134 | 3.7 |
| 135 | 3.8 |
| 136 | 3.2 |
| 137 | 4.5 |
| 138 | 41 |
| 139 | 3.6 |
| 140 | 5.0 |
| 141 | 3.5 |
| 142 | 1760 |
| 143 | 2.9 |
| 144 | 48 |
| 145 | 3.2 |
| 146 | 19 |
| 147 | 55 |
| 148 | 339 |
| 149 | 429 |
| 150 | 740 |
| 151 | 17 |
| 152 | 6.0 |
| 153 | 3.8 |
| 154 | 3.5 |
| 155 | 29 |
| 156 | 3.2 |
| 157 | 20 |
| 158 | 11 |
| 159 | 14 |
| 160 | 11 |
| 161 | 5.8 |
| 162 | 8.9 |
| 163 | 3.5 |
| 164 | 13 |
| 165 | 4.9 |
| 166 | 6.7 |
| 167 | 7.2 |
| 168 | 3.9 |
| 169 | 13.0 |
| 170 | 4.3 |
| 171 | 18 |
| 172 | 25 |
| 173 | 6.2 |
| 174 | 13 |
| 175 | 34 |
| 176 | 127 |
| 177 | 8.5 |
| 178 | 3.8 |
| 179 | 11 |
| 180 | 27 |
| 181 | 4.0 |
| 182 | 3.1 |
| 183 | 2.7 |
| 184 | 57 |
| 185 | 17 |
| 186 | 25 |
| 187 | 27 |
| 188 | 17 |
| 189 | 19 |
| 190 | 6.5 |
| 191 | 20 |
| 192 | 6.5 |
| 193 | 29 |
| 194 | 17 |
| 195 | 40 |
| 196 | 27 |
| 197 | 29 |
| 198 | 63 |
| 199 | 230 |
| 200 | 155 |
| 201 | 2.6 |
| 202 | 146 |
| 203 | 47 |
| 204 | 123 |
| 205 | 4.0 |
| 206 | 3.4 |
| 207 | 21 |
| 208 | 4.4 |
| 209 | 3.9 |
| 210 | 2.9 |
| 211 | 4.0 |
| 212 | 4.8 |
| 213 | 7.9 |
| 214 | 4.7 |
| 215 | 44 |
| 216 | 21 |
| 217 | >4034 |
| 218 | >4034 |
| 219 | 2410 |
| 220 | 615 |
| 221 | 710 |
| 222 | 1370 |
| 223 | 411 |
| 224 | 842 |
| 225 | 13 |
| 226 | 13 |
| 227 | 12 |

Plasma stability of the compounds was evaluated in frozen human plasma at 37° C. in a 10% CO₂ environment. The stability of the analyte was assessed by determining the percentage of drug loss over the course of 0, 0.25, 0.5, 1, and 3 hr. In addition, the appearance of Compound A was determined for the same time course. Percent of drug loss as based on ratio with internal standard. Analyte concentrations were measured by LC/MS/MS. Linear regression was performed with log transformed percent of average TO values and linear time values. The elimination rate constant (ke) was calculated from the slope of this regression line and the half-life (T½) calculated based on the elimination rate constant.

TABLE 3
Stability for Examples reported
as half-life (T1/2) in the
presence of human plasma.

| | T1/2 Human |
|---|---|
| Example | Plasma (hours) |
| 1 | 19.1 |
| 2 | 48.4 |
| 3 | 16.7 |
| 4 | ND |
| 5 | 57.7 |
| 6 | 1.1 |
| 7 | 0.036 |
| 8 | 13.3 |
| 9 | 0.026 |
| 11 | 0.042 |
| 12 | 0.41 |
| 14 | <0.25 |
| 15 | <0.25 |
| 23 | 0.028 |
| 40 | 1.9 |
| 42 | <0.25 |
| 44 | 2.2 |
| 45 | 19.4 |

### In Vitro Metabolic Stability of Prodrugs to Form Compound A

The formation of Compound A from 14 of the disclosed prodrug compounds was measured in the presence of cryopreserved hepatocytes from humans. The incubations were carried out in Williams E Media with 4 mM L-glutamate at a cell density of 1×10⁶ cells/mL in a 37° C. incubator with a controlled atmosphere of 5% CO₂. Aliquots were taken at 0, 30, and 60 minutes, and quenched with cold acetonitrile containing an appropriate internal standard. The samples were then vortex-mixed and centrifuged at ˜2,900×g for 15 minutes. For analysis, the supernatants were diluted with 0.1% (v/v) formic acid in water and analyzed using LC/MS-MS.

The results are reported in Table 4 and establish that Examples 10, 18, 20, 21, 24, 27, 34, 36, 42, 48, 60, 61, 64 and 68 can be efficiently converted to Compound A by human hepatocytes. Specifically, all of these prodrug compounds exhibited a formation of Compound A in the presence of cryopreserved hepatocytes from humans above 75%. That is, over 75% of prodrug compound added to the hepatocytes underwent conversion to Compound A.

TABLE 4
Formation of Compound
A in the presence of
human hepatocytes.

| | % Formation of |
|---|---|
| | Compound A |
| Example | after 60 minutes |
| 10 | 87% |
| 18 | 112% |
| 20 | 96% |
| 21 | 104% |
| 24 | 120% |
| 27 | 99% |
| 34 | 138% |
| 36 | 108% |
| 42 | 76% |
| 48 | 58% |
| 60 | 100% |
| 61 | 87% |
| 64 | 102% |
| 68 | 94% |

### Plasma Exposures Following Intramuscular Administration of Prodrugs to Wistar Han Rats

The exposures of parent Compound A were evaluated in Wistar Han rats after a single intramuscular (IM) administration of 47 mg/kg of one of the following compounds: Compound A, Example 10, Example 18, Example 20, Example 21, Example 24, Example 27, Example 34, Example 36, Example 42, Example 48, Example 60, Example 61, Example 64 or Example 68. The test compounds were formulated as homogenous suspensions at concentrations of 200 mg/mL, containing functional excipients that include a surfactant, a suspending agent and a buffer, with the exception of Example 68 which was formulated as a solution (also at 200 mg/mL).

A single dose of 14 mg of each test compound was administered to Wistar Han rats (n=4) by intramuscular injection (0.07 mL). Blood samples were collected at 0.5, 1, 2, 4, 6, 24, 48, 120 (5 days), and 168 hours (7 days) post-dose. Blood (approximately 0.25 mL) was collected into pre-chilled blood collection tubes containing 12.5 μL of 2 mM Dichlorvos solution in water. Plasma was processed by centrifugation at 6,000 rpm for 10 min and plasma samples were frozen and maintained at −80° C. until analyzed. For analysis, plasma (50 mL) was mixed with 200 mL acetonitrile containing internal standards and analyzed by LC/MS-MS.

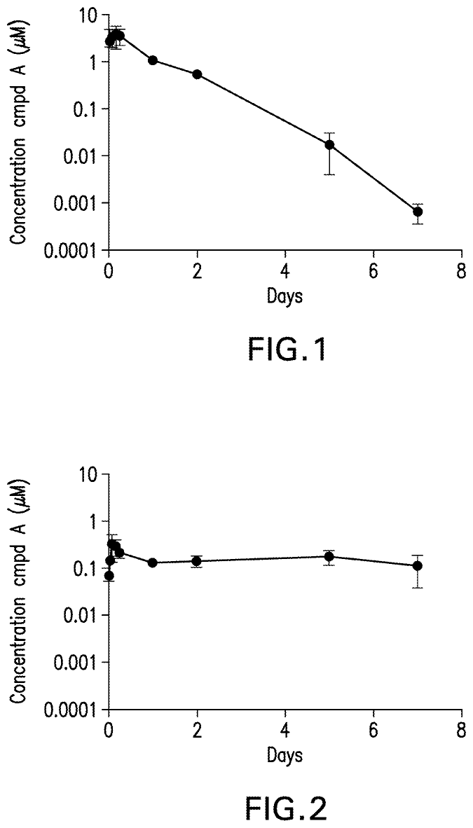

The results of these pharmacokinetic studies are shown in the concentration vs. time plots of FIGS. 1-15. FIGS. 2-15 show the release of Compound A into systemic circulation over 168 hours for Examples 10, 18, 20, 21, 24, 27, 34, 36, 42, 48, 60, 61, 64 and 68. This data establishes that these compounds can be readily delivered intramuscularly and can efficiently release Compound A into systemic circulation in mammals in vivo.

What is claimed is:

1. A compound of Formula (VI): 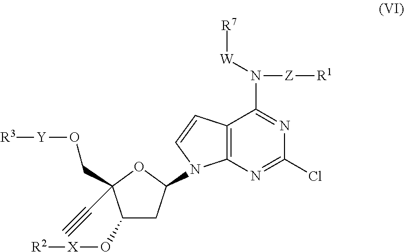
or a pharmaceutically acceptable salt thereof, wherein:
X is selected from —C(═O)—, —C(═O)—O—, —CR⁵R⁶—O—C(═O)—, —CR⁵R⁶—O—C(═O)—O—, and a bond;
Y is selected from —C(═O)—, —C(═O)—O—, —CR⁵R⁶—O—C(═O)—, —CR⁵R⁶—O—C(═O)—O—, —P(═O)(O—C₆₋₁₂ aryl)-NH—CR⁴C(═O)—O—, and a bond;
Z is selected from —C(═O)—, —C(═O)—O—, and a bond;
W is selected from —C(═O)—, —C(═O)—O—, and a bond;
R¹ is selected from the group consisting of hydrogen, C₁₋₂₁ alkyl, (CR⁵R⁶)_{z}—C₃₋₁₂cycloalkyl, (CR⁵R⁶)_{z}—C₅₋₁₂heterocyclyl, (CR⁵R⁶)_{z}—C₆₋₁₂ aryl, (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy;
R² is selected from the group consisting of hydrogen, C₁₋₂₁ alkyl, (CR⁵R⁶)_{z}—C₃₋₁₂cycloalkyl, (CR⁵R⁶)_{z}—C₅₋₁₂heterocyclyl, (CR⁵R⁶)_{z}—C₆₋₁₂ aryl, (CR⁵R⁶)_{z}—C₅₋₁₂ heteroaryl, and (CR⁵R⁶)_{z}—C₁₋₆ alkyl ester, wherein said alkyl, cycloalkyl, heterocyclyl, aryl or heteroaryl group can be optionally substituted with one to three groups independently selected from halo, oxo, C₁₋₆ alkyl, C₃₋₁₂ cycloalkyl and hydroxy;
R³ is selected from the group consisting of hydrogen, C₁₋₂₁ alkyl, (CR⁵R⁶)_{z}—C₃₋₁₂cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, or heteroaryl group can be optionally substituted with one to three groups independently selected from halo, oxo, C₁₋₆ alkyl, C₃₋₁₂ cycloalkyl, or hydroxy, and wherein said aryl can be optionally substituted with one to three groups independently selected from halo, oxo, C₁₋₆ alkyl, C₃₋₁₂ cycloalkyl, hydroxy, and C₁₋₆ alkyl ester;
R⁴ is selected from the group consisting of hydrogen, C₁₋₃ alkyl, and (CH₂)_{z}-cycloalkyl;
R⁵ and R⁶ are each independently selected from hydrogen, a halogen, C₁₋₃ alkyl, and C₃₋₆ cycloalkyl; and
R⁷ is selected from hydrogen, C₁₋₂₁ alkyl and (CR⁵R⁶)_{z}—C₅₋₁₂cycloalkyl, wherein at least one of R¹, R² and R³ is not hydrogen; and z is 0, 1, 2, 3, 4, 5, or 6.

2. A compound of Formula (I): 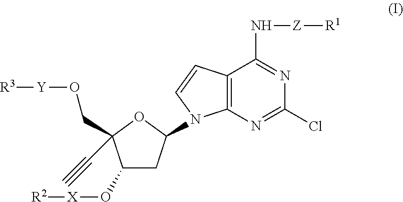
or a pharmaceutically acceptable salt thereof, wherein:
X is selected from —C(═O)—, —C(═O)—O—, —CR⁵R⁶—O—C(═O)—, —CR⁵R⁶—O—C(═O)—O—, and a bond;
Y is selected from —C(═O)—, —C(═O)—O—, —CR⁵R⁶—O—C(═O)—, —CR⁵R⁶—O—C(═O)—O—, —P(═O)(O—C₆₋₁₂ aryl)-NH—CR⁴C(═O)—O—, and a bond;
Z is selected from —C(═O)— and a bond;
R¹ is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy;
R² is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy;
R³ is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy;
R⁴ is selected from the group consisting of hydrogen, C1.3 alkyl and (CH₂)_{z}-cycloalkyl;
R⁵ and R⁶ are each independently selected from hydrogen, a halogen, C₁₋₃ alkyl, and C₃₋₆ cycloalkyl; and
wherein at least one of R¹, R² and R³ is not hydrogen; and z is 0, 1, 2, 3, 4, 5, or 6.

3. The compound of claim 1, wherein the compound is of Formula (II): 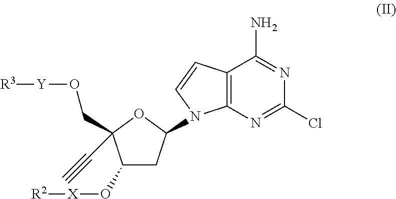
or a pharmaceutically acceptable salt thereof.

4. The compound of claim 1, wherein the compound is of Formula (III): 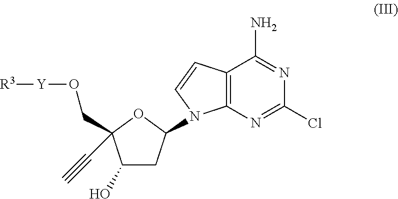
or a pharmaceutically acceptable salt thereof.

5. The compound of claim 1, wherein the compound is of Formula (IV): 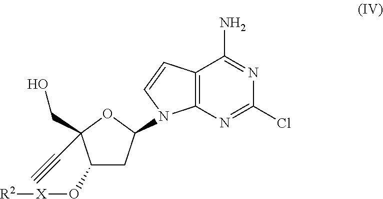
or a pharmaceutically acceptable salt thereof.

6. The compound of claim 1, or a pharmaceutically acceptable salt thereof, wherein
X is selected from —C(═O)—, —CR⁵R⁶—O—C(═O)—, and a bond;
Y is selected from —C(═O)—, —CR⁵R⁶—O—C(═O)—, and a bond;
Z is a bond, and R¹ is hydrogen;
R² is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy; and
R³ is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy,
wherein z is 0, 1, 2, 3, 4, 5, or 6.

7. The compound of claim 1, or a pharmaceutically acceptable salt thereof, wherein
X is selected from —C(═O)—O—, —CR⁵R⁶—O—C(═O)—O—, and a bond;
Y is selected from —C(═O)—O—, —CR⁵R⁶—O—C(═O)—O—, and a bond;
Z is a bond, and R¹ is hydrogen;
R² is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy; and
R³ is selected from the group consisting of hydrogen, C₁₋₂₁ alkyl, (CR⁵R⁶)_{z}—C₃₋₁₂ cycloalkyl, (CR⁵R⁶)_{z}—C₅₋₁₂heterocyclyl, (CR⁵R⁶)_{z}—C₆₋₁₂ aryl, and (CR⁵R⁶)_{z}—C₅₋₁₂ heteroaryl, wherein said alkyl, cycloalkyl, heterocyclyl, aryl or heteroaryl group can be optionally substituted with one to three groups independently selected from halo, C₁₋₆ alkyl, C₃₋₁₂ cycloalkyl and hydroxy,
wherein z is 0, 1, 2, 3, 4, 5, or 6.

8. The compound of claim 1, wherein the compound is of Formula (V): 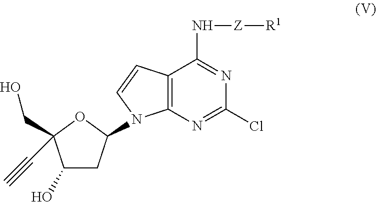
or a pharmaceutically acceptable salt thereof.

9. The compound of claim 1, or a pharmaceutically acceptable salt thereof, wherein:
X is selected from —C(═O)—, —C(═O)—O—, and a bond;
Y is selected from —C(═O)—, —C(═O)—O—, and a bond;
Z is a bond, and R¹ is hydrogen;
R¹ is selected from hydrogen and C₁₋₁₄ alkyl;
R² is selected from C₁₋₁₀ alkyl, (CR⁵R⁶)_{z}—C₃₋₁₂cycloalkyl, and (CR⁵R⁶)_{z}—C₆ aryl, wherein said aryl can be optionally substituted with a halo; and
R³ is selected from C₁₋₁₀ alkyl, (CR⁵R⁶)_{z}—C₃₋₁₂cycloalkyl, and (CR⁵R⁶)_{z}—C₆ aryl, wherein said aryl can be optionally substituted with a halo, and
wherein R⁵ and R⁶ are hydrogen,
wherein z is 0, 1, 2, 3, 4, 5, or 6.

10. The compound of claim 1 having the structure: 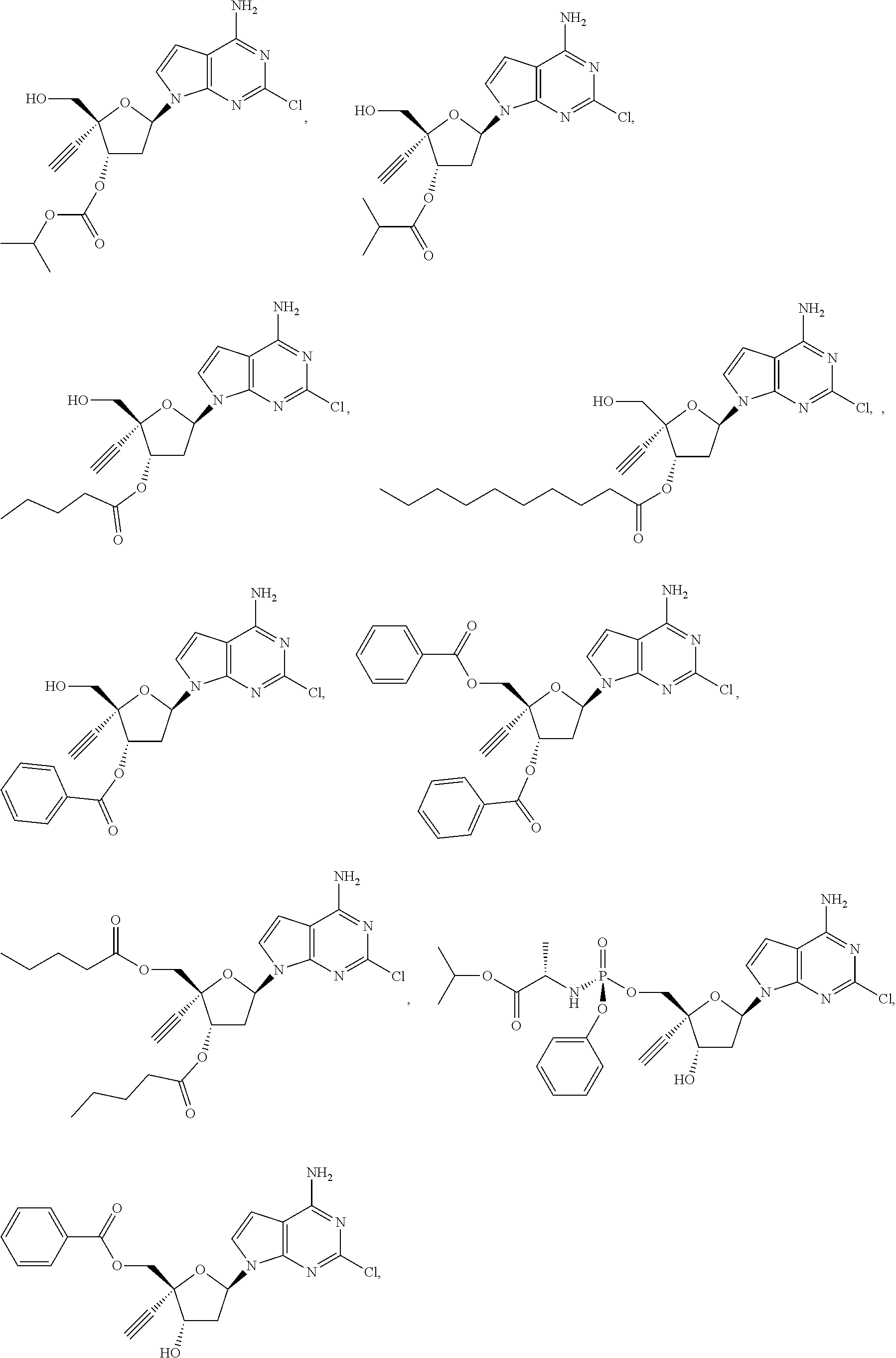 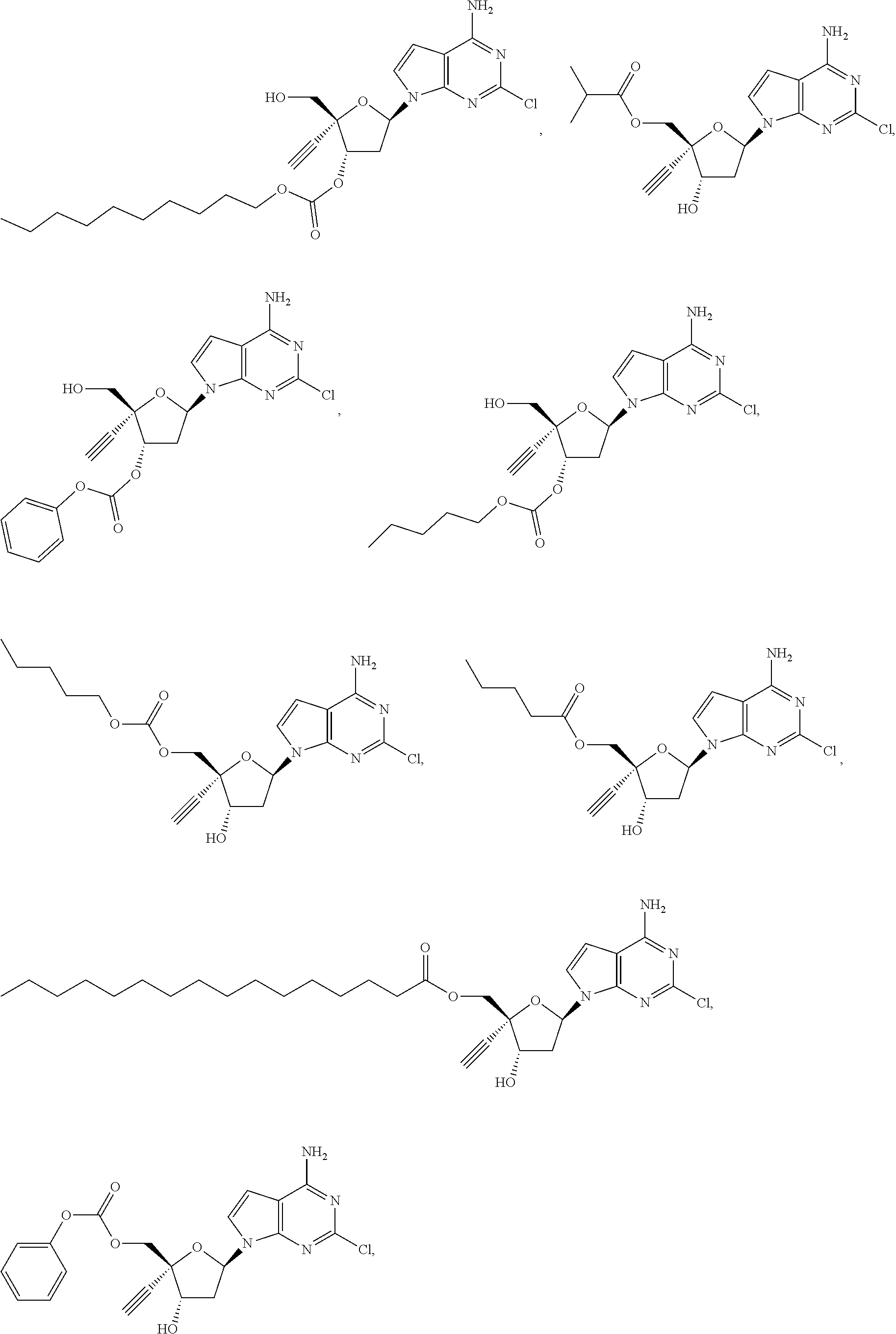 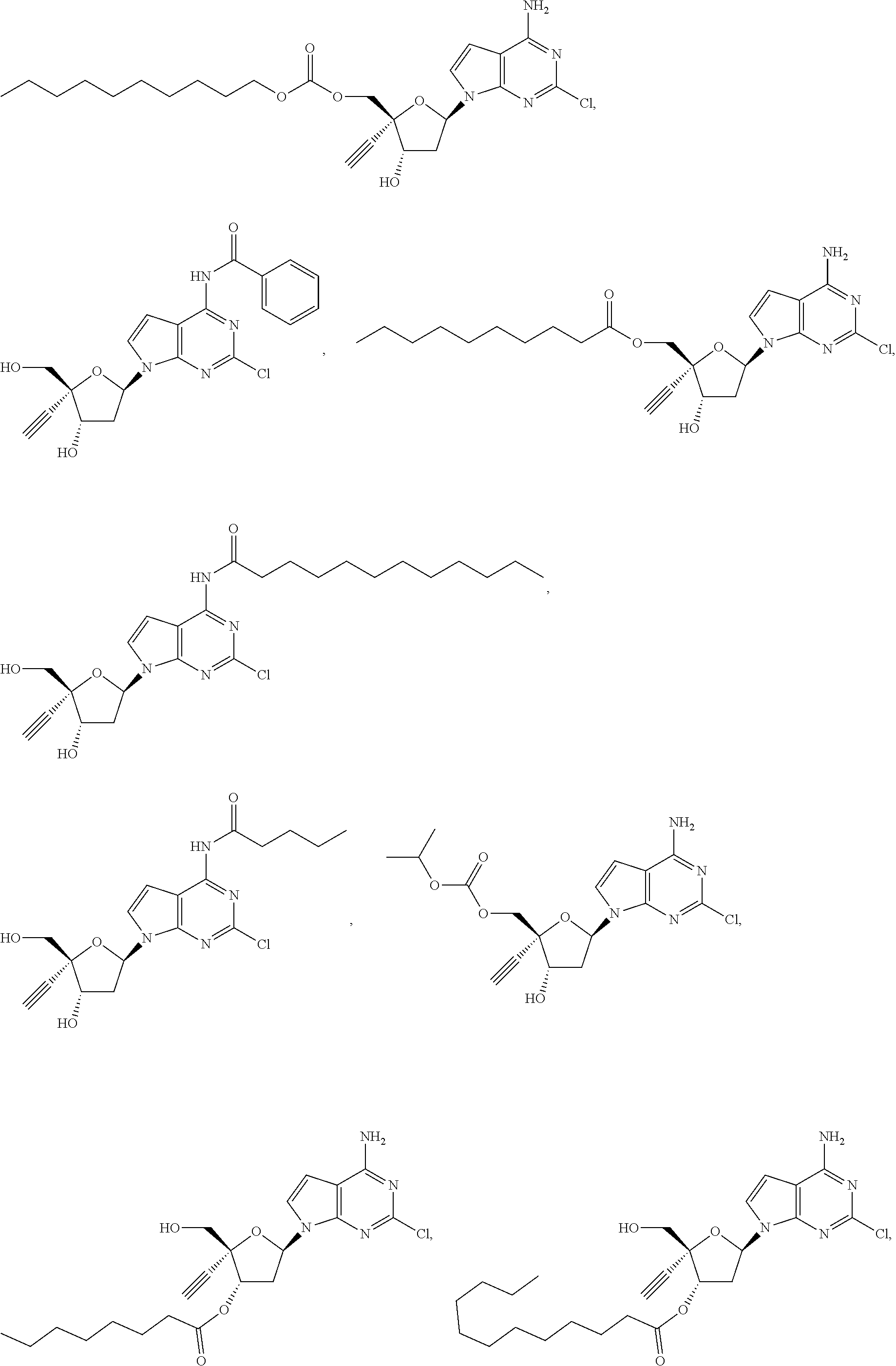 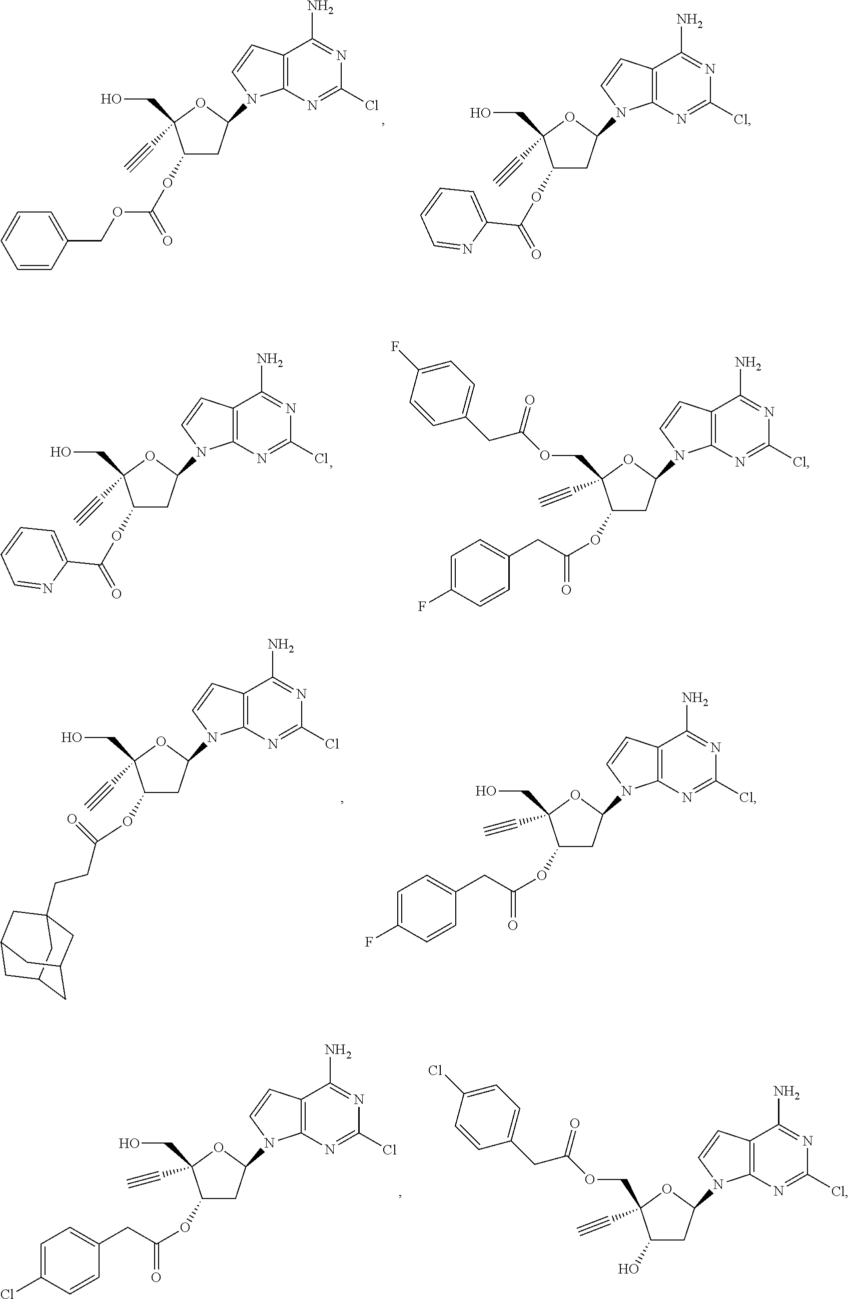 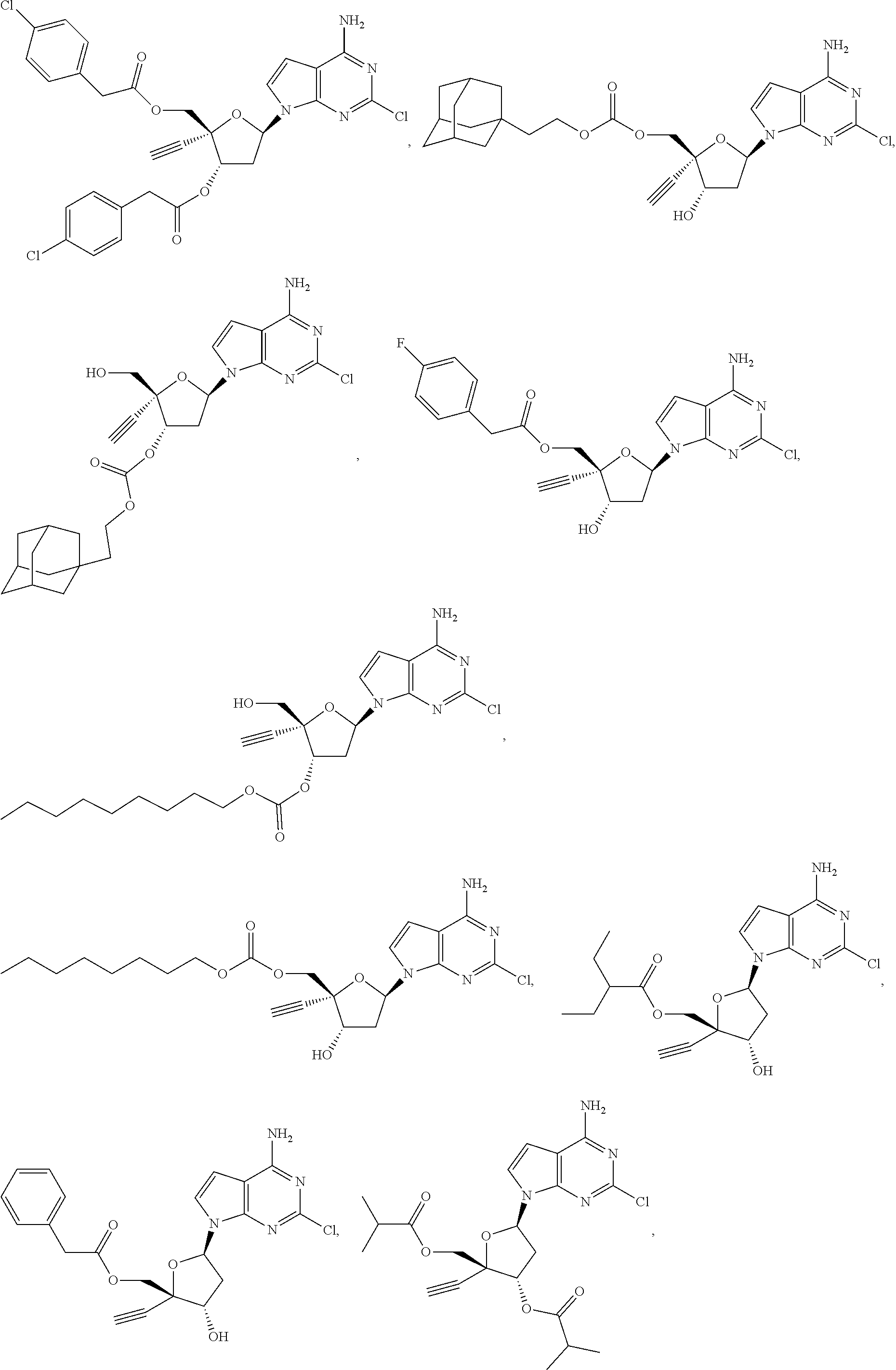 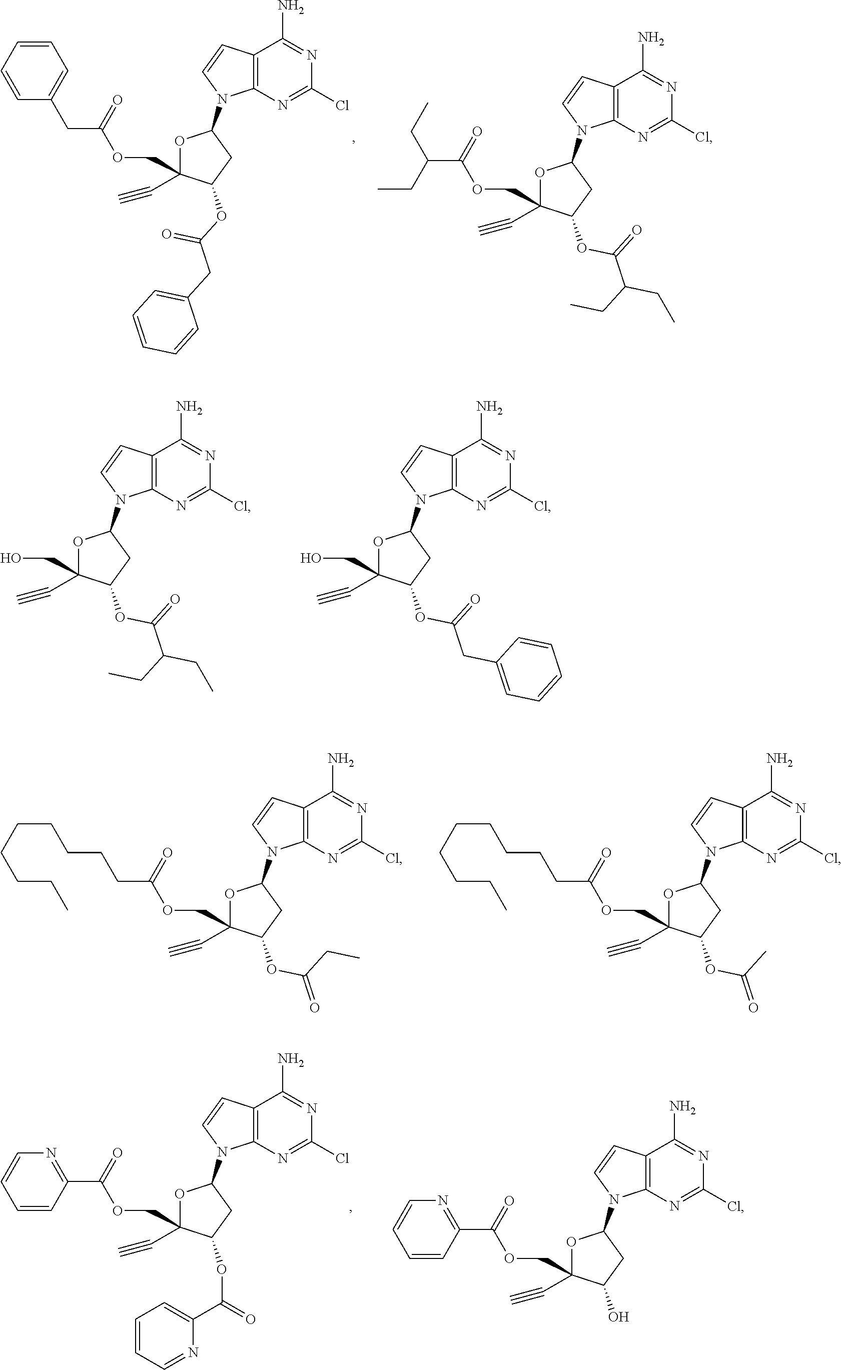 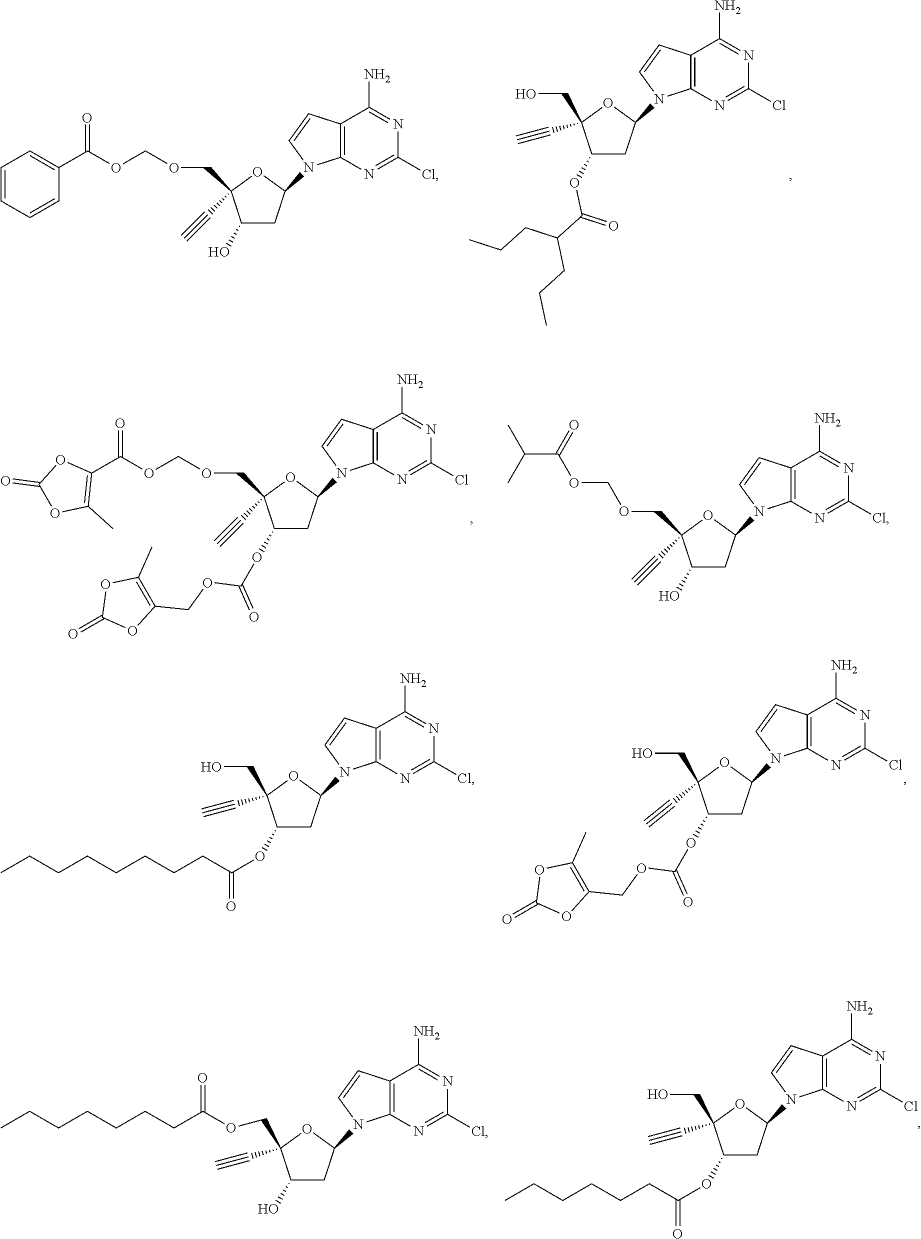 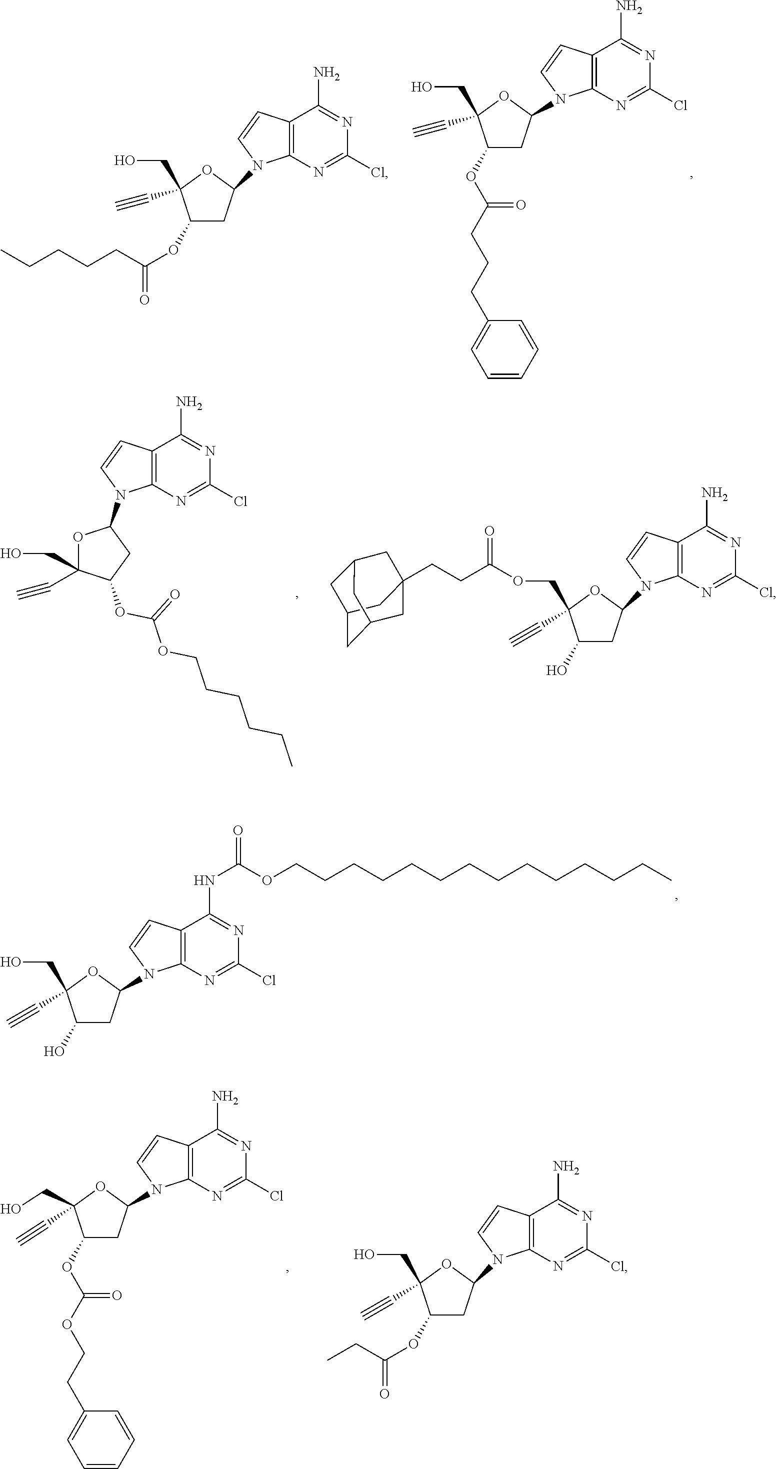 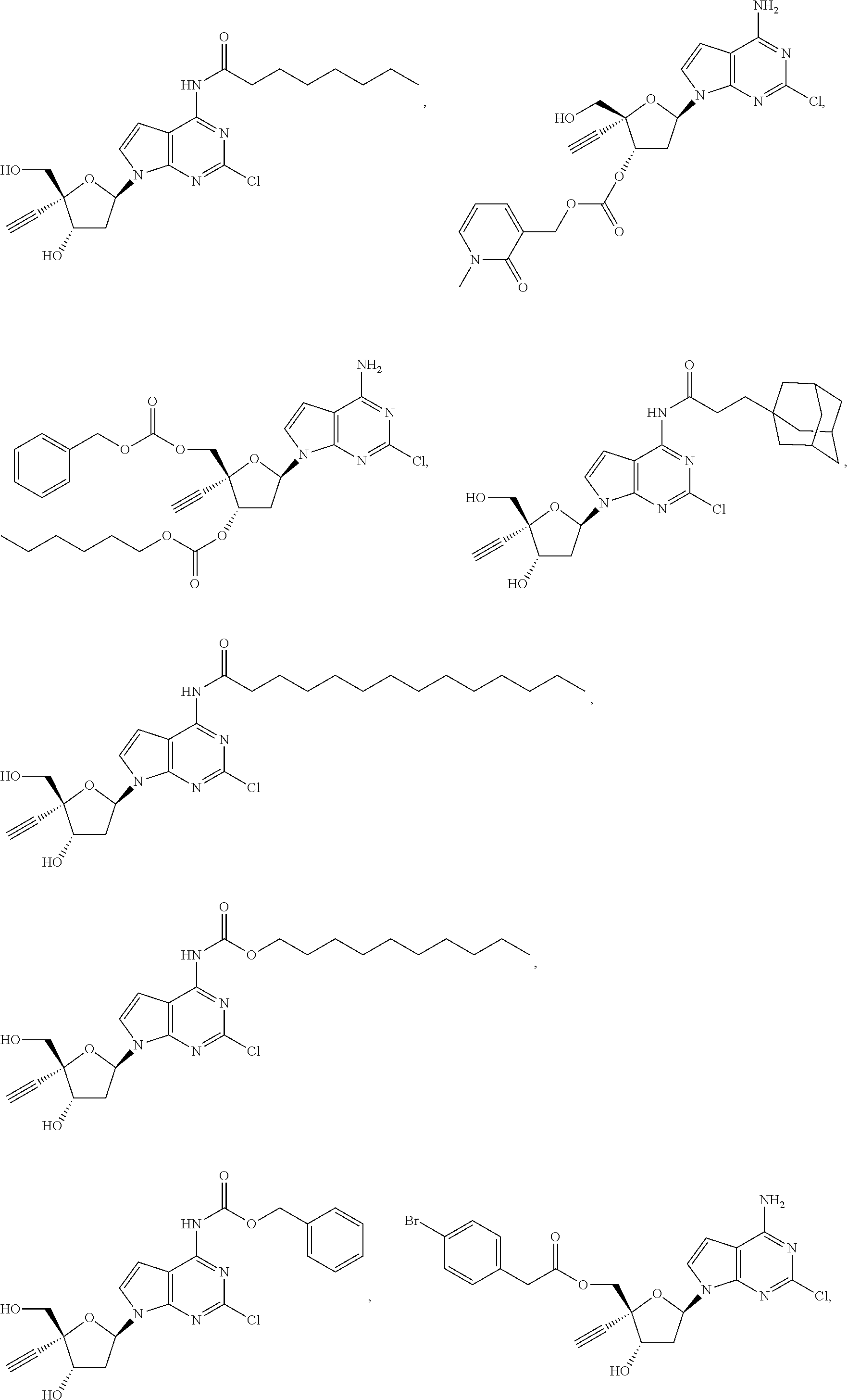 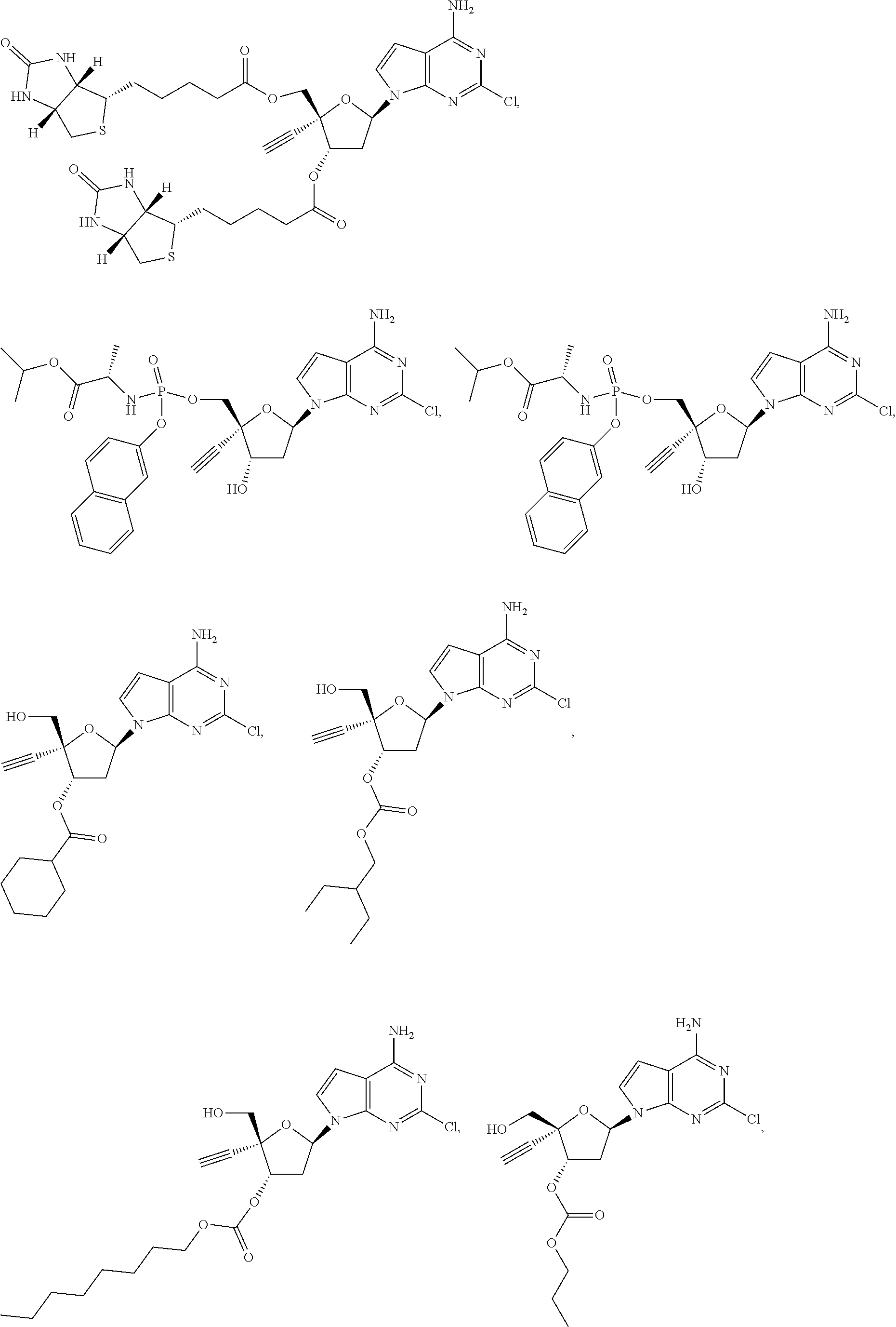 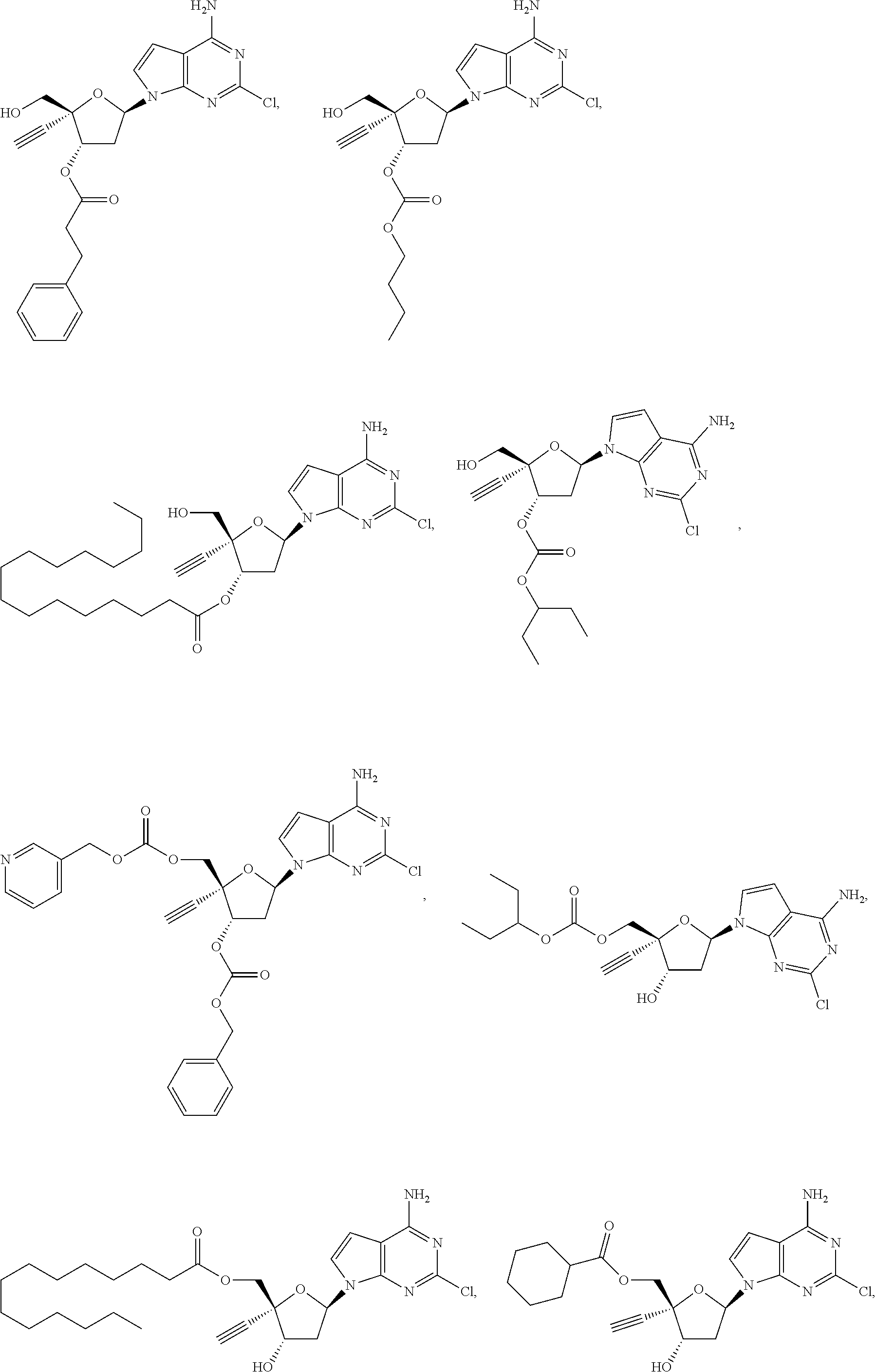 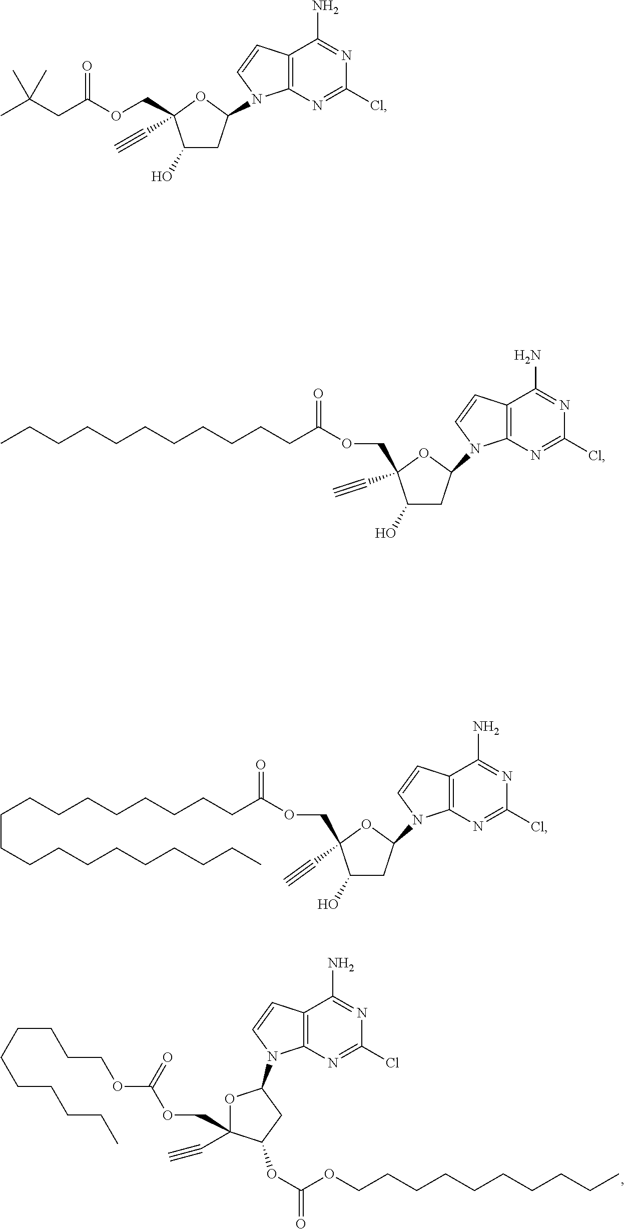 [SMILES: C#C[C@]1(COC(=O)CC(C)(C)C)O[C@@H](n2ccc3c(N)nc(Cl)nc32)C[C@@H]1O ; C#C[C@]1(COC(=O)CCCCCCCCCCC)O[C@@H](n2ccc3c(N)nc(Cl)nc32)C[C@@H]1O ; C#C[C@]1(COC(=O)CCCCCCCCCCCCCCCCCCC)O[C@@H](n2ccc3c(N)nc(Cl)nc32)C[C@@H]1O ; C#C[C@]1(COC(=O)OCCCCCCCCCC)O[C@@H](n2ccc3c(N)nc(Cl)nc32)C[C@@H]1OC(=O)OCCCCCCCCCC] 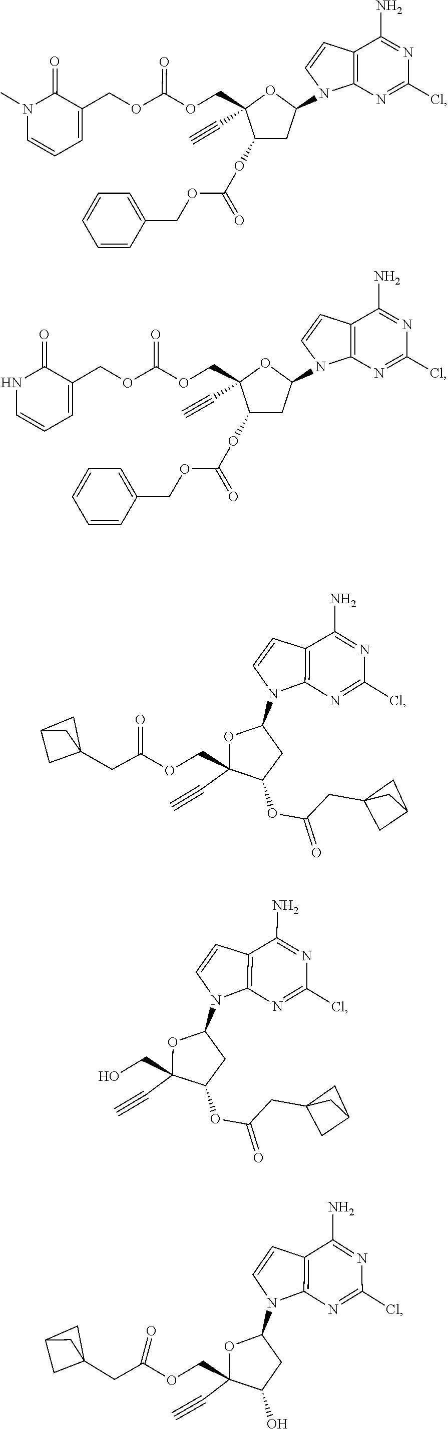 [SMILES: C#C[C@]1(COC(=O)OCc2cccn(C)c2=O)O[C@@H](n2ccc3c(N)nc(Cl)nc32)C[C@@H]1OC(=O)OCc1ccccc1 ; C#C[C@]1(COC(=O)OCc2ccc[nH]c2=O)O[C@@H](n2ccc3c(N)nc(Cl)nc32)C[C@@H]1OC(=O)OCc1ccccc1 ; C#C[C@]1(COC(=O)CC23CC(C2)C3)O[C@@H](n2ccc3c(N)nc(Cl)nc32)C[C@@H]1OC(=O)CC12CC(C1)C2 ; C#C[C@]1(CO)O[C@@H](n2ccc3c(N)nc(Cl)nc32)C[C@@H]1OC(=O)CC12CC(C1)C2 ; C#C[C@]1(COC(=O)CC23CC(C2)C3)O[C@@H](n2ccc3c(N)nc(Cl)nc32)C[C@@H]1O] 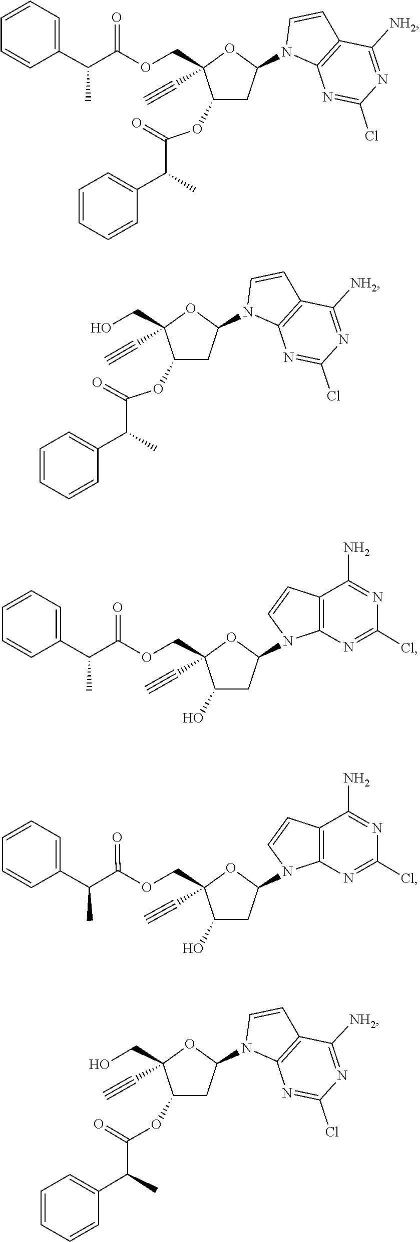 [SMILES: C#C[C@]1(COC(=O)[C@H](C)c2ccccc2)O[C@@H](n2ccc3c(N)nc(Cl)nc32)C[C@@H]1OC(=O)[C@H](C)c1ccccc1 ; C#C[C@]1(CO)O[C@@H](n2ccc3c(N)nc(Cl)nc32)C[C@@H]1OC(=O)[C@H](C)c1ccccc1 ; C#C[C@]1(COC(=O)[C@H](C)c2ccccc2)O[C@@H](n2ccc3c(N)nc(Cl)nc32)C[C@@H]1O ; C#C[C@]1(COC(=O)[C@@H](C)c2ccccc2)O[C@@H](n2ccc3c(N)nc(Cl)nc32)C[C@@H]1O ; C#C[C@]1(CO)O[C@@H](n2ccc3c(N)nc(Cl)nc32)C[C@@H]1OC(=O)[C@@H](C)c1ccccc1] 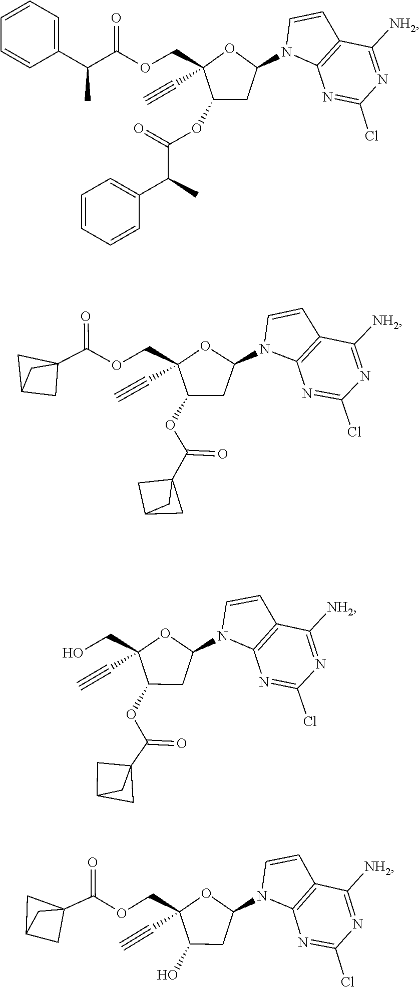 [SMILES: C#C[C@]1(COC(=O)[C@@H](C)c2ccccc2)O[C@@H](n2ccc3c(N)nc(Cl)nc32)C[C@@H]1OC(=O)[C@@H](C)c1ccccc1 ; C#C[C@]1(COC(=O)C23CC(C2)C3)O[C@@H](n2ccc3c(N)nc(Cl)nc32)C[C@@H]1OC(=O)C12CC(C1)C2 ; C#C[C@]1(CO)O[C@@H](n2ccc3c(N)nc(Cl)nc32)C[C@@H]1OC(=O)C12CC(C1)C2 ; C#C[C@]1(COC(=O)C23CC(C2)C3)O[C@@H](n2ccc3c(N)nc(Cl)nc32)C[C@@H]1O] 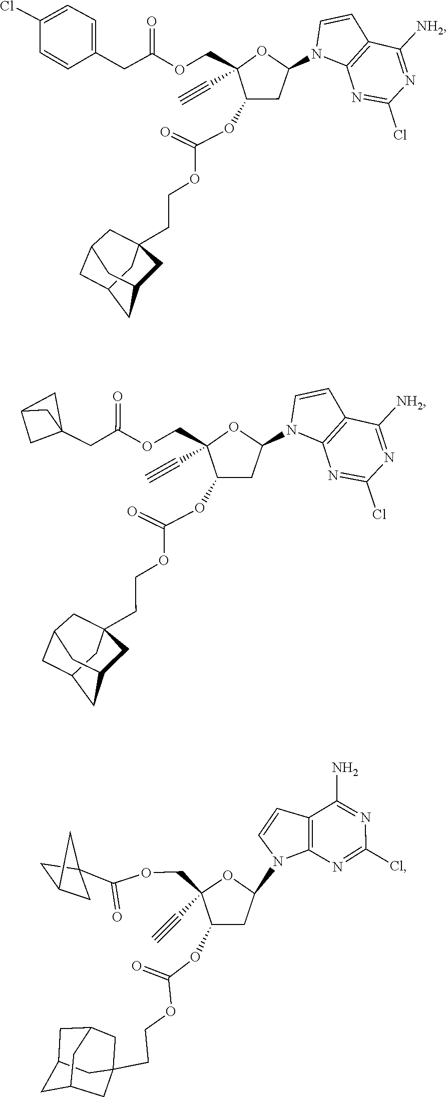 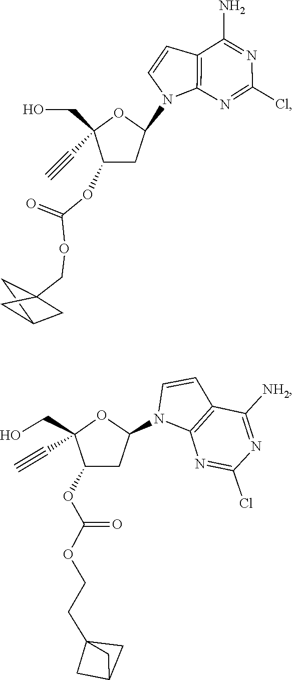 [SMILES: C#C[C@]1(CO)O[C@@H](n2ccc3c(N)nc(Cl)nc32)C[C@@H]1OC(=O)OCC12CC(C1)C2 ; C#C[C@]1(CO)O[C@@H](n2ccc3c(N)nc(Cl)nc32)C[C@@H]1OC(=O)OCCC12CC(C1)C2] 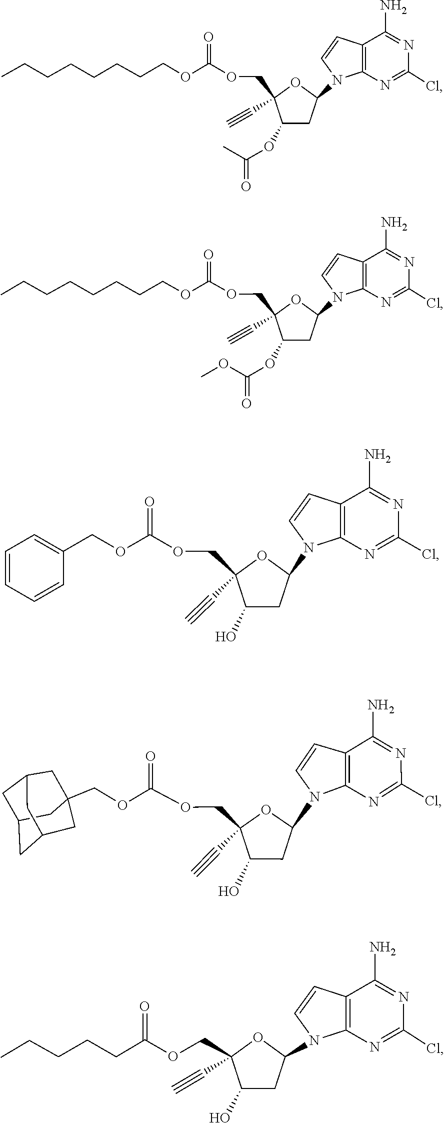 [SMILES: C#C[C@]1(COC(=O)OCCCCCCCC)O[C@@H](n2ccc3c(N)nc(Cl)nc32)C[C@@H]1OC(C)=O ; C#C[C@]1(COC(=O)OCCCCCCCC)O[C@@H](n2ccc3c(N)nc(Cl)nc32)C[C@@H]1OC(=O)OC ; C#C[C@]1(COC(=O)OCc2ccccc2)O[C@@H](n2ccc3c(N)nc(Cl)nc32)C[C@@H]1O ; C#C[C@]1(COC(=O)OCC23CC4CC(CC(C4)C2)C3)O[C@@H](n2ccc3c(N)nc(Cl)nc32)C[C@@H]1O ; C#C[C@]1(COC(=O)CCCCC)O[C@@H](n2ccc3c(N)nc(Cl)nc32)C[C@@H]1O] 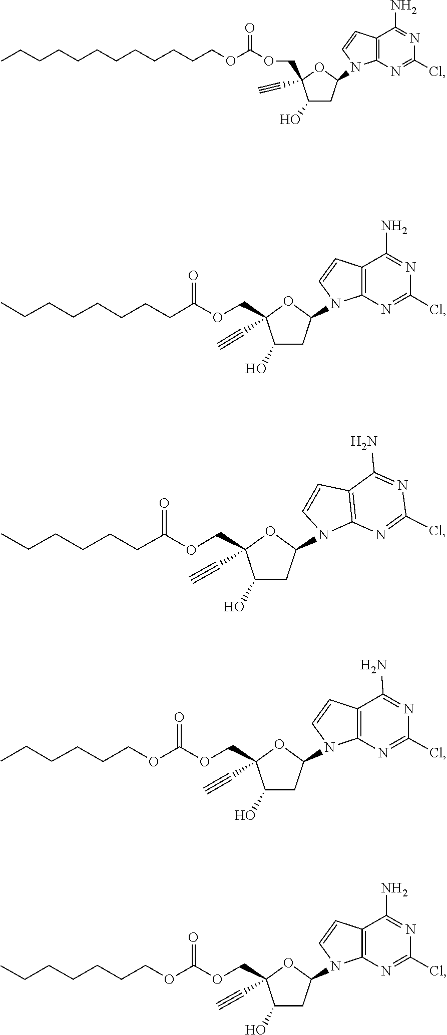 [SMILES: C#C[C@]1(COC(=O)OCCCCCCCCCCCC)O[C@@H](n2ccc3c(N)nc(Cl)nc32)C[C@@H]1O ; C#C[C@]1(COC(=O)CCCCCCCC)O[C@@H](n2ccc3c(N)nc(Cl)nc32)C[C@@H]1O ; C#C[C@]1(COC(=O)CCCCCC)O[C@@H](n2ccc3c(N)nc(Cl)nc32)C[C@@H]1O ; C#C[C@]1(COC(=O)OCCCCCC)O[C@@H](n2ccc3c(N)nc(Cl)nc32)C[C@@H]1O ; C#C[C@]1(COC(=O)OCCCCCCC)O[C@@H](n2ccc3c(N)nc(Cl)nc32)C[C@@H]1O] 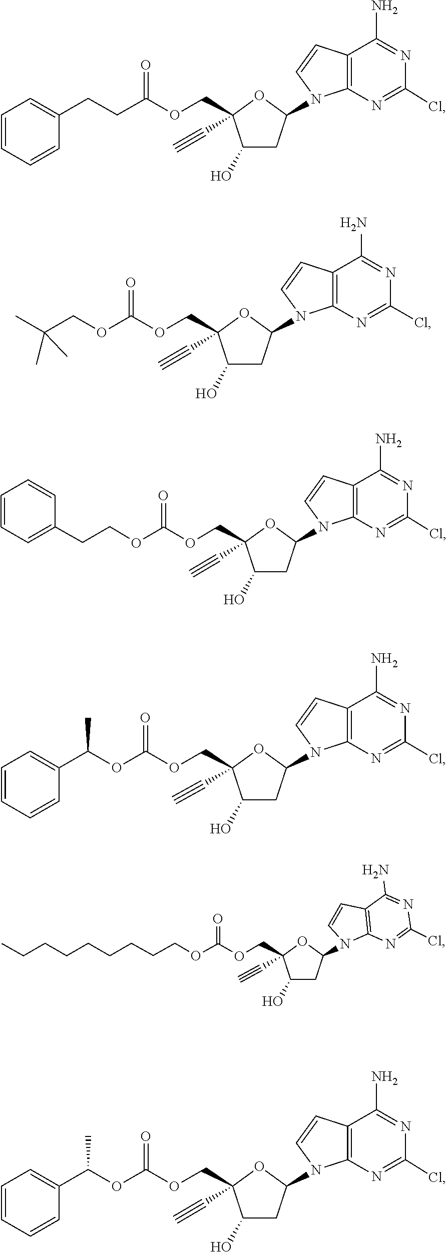 [SMILES: C#C[C@]1(COC(=O)CCc2ccccc2)O[C@@H](n2ccc3c(N)nc(Cl)nc32)C[C@@H]1O ; C#C[C@]1(COC(=O)OCC(C)(C)C)O[C@@H](n2ccc3c(N)nc(Cl)nc32)C[C@@H]1O ; C#C[C@]1(COC(=O)OCCc2ccccc2)O[C@@H](n2ccc3c(N)nc(Cl)nc32)C[C@@H]1O ; C#C[C@]1(COC(=O)O[C@H](C)c2ccccc2)O[C@@H](n2ccc3c(N)nc(Cl)nc32)C[C@@H]1O ; C#C[C@]1(COC(=O)OCCCCCCCCC)O[C@@H](n2ccc3c(N)nc(Cl)nc32)C[C@@H]1O ; C#C[C@]1(COC(=O)O[C@@H](C)c2ccccc2)O[C@@H](n2ccc3c(N)nc(Cl)nc32)C[C@@H]1O] 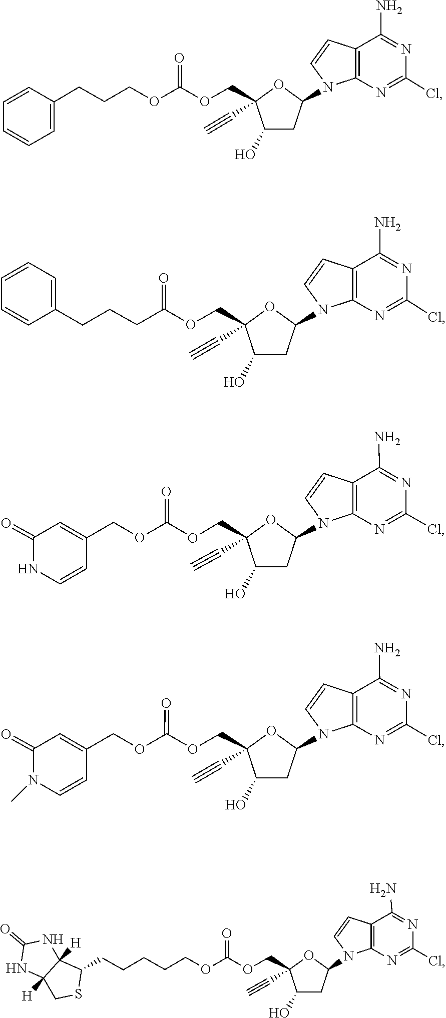 [SMILES: C#C[C@]1(COC(=O)OCCCc2ccccc2)O[C@@H](n2ccc3c(N)nc(Cl)nc32)C[C@@H]1O ; C#C[C@]1(COC(=O)CCCc2ccccc2)O[C@@H](n2ccc3c(N)nc(Cl)nc32)C[C@@H]1O ; C#C[C@]1(COC(=O)OCc2cc[nH]c(=O)c2)O[C@@H](n2ccc3c(N)nc(Cl)nc32)C[C@@H]1O ; C#C[C@]1(COC(=O)OCc2ccn(C)c(=O)c2)O[C@@H](n2ccc3c(N)nc(Cl)nc32)C[C@@H]1O ; C#C[C@]1(COC(=O)OCCCCC[C@@H]2SC[C@@H]3NC(=O)N[C@@H]32)O[C@@H](n2ccc3c(N)nc(Cl)nc32)C[C@@H]1O] 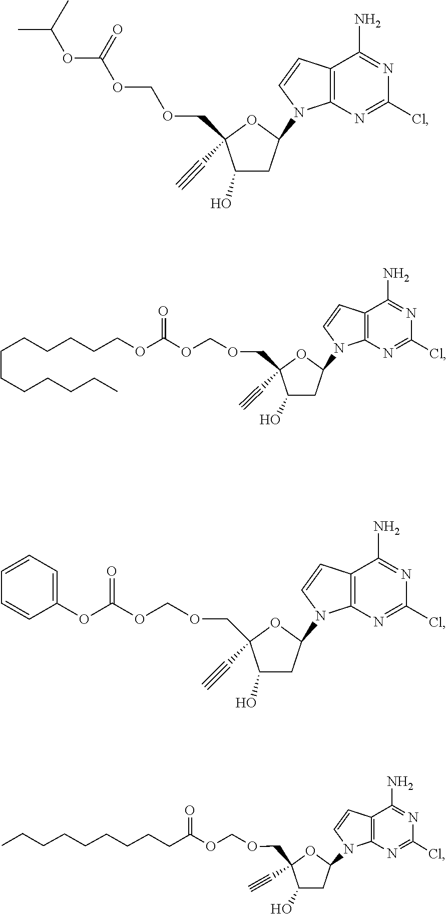 [SMILES: C#C[C@]1(COCOC(=O)OC(C)C)O[C@@H](n2ccc3c(N)nc(Cl)nc32)C[C@@H]1O ; C#C[C@]1(COCOC(=O)OCCCCCCCCCCCC)O[C@@H](n2ccc3c(N)nc(Cl)nc32)C[C@@H]1O ; C#C[C@]1(COCOC(=O)Oc2ccccc2)O[C@@H](n2ccc3c(N)nc(Cl)nc32)C[C@@H]1O ; C#C[C@]1(COCOC(=O)CCCCCCCCC)O[C@@H](n2ccc3c(N)nc(Cl)nc32)C[C@@H]1O] 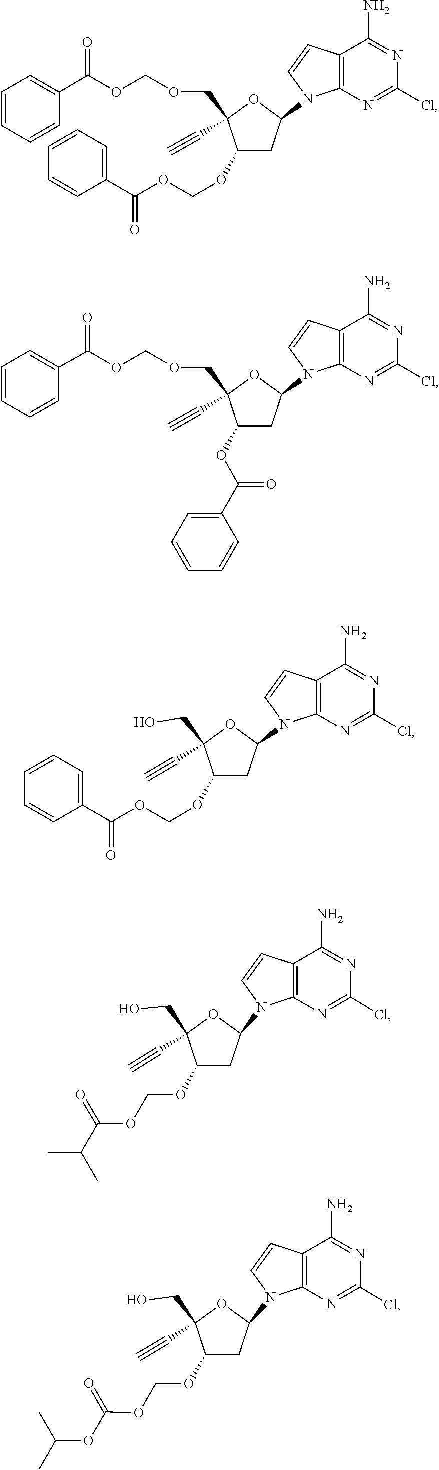 [SMILES: C#C[C@]1(COCOC(=O)c2ccccc2)O[C@@H](n2ccc3c(N)nc(Cl)nc32)C[C@@H]1OCOC(=O)c1ccccc1 ; C#C[C@]1(COCOC(=O)c2ccccc2)O[C@@H](n2ccc3c(N)nc(Cl)nc32)C[C@@H]1OC(=O)c1ccccc1 ; C#C[C@]1(CO)O[C@@H](n2ccc3c(N)nc(Cl)nc32)C[C@@H]1OCOC(=O)c1ccccc1 ; C#C[C@]1(CO)O[C@@H](n2ccc3c(N)nc(Cl)nc32)C[C@@H]1OCOC(=O)C(C)C ; C#C[C@]1(CO)O[C@@H](n2ccc3c(N)nc(Cl)nc32)C[C@@H]1OCOC(=O)OC(C)C] 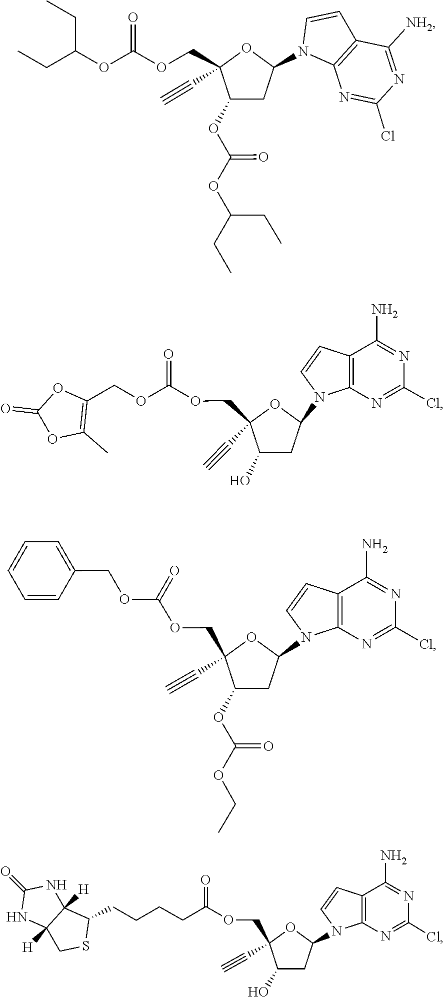 [SMILES: C#C[C@]1(COC(=O)OC(CC)CC)O[C@@H](n2ccc3c(N)nc(Cl)nc32)C[C@@H]1OC(=O)OC(CC)CC ; C#C[C@]1(COC(=O)OCc2oc(=O)oc2C)O[C@@H](n2ccc3c(N)nc(Cl)nc32)C[C@@H]1O ; C#C[C@]1(COC(=O)OCc2ccccc2)O[C@@H](n2ccc3c(N)nc(Cl)nc32)C[C@@H]1OC(=O)OCC ; C#C[C@]1(COC(=O)CCCC[C@@H]2SC[C@@H]3NC(=O)N[C@@H]32)O[C@@H](n2ccc3c(N)nc(Cl)nc32)C[C@@H]1O] 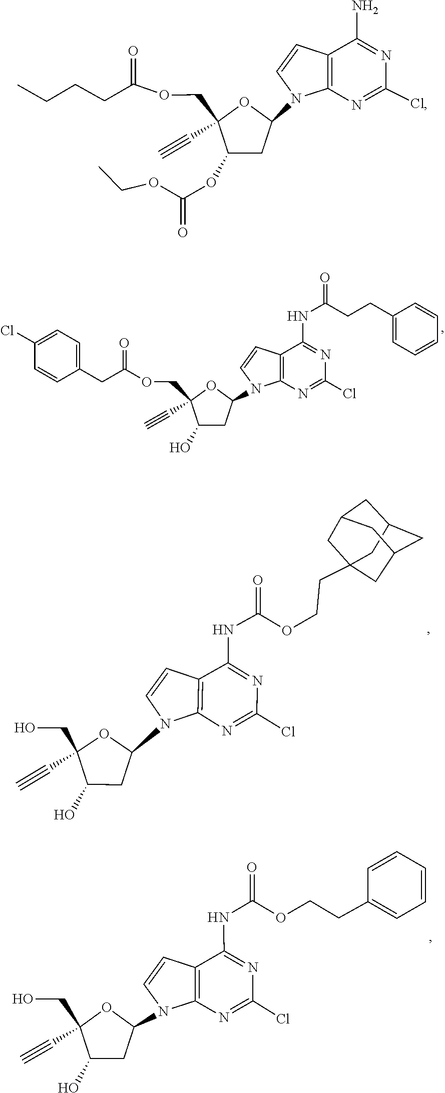 [SMILES: C#C[C@]1(COC(=O)CCCC)O[C@@H](n2ccc3c(N)nc(Cl)nc32)C[C@@H]1OC(=O)OCC ; C#C[C@]1(COC(=O)Cc2ccc(Cl)cc2)O[C@@H](n2ccc3c(NC(=O)CCc4ccccc4)nc(Cl)nc32)C[C@@H]1O ; C#C[C@]1(CO)O[C@@H](n2ccc3c(NC(=O)OCCC45CC6CC(CC(C6)C4)C5)nc(Cl)nc32)C[C@@H]1O ; C#C[C@]1(CO)O[C@@H](n2ccc3c(NC(=O)OCCc4ccccc4)nc(Cl)nc32)C[C@@H]1O] 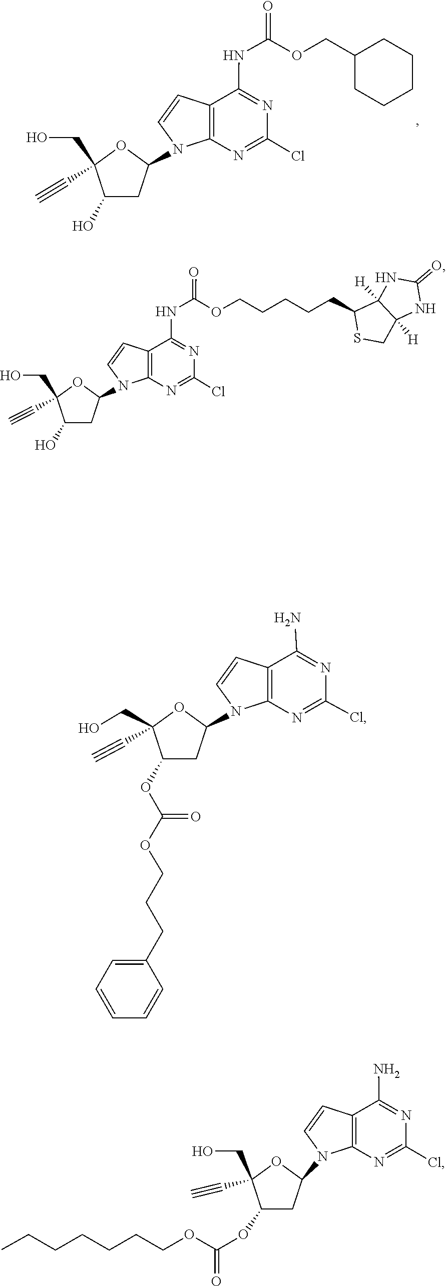 [SMILES: C#C[C@]1(CO)O[C@@H](n2ccc3c(NC(=O)OCC4CCCCC4)nc(Cl)nc32)C[C@@H]1O ; C#C[C@]1(CO)O[C@@H](n2ccc3c(NC(=O)OCCCCC[C@@H]4SC[C@@H]5NC(=O)N[C@@H]54)nc(Cl)nc32)C[C@@H]1O ; C#C[C@]1(CO)O[C@@H](n2ccc3c(N)nc(Cl)nc32)C[C@@H]1OC(=O)OCCCc1ccccc1 ; C#C[C@]1(CO)O[C@@H](n2ccc3c(N)nc(Cl)nc32)C[C@@H]1OC(=O)OCCCCCCC] 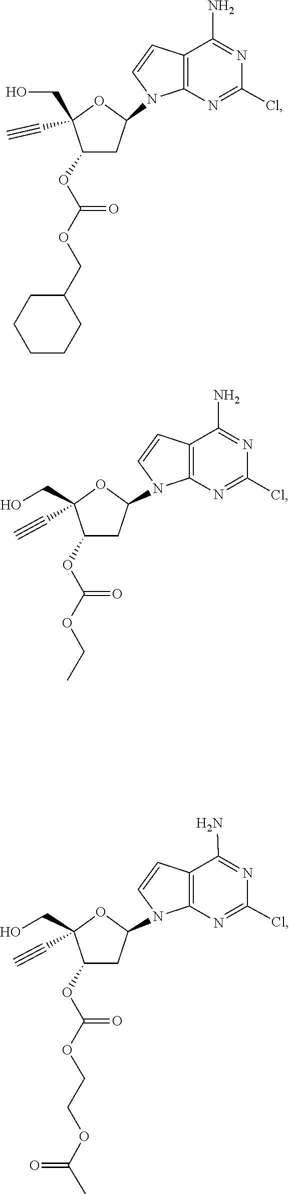 [SMILES: C#C[C@]1(CO)O[C@@H](n2ccc3c(N)nc(Cl)nc32)C[C@@H]1OC(=O)OCC1CCCCC1 ; C#C[C@]1(CO)O[C@@H](n2ccc3c(N)nc(Cl)nc32)C[C@@H]1OC(=O)OCC ; C#C[C@]1(CO)O[C@@H](n2ccc3c(N)nc(Cl)nc32)C[C@@H]1OC(=O)OCCOC(C)=O] 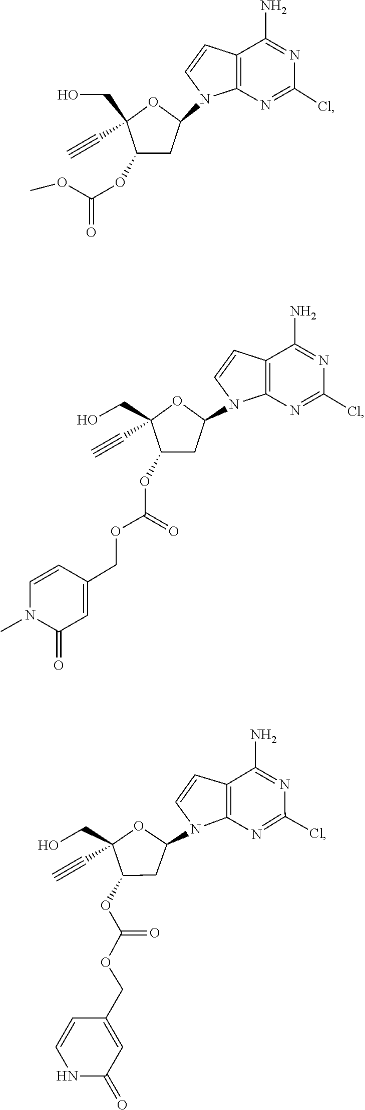 [SMILES: C#C[C@]1(CO)O[C@@H](n2ccc3c(N)nc(Cl)nc32)C[C@@H]1OC(=O)OC ; C#C[C@]1(CO)O[C@@H](n2ccc3c(N)nc(Cl)nc32)C[C@@H]1OC(=O)OCc1ccn(C)c(=O)c1 ; C#C[C@]1(CO)O[C@@H](n2ccc3c(N)nc(Cl)nc32)C[C@@H]1OC(=O)OCc1cc[nH]c(=O)c1] 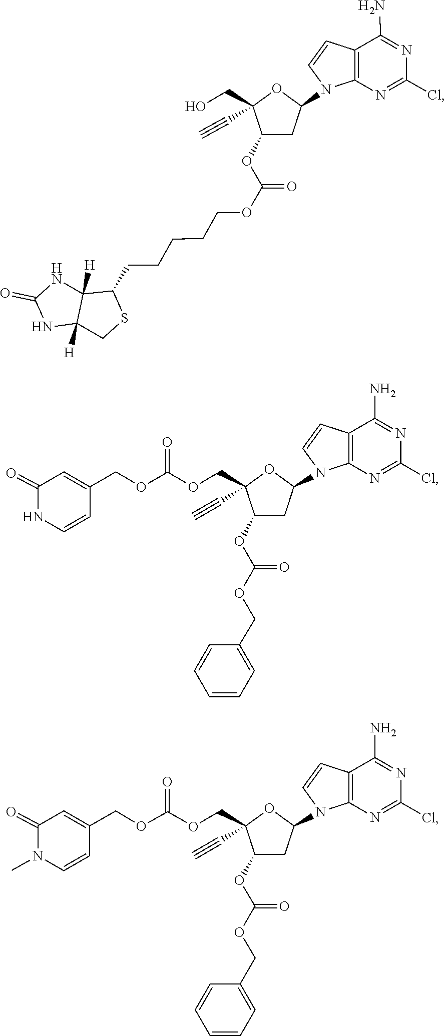 [SMILES: C#C[C@]1(CO)O[C@@H](n2ccc3c(N)nc(Cl)nc32)C[C@@H]1OC(=O)OCCCCC[C@@H]1SC[C@@H]2NC(=O)N[C@@H]21 ; C#C[C@]1(COC(=O)OCc2cc[nH]c(=O)c2)O[C@@H](n2ccc3c(N)nc(Cl)nc32)C[C@@H]1OC(=O)OCc1ccccc1 ; C#C[C@]1(COC(=O)OCc2ccn(C)c(=O)c2)O[C@@H](n2ccc3c(N)nc(Cl)nc32)C[C@@H]1OC(=O)OCc1ccccc1] 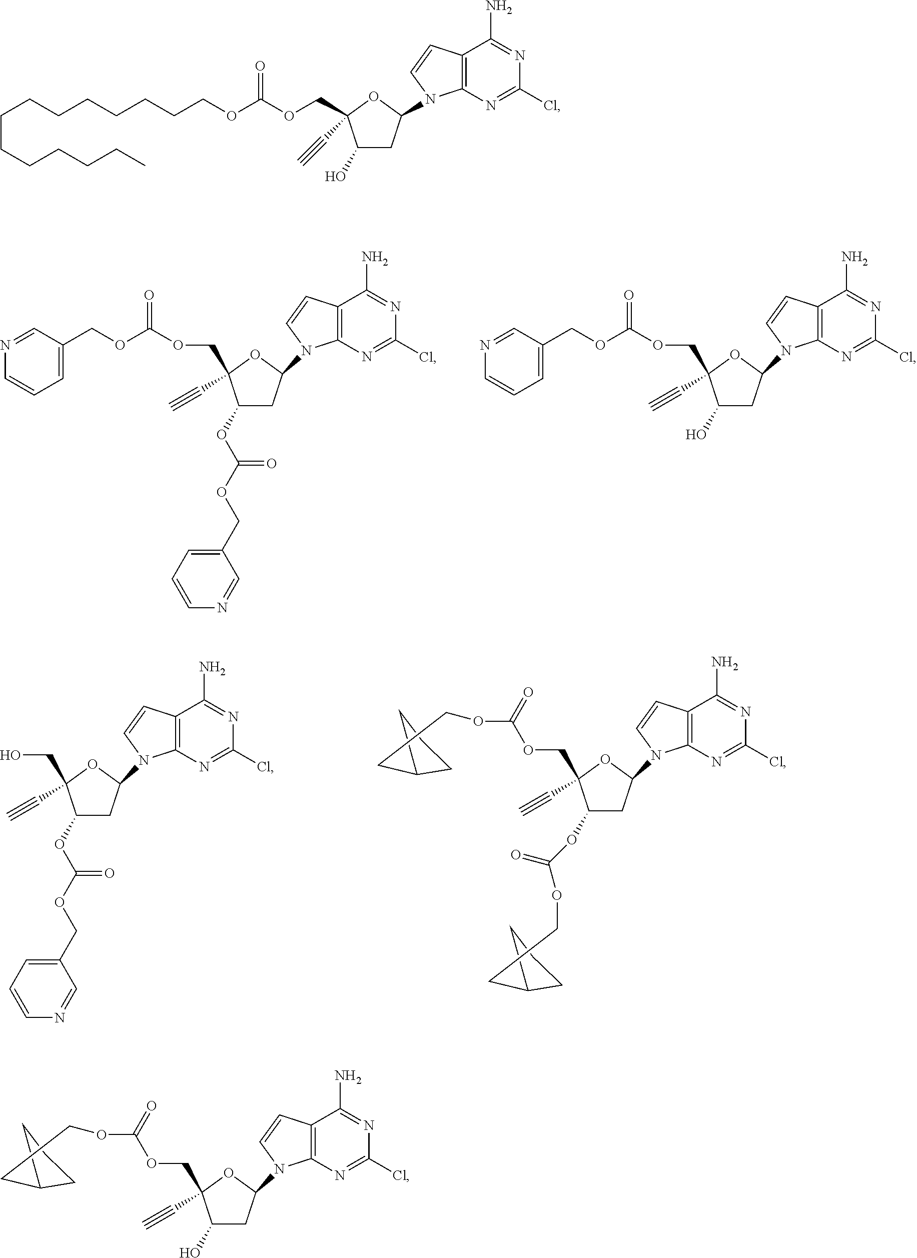 [SMILES: C#C[C@]1(COC(=O)OCCCCCCCCCCCCCC)O[C@@H](n2ccc3c(N)nc(Cl)nc32)C[C@@H]1O ; C#C[C@]1(COC(=O)OCc2cccnc2)O[C@@H](n2ccc3c(N)nc(Cl)nc32)C[C@@H]1OC(=O)OCc1cccnc1 ; C#C[C@]1(COC(=O)OCc2cccnc2)O[C@@H](n2ccc3c(N)nc(Cl)nc32)C[C@@H]1O ; C#C[C@]1(CO)O[C@@H](n2ccc3c(N)nc(Cl)nc32)C[C@@H]1OC(=O)OCc1cccnc1 ; C#C[C@]1(COC(=O)OCCC2CCC2)O[C@@H](n2ccc3c(N)nc(Cl)nc32)C[C@@H]1OC(=O)OCCC1CCC1 ; C#C[C@]1(COC(=O)OCCC2CCC2)O[C@@H](n2ccc3c(N)nc(Cl)nc32)C[C@@H]1O] 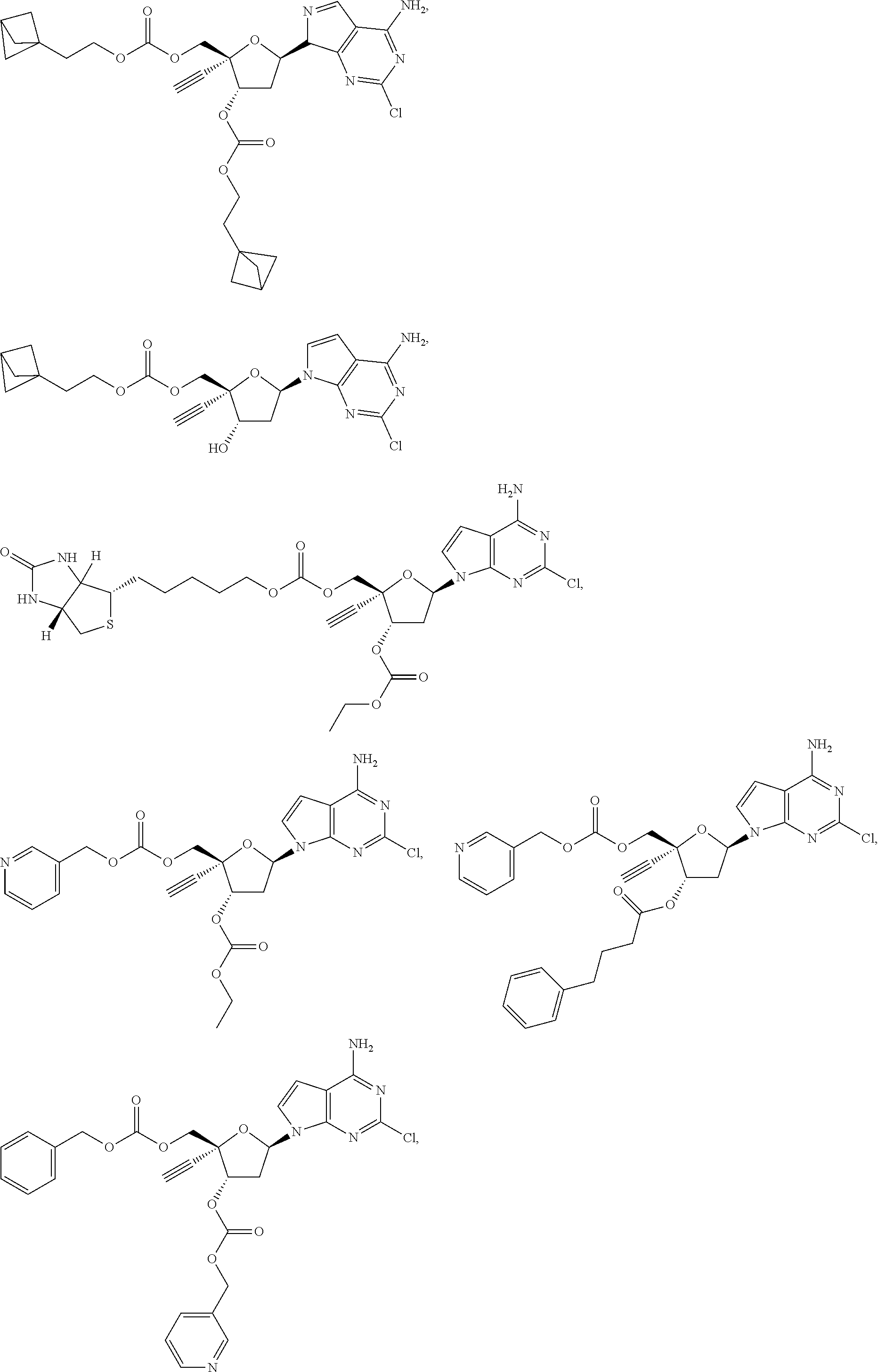 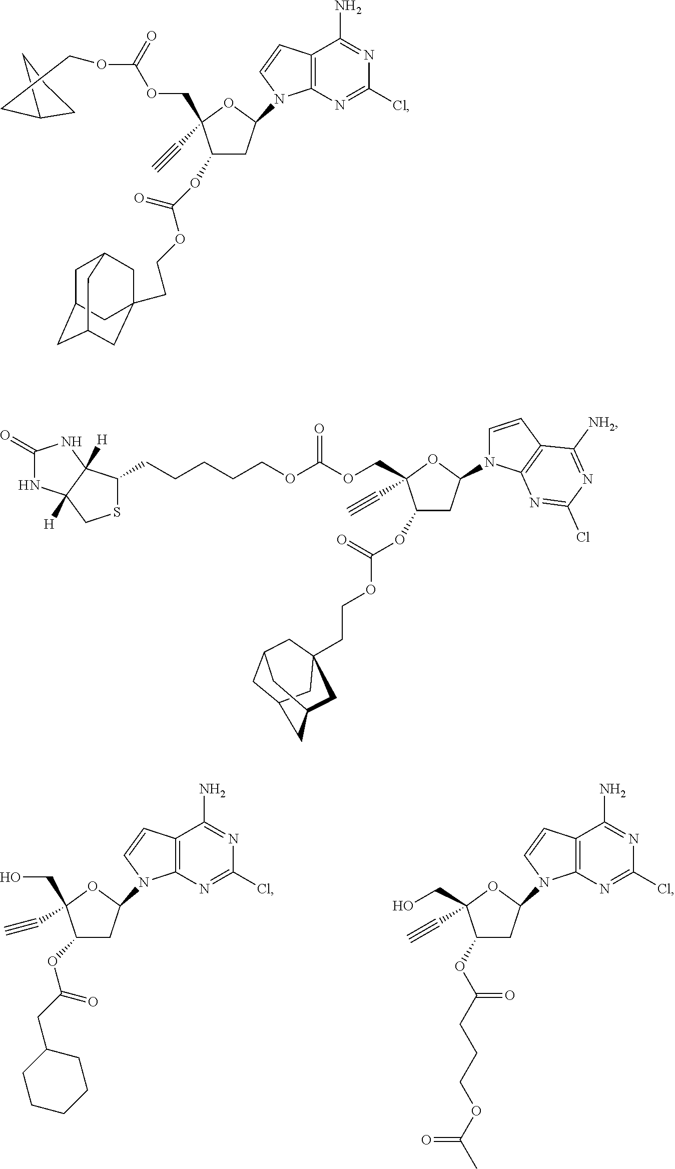 [SMILES: C#C[C@]1(COC(=O)OCCC2CCC2)O[C@@H](n2ccc3c(N)nc(Cl)nc32)C[C@@H]1OC(=O)OCCC12CC3CC(CC(C3)C1)C2 ; C#C[C@]1(COC(=O)OCCCCC[C@@H]2SC[C@@H]3NC(=O)N[C@@H]32)O[C@@H](n2ccc3c(N)nc(Cl)nc32)C[C@@H]1OC(=O)OCC[C@]12CC3CC(C[C@@H](C3)C1)C2 ; C#C[C@]1(CO)O[C@@H](n2ccc3c(N)nc(Cl)nc32)C[C@@H]1OC(=O)CC1CCCCC1 ; C#C[C@]1(CO)O[C@@H](n2ccc3c(N)nc(Cl)nc32)C[C@@H]1OC(=O)CCCOC(C)=O] 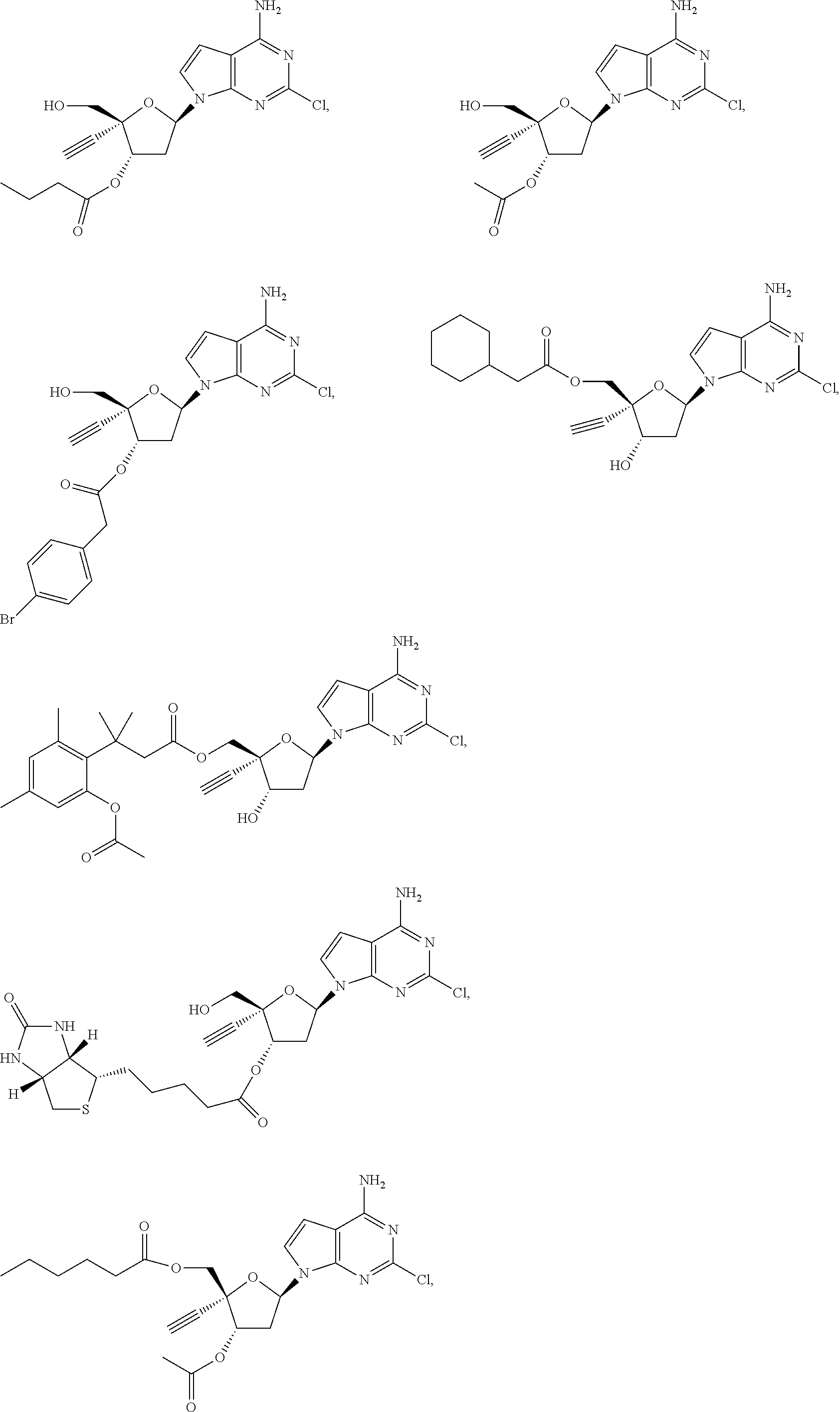 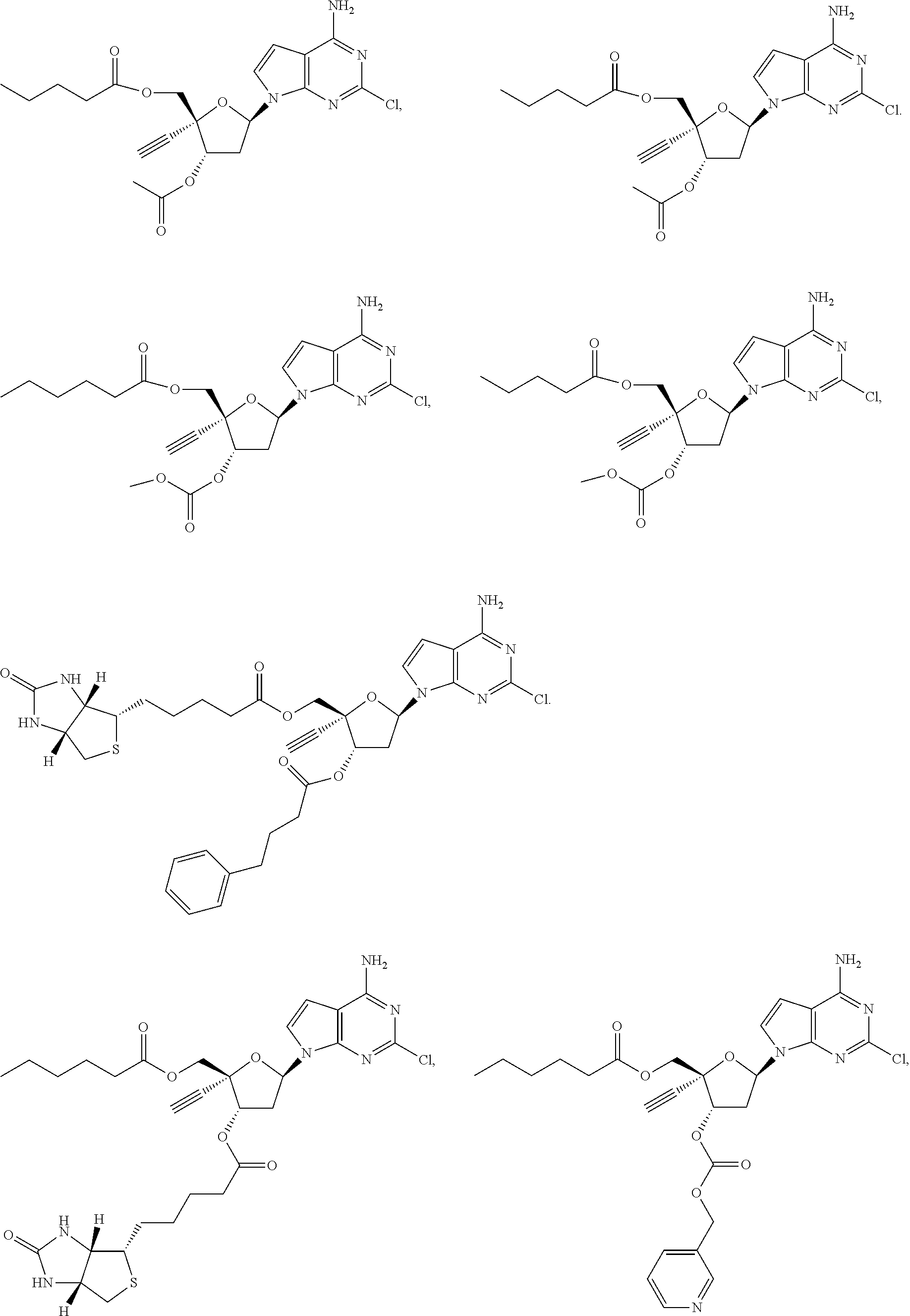 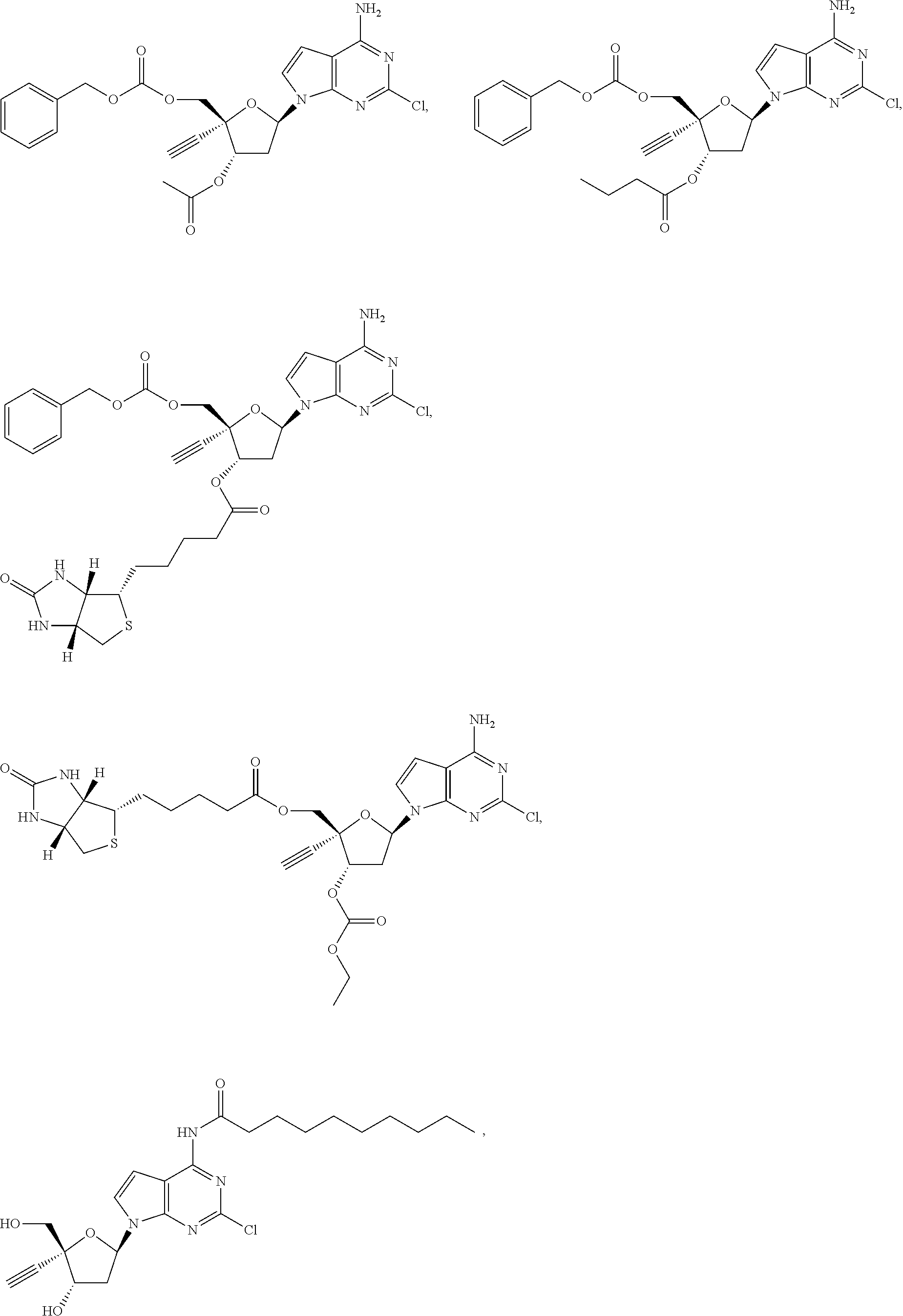 [SMILES: C#C[C@]1(COC(=O)OCc2ccccc2)O[C@@H](n2ccc3c(N)nc(Cl)nc32)C[C@@H]1OC(C)=O ; C#C[C@]1(COC(=O)OCc2ccccc2)O[C@@H](n2ccc3c(N)nc(Cl)nc32)C[C@@H]1OC(=O)CCC ; C#C[C@]1(COC(=O)OCc2ccccc2)O[C@@H](n2ccc3c(N)nc(Cl)nc32)C[C@@H]1OC(=O)CCCC[C@@H]1SC[C@@H]2NC(=O)N[C@@H]21 ; C#C[C@]1(COC(=O)CCCC[C@@H]2SC[C@@H]3NC(=O)N[C@@H]32)O[C@@H](n2ccc3c(N)nc(Cl)nc32)C[C@@H]1OC(=O)OCC ; C#C[C@]1(CO)O[C@@H](n2ccc3c(NC(=O)CCCCCCCCC)nc(Cl)nc32)C[C@@H]1O] 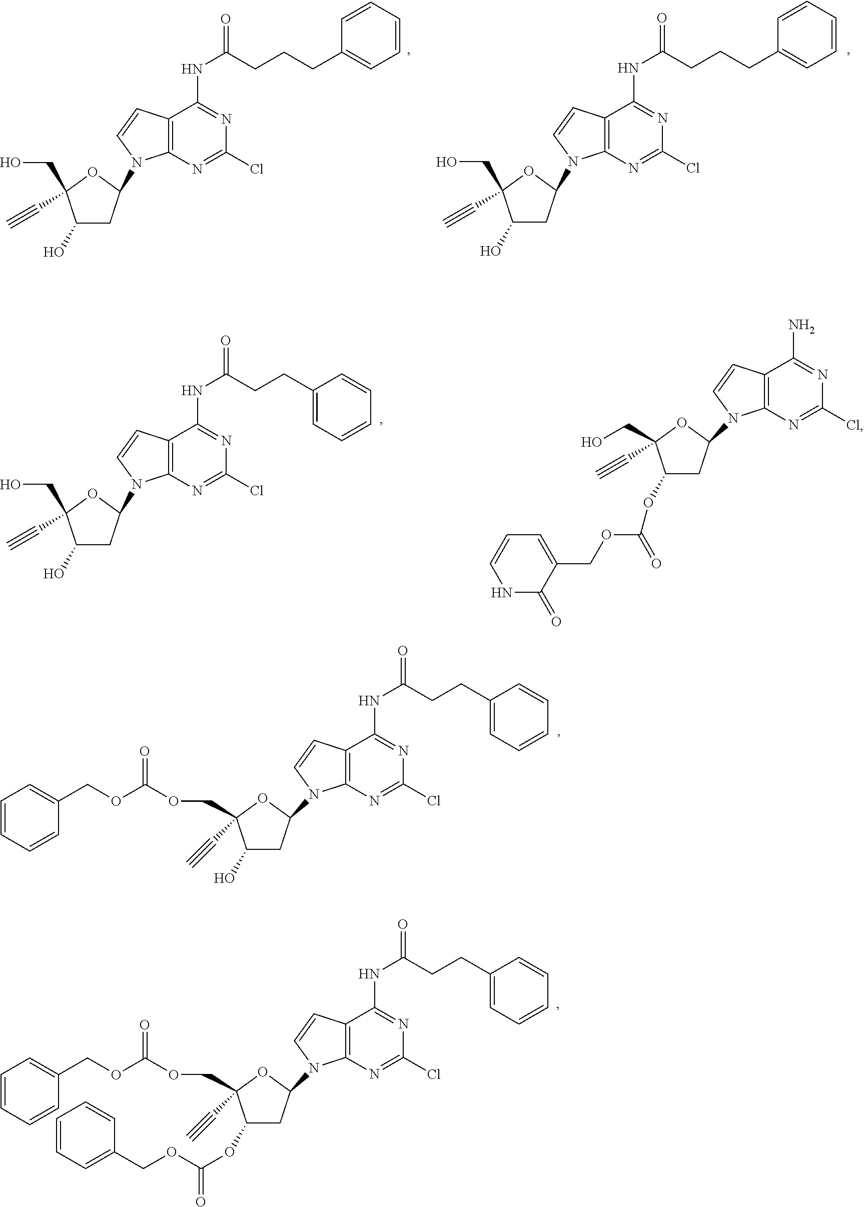 [SMILES: C#C[C@]1(CO)O[C@@H](n2ccc3c(NC(=O)CCCc4ccccc4)nc(Cl)nc32)C[C@@H]1O ; C#C[C@]1(CO)O[C@@H](n2ccc3c(NC(=O)CCCc4ccccc4)nc(Cl)nc32)C[C@@H]1O ; C#C[C@]1(CO)O[C@@H](n2ccc3c(NC(=O)CCc4ccccc4)nc(Cl)nc32)C[C@@H]1O ; C#C[C@]1(CO)O[C@@H](n2ccc3c(N)nc(Cl)nc32)C[C@@H]1OC(=O)OCc1ccc[nH]c1=O ; C#C[C@]1(COC(=O)OCc2ccccc2)O[C@@H](n2ccc3c(NC(=O)CCc4ccccc4)nc(Cl)nc32)C[C@@H]1O ; C#C[C@]1(COC(=O)OCc2ccccc2)O[C@@H](n2ccc3c(NC(=O)CCc4ccccc4)nc(Cl)nc32)C[C@@H]1OC(=O)OCc1ccccc1] 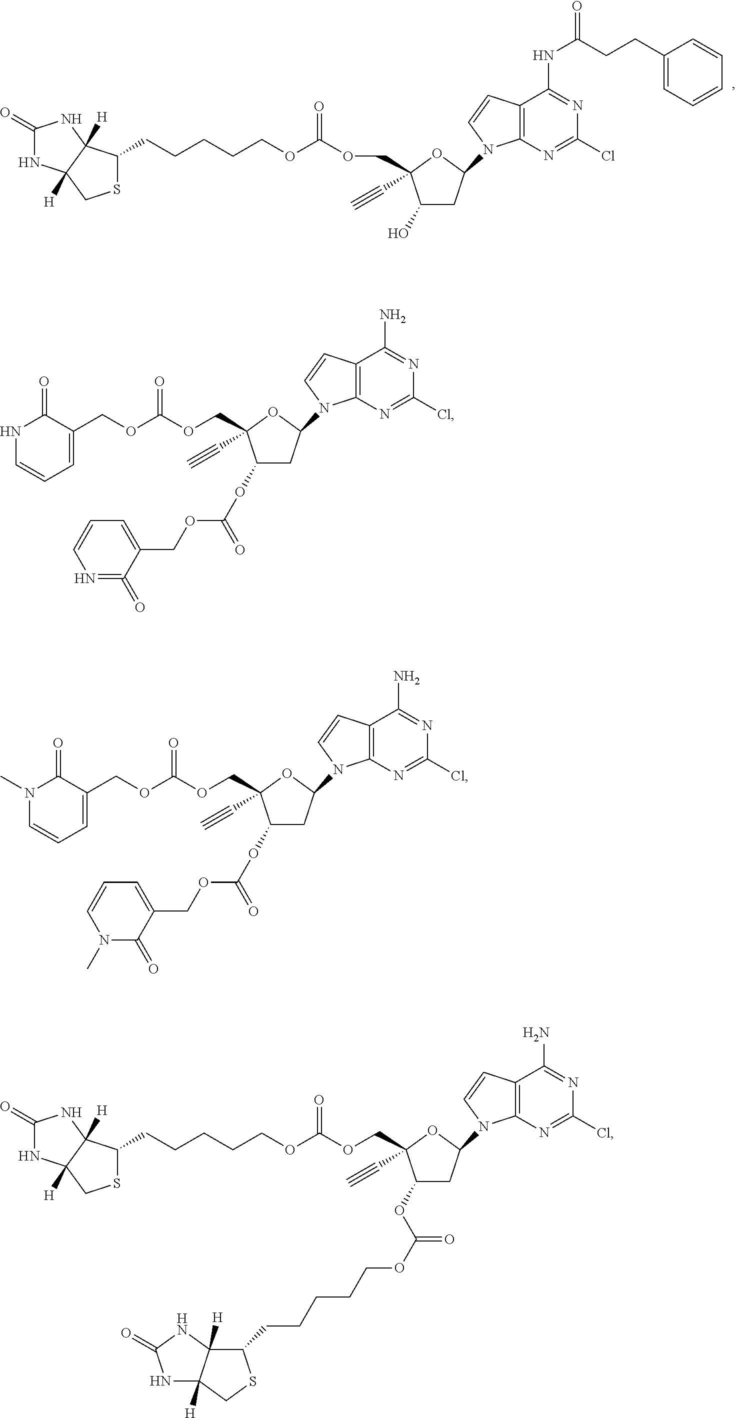 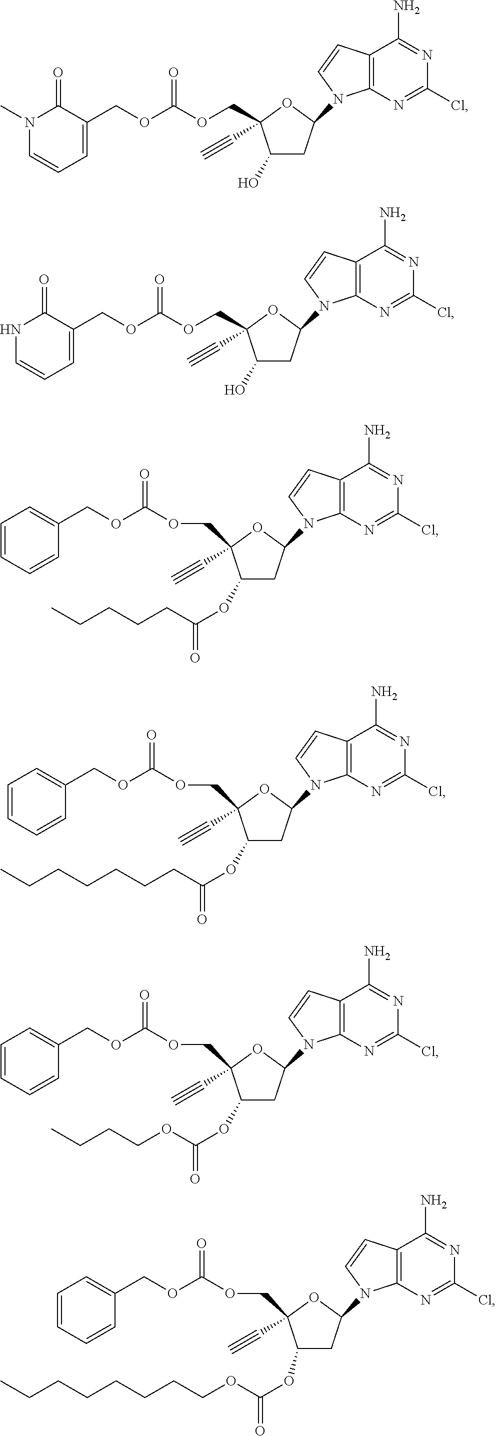 [SMILES: C#C[C@]1(COC(=O)OCc2cccn(C)c2=O)O[C@@H](n2ccc3c(N)nc(Cl)nc32)C[C@@H]1O ; C#C[C@]1(COC(=O)OCc2ccc[nH]c2=O)O[C@@H](n2ccc3c(N)nc(Cl)nc32)C[C@@H]1O ; C#C[C@]1(COC(=O)OCc2ccccc2)O[C@@H](n2ccc3c(N)nc(Cl)nc32)C[C@@H]1OC(=O)CCCCC ; C#C[C@]1(COC(=O)OCc2ccccc2)O[C@@H](n2ccc3c(N)nc(Cl)nc32)C[C@@H]1OC(=O)CCCCCCC ; C#C[C@]1(COC(=O)OCc2ccccc2)O[C@@H](n2ccc3c(N)nc(Cl)nc32)C[C@@H]1OC(=O)OCCCC ; C#C[C@]1(COC(=O)OCc2ccccc2)O[C@@H](n2ccc3c(N)nc(Cl)nc32)C[C@@H]1OC(=O)OCCCCCCCC] 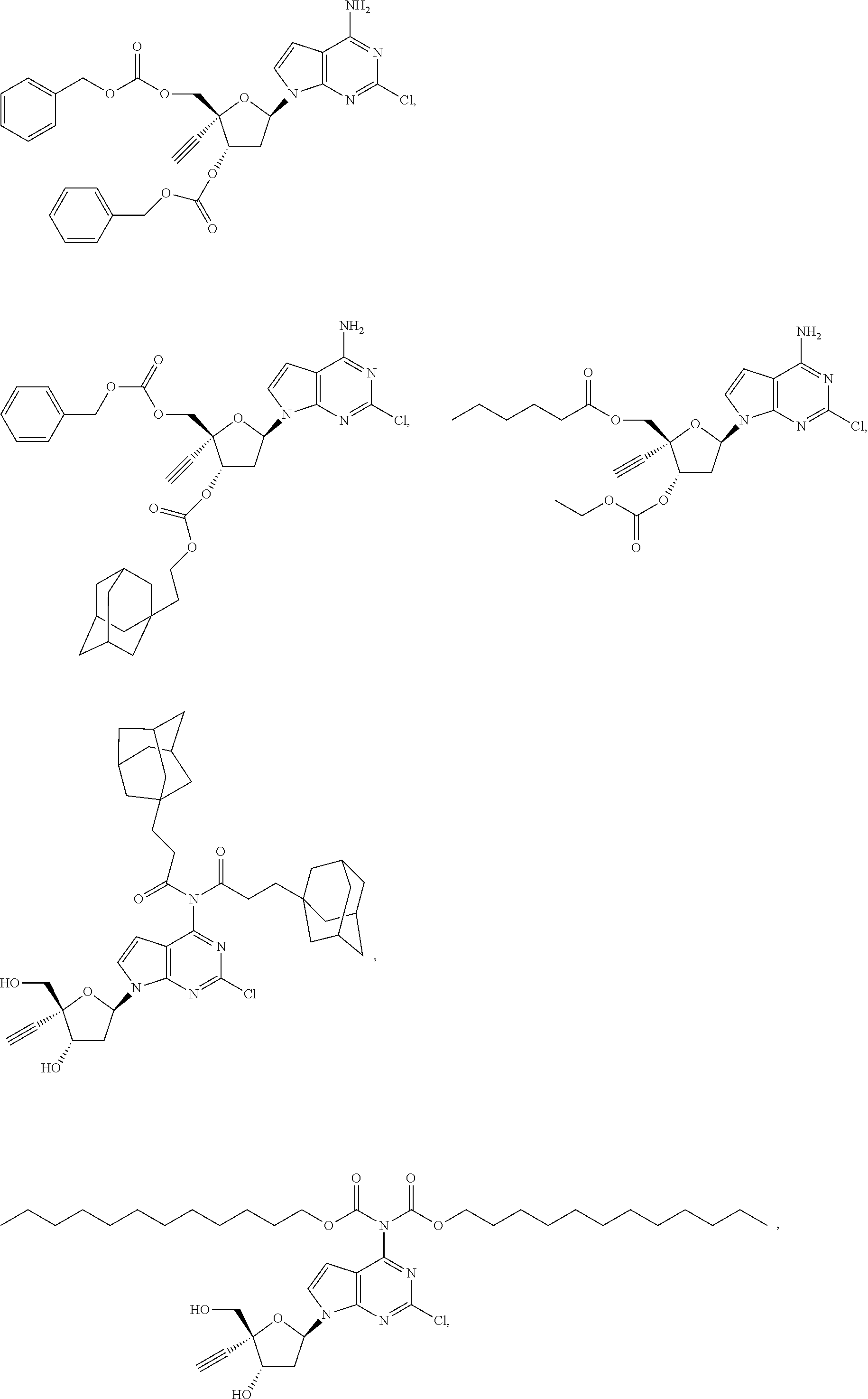 [SMILES: C#C[C@]1(COC(=O)OCc2ccccc2)O[C@@H](n2ccc3c(N)nc(Cl)nc32)C[C@@H]1OC(=O)OCc1ccccc1 ; C#C[C@]1(COC(=O)OCc2ccccc2)O[C@@H](n2ccc3c(N)nc(Cl)nc32)C[C@@H]1OC(=O)OCCC12CC3CC(CC(C3)C1)C2 ; C#C[C@]1(COC(=O)CCCCC)O[C@@H](n2ccc3c(N)nc(Cl)nc32)C[C@@H]1OC(=O)OCC ; C#C[C@]1(CO)O[C@@H](n2ccc3c(N(C(=O)CCC45CC6CC(CC(C6)C4)C5)C(=O)CCC45CC6CC(CC(C6)C4)C5)nc(Cl)nc32)C[C@@H]1O ; C#C[C@]1(CO)O[C@@H](n2ccc3c(N(C(=O)OCCCCCCCCCCCC)C(=O)OCCCCCCCCCCCC)nc(Cl)nc32)C[C@@H]1O] 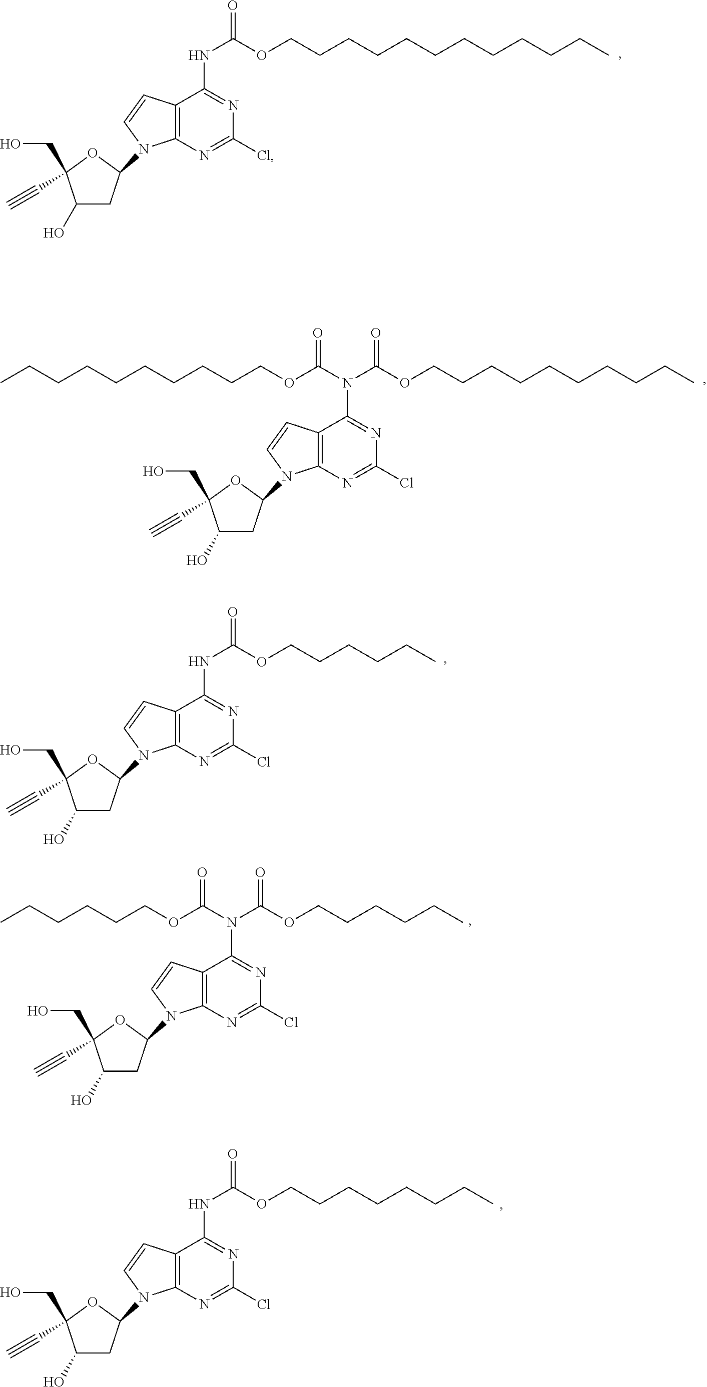 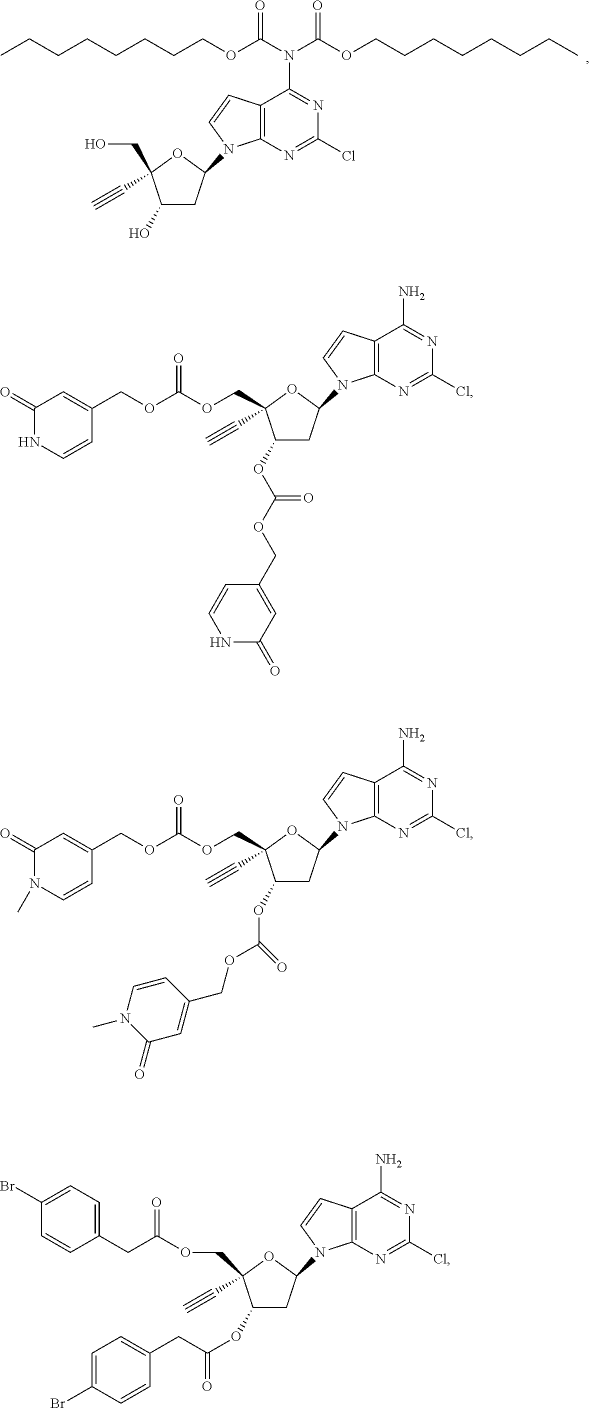 [SMILES: C#C[C@]1(CO)O[C@@H](n2ccc3c(N(C(=O)OCCCCCCCC)C(=O)OCCCCCCCC)nc(Cl)nc32)C[C@@H]1O ; C#C[C@]1(COC(=O)OCc2cc[nH]c(=O)c2)O[C@@H](n2ccc3c(N)nc(Cl)nc32)C[C@@H]1OC(=O)OCc1cc[nH]c(=O)c1 ; C#C[C@]1(COC(=O)OCc2ccn(C)c(=O)c2)O[C@@H](n2ccc3c(N)nc(Cl)nc32)C[C@@H]1OC(=O)OCc1ccn(C)c(=O)c1 ; C#C[C@]1(COC(=O)Cc2ccc(Br)cc2)O[C@@H](n2ccc3c(N)nc(Cl)nc32)C[C@@H]1OC(=O)Cc1ccc(Br)cc1]
or a pharmaceutically acceptable salt thereof.

11. The compound of claim 1 having the structure: 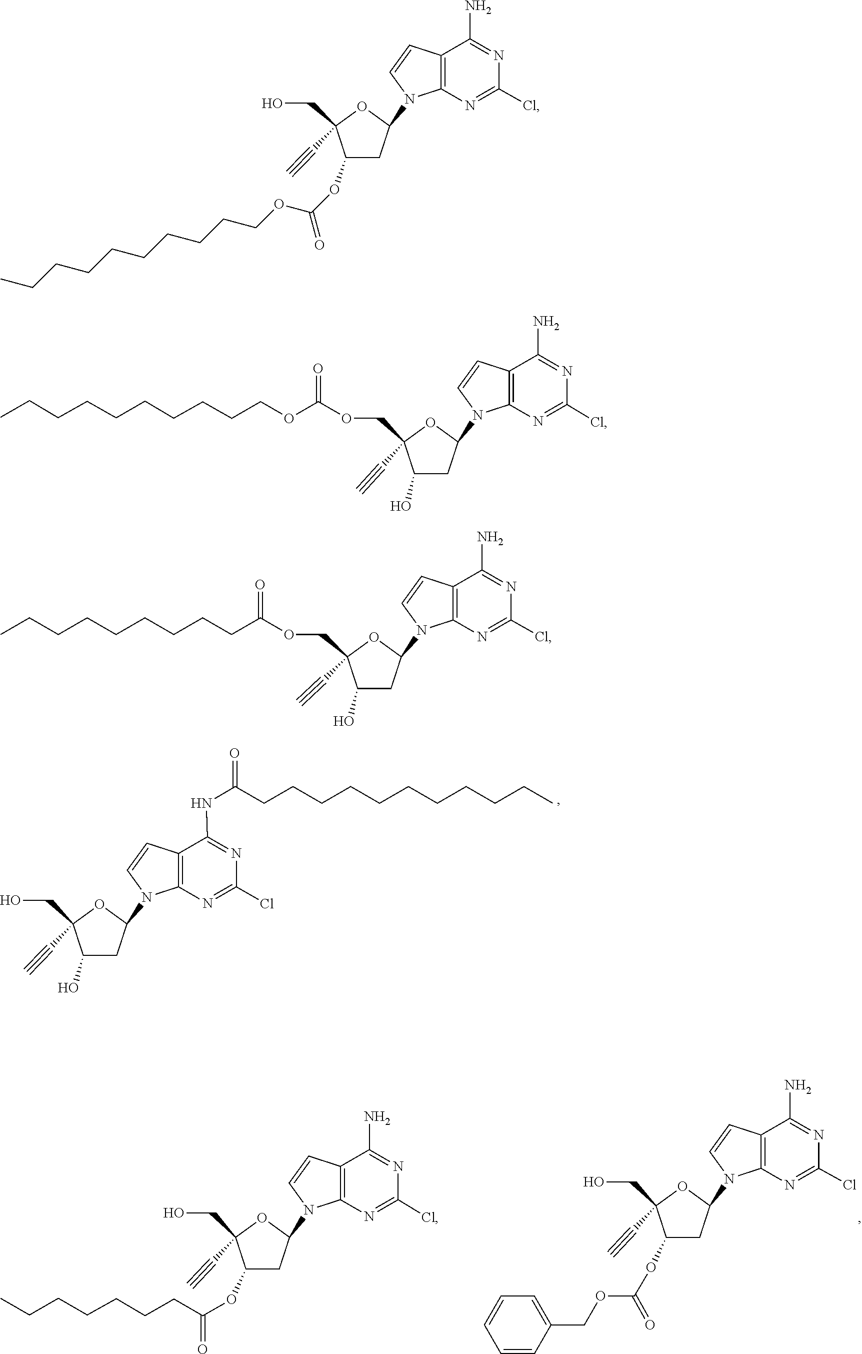 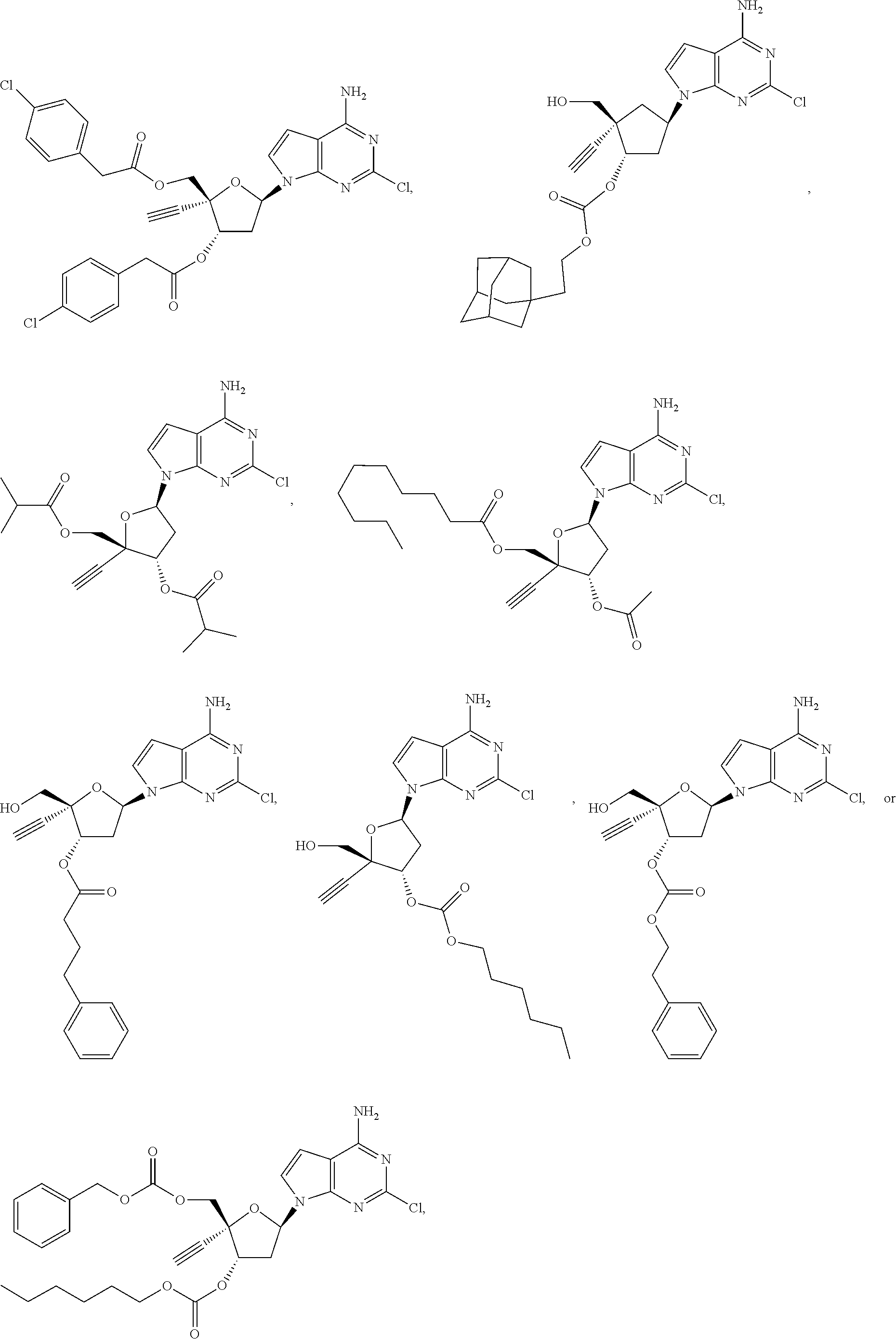
or a pharmaceutically acceptable salt thereof.

12. A pharmaceutical composition comprising a compound or salt of claim 1, and a pharmaceutically acceptable carrier.

13. The pharmaceutical composition of claim 12, wherein the composition is injectable, or adapted for injection.

14. A pharmaceutical composition of claim 12, further comprising one or more additional therapeutic agents selected from the group consisting of lenacapavir, raltegravir, lamivudine, abacavir, ritonavir, dolutegravir, darunavir, atazanavir, emtricitabine, tenofovir, elvitegravir, rilpivirine, lopinavir, doravirine and islatravir.

15. A method for the inhibition of HIV reverse transcriptase in a subject in need thereof which comprises administering to the subject an effective amount of the compound or salt of claim 1.

16. A method for the treatment of infection by HIV or the treatment, prophylaxis, or delay in onset or progression of AIDS in a subject in need thereof, comprising administering to the subject an effective amount of the compound or salt of claim 1.

17. The method of claim 16 further comprising administering to the subject one or more additional therapeutic agents selected from the group consisting of lenacapavir, raltegravir, lamivudine, abacavir, ritonavir, dolutegravir, darunavir, atazanavir, emtricitabine, tenofovir, elvitegravir, rilpivirine, lopinavir, and doravirine, wherein the amounts administered of the compound or salt and the one or more additional therapeutic agents are together effective to treat infection by HIV or to treat, prevent or delay the onset or progression of AIDS.

18. The method of claim 17, wherein the additional therapeutic agent is lenacapavir.

19. The method of claim 15, wherein the method comprises administering the compound or salt in accordance with a dosing interval range from about once per month to about once per every twelve months.

20. The method of claim 19, wherein the dosing interval range is about once per every three months, once per every six months, or once per every twelve months.
